SEQUENCE LISTING (10 sequences)

SEQ ID NO: 1 - DNA, 66 bp, Artificial
  Synthetic
gctgaaggtcgtcactccaccggtgctatgggatccgctggctccgctgctggttctggcgaatag

SEQ ID NO: 2 - DNA, 66 bp, Artificial
  Synthetic
gctgaaggtcgtcactccaccggtgctatggggtcggcgggtagtgcagcaggaagtggtgaatag

SEQ ID NO: 3 - DNA, 66 bp, Artificial
  Synthetic
gctgaaggtcgtcactccaccggtgctatgggtagtgcaggttgcgcagcaggcagcggcgaatag

SEQ ID NO: 4 - DNA, 66 bp, Artificial
  Synthetic
gctgaaggtcgtcactccaccggtgctatgggttcagccgggagtgctgccggcagtggtgaatag

SEQ ID NO: 5 - DNA, 66 bp, Artificial
  Synthetic
gctgaaggtcgtcactccaccggtgctatggggagtgcagggagtgcagcggggtcgggtgaatag

SEQ ID NO: 6 - DNA, 66 bp, Artificial
  Synthetic
gctgaaggtcgtcactccaccggtgctatgggtagtgcgggttcagcggccgggtcaggtgaatag

SEQ ID NO: 7 - DNA, 66 bp, Artificial
  Synthetic
gctgaaggtcgtcactccaccggtgctatgggatcagctggctccgcagccggatcgggtgaatag

SEQ ID NO: 8 - DNA, 66 bp, Artificial
  Synthetic
gctgaaggtcgtcactccaccggtgctatgggttcagcagggagtgcggcaggttcaggagagtag

SEQ ID NO: 9 - DNA, 66 bp, Artificial
  Synthetic
gctgaaggtcgtcactccaccggtgctatggggagtgcggggagcgcggcagggtcgggagagtag

SEQ ID NO: 10 - DNA, 66 bp, Artificial
  Synthetic
gctgaaggtcgtcactccaccggtgctatgggctcggcgggaagcgcagctggttccggggagtac

(19) United States
(12) Patent Application Publication
(10) Pub. No.: US 2022/0162595 A1
(43) Pub. Date: May 26, 2022

(54) METHODS FOR MODIFYING TRANSLATION

(71) Applicant: RAMOT AT TEL-AVIV UNIVERSITY LTD., Tel Aviv (IL)

(72) Inventors: Tamir TULLER, Tel Aviv (IL); Shir BAHIRI, Tel Aviv (IL); Boaz APT, Tel Aviv (IL)

(21) Appl. No.: 17/486,936

(22) Filed: Sep. 28, 2021

Related U.S. Application Data
(63) Continuation of application No. PCT/IL2020/050367, filed on Mar. 26, 2020.
(60) Provisional application No. 62/825,143, filed on Mar. 28, 2019.

(51) Int. Cl. C12N 15/10; C12N 15/67

(52) U.S. Cl. CPC C12N 15/1089; C12N 15/67

(57) ABSTRACT

Nucleic acid molecules comprising a mutation that mutation modulates the interaction strength of the nucleic acid molecule to a 16S ribosomal RNA are provided. Methods of improving the translation process of a nucleic acid molecule and producing a nucleic acid molecule optimized for translation, as well as cells comprising the nucleic acid molecules are also provided.

### CROSS-REFERENCE TO RELATED APPLICATIONS

This application is a continuation of PCT Patent Application No. PCT/IL2020/050367 having International filing date of Mar. 26, 2020, which claims the benefit of priority of U.S. Provisional Patent Application No. 62/825,143 filed Mar. 28, 2019, both titled “METHODS FOR MODIFYING TRANSLATION”, the contents of which are all incorporated herein by reference in their entirety.

### FIELD OF INVENTION

The present invention is directed to the field of translation optimization.

### BACKGROUND OF THE INVENTION

The region approximately 8-10 nucleotides upstream of the translational start site in prokaryotic mRNA tends to include a purine-rich sequence. This sequence is named the Shine-Dalgarno (SD) sequence or ribosome binding site (RBS), and is believed to be involved in prokaryotic translation initiation via base-pairing to a complementary sequence in the 16S rRNA component of the small ribosomal subunit, namely the anti-Shine-Dalgarno sequence (aSD).

Recent studies have also suggested that sequences (motifs) within the coding regions that interact with the aSD, similarly to the SD, can slow down or pause translation elongation in E. coli. Thus, such sequences in the coding regions decrease the overall translation elongation rate and can generally be considered deleterious. Other studies have suggested that selection against internal SD-like sequences which promote rRNA-mRNA interactions can act against codons that tend to compose such motifs. A comprehensive understanding of rRNA-mRNA interactions is however lacking, and methods of optimizing mRNA sequences for enhanced or decreased translation are greatly needed.

### SUMMARY OF THE INVENTION

The present invention provides, in some embodiments, nucleic acid molecules comprising a mutation that modulates the interaction strength of the nucleic acid molecule to a 16S ribosomal RNA. Methods of improving the translation process of a nucleic acid molecule and producing a nucleic acid molecule optimized for translation, as well as cells comprising the nucleic acid molecules and computer program products are also provided.

According to a first aspect, there is provided a nucleic acid molecule comprising a coding sequence, wherein the nucleic acid molecule comprises at least one mutation within a region of the molecule, wherein the mutation modulates the interaction strength of the nucleic acid molecule to a 16S ribosomal RNA (rRNA); and wherein the region is selected from the group consisting of:
a. positions −8 through −17 upstream of a translational start site (TSS) of the coding sequence and the mutation increases interaction strength;
b. positions −1 upstream of a TSS through position 5 downstream of the TSS of the coding sequence and the mutation increases interaction strength;
c. positions 6 through 25 downstream of a TSS of the coding sequence and the mutation decreases interaction strength;
d. positions 26 downstream of a TSS of the coding sequence through position −13 upstream of a translational termination site (TTS) of the coding sequence and the mutation modulates interaction strength to an intermediate interaction strength;
e. positions −8 through −17 upstream of a TTS of the coding sequence and the mutation increases interaction strength; and
f. a position downstream of a TTS of the coding sequence and the mutation increases interaction strength.

According to another aspect, there is provided a cell comprising a nucleic acid molecule of the invention.

According to another aspect, there is provided a method for improving the translation potential of a coding sequence, the method comprising introducing at least one mutation into a nucleic acid molecule comprising the coding sequence, wherein the mutation modulates the interaction strength of the nucleic acid molecule to a 16S rRNA, thereby improving the translation potential of a coding sequence.

According to another aspect, there is provided a method of modifying a cell, the method comprising expressing a nucleic acid molecule of the invention or an improved nucleic acid molecule produced by a method of the invention, within the cell, thereby modifying a cell.

According to another aspect, there is provided a computer program product for modulating translation potential of a coding sequence in a nucleic acid molecule, comprising a non-transitory computer-readable storage medium having program code embodied thereon, the program code executable by at least one hardware processor to:
a. receive a sequence of the nucleic acid molecule;
b. calculate the interaction strength of a 6-nucleotide long subregion of the nucleic acid molecule to an aSD of a 16S rRNA of a target bacterium;
c. calculate the cumulative alteration to interaction strength between the subregion and the aSD caused by a mutation within the subregion; and
d. provide an output modified sequence of the nucleic acid molecule comprising at least a mutation that increases or decreases translation potential.

According to some embodiments, the mutation modulates the interaction strength of a six-nucleotide sequence containing the mutation to the 16S rRNA.

According to some embodiments, the interaction strength to a 16S rRNA is to an anti-Shine Dalgarno (aSD) sequence of the 16S rRNA.

According to some embodiments, the interaction strength of a sequence of the nucleic acid molecule to the aSD sequence is determined from Table 3.

According to some embodiments, the increasing increases interaction strength to a strong interaction strength, decreasing decreases interaction strength to a weak interaction strength and wherein strong, weak and intermediate interaction strengths are determined from Table 1.

According to some embodiments, the region from position 26 downstream of the TSS through position −13 upstream of the TTS comprises the first 400 base pairs of the region.

According to some embodiments, the nucleic acid molecule of the invention comprises at least a second mutation, wherein the second mutation is in a different region than the at least one mutation.

According to some embodiments, the at least one mutation is within the coding sequence and mutates a codon of the coding sequence to a synonymous codon.

According to some embodiments, the mutation improves the translation potential of the coding sequence.

According to some embodiments, the improving comprises at least one of: increasing translation initiation efficiency, increasing translation initiation rate, increasing diffusion of the small subunit to the initiation site, increasing elongation rate, optimization of ribosomal allocation, increasing chaperon recruitment, increasing termination accuracy, decreasing translational read-through and increasing protein yield.

According to some embodiments, the nucleic acid molecule is a messenger RNA (mRNA).

According to some embodiments, the cell is a bacterial cell.

According to some embodiments, the bacteria is selected from a bacterium recited in Table 1.

According to some embodiments, the bacterium is selected from Escherichia Coli, Alphprotebacteria, Spriochaete, Purple bacteris, Gammaproteoaceteria, deltaproteobacteria and Betaproteobacteria.

According to some embodiments, the bacterium is not a Cyanobacteria or Gram-positive bacteria.

According to some embodiments, the nucleic acid molecule is endogenous to the cell.

According to some embodiments, the nucleic acid molecule is exogenous to the cell.

According to some embodiments, the mutation is located at a region selected from the group consisting of:
a. positions −8 through −17 upstream of a translational start site (TSS) of the coding sequence and the mutation increases interaction strength;

b. positions −1 upstream of a TSS through position 5 downstream of the TSS of the coding sequence and the mutation increases interaction strength;

c. positions 6 through 25 downstream of a TSS of the coding sequence and the mutation decreases interaction strength;

d. positions 26 downstream of a TSS of the coding sequence through position −13 upstream of a translational termination site (TTS) of the coding sequence and the mutation modulates interaction strength to an intermediate interaction strength;

e. positions −8 through −17 upstream of a TTS of the coding sequence and the mutation increases interaction strength; and

f. a position downstream of a TTS of the coding sequence and the mutation increases interaction strength.

According to some embodiments, the nucleic acid molecule is a nucleic acid molecule of the invention.

According to some embodiments,
a. the region is located at positions −8 through −17 upstream of a TSS, and wherein the increased interaction strength results in improved translation initiation;
b. the region is located at positions −1 upstream of a TSS through position 5 downstream of a TSS, and wherein the increased interaction results in improved optimization of ribosomal allocation or increased chaperon recruitment;
c. the region is located at positions 5 through 25 downstream of a TSS, and wherein the decreased interaction strength results in an improved translation initiation efficiency;
d. the region is located at positions 26 downstream of a TSS through position −13 upstream of a TTS, and wherein the modulated interaction strength to an intermediate interaction strength results in increased diffusion of the small subunit to the initiation site, improved translation initiation efficiency, optimized pre-initiation diffusion or increase protein level;
e. the region is located at positions −8 through −17 upstream of a TTS, and wherein the increased interaction strength results in increased termination efficiency, termination accuracy or decreased translation read-through; or
f. the region is located downstream of a TTS, and wherein the increased interaction strength results in improving the recycling of ribosomes in the translation process.

According to some embodiments, the method of the invention further comprises introducing at least a second mutation in a different region from the at least one mutation.

According to some embodiments, introducing a mutation comprises:
a. profiling interaction strengths of each 6-nucleotide long subregion of the nucleic acid molecule to the 16S rRNA;
b. profiling an interaction strength of each 6-nucleotide long subregion comprising a potential mutation of the nucleic acid molecule; and
c. introducing to the nucleic acid molecule the mutation wherein the cumulative change in interaction strength of all of the 6-nucleotide long subregions comprising the mutation modulates an interaction strength to the 16S ribosomal RNA.

According to some embodiments, the calculating comprises calculating interaction strength of a plurality of 6-nucleotide long subregions with a region of the nucleic acid molecule, wherein the region is selected from:
a. positions −8 through −17 upstream of a translational start site (TSS);
b. positions −1 upstream of a TSS through position 5 downstream of the TSS;
c. positions 6 through 25 downstream of a TSS;
d. positions 25 downstream of a TSS through position −13 upstream of a translational termination site (TTS);
e. positions −8 through −17 upstream of a TTS; and
f. a position downstream of a TTS.

According to some embodiments, the calculating comprises calculating the interaction strength of each 6-nucleotide long subregion within the region.

According to some embodiments, the output modified sequence of the nucleic acid molecule comprises at least the top 5 mutations within the nucleic acid molecule that increase or decrease translation potential.

According to some embodiments, the output modified sequence of the nucleic acid molecule comprises at least the top 5 mutations within the region that increase or decrease translation potential.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee

FIGS. 1A-1E. Prediction of rRNA-mRNA interaction strength and selection for or against strong rRNA-mRNA interactions at the 5′UTR and at the beginning of the coding region. (FIG. 1A) The three statistical tests to detect evolutionary selection for different rRNA-mRNA interaction strength. 1. Enrichment of sub-sequences with weak rRNA-mRNA interactions. 2. Enrichment of sub-sequences with intermediate rRNA-mRNA interactions. 3. Enrichment of sub-sequences with strong rRNA-mRNA interactions. In each of the three cases we look at sub-sequences with certain rRNA-mRNA interaction strengths (right column: weak, intermediate, or strong) and tested if their number is significantly higher than expected by the null model (left column). (FIG. 1B) Strong rRNA-mRNA interaction strength significant positions distribution in the 5′UTR and first 20 nucleotides of the coding region. Each row represents a prokaryotic bacterium and the rows are clusters based on their phyla, and each column is a position in all the transcripts in the analyzed organisms. A red/green position indicates a position with significant selection for/against strong rRNA-mRNA interaction, in comparison to the null model respectively (Methods). A black pixel represents a bacterium for which the number of significant positions with selection for strong interactions was significantly higher than the null model in the 5′UTR; a blue pixel represents a bacterium for which the number of significant positions with selection for strong interactions was significantly higher than the null model in the last nucleotide of the 5′UTR and the first 5 nucleotides of the coding region. (FIG. 1C) Illustration of the way strong rRNA-mRNA interactions affect translation initiation: The rRNA-mRNA interactions upstream of the translational start site initiate translation by aligning the small subunit of the ribosome to the canonical translational start site. (FIG. 1D) Illustration: Strong interactions at the first steps of elongation slow down the ribosome movement. (FIG. 1E) Z-score for rRNA-mRNA interaction strength at the last 20 nucleotides of the 5′UTR and at the first 20 nucleotides of the coding regions in highly and lowly expressed genes in E. coli. Highly and lowly genes were selected according to protein abundance. Lower/higher Z-scores mean selection for/against strong rRNA-mRNA interactions respectively, in comparison to what is expected by the null model. On the right side, two bar graphs can be seen. The bar graphs represent the strongest (lowest Z-score value) position in highly and lowly expressed genes in the two regions of the reported signals.

FIGS. 2A-2F. Selection for/or against strong rRNA-mRNA interactions in the coding regions. (FIG. 2A) Strong rRNA-mRNA interaction strength significant positions distribution in the coding regions (first 400 nt). Each row represents a prokaryotic bacterium and the rows are clusters based on their phyla, and each column is a position in all the transcripts in the analyzed organisms. Red/green indicates a position with significant selection for/against strong rRNA-mRNA interactions in comparison to the null model respectively (Methods). A black pixel at the right side of the plot represents a bacterium for which the number of significant positions with selection against strong interactions was significantly higher than the null model. (FIG. 2B) Z-score for rRNA-mRNA interaction strength at the first 400 nucleotides of the coding regions in highly and lowly expressed genes according to protein abundance in E. coli. Lower/higher Z-scores mean selection for/against strong rRNA-mRNA Interactions respectively, in comparison to what is expected by the null model. The black/red line represents the average Z-score in a window of 40 nucleotides in highly/lowly expressed genes respectively. (FIG. 2C) Significant strong rRNA-mRNA interaction strength positions distribution in the 3′ UTR. Each row represents a bacterium; rows are clustered into to bacterial phylum and each column is a position in the bacteria's transcripts. Red/green indicates a position with significant selection for/against strong rRNA-mRNA interactions in comparison to the null model respectively (Methods). A black pixel represents a bacterium for which the number of significant positions with selection against strong interactions was significantly higher than the null model. (FIG. 2D) Illustration: Strong rRNA-mRNA interactions effect on translation elongation in the coding region: strong rRNA-mRNA interactions can slow down the movement of the ribosome and delay the translation process. (FIG. 2E) Strong and intermediate rRNA-mRNA interaction strength significant positions distribution in the coding region (first 100 nt). Each row represents a prokaryotic bacterium and the rows are clustered according to bacterial phylums and each column is a position in the transcripts. Red/green indicates a position with significant selection for/against strong rRNA-mRNA interactions in comparison to the null model respectively (Methods). A black pixel represents a bacterium where the number of significant positions with selection against strong interaction was significantly higher than the null model. For each bacterium, we calculated in a sliding window of 40 nucleotides, the number of positions in the window with selection against strong and intermediate interactions. The bars represent the average number of windows that had higher significant positions in comparison to the rest of the transcript, in every bacterial family with the proper standard deviation. The periodicity in the signal is related to the genetic code. (FIG. 2F) Illustration: strong and intermediate interactions at the first 25 nucleotides can be deleterious and can promote initiation from erroneous positions.

FIGS. 3A-3H. Selection for/or against strong rRNA-mRNA interactions at the end of the coding regions. (FIG. 3A) Strong rRNA-mRNA interaction strength significant positions distribution in the coding region (last 400 nt). Each row represents a prokaryotic bacterium; rows are clustered according to the bacterial Phylum, and each column is a position in the bacterial transcripts. Red/green indicates a position with significant selection for/against strong rRNA-mRNA interaction in comparison to the null model respectively (Methods). A black pixel represents a bacterium where the number of significant positions with selection for strong interactions was significantly higher than the null model. (FIG. 3B) Most significant positions in the last 20 nt of the coding region. For each position in this region, we counted the number of bacteria exhibit a significant signal of selection for strong rRNA-mRNA interactions in that specific position. (FIG. 3C) Strongest position in the last 20 nt of the coding region. We calculated the Z-score value profile for rRNA-mRNA interaction strength in each bacterium at the last 20 nt of the coding region. Each bar represents the number of bacteria that exhibit the minimum Z-score value in that position. (FIG. 3D) Division of E. coli genes according to their expression levels (protein abundance). Each bar represents the minimum Z-score value for rRNA-mRNA interaction strength at the last 400 nucleotides of the coding region according to the gene expression levels. (FIG. 3E) Ribo-seq analysis, average read counts distributions at the beginning of the 3′UTR of genes with strong (gray bars)/weak (orange bars) rRNA-mRNA interactions at the end of the coding sequence (Methods). (FIG. 3F) Illustration: strong interactions at the end of the coding region affect the correct recognition of the translational termination site and aid in translation termination. (FIG. 3G) The experiment construct, an RFP gene connected to a GFP gene. We tested the effect of different rRNA-mRNA interaction strengths in the last 35 nt of the RFP gene by creating variants with different folding in the last 40 nt. (FIG. 3H) Bar graph of values proportional to GFP/RFP fluorescence levels in the 9 variants (see Methods) grouped according to their local folding energies.

FIGS. 4A-4H. Selection for/or against intermediate rRNA-mRNA interactions in the coding regions. (FIG. 4A) Intermediate rRNA-mRNA interaction strength definition and thresholds validation in E. coli. Two distributions are shown: 1. Minimum rRNA-mRNA interaction strength distribution of the strong interaction strength region (related to region (1), blue bars). 2. Minimum rRNA-mRNA interaction strength distribution in the weak/devoid interaction region (related to region (2), orange bars). Depicted are also the selected thresholds that define intermediate interactions (Methods). (FIG. 4B) Intermediate rRNA-mRNA interaction strength significant positions distribution in the coding region (first 400 nt). Each row represents a prokaryotic bacterium; rows are clustered according to the bacterial phylum and each column is a position in the transcripts. Red/green indicates a position with significant selection for/against strong rRNA-mRNA interaction in comparison to the null model respectively (Methods). A black pixel represents a bacterium where the number of significant positions with selection for intermediate interactions was significantly higher than the null model. (FIG. 4C) Intermediate rRNA-mRNA interaction strength significant positions distribution in the 3′ UTR. Each row is a prokaryotic bacterium according to bacteria families, and each column is a position in the transcript. Red/green indicates a position with significant selection for/against strong rRNA-mRNA interaction in comparison to the null model respectively (Methods). A black pixel represents a bacterium where the number of significant positions with selection for intermediate interaction was significantly higher than the null model. (FIG. 4D) Distribution of the area ratio. A ratio larger than 1 suggests that it is more probable that the inferred definitions are related to (intermediate) rRNA-mRNA interactions, and not to a lack of interaction. (FIG. 4E) The number of intermediate sequences and PA correlation in GFP variants, where the GFP are divided into six groups according to their FE. On the right side, there is a correlation between PA and the number of intermediate interaction sequences for the strongest FE group. (FIG. 4F) Illustration of intermediate interaction effect on translation initiation. 1) Intermediate interactions in the coding sequence. 2) Intermediate interactions in the coding sequence aid initiation when there is strong mRNA folding in the region surrounding the translational start site. (FIG. 4G) An illustration of the biophysical model. Each site's parameters are determined by its rRNA-mRNA interaction strength. There is an attachment rate to the site, detachment rate from the site, movement forward to the site and from it and movement backward from the site and to it. This model allows for deduction of the initiation rate for insertion into the elongation model. H. An illustration of the rRNA-mRNA interaction strength extended model. The density of each site is determined by k sites before it and k sites after it. (Supplementary section S9).

FIG. 5. Division of the bacteria according to their growth rates (doubling time). Each bar represents the minimum Z-score value for rRNA-mRNA interaction strength in positions −8 through −17 at the end of the coding region according to doubling time groups.

FIG. 6. Non-canonical aSD strong rRNA-mRNA interaction strength significant positions distribution in the 5′UTR. Each row is a bacterium clustered according to bacteria phylum, and each column is a position in the transcript. A red/green position indicates a position with significant selection for/against strong rRNA-mRNA interactions in comparison to the null model respectively.

FIG. 7. Non-canonical aSD strong rRNA-mRNA interaction strength significant positions distribution in the coding region (first 400 nt). Each row is a bacterium clustered according to bacteria phylum, and each column is a position in the transcript. A red/green position indicates a position with significant selection for/against strong rRNA-mRNA interactions in comparison to the null model respectively.

FIG. 8. Non-canonical aSD strong rRNA-mRNA interaction strength significant positions distribution in the 3′UTR. Each row is a bacterium clustered according to bacteria phylum, and each column is a position in the transcript. A red/green position indicates a position with significant selection for/against strong rRNA-mRNA interaction in comparison to the null model respectively.

FIG. 9. Non-canonical aSD strong rRNA-mRNA interaction strength significant positions distribution in the coding region (last 400 nt). Each row is a bacterium clustered according to bacteria phylum, and each column is a position in the transcript. A red/green position indicates a position with significant selection for/against strong rRNA-mRNA interactions in comparison to the null model respectively.

FIG. 10. Non-canonical aSD intermediate rRNA-mRNA interaction strength significant positions distribution in the first 400 nucleotides of the coding region. Each row is a bacterium clustered according to bacteria phylum, and each column is a position in the transcript. A red/green position indicates a position with significant selection for/against strong rRNA-mRNA interactions in comparison to the null model respectively.

FIG. 11. Non-canonical aSD intermediate rRNA-mRNA interaction strength significant positions distribution in the 3′ UTR. Each row is a bacterium clustered according to bacteria phylum, and each column is a position in the transcript. A red/green position indicates a position with significant selection for/against strong rRNA-mRNA interaction in comparison to the null model respectively.

FIG. 12(A) Average number of significant positions in the coding region in bacteria according to groups of doubling time. (FIG. 12B) Average number of significant positions in the coding region in E. coli according to groups of translation efficiency (PA/mRNA levels).

FIG. 13. The optimization process to find new “aSD” sequences.

FIG. 14. Distribution of the optimal non-canonical “aSD” that were inferred by our optimization model in the 64 bacteria.

FIG. 15. The number of sequences in a specific hybridization energy group and PA correlation in GFP variants.

FIG. 16. Illustration of all known and new rules related to rRNA-mRNA interaction in all stages and sub-stages of the translation process.

FIG. 17. Significant position for/against strong interactions in the coding region of E. coli. The top row refers to a genome (real and random) when we eliminated from the analysis position upstream to an AUG (up to 14 nt upstream to an AUG). The bottom row refers to the original genomes (real and random). Each column is a position in the transcript. A red/green position indicates a position with significant selection for/against strong rRNA-mRNA interaction in comparison to the null model respectively.

FIGS. 18A-B. (18A) Z-score for rRNA-mRNA interaction strength at the last 200 nucleotides of the coding regions in the first middle last genes of operons in E. coli. Lower/higher Z-scores mean stronger/weaker rRNA-mRNA interactions respectively in comparison to what is expected by the null model. (18B) Z-score for rRNA-mRNA interaction strength at the last 200 nucleotides of the coding regions in a single gene operons of E. coli. Lower/higher Z-scores mean stronger/weaker rRNA-mRNA interactions respectively in comparison to what is expected by the null model.

FIGS. 19A-C. (19A). All variants values of folding and interaction strength. (19B) Alignment of all variants from the original sequence to var9. Mutations that were made are marked. (19C) Fluorescence ratios of the GFP and RFP in all variants at late log/stationary phase of growth.

FIGS. 20A-C. (20A) The time to translate a codon in a certain position for different variant with various rRNA-mRNA interaction strengths. (20B) The increase in initiation rate when adding more intermediate interactions to the coding sequence. (20C) The increase in translation rate when adding more intermediate interactions to the coding sequence.

### DETAILED DESCRIPTION OF THE INVENTION

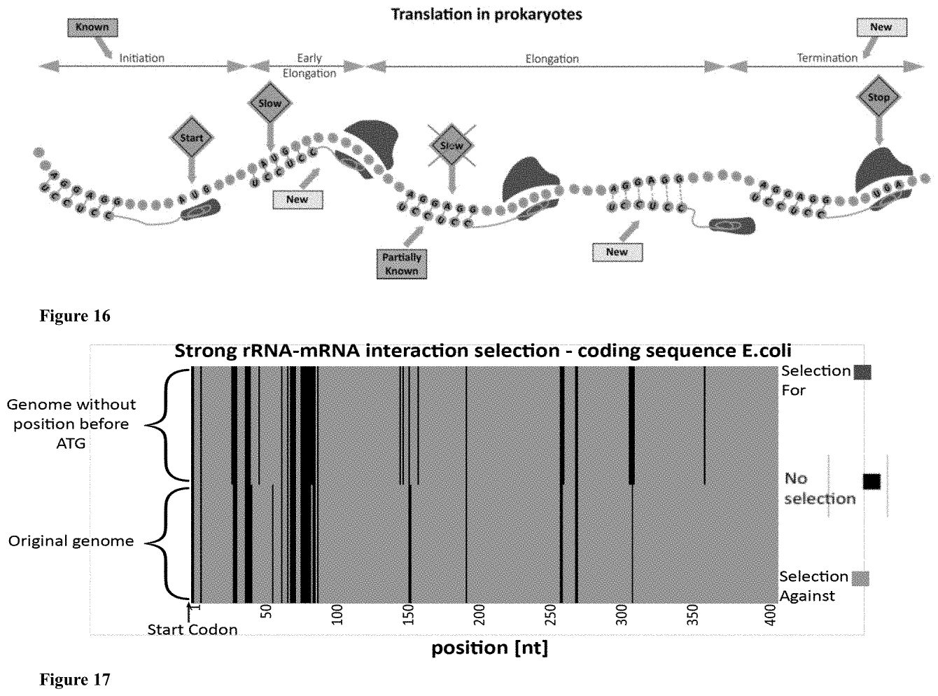

The invention is based on the surprising findings that strong, weak and intermediate interactions between mRNAs and the 16S rRNA are selected for in particular regions of an mRNA. Further, these selected for interactions enhance translation and the introduction of mutations that alter interaction strengths in these regions in turn alter the translation efficiency of the mutated mRNA. It was found that in addition to the canonical rRNA-mRNA interaction that triggers initiation the following rules appear in many bacteria across the tree of life in different stages and sub-stages of the translation process (FIG. 16).

Early elongation—at the beginning of the coding region there is evidence of selection for strong rRNA-mRNA interactions that slow down the early translation elongation.

Elongation 1—inside the coding region there is evidence of selection against strong rRNA-mRNA interactions. This signal is related also to improving translation elongation (and not only to prevent incorrect initiation).

Elongation 2—there is evidence of selection inside the transcript for intermediate rRNA-mRNA interactions to improve pre-initiation.

Termination—there is evidence of selection for strong rRNA-mRNA interactions upstream of the STOP codon to prevent ribosomal read-trough.

The findings disclosed herein are based on the comprehensive analysis of 551 prokaryotic genomes. We show that the current knowledge regarding the functional rRNA-mRNA interactions during translation is only the ‘tip of the iceberg’: in most of the analyzed prokaryotes, rRNA-mRNA interactions seem to be involved in all sub-stages of translation, via corresponding sequence signatures encoded across the entire transcript. Thus, rRNA-mRNA interactions affect the way evolution shapes the nucleotide composition along the entire transcript to optimize translation.

### Nucleic Acid Molecules

By a first aspect, there is provided a nucleic acid molecule comprising a coding sequence, the nucleic acid molecule comprising at least one mutation that modulates the interaction strength of the nucleic acid molecule to a ribosomal RNA.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to modified and unmodified single-stranded RNA (ssRNA) or single-stranded DNA (ssDNA) having both a coding region and a noncoding region. In some embodiments, the nucleic acid molecule is DNA. In some embodiments, the nucleic acid molecule is RNA. In some embodiments, the DNA is single stranded DNA. In some embodiments, the DNA is double stranded DNA. In some embodiments, the DNA is plasmid DNA. In some embodiments, the RNA is single stranded RNA. In some embodiments, the RNA is plasmid RNA. In some embodiments, the RNA is messenger RNA (mRNA). In some embodiments, the RNA is pre-mRNA. mRNA is well known in the art. In some embodiments, mRNA comprises a 5′ cap. In some embodiments, the mRNA is devoid of a 5′ cap. In some embodiments, the cap is a 7-methylguanasine cap. In some embodiments, mRNA comprises a 3′ polyA tail. In some embodiments, mRNA is polyadenylated. In some embodiments, mRNA comprises a 3′ oligouridine tail. In some embodiments, mRNA is oligouridylated. In some embodiments, the mRNA is monocistronic. In some embodiments, the mRNA is polycistronic. In some embodiments, the nucleic acid molecule comprises a plurality of coding sequences.

As used herein, the phrases “Coding sequence” and “coding region” are interchangeably used herein to refer to a nucleic acid sequence that when translated results in an expression product, such as a polypeptide, protein, or enzyme. In some embodiments, the coding sequence is to be used as a basis for making codon alterations. In some embodiments, the coding sequence is a bacterial gene. In some embodiments, the coding sequence is a viral gene. In some embodiments, the coding sequence is a mammalian gene. In some embodiments, the coding sequence is a human gene. In some embodiments, the coding sequence is a portion of one of the above listed genes. In some embodiments, the coding sequence is a heterologous transgene. In some embodiments, the above listed genes are wild type, endogenously expressed genes. In some embodiments, the above listed genes have been genetically modified or in some way altered from their endogenous formulation.

The term “heterologous transgene” as used herein refers to a gene that originated in one species and is being expressed in another. In some embodiments, the transgene is a part of a gene originating in another organism. In some embodiments, the heterologous transgene is a gene to be overexpressed. In some embodiments, expression of the heterologous transgene in a wild-type cell reduces global translation in the wild-type cell.

In some embodiments, the nucleic acid molecule further comprises a non-coding region. In some embodiments, the non-coding region is an untranslated region (UTR). In some embodiments, the UTR is 5′ to the coding sequence. In some embodiments, the UTR is 3′ to the coding sequence. In some embodiments, the nucleic acid molecule comprises a 5′ UTR and a 3′ UTR. In some embodiments, the UTR is the endogenous UTR associated with the coding sequence. In some embodiments, the UTR comprises at least one regulatory element that regulates translation of the coding sequence. In some embodiments, the UTR is transcribed with the coding sequence. In some embodiments, an mRNA transcribed from the nucleic acid molecule is a functional mRNA. In some embodiments, a functional mRNA is an mRNA that is capable of being translated. In some embodiments, the nucleic acid molecule is an mRNA. In some embodiments, the nucleic acid molecule is a functional mRNA.

As used herein, the phrases “noncoding sequence” and “noncoding region” are interchangeably used herein to refer to sequences upstream of the translational start site (TSS) or downstream of the translational termination site (TTS). The noncoding region can be at least 1, 5, 10, 25, 50, 100, 200, 500, 1000, 2000, 5000 or 10000 base pairs upstream of the TSS or downstream of the TTS.

In some embodiments of the invention, the noncoding sequence upstream of the TSS refers to a 5′ untranslated region also referred to as 5′ UTR. According to some embodiments, the 5′UTR includes a ribosome binding site (RBS). In some embodiments, the RBS comprises a Shine-Dalgarno (SD) sequence. In some embodiments, the SD sequence is a canonical SD sequence. In some embodiments, the SD sequence is a non-canonical SD sequence. In some embodiments, the RBS does not comprise a SD sequence. In some embodiments, the canonical SD sequence comprises the sequence AGGAGG. In some embodiments, the SD sequence comprises the sequence AGGAGGU. The SD sequence is involved in prokaryotic translation initiation via base-pairing to a complementary sequence named the anti-SD (aSD) sequence on the 3′ tail of the 16S rRNA component of the small ribosomal subunit. In some embodiments, the aSD sequence comprises and/or consists of the sequence ACCUCCUUA. In some embodiments, the E. coli aSD sequence comprises and/or consists of the sequence ACCUCCUUA. In some embodiments, the aSD comprises a 6-nucleotide long subregion. In some embodiments, interaction strength is the binding strength to the subregion. In some embodiments the canonical subregion comprises and/or consists of CCUCCU. In some embodiments the canonical subregion comprises and/or consists of CCTCCT. In some embodiments, the aSD subregion comprises and/or consists of a sequence selected from: GCCGCG, CGGCTG, CTCCTT, GCCGTA, GCGGCT, GTGGCT, and GGCTGG. U and T are used interchangeably herein.

In some embodiments of the invention, the noncoding sequence downstream of the TTS refers to a 3′ untranslated region also referred to as 3′ UTR.

In some embodiments, the ribosomal RNA is a small ribosome subunit. According to some embodiments, the ribosomal RNA may be a 30S small subunit of a ribosome. According to other embodiments, the ribosomal RNA is a 16S ribosomal RNA. According to some embodiments of the invention, the 16S ribosomal RNA has an aSD sequence. In some embodiments, interaction strength is calculated to the aSD. In some embodiments, interaction strength is calculated to a subregion of the aSD.

The term “interaction strength” as used herein refers to hybridization free energy between a nucleic acid molecule and a ribosomal RNA. Lower and more negative free energy is related to stronger hybridization and stronger interaction strength. Hybridization free energy can be computed based on the Vienna package RNAcoFold, which computes a common secondary structure of two RNA molecules. According to some embodiments, the interaction strength can be defined by a scale of strong, intermediate and weak.

The term “hybridization” or “hybridizes” as used herein refers to the formation of a duplex between nucleotide sequences which are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T (U), G and C of one sequence is then aligned with a T (U), A, C and G, respectively, of the other sequence. RNA sequences can also include complementary G=U or U=G base pairs. Thus, two sequences need not have perfect homology to be “complementary” under the invention.

As used herein, the term “free energy” refers is made to the Gibbs free energy (AG), referring to the thermodynamic potential that measures the hybridization reaction between a given oligonucleotide and its DNA or RNA complement.

In some embodiments, the nucleic acid molecule comprises a mutation. In some embodiments, a mutation is introduced into the nucleic acid molecule. In some embodiments, the mutation is in the coding sequence. In some embodiments, the mutation is in the noncoding sequence of the nucleic acid molecule. In some embodiments, the mutation results in modulated interaction strength between a nucleic acid molecule region and a ribosomal RNA compared to the interaction strength between an unmodified nucleic acid molecule and a ribosomal RNA. In some embodiments, the mutation modulates local interaction strength. In some embodiments, the mutation modulates interaction strength at the mutated nucleotide. In some embodiments, the mutation is a mutation to a nucleotide with stronger interaction. In some embodiments, the mutation is a mutation to a nucleotide with a weaker interaction. In some embodiments, the mutation modulates interaction strength in a particular region. In some embodiments, the mutation modulates interaction strength in a particular subregion. In some embodiments, the mutation modulates interaction strength of a subregion of the mRNA that is bound by the aSD sequence of a small ribosomal subunit.

In some embodiments, at least one mutation is introduced to at least one region of the nucleic acid molecule. In some embodiments, the mutation is in a region. In some embodiments, the region is selected from the group consisting of:
a. positions −8 through −17 upstream of a translational start site (TSS);
b. positions −1 upstream of a TSS through position 5 downstream of the TSS;
c. positions 6 through 25 downstream of a TSS;
d. positions 26 downstream of a TSS through position −13 upstream of a translational termination site (TTS);
e. positions −8 through −17 upstream of a TTS; and
f. a position downstream of a TTS.

In some embodiments, the mutation is in a region comprising positions −8 through −17 upstream of a TSS. In some embodiments, the mutation is in a region comprising positions −1 upstream of a translational start site through position 5 downstream of the translational start site.

In some embodiments, the mutation is in a region comprising positions 6 through 25 downstream of a TSS. In some embodiments, the mutation is in a region comprising positions 26 downstream of a TSS through position −13 upstream of a translational termination site.

In some embodiments, the mutation is in a region comprising positions −8 through −17 upstream of a TTS. In some embodiments, the mutation is in a region comprising positions −9 through −12 upstream of a TTS. In some embodiments, the region comprising positions −8 through −17 upstream of the TTS is a region comprising position −9 through −12 upstream of the TTS. In some embodiments, the mutation is in a region comprising positions downstream of a TTS. In some embodiments, the region from position 26 downstream of the TSS through position −13 upstream of the TSS comprises at most 400 nucleotides. In some embodiments, the region from position 26 downstream of the TSS through position −13 upstream of the TSS comprises or consists of position 26 though position 400 downstream of the TSS.

In some embodiments, the mutation is in a region comprising positions −8 through −17 upstream of a TSS, increases interaction strength and enhances translation potential. In some embodiments, the mutation is in a region comprising positions −8 through −17 upstream of a TSS, decreases interaction strength and decreases translation potential. In some embodiments, the mutation is in a region comprising positions −1 upstream of a TSS through position 5 downstream of the TSS, increases interaction strength and increases translation potential. In some embodiments, the mutation is in a region comprising positions −1 upstream of a TSS through position 5 downstream of the TSS, decreases interaction strength and decreases translation potential. In some embodiments, the mutation is in a region comprising positions 6 through 25 downstream of a TSS, increases interaction strength and decreases translation potential. In some embodiments, the mutation is in a region comprising positions 6 through 25 downstream of a TSS, decreases interaction strength and increases translation potential. In some embodiments, the mutation is in a region comprising positions 26 downstream of a TSS through position −13 upstream of a translational termination site, increases interaction strength and decreases translation potential. In some embodiments, the mutation is in a region comprising positions 26 downstream of a TSS through position −13 upstream of a translational termination site, decreases interaction strength and increases translation potential. In some embodiments, the mutation is in a region comprising positions −8 through −17 upstream of a TTS, increases interaction strength and increases translation potential. In some embodiments, the mutation is in a region comprising positions −8 through −17 upstream of a TTS, decreases interaction strength and decreases translation potential. In some embodiments, the mutation is in a region comprising positions downstream of a TTS. increases interaction strength and decreases translation potential. In some embodiments, the mutation is in a region comprising positions downstream of a TTS. decreases interaction strength and increases translation potential. Thus, it can be understood that interaction strength and translation potential are correlated in regions between −8 and −17 in the 5′ UTR, between −1 of the 5′ UTR and +5 of the coding region, and between −8 to −17 relative to the TTS; whereas interaction strength and translation potential are inversely related in the middle regions of the coding region (from +6 relative to the TSS to −12 relative to the TTS) and in the 3′ UTR. This is particularly true from +6 to +25 relative to the TSS. “Interaction strength modulation” refers to increasing or decreasing the interaction strength between a nucleic acid molecule and a ribosomal RNA sequence. In some embodiments, the interaction strength is modulated at the site of the mutation. In some embodiments, the interaction strength is modulated in the region comprising the mutation. In some embodiments, the interaction strength is modulated in a subregion comprising the mutation.

According to some embodiments, interaction strength modulation may result in modifying at least one step of the translation process including, but not limited to increased translation initiation efficiency, decreased translation initiation efficiency, increased translation initiation rate, decreased translation initiation rate, increased diffusion of the small ribosomal subunit to the initiation site, decreased diffusion of the small subunit to the initiation site, increased elongation rate, decreased elongation rate, optimization of ribosomal allocation, deoptimization of ribosomal allocation, increased chaperon recruitment, decreased chaperon recruitment, increased termination accuracy, decreased termination accuracy, increased translational read-through, decreased translational read-through, increase protein level and decreased protein level. Each possibility represents a separate embodiment of the invention. In some embodiments, modulating interaction strength alters translation potential.

As used herein, the term “translation potential” refers to the potential translation that would occur if the nucleic acid were introduced into a system competent to translate the nucleic acid. In some embodiments, translation potential comprises translation rate. In some embodiments, translation potential comprises translation efficiency. In some embodiments, translation potential comprises translation initiation rate or efficiency. In some embodiments, translation potential comprises ribosome diffusion. In some embodiments, translation potential comprises, ribosomal allocation. In some embodiments, translation potential comprises termination accuracy. In some embodiments, translation potential comprises termination efficiency. In some embodiments, translation potential comprises termination rate. In some embodiments, translation potential comprises total protein yield.

In some embodiments, translation is in vivo translation. In some embodiments, translation is in vitro translation. In vitro translation systems are well known in the art, and include for example, rabbit reticulocyte lysates. In some embodiments, translation comprises translation pre-initiation. In some embodiments, translation comprises translation initiation. In some embodiments, translation comprises early elongation. In some embodiments, translation comprise elongation. In some embodiments, translation comprises translation termination.

In some embodiments, the interaction strength is increased by at least 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 1000%, or 10000% relative to an unmodified region of a nucleic acid molecule and a ribosomal RNA. Each possibility represents a separate embodiment of the invention.

In some embodiments, a strong interaction is an interaction of at least 1.3, 1.5, 1.7, 1.8, 1.9, 2.0. 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2 or 7.3 kcal/mol. Each possibility represents a separate embodiment of the invention According to some embodiments, the interaction strength is increased to a strong interaction strength. Organism specific interaction strengths are provided in Table 1. In some embodiments, the interaction strength (Hybridization energy value or “H.E.V”) of specific 6-nucleotide long subregions of an mRNA to canonical and non-canonical aSD sequences are as provided in Table 3. Organisms specific aSD sequences are known in the art and can be determined for each organism selected.

TABLE 1
Interaction strengths per organism

| | Strong | | Weak |
|---|---|---|---|
| Bacteria name | interaction | Intermediate interaction | interaction |
| Achromobacter denitrificans | <−2.658255 | −2.658255 < and < −1.100000 | >−1.100000 |
| Acidovorax avenae subsp | <−4.200000 | −4.200000 < and < −0.100000 | >−0.100000 |
| Advenella kashmirensis WT001 | <−2.700000 | −2.700000 < and < −1.500000 | >−1.500000 |
| Alcaligenaceae bacterium LMG | <−2.535297 | −2.535297 < and < −1.200000 | >−1.200000 |
| Alcalis faecalis | <−3.400000 | −3.400000 < and < −0.500000 | >−0.500000 |
| Alicycliphilus denitrificans BC | <−2.738992 | −2.738992 < and < −1.300000 | >−1.300000 |
| Aquabacterium sp NJ1 | <−3.600000 | −3.600000 < and < −0.600000 | >−0.600000 |
| Aquaspirillum sp LM1 | <−2.500000 | −2.500000 < and < −1.000000 | >−1.000000 |
| Azoarcus aromaticum EbN1 | <−3.120081 | −3.120081 < and < −1.400000 | >−1.400000 |
| Betaproteobacteria bacterium GR1643 | <−3.000000 | −3.000000 < and < −0.700000 | >−0.700000 |
| Blood disease bacterium | <−2.608817 | −2.608817 < and < −1.200000 | >−1.200000 |
| Bordetella avium 197N | <−2.390569 | −2.390569 < and < −1.000000 | >−1.000000 |
| Burkholderia ambifaria | <−2.778567 | −2.778567 < and < −1.100000 | >−1.100000 |
| Burkholderiales bacterium 23 | <−2.557916 | −2.557916 < and < −0.900000 | >−0.900000 |
| Candidatus Accumulibacter | <−2.818943 | −2.818943 < and < −1.100000 | >−1.100000 |
| phosphatis | | | |
| Castellaniella defragrans 65Phen | <−2.886602 | −2.886602 < and < −1.200000 | >−1.200000 |
| Chromobacterium sphagni | <−2.796367 | −2.796367 < and < −1.100000 | >−1.100000 |
| Collimonas arenae | <−2.199146 | −2.199146 < and < −1.400000 | >−1.400000 |
| Comamonas aquatica | <−3.500000 | −3.500000 < and < −0.700000 | >−0.700000 |
| Cupriavidus basilensis | <−3.200000 | −3.200000 < and < −1.800000 | >−1.800000 |
| Curvibacter sp AEP13 | <−3.800000 | −3.800000 < and < −0.700000 | >−0.700000 |
| Dechloromonas agitata is5 | <−2.590102 | −2.590102 < and < −1.000000 | >−1.000000 |
| Dechlorosoma suillum PS | <−2.900000 | −2.900000 < and < −1.000000 | >−1.000000 |
| Delftia acidovorans | <−2.600000 | −2.600000 < and < −0.600000 | >−0.600000 |
| Diaphorobacter | <−2.490329 | −2.490329 < and < −1.500000 | >−1.500000 |
| polyhydroxybutyrativorans | | | |
| Gallionella capsiferriformans ES2 | <−2.445054 | −2.445054 < and < −1.000000 | >−1.000000 |
| Herbaspirillum frisingense | <−2.630458 | −2.630458 < and < −1.400000 | >−1.400000 |
| Herminiimonas arsenicoxydans | <−2.159737 | −2.159737 < and < −1.100000 | >−1.100000 |
| Hydrogenophaga crassostreae | <−4.100000 | −4.100000 < and < −0.500000 | >−0.500000 |
| Janthinobacterium agaricidamnosum | <−2.400000 | −2.400000 < and < −1.000000 | >−1.000000 |
| NBRC | | | |
| Jeongeupia sp USM3 | <−2.729392 | −2.729392 < and < −1.000000 | >−1.000000 |
| Laribacter hongkongensis | <−2.699938 | −2.699938 < and < −1.300000 | >−1.300000 |
| LHGZ1 complete | | | |
| Leptothrix cholodnii SP6 | <−4.500000 | −4.500000 < and < −0.100000 | >−0.100000 |
| Limnohabitans sp 63ED372 | <−4.400000 | −4.400000 < and < −0.700000 | >−0.700000 |
| Massilia putida | <−2.594815 | −2.594815 < and < −1.100000 | >−1.100000 |
| Methylibium petroleiphilum PM1 | <−3.900000 | −3.900000 < and < −0.100000 | >−0.100000 |
| Methylophilus sp 5 | <−2.049198 | −2.049198 < and < −1.000000 | >−1.000000 |
| Methylotenera versatilis 301 | <−1.750000 | −1.750000 < and < −1.000000 | >−1.000000 |
| Methyloversatilis discipulorum | <−2.698209 | −2.698209 < and < −1.500000 | >−1.500000 |
| Mitsuaria sp 7 | <−3.900000 | −3.900000 < and < −0.100000 | >−0.100000 |
| Nitrosomonas communis | <−2.184474 | −2.184474 < and < −1.300000 | >−1.300000 |
| Nitrosospira briensis C128 | <−2.800000 | −2.800000 < and < −1.900000 | >−1.900000 |
| Noviherbaspirillum autotrophicum | <−2.412543 | −2.412543 < and < −1.100000 | >−1.100000 |
| Paraburkholderia caballeronis | <−2.819684 | −2.819684 < and < −1.800000 | >−1.800000 |
| Paucibacter sp KCTC | <−4.200000 | −4.200000 < and < −0.500000 | >−0.500000 |
| Polaromonas glacialis | <−3.800000 | −3.800000 < and < −0.700000 | >−0.700000 |
| Pseudogulbenkiania sp MAI1 | <−3.179329 | −3.179329 < and < −1.100000 | >−1.100000 |
| Pusillimonas sp T77 | <−2.500000 | −2.500000 < and < −0.600000 | >−0.600000 |
| Ralstonia eutropha H16 | <−2.832328 | −2.832328 < and < −1.200000 | >−1.200000 |
| Ramlibacter tataouinensis | <−4.200000 | −4.200000 < and < −0.700000 | >−0.700000 |
| Rhizobacter gummiphilus | <−3.900000 | −3.900000 < and < −0.100000 | >−0.100000 |
| Rhodoferax antarcticus | <−3.800000 | −3.800000 < and < −0.700000 | >−0.700000 |
| Roseateles depolymerans | <−3.600000 | −3.600000 < and < −0.700000 | >−0.700000 |
| Rubrivivax gelatinosus IL144 | <−3.800000 | −3.800000 < and < −0.100000 | >−0.100000 |
| Sideroxydans lithotrophicus ES1 | <−2.747522 | −2.747522 < and < −1.200000 | >−1.200000 |
| Sulfuricella denitrificans skB26 | <−2.900000 | −2.900000 < and < −1.700000 | >−1.700000 |
| Sulfuritalea hydrogenivorans sk43H | <−2.500000 | −2.500000 < and < −1.100000 | >−1.100000 |
| Thauera chlorobenzoica | <−3.060218 | −3.060218 < and < −1.200000 | >−1.200000 |
| Thiomonas sp str | <−2.354410 | −2.354410 < and < −1.000000 | >−1.000000 |
| UNVERIFIED Burkholderia sp | <−2.753771 | −2.753771 < and < −1.100000 | >−1.100000 |
| Variovorax boronicumulans | <−3.900000 | −3.900000 < and < −0.100000 | >−0.100000 |
| Verminephrobacter eiseniae EF012 | <−4.200000 | −4.200000 < and < −0.100000 | >−0.100000 |
| Vitreoscilla filiformis | <−5.000000 | −5.000000 < and < −0.700000 | >−0.700000 |
| Vogesella sp LIG4 | <−2.813571 | −2.813571 < and < −1.000000 | >−1.000000 |
| Polyangium brachysporum | <−3.900000 | −3.900000 < and < −0.900000 | >−0.900000 |
| Pseudomonas mesoacidophila | <−2.718895 | −2.718895 < and < −1.100000 | >−1.100000 |
| Nostoc azollae 0708 | <−2.100000 | −2.100000 < and < −1.000000 | >−1.000000 |
| Acaryochloris marina MBIC11017 | <−2.600000 | −2.600000 < and < −1.100000 | >−1.100000 |
| Anabaena cylindrica PCC | <−2.000000 | −2.000000 < and < −1.100000 | >−1.100000 |
| Anabaenopsis circularis NIES21 | <−1.800000 | −1.800000 < and < −1.000000 | >−1.000000 |
| Arthrospira platensis C1 | <−2.900000 | −2.900000 < and < −1.000000 | >−1.000000 |
| Aulosira laxa NIES50 | <−2.600000 | −2.600000 < and < −1.100000 | >−1.100000 |
| Calothrix brevissima NIES22 | <−2.600000 | −2.600000 < and < −1.200000 | >−1.200000 |
| Chamaesiphon minutus PCC | <−2.100000 | −2.100000 < and < −1.700000 | >−1.700000 |
| Chondrocystis sp NIES4102 | <−2.600000 | −2.600000 < and < −1.100000 | >−1.100000 |
| Chroococcidiopsis thermalis PCC | <−1.900000 | −1.900000 < and < −1.000000 | >−1.000000 |
| Crinalium epipsammum PCC | <−2.100000 | −2.100000 < and < −1.000000 | >−1.000000 |
| Cyanobacterium aponinum PCC | <−3.100000 | −3.100000 < and < −1.000000 | >−1.000000 |
| Cyanobium gracile PCC | <−3.927679 | −3.927679 < and < −2.000000 | >−2.000000 |
| Cyanothece sp ATCC | <−2.100000 | −2.100000 < and < −1.000000 | >−1.000000 |
| Cylindrospermopsis raciborskii CS505 | <−2.800000 | −2.800000 < and < −1.400000 | >−1.400000 |
| Cylindrospermum stagnale PCC | <−1.800000 | −1.800000 < and < −1.100000 | >−1.100000 |
| Dactylococcopsis salina PCC | <−2.600000 | −2.600000 < and < −1.400000 | >−1.400000 |
| Dichlorospermum compactum | <−2.800000 | −2.800000 < and < −1.000000 | >−1.000000 |
| NIES806 | | | |
| Filamentous cyanobacterium ESFC1 | <−2.000000 | −2.000000 < and < −1.000000 | >−1.000000 |
| Fischerella sp NIES3754 | <−2.600000 | −2.600000 < and < −1.200000 | >−1.200000 |
| Fortiea contorta PCC | <−1.900000 | −1.900000 < and < −1.000000 | >−1.000000 |
| Fremyella diplosiphon NIES3275 | <−2.600000 | −2.600000 < and < −1.200000 | >−1.200000 |
| Geitlerinema sp PCC | <−2.000000 | −2.000000 < and < −1.000000 | >−1.000000 |
| Geminocystis herdmanii PCC | <−2.600000 | −2.600000 < and < −1.400000 | >−1.400000 |
| Gloeobacter kilaueensis JS1 | <−2.480884 | −2.480884 < and < −1.100000 | >−1.100000 |
| Gloeocapsa sp PCC | <−1.900000 | −1.900000 < and < −1.500000 | >−1.500000 |
| Gloeomargarita lithophora | <−4.600000 | −4.600000 < and < −1.900000 | >−1.900000 |
| AlchichicaD10 | | | |
| Halomicronema hongdechloris C2206 | <−2.600000 | −2.600000 < and < −1.100000 | >−1.100000 |
| Halothece sp PCC | <−2.800000 | −2.800000 < and < −1.000000 | >−1.000000 |
| Leptolyngbya boryana dg5 | <−2.000000 | −2.000000 < and < −1.100000 | >−1.100000 |
| Lyngbya confervoides BDU141951 | <−2.500000 | −2.500000 < and < −1.000000 | >−1.000000 |
| Mastigocladopsis repens PCC | <−2.000000 | −2.000000 < and < −1.100000 | >−1.100000 |
| Microcoleus sp PCC | <−2.600000 | −2.600000 < and < −1.000000 | >−1.000000 |
| Microcystis aeruginosa NIES2481 | <−3.000000 | −3.000000 < and < −1.200000 | >−1.200000 |
| Moorea bouillonii PNG | <−2.800000 | −2.800000 < and < −1.000000 | >−1.000000 |
| Nodosilinea nodulosa PCC | <−3.800000 | −3.800000 < and < −0.700000 | >−0.700000 |
| Nodularia sp NIES3585 | <−2.800000 | −2.800000 < and < −1.000000 | >−1.000000 |
| Nostoc carneum NIES2107 | <−2.000000 | −2.000000 < and < −1.000000 | >−1.000000 |
| Nostocales cyanobacterium HT582 | <−2.600000 | −2.600000 < and < −1.200000 | >−1.200000 |
| Oscillatoria acuminata PCC | <−3.000000 | −3.000000 < and < −1.000000 | >−1.000000 |
| Oscillatoriales cyanobacterium JSC12 | <−2.400000 | −2.400000 < and < −1.000000 | >−1.000000 |
| Planktothrix agrdhii NIVACYA | <−2.800000 | −2.800000 < and < −1.000000 | >−1.000000 |
| Pleurocapsa sp PCC | <−2.700000 | −2.700000 < and < −0.400000 | >−0.400000 |
| Pseudanabaena sp PCC | <−2.600000 | −2.600000 < and < −1.000000 | >−1.000000 |
| Raphidiopsis curvata NIES932 | <−2.700000 | −2.700000 < and < −1.000000 | >−1.000000 |
| Rivularia sp PCC | <−2.000000 | −2.000000 < and < −1.100000 | >−1.100000 |
| Scytonema hofmannii PCC | <−1.900000 | −1.900000 < and < −1.000000 | >−1.000000 |
| Sphaerospermopsis kisseleviana | <−2.600000 | −2.600000 < and < −1.400000 | >−1.400000 |
| NIES73 | | | |
| Spirulina major PCC | <−2.900000 | −2.900000 < and < −1.000000 | >−1.000000 |
| Stanieria cyanosphaera PCC | <−2.000000 | −2.000000 < and < −1.100000 | >−1.100000 |
| Synechococcus sp 60AY4M2 | <−4.600000 | −4.600000 < and < −1.600000 | >−1.600000 |
| Synechocystis sp PCC | <−3.800000 | −3.800000 < and < −1.500000 | >−1.500000 |
| Tolypothrix tenuis PCC | <−2.100000 | −2.100000 < and < −1.000000 | >−1.000000 |
| Trichodesmium erythraeum IMS101 | <−2.000000 | −2.000000 < and < −1.100000 | >−1.100000 |
| Scytonema hofmanm UTEX | <−2.700000 | −2.700000 < and < −1.000000 | >−1.000000 |
| Anaeromyxobacter dehalogenans | <−3.749150 | −3.749150 < and < −2.300000 | >−2.300000 |
| 2CP1 | | | |
| Bilophila wadsworthia 316 | <−4.129102 | −4.129102 < and < −1.300000 | >−1.300000 |
| Chondromyces crocatus | <−3.500000 | −3.500000 < and < −0.800000 | >−0.800000 |
| Deferrisoma camini S3R1 | <−7.000000 | −7.000000 < and < −0.100000 | >−0.100000 |
| Desulfarculus baarsii DSM | <−4.100000 | −4.100000 < and < −1.700000 | >−1.700000 |
| Desulfatibacillum alkenivorans AK01 | <−6.000000 | −6.000000 < and < −0.900000 | >−0.900000 |
| Desulfobacca acetoxidans DSM | <−4.600000 | −4.600000 < and < −1.200000 | >−1.200000 |
| Desulfobacter postgatei 2ac9 | <−3.226775 | −3.226775 < and < −0.800000 | >−0.800000 |
| Desulfobacterium autotrophicum | <−3.678644 | −3.678644 < and < −0.800000 | >−0.800000 |
| HRM2 | | | |
| Desulfobacula toluolica Tol2 | <−3.400000 | −3.400000 < and < −0.800000 | >−0.800000 |
| Desulfocapsa sulfexigens DSM | <−2.622610 | −2.622610 < and < −1.700000 | >−1.700000 |
| Desulfococcus multivorans | <−6.400000 | −6.400000 < and < −0.800000 | >−0.800000 |
| Desulfomicrobium baculatum DSM | <−5.200000 | −5.200000 < and < −0.800000 | >−0.800000 |
| Desulfomonile tiedjei DSM | <−3.651857 | −3.651857 < and < −0.300000 | >−0.300000 |
| Desulfonatronumlacustre DSM | <−4.300000 | −4.300000 < and < −0.700000 | >−0.700000 |
| Desulfotalea psychrophila LSv54 | <−4.600000 | −4.600000 < and < −0.500000 | >−0.500000 |
| Desulfotignum balticum DSM | <−3.476666 | −3.476666 < and < −0.500000 | >−0.500000 |
| Desulfovibrio africanus str | <−4.446524 | −4.446524 < and < −0.800000 | >−0.800000 |
| Desulfurivibrio alkaliphilus AHT2 | <−3.550432 | −3.550432 < and < −2.000000 | >−2.000000 |
| Desulfuromonas soudanensis | <−6.300000 | −6.300000 < and < −2.000000 | >−2.000000 |
| Geoalkalibacter subterraneus | <−3.911379 | −3.911379 < and < −1.600000 | >−1.600000 |
| Geobacter anodireducens | <−5.400000 | −5.400000 < and < −1.800000 | >−1.800000 |
| Geopsychrobacter electrodiphilus | <−3.730890 | −3.730890 < and < −1.600000 | >−1.600000 |
| DSM | | | |
| Haliangium ochraceum DSM | <−2.354149 | −2.354149 < and < −1.200000 | >−1.200000 |
| Melittangium boletus DSM | <−4.000000 | −4.000000 < and < −0.100000 | >−0.100000 |
| Nannocystis exedens | <−4.100000 | −4.100000 < and < −0.100000 | >−0.100000 |
| Pelobacter acetylenicus | <−4.083639 | −4.083639 < and < −1.900000 | >−1.900000 |
| Pseudodesulfovibrio indicus | <−5.100000 | −5.100000 < and < −0.600000 | >−0.600000 |
| Sandaracinus amylolyticus | <−2.600000 | −2.600000 < and < −0.400000 | >−0.400000 |
| Sorangium cellulosum So | <−2.968613 | −2.968613 < and < −1.200000 | >−1.200000 |
| Syntrophobacter fumaroxidans MPOB | <−3.982968 | −3.982968 < and < −2.200000 | >−2.200000 |
| Syntrophorhabdus aromaticivorans UI | <−5.100000 | −5.100000 < and < −0.700000 | >−0.700000 |
| Syntrophus aciditrophicus SB | <−3.495430 | −3.495430 < and < −1.100000 | >−1.100000 |
| Vulgatibacter incomptus | <−3.292169 | −3.292169 < and < −1.100000 | >−1.100000 |
| Acidihalobacter ferrooxidans | <−2.832404 | −2.832404 < and < −1.000000 | >−1.000000 |
| Acinetobacter baumannii | <−2.400000 | −2.400000 < and < −0.400000 | >−0.400000 |
| Aeromonas aquatica | <−3.219221 | −3.219221 < and < −1.200000 | >−1.200000 |
| Agarilytica rhodophyticola | <−1.997972 | −1.997972 < and < −1.000000 | >−1.000000 |
| Agarivorans gilvus | <−2.540806 | −2.540806 < and < −1.000000 | >−1.000000 |
| Alcanivorax borkumensis SK2 | <−3.115972 | −3.115972 < and < −0.400000 | >−0.400000 |
| Algiphilus aromaticivorans DG1253 | <−2.753123 | −2.753123 < and < −1.200000 | >−1.200000 |
| Aliivibrio salmonicida LFI1238 | <−2.139238 | −2.139238 < and < −0.400000 | >−0.400000 |
| Alkalilimnicola ehrlichii MLHE1 | <−5.100000 | −5.100000 < and < −1.900000 | >−1.900000 |
| Allochromatium vinosum DSM | <−2.798376 | −2.798376 < and < −1.200000 | >−1.200000 |
| Alteromonadaceae bacterium Bs12 | <−2.112636 | −2.112636 < and < −1.000000 | >−1.000000 |
| Alteromonas addita | <−2.377234 | −2.377234 < and < −1.000000 | >−1.000000 |
| Azotobacter chroococcum | <−3.312078 | −3.312078 < and < −1.100000 | >−1.100000 |
| Bacterioplanes sanyensis | <−2.672064 | −2.672064 < and < −1.000000 | >−1.000000 |
| Beggiatoa alba B18LD | <−2.600000 | −2.600000 < and < −1.400000 | >−1.400000 |
| Brenneria goodwinii | <−3.074380 | −3.074380 < and < −1.700000 | >−1.700000 |
| Budvicia aquatica | <−2.737490 | −2.737490 < and < −1.500000 | >−1.500000 |
| Candidatus Sodalis pierantonius | <−2.600000 | −2.600000 < and < −1.000000 | >−1.000000 |
| Cedecea davisae DSM | <−3.122220 | −3.122220 < and < −1.200000 | >−1.200000 |
| Cellvibrio japonicus Ueda107 | <−3.100000 | −3.100000 < and < −1.000000 | >−1.000000 |
| Chania multitudinisentens RB25 | <−3.110041 | −3.110041 < and < −1.200000 | >−1.200000 |
| Chromatiaceae bacterium | <−2.415316 | −2.415316 < and < −1.200000 | >−1.200000 |
| 2141TSTBD0c01a | | | |
| Chromohalobacter salexigens DSM | <−3.714924 | −3.714924 < and < −1.100000 | >−1.100000 |
| Citrobacter amalonaticus | <−3.218830 | −3.218830 < and < −1.000000 | >−1.000000 |
| Cobetia marina | <−3.244064 | −3.244064 < and < −1.000000 | >−1.000000 |
| Colwellia beringensis | <−2.016915 | −2.016915 < and < −1.000000 | >−1.000000 |
| Congregibacter litoralis KT71 | <−3.000000 | −3.000000 < and < −0.700000 | >−0.700000 |
| Cronobacter condimenti 1330 | <−3.295622 | −3.295622 < and < −1.500000 | >−1.500000 |
| Dokdonella koreensis DS123 | <−5.300000 | −5.300000 < and < −0.800000 | >−0.800000 |
| Dyella japonica A8 | <−4.000000 | −4.000000 < and < −0.500000 | >−0.500000 |
| Ectothiorhodospira sp BSL9 | <−4.600000 | −4.600000 < and < −0.700000 | >−0.700000 |
| Edwardsiella anguillarum ET080813 | <−3.402271 | −3.402271 < and < −1.000000 | >−1.000000 |
| Endozoicomonas elysicola | <−2.400000 | −2.400000 < and < −0.400000 | >−0.400000 |
| Enterobacter asburiae | <−3.215383 | −3.215383 < and < −1.500000 | >−1.500000 |
| Enterobacteriaceae bacterium | <−3.041843 | −3.041843 < and < −1.700000 | >−1.700000 |
| 9254FAA | | | |
| Erwinia amylovora | <−2.907515 | −2.907515 < and < −1.000000 | >−1.000000 |
| Escherichia albertii | <−3.167984 | −3.167984 < and < −1.600000 | >−1.600000 |
| Ferrimonas balearica DSM | <−3.262029 | −3.262029 < and < −1.600000 | >−1.600000 |
| Flavobacterium sp 29 | <−2.984477 | −2.984477 < and < −1.100000 | >−1.100000 |
| Fluoribacter dumoffii NY | <−3.600000 | −3.600000 < and < −0.500000 | >−0.500000 |
| Frateuria aurantia DSM | <−5.200000 | −5.200000 < and < −0.700000 | >−0.700000 |
| Gibbsiella quercinecans | <−3.253279 | −3.253279 < and < −1.100000 | >−1.100000 |
| Gilliamella apicola | <−2.289776 | −2.289776 < and < −0.500000 | >−0.500000 |
| Gilvimarinus agarilyticus | <−2.602257 | −2.602257 < and < −1.100000 | >−1.100000 |
| Glaciecola nitratireducens FR1064 | <−2.187655 | −2.187655 < and < −1.000000 | >−1.000000 |
| Granulosicoccus antarcticus | <−4.100000 | −4.100000 < and < −0.700000 | >−0.700000 |
| IMCC3135 | | | |
| Grimontia hollisae | <−2.879328 | −2.879328 < and < −1.200000 | >−1.200000 |
| Gynuella sunshinyii YC6258 | <−2.500000 | −2.500000 < and < −1.600000 | >−1.600000 |
| Hafnia alvei | <−3.010037 | −3.010037 < and < −1.400000 | >−1.400000 |
| Hahella chejuensis KCTC | <−2.861378 | −2.861378 < and < −1.900000 | >−1.900000 |
| Halioglobus japonicus | <−2.526132 | −2.526132 < and < −1.000000 | >−1.000000 |
| Halomonas aestuarii | <−3.925218 | −3.925218 < and < −2.200000 | >−2.200000 |
| Halotalea alkalilenta | <−3.393394 | −3.393394 < and < −1.100000 | >−1.100000 |
| Idiomarina sp 513 | <−2.423055 | −2.423055 < and < −1.000000 | >−1.000000 |
| Immundisolibacter cernigliae | <−2.814424 | −2.814424 < and < −1.000000 | >−1.000000 |
| Klebsiella aeros | <−3.263021 | −3.263021 < and < −1.000000 | >−1.000000 |
| Kluyvera intermedia | <−3.268280 | −3.268280 < and < −1.600000 | >−1.600000 |
| Kosakonia cowanii | <−3.295651 | −3.295651 < and < −1.000000 | >−1 .000000 |
| Kushneria sp X49 | <−3.102146 | −3.102146 < and < −1.500000 | >−1.500000 |
| Lacimicrobium alkaliphilum | <−2.700000 | −2.700000 < and < −1.500000 | >−1.500000 |
| Leclercia adecarboxylata | <−3.245500 | −3.245500 < and < −1.500000 | >−1.500000 |
| Legionella anisa | <−3.500000 | −3.500000 < and < −0.100000 | >−0.100000 |
| Lelliottia amnigena | <−3.241161 | −3.241161 < and < −1.500000 | >−1.500000 |
| Photobacterium damselae subsp | <−3.400000 | −3.400000 < and < −0.400000 | >−0.400000 |
| gamma proteobacterium HdN1 | <−2.558180 | −2.558180 < and < −1.100000 | >−1.100000 |
| Acetobacterium woodii DSM | <−4.502335 | −4.502335 < and < −1.100000 | >−1.100000 |
| Acutalibacter muris | <−6.600000 | −6.600000 < and < −0.500000 | >−0.500000 |
| Aeribacillus pallidus | <−4.687457 | −4.687457 < and < −1.600000 | >−1.600000 |
| Alicyclobacillus acidocaldarius subsp | <−5.903231 | −5.903231 < and < −0.600000 | >−0.600000 |
| Alkaliphilus metalliredigens QYMF | <−5.500511 | −5.500511 < and < −0.700000 | >−0.700000 |
| Anaeromassilibacillus sp | <−5.200000 | −5.200000 < and < −0.900000 | >−0.900000 |
| MarseilleP3371 | | | |
| Anaerostipes hadrus | <−4.499630 | −4.499630 < and < −1.700000 | >−1.700000 |
| Aneurinibacillus migulanus | <−4.916336 | −4.916336 < and < −1.000000 | >−1.000000 |
| Anoxybacillus sp B2M1 | <−5.295424 | −5.295424 < and < −1.800000 | >−1.800000 |
| Blautia coccoides | <−5.100000 | −5.100000 < and < −1.000000 | >−1.000000 |
| Brevibacillus brevis | <−5.561512 | −5.561512 < and < −1.100000 | >−1.100000 |
| Butyrivibrio hungatei | <−4.388547 | −4.388547 < and < −0.300000 | >−0.300000 |
| Carnobacterium gallinarum DSM | <−4.953787 | −4.953787 < and < −1.600000 | >−1.600000 |
| Clostridioides difficile | <−5.361239 | −5.361239 < and < −0.400000 | >−0.400000 |
| Cohnella panacarvi Gsoil | <−5.051972 | −5.051972 < and < −1.700000 | >−1.700000 |
| Dehalobacter sp CF | <−5.193446 | −5.193446 < and < −1.100000 | >−1.100000 |
| Dehalobacterium formicoaceticum | <−7.200000 | −7.200000 < and < −0.500000 | >−0.500000 |
| Desulfitobacterium dehalogenans | <−5.642733 | −5.642733 < and < −1.000000 | >−1.000000 |
| ATCC | | | |
| Desulfosporosinus acidiphilus SJ4 | <−5.322331 | −5.322331 < and < −0.600000 | >−0.600000 |
| Eisenbergiella tayi | <−5.011039 | −5.011039 < and < −0.900000 | >−0.900000 |
| Erysipelotrichaceae bacterium 146 | <−7.300000 | −7.300000 < and < −1.000000 | >−1.000000 |
| Ethanolins harbinense YUAN3 | <−4.738622 | −4.738622 < and < −2.200000 | >−2.200000 |
| Exiguobacterium acetylicum DSM | <−5.444853 | −5.444853 < and < −1.300000 | >−1.300000 |
| Faecalibacterium prausnitzii | <−5.800000 | −5.800000 < and < −0.500000 | >−0.500000 |
| Fictibacillus arsenicus | <−5.097186 | −5.097186 < and < −1.700000 | >−1.700000 |
| Flavonifractor plautii | <−6.700000 | −6.700000 < and < −1.000000 | >−1.000000 |
| Geobacillus genomosp 3 | <−5.696032 | −5.696032 < and < −2.000000 | >−2.000000 |
| Geosporobacter ferrireducens | <−5.416940 | −5.416940 < and < −1.000000 | >−1.000000 |
| Gottschalkia acidurici 9a | <−5.071164 | −5.071164 < and < −0.400000 | >−0.400000 |
| Halobacillus halophilus | <−5.507263 | −5.507263 < and < −1.200000 | >−1.200000 |
| Heliobacterium modesticaldum Ice1 | <−5.200000 | −5.200000 < and < −2.200000 | >−2.200000 |
| Herbivorax saccincola | <−4.745131 | −4.745131 < and < −0.800000 | >−0.800000 |
| Hungatella hathewayi WAL18680 | <−1.500000 | −1.500000 < and < −1.300000 | >−1.300000 |
| Intestinimonas butyriciproducens | <−7.300000 | −7.300000 < and < −1.000000 | >−1.000000 |
| Jeotgalibacillus malaysiensis | <−5.114980 | −5.114980 < and < −1.100000 | >−1.100000 |
| Kyrpidia sp EA1 | <−5.500000 | −5.500000 < and < −0.500000 | >−0.500000 |
| Lachnoclostridium phytofermentans | <−4.985131 | −4.985131 < and < −1.000000 | >−1.000000 |
| ISDg | | | |
| Lactobacillus casei | <−5.223797 | −5.223797 < and < −2.200000 | >−2.200000 |
| Lentibacillus amyloliquefaciens | <−5.129462 | −5.129462 < and < −1.000000 | >−1.000000 |
| Limnochorda pilosa | <−5.037825 | −5.037825 < and < −0.500000 | >−0.500000 |
| Listeria innocua Clip11262 | <−5.356949 | −5.356949 < and < −1.700000 | >−1.700000 |
| Lysinibacillus fusiformis | <−5.187337 | −5.187337 < and < −1.200000 | >−1.200000 |
| Mahella australiensis 501 | <−4.875491 | −4.875491 < and < −1.400000 | >−1.400000 |
| Niameybacter massiliensis | <−5.250898 | −5.250898 < and < −0.400000 | >−0.400000 |
| Novibacillus thermophilus | <−4.894576 | −4.894576 < and < −1.700000 | >−1.700000 |
| Numidum massiliense | <−4.968859 | −4.968859 < and < −2.200000 | >−2.200000 |
| Oceanobacillus iheyensis HTE831 | <−5.410572 | −5.410572 < and < −1.200000 | >−1.200000 |
| Oscillibacter valericis Sjm1820 | <−6.000000 | −6.000000 < and < −0.900000 | >−0.900000 |
| Paenibacillaceae bacterium GAS479 | <−6.000000 | −6.000000 < and < −1.000000 | >−1.000000 |
| Paeniclostridium sordellii | <−5.552346 | −5.552346 < and < −0.700000 | >−0.700000 |
| Parageobacillus genomosp 1 | <−5.432032 | −5.432032 < and < −2.400000 | >−2.400000 |
| Pelosinus fermentans | <−5.557346 | −5.557346 < and < −1.800000 | >−1.800000 |
| Peptoclostridium difficile | <−5.371230 | −5.371230 < and < −0.400000 | >−0.400000 |
| Peptostreptococcaceae bacterium VA2 | <−5.183566 | −5.183566 < and < −0.500000 | >−0.500000 |
| Planococcus antarcticus DSM | <−5.178283 | −5.178283 < and < −1.200000 | >−1.200000 |
| Planomicrobium sp ES2 | <−5.312056 | −5.312056 < and < −1.000000 | >−1.000000 |
| Pseudobacteroides cellulosolvens | <−4.714095 | −4.714095 < and < −0.500000 | >−0.500000 |
| ATCC | | | |
| Robinsoniella sp KNHs210 | <−5.128143 | −5.128143 < and < −1.100000 | >−1.100000 |
| Roseburia hominis A2183 | <−4.930933 | −4.930933 < and < −1.100000 | >−1.100000 |
| Ruminiclostridium sp KB18 | <−6.000000 | −6.000000 < and < −0.500000 | >−0.500000 |
| Ruminococcaceae bacterium AE2021 | <−4.485370 | −4.485370 < and < −0.200000 | >−0.200000 |
| Ruminococcus albus 7 | <−4.920149 | −4.920149 < and < −0.800000 | >−0.800000 |
| Rummeliibacillus stabekisii | <−4.988144 | −4.988144 < and < −1.400000 | >−1.400000 |
| Saccharibacillus sacchari DSM | <−5.232030 | −5.232030 < and < −1.800000 | >−1.800000 |
| Salipaludibacillus agaradhaerens | <−5.258092 | −5.258092 < and < −1.700000 | >−1.700000 |
| Sediminibacillus massiliensis isolate | <−5.300346 | −5.300346 < and < −1.100000 | >−1.100000 |
| Selenomonas ruminantium subsp | <−6.300000 | −6.300000 < and < −1.000000 | >−1.000000 |
| Solibacillus silvestris | <−5.351237 | −5.351237 < and < −1.100000 | >−1.100000 |
| Sporolactobacillus pectinivorans | <−4.633930 | −4.633930 < and < −1.100000 | >−1.100000 |
| Sporosarcina globispora | <−5.217115 | −5.217115 < and < −0.800000 | >−0.800000 |
| Staphylococcus aureus | <−4.389897 | −4.389897 < and < −1.900000 | >−1.900000 |
| Sulfobacillus thermosulfidooxidans | <−4.736683 | −4.736683 < and < −2.300000 | >−2.300000 |
| Symbiobacterium thermophilum IAM | <−5.800000 | −5.800000 < and < −1.400000 | >−1.400000 |
| Syntrophobotulus glycolicus DSM | <−6.000000 | −6.000000 < and < −0.700000 | >−0.700000 |
| Terribacillus aidingensis | <−5.211959 | −5.211959 < and < −1.300000 | >−1.300000 |
| Thalassobacillus sp TM1 | <−5.383013 | −5.383013 < and < −1.200000 | >−1.200000 |
| Thermanaeromonas toyohensis ToBE | <−5.800000 | −5.800000 < and < −0.500000 | >−0.500000 |
| Thermicanus aegyptius DSM | <−7.300000 | −7.300000 < and < −0.900000 | >−0.900000 |
| Thermincola potens JR | <−5.800000 | −5.800000 < and < −0.800000 | >−0.800000 |
| Thermoanaerobacterium sp RBIITD | <−5.000160 | −5.000160 < and < −1.600000 | >−1.600000 |
| Thermobacillus composti KWC4 | <−5.288205 | −5.288205 < and < −1.700000 | >−1.700000 |
| Tumebacillus algifaecis | <−5.283635 | −5.283635 < and < −2.800000 | >−2.800000 |
| Ureibacillus thermosphaericus | <−4.801140 | −4.801140 < and < −1.100000 | >−1.100000 |
| Virgibacillus dokdonensis | <−5.700000 | −5.700000 < and < −1.000000 | >−1.000000 |
| Viridibacillus sp OK051 | <−4.783024 | −4.783024 < and < −1.100000 | >−1.100000 |
| Desulfotomaculum guttoideum | <−7.300000 | −7.300000 < and < −0.800000 | >−0.800000 |
| Eubacterium cellulosolvens 6 | <−5.100000 | −5.100000 < and < −1.100000 | >−1.100000 |
| Bacillus abyssalis | <−5.014457 | −5.014457 < and < −1.400000 | >−1.400000 |
| Clostridium difficile CD196 | <−5.341238 | −5.341238 < and < −0.500000 | >−0.500000 |
| Desulfotomaculum acetoxidans DSM | <−6.300000 | −6.300000 < and < −0.800000 | >−0.800000 |
| Eubacterium limosum | <−5.100000 | −5.100000 < and < −0.500000 | >−0.500000 |
| Bacillus thuringiensis serovar | <−1.300000 | −1.300000 < and < −0.400000 | >−0.400000 |
| Bacillus clarkii | <−5.500000 | −5.500000 < and < −0.800000 | >−0.800000 |
| Brevibacterium frigoritolerans | <−5.114792 | −5.114792 < and < −1.000000 | >−1.000000 |
| Acidithiobacillus ferrivorans isolate | <−3.505502 | −3.505502 < and < −1.600000 | >−1.600000 |
| Arcobacter nitrofigilis DSM | <−2.651683 | −2.651683 < and < −0.600000 | >−0.600000 |
| Bacteriovorax marinus SJ | <−2.551550 | −2.551550 < and < −0.400000 | >−0.400000 |
| Bdellovibrio bacteriovorus | <−3.400000 | −3.400000 < and < −0.800000 | >−0.800000 |
| Halobacteriovorax marinus | <−2.600000 | −2.600000 < and < −0.300000 | >−0.300000 |
| Leucothrix mucor DSM | <−2.474812 | −2.474812 < and < −1.200000 | >−1.200000 |
| Luminiphilus syltensis NOR51B | <−2.718423 | −2.718423 < and < −1.000000 | >−1.000000 |
| Luteibacter sp 9133 | <−5.200000 | −5.200000 < and < −0.900000 | >−0.900000 |
| Luteimonas abyssi | <−3.900000 | −3.900000 < and < −0.100000 | >−0.100000 |
| Lysobacter antibioticus | <−2.782055 | −2.782055 < and < −1.200000 | >−1.200000 |
| Marichromatium purpuratum 984 | <−3.297203 | −3.297203 < and < −1.200000 | >−1.200000 |
| Marinobacter adhaerens HP15 | <−3.361872 | −3.361872 < and < −1.500000 | >−1.500000 |
| Marinobacterium sp ST5810 | <−2.990056 | −2.990056 < and < −1.700000 | >−1.700000 |
| Marinomonas mediterranea MMB1 | <−2.761546 | −2.761546 < and < −1.400000 | >−1.400000 |
| Methylobacter luteus IMVB3098 | <−2.700000 | −2.700000 < and < −1.000000 | >−1.000000 |
| Methylococcus capsulatus str | <−2.751139 | −2.751139 < and < −1.600000 | >−1.600000 |
| Methylomagnum ishizawai | <−5.200000 | −5.200000 < and < −0.700000 | >−0.700000 |
| Methylomarinum vadi | <−2.700000 | −2.700000 < and < −1.000000 | >−1.000000 |
| Methylomicrobium agile | <−2.202542 | −2.202542 < and < −1.100000 | >−1.100000 |
| Methylomonas denitrificans | <−2.500000 | −2.500000 < and < −1.600000 | >−1.600000 |
| Methylophaga nitratireducenticrescens | <−2.800000 | −2.800000 < and < −1.000000 | >−1.000000 |
| Methylosarcina fibrata AMLC10 | <−2.800000 | −2.800000 < and < −1.900000 | >−1.900000 |
| Methylovulum miyakonense HT12 | <−2.600000 | −2.600000 < and < −0.800000 | >−0.800000 |
| Microbulbifer agarilyticus | <−2.612471 | −2.612471 < and < −1.000000 | >−1.000000 |
| Morganella morganii | <−3.054825 | −3.054825 < and < −1.600000 | >−1.600000 |
| Moritella viscosa | <−2.346008 | −2.346008 < and < −1.200000 | >−1.200000 |
| Neptunomonas phycophila | <−2.598454 | −2.598454 < and < −1.000000 | >−1.000000 |
| Nitrococcus mobilis Nb231 | <−2.944541 | −2.944541 < and < −1.200000 | >−1.200000 |
| Nitrosococcus halophilus Nc4 | <−3.000000 | −3.000000 < and < −1.000000 | >−1.000000 |
| Obesumbacterium proteus | <−3.035412 | −3.035412 < and < −1.200000 | >−1.200000 |
| Oceanicoccus sagamiensis | <−2.110972 | −2.110972 < and < −1.000000 | >−1.000000 |
| Oceanimonas sp GK1 | <−3.299087 | −3.299087 < and < −1.000000 | >−1.000000 |
| Oceanisphaera profunda | <−2.832581 | −2.832581 < and < −1.400000 | >−1.400000 |
| Oleiphilus messinensis | <−3.000000 | −3.000000 < and < −1.100000 | >−1.100000 |
| Oleispira antarctica | <−1.945382 | −1.945382 < and < −1.000000 | >−1.000000 |
| Pantoea agglomerans | <−3.219117 | −3.219117 < and < −1.000000 | >−1.000000 |
| Paraglaciecola psychrophila 170 | <−1.881160 | −1.881160 < and < −0.800000 | >−0.800000 |
| Pectobacterium atrosepticum | <−3.132863 | −3.132863 < and < −1.000000 | >−1.000000 |
| Photorhabdus asymbiotica | <−3.100000 | −3.100000 < and < −1.500000 | >−1.500000 |
| ATCC43949 | | | |
| Plautia stali symbiont | <−3.110319 | −3.110319 < and < −1.100000 | >−1.100000 |
| Plesiomonas shigelloides | <−2.876276 | −2.876276 < and < −1.000000 | >−1.000000 |
| Pluralibacter gergoviae | <−3.365271 | −3.365271 < and < −1.600000 | >−1.600000 |
| Polycyclovorans algicola TG408 | <−2.400000 | −2.400000 < and < −1.000000 | >−1.000000 |
| Pragia fontium | <−2.738318 | −2.738318 < and < −1.600000 | >−1 .600000 |
| Proteus mirabilis | <−2.885216 | −2.885216 < and < −1.400000 | >−1.400000 |
| Providencia alcalifaciens | <−2.805076 | −2.805076 < and < −1.000000 | >−1.000000 |
| Pseudoalteromonas agarivorans DSM | <−2.308131 | −2.308131 < and < −1.100000 | >−1.100000 |
| Pseudohongiella spirulinae | <−2.600000 | −2.600000 < and < −1.100000 | >−1.100000 |
| Pseudoxanthomonas spadix BDa59 | <−5.600000 | −5.600000 < and < −0.700000 | >−0.700000 |
| Psychrobacter alimentarius | <−2.316710 | −2.316710 < and < −1.000000 | >−1.000000 |
| Psychromonas ingrahamii 37 | <−2.437604 | −2.437604 < and < −1.000000 | >−1.000000 |
| Rahnella aquatilis CIP | <−3.042640 | −3.042640 < and < −1.500000 | >−1.500000 |
| Raoultella ornithinolytica | <−3.325168 | −3.325168 < and < −1.600000 | >−1.600000 |
| Reinekea forsetii | <−2.534190 | −2.534190 < and < −1.500000 | >−1.500000 |
| Rhodanobacter denitrificans | <−3.900000 | −3.900000 < and < −1.600000 | >−1.600000 |
| Rhodobaca barguzinensis | <−3.165517 | −3.165517 < and < −0.700000 | >−0.700000 |
| Rhodobacter capsulatus SB | <−3.940852 | −3.940852 < and < −2.200000 | >−2.200000 |
| Rhodobacteraceae bacterium | <−2.737199 | −2.737199 < and < −1.500000 | >−1.500000 |
| HTCC2083 | | | |
| Rhodobacterales bacterium Y4I | <−3.745547 | −3.745547 < and < −1.600000 | >−1.600000 |
| Rhodomicrobium vannielii ATCC | <−2.877063 | −2.877063 < and < −1.200000 | >−1.200000 |
| Rhodoplanes sp Z2YC6860 | <−2.778921 | −2.778921 < and < −1.000000 | >−1.000000 |
| Rhodopseudomonas palustris BisA53 | <−2.981119 | −2.981119 < and < −1.100000 | >−1.100000 |
| Rhodovibrio salinarum DSM | <−3.296529 | −3.296529 < and < −1.000000 | >−1.000000 |
| Rhodovulum sp ES010 | <−3.922936 | −3.922936 < and < −1.400000 | >−1.400000 |
| Roseibacterium elongatum DSM | <−3.524928 | −3.524928 < and < −1.500000 | >−1.500000 |
| Roseobacter denitrificans OCh | <−3.196068 | −3.196068 < and < −0.800000 | >−0.800000 |
| Roseomonas gilardii | <−3.344185 | −3.344185 < and < −2.400000 | >−2.400000 |
| Roseovarius mucosus | <−3.435302 | −3.435302 < and < −0.600000 | >−0.600000 |
| Ruegeria mobilis F1926 | <−3.468672 | −3.468672 < and < −1.700000 | >−1.700000 |
| Saccharophagus degradans 240 | <−2.238156 | −2.238156 < and < −1.500000 | >−1.500000 |
| Sagittula sp P11 | <−3.900000 | −3.900000 < and < −2.600000 | >−2.600000 |
| Salmonella bongori N26808 | <−3.197458 | −3.197458 < and < −1.700000 | >−1.700000 |
| Sedimenticola thiotaurini | <−2.834295 | −2.834295 < and < −1.600000 | >−1.600000 |
| Sedimentitalea nanhaiensis DSM | <−3.175187 | −3.175187 < and < −0.800000 | >−0.800000 |
| Serratia ficaria | <−3.364721 | −3.364721 < and < −1.700000 | >−1.700000 |
| Shewanella algae | <−3.100000 | −3.100000 < and < −0.200000 | >−0.200000 |
| Shigella dysenteriae Sd197 | <−3.700000 | −3.700000 < and < −0.100000 | >−0.100000 |
| Shimwellia blattae DSM | <−3.364894 | −3.364894 < and < −1.700000 | >−1.700000 |
| Shinella sp HZN7 | <−3.602524 | −3.602524 < and < −1.200000 | >−1.200000 |
| Silicibacter lacuscaerulensis ITI1157 | <−3.443613 | −3.443613 < and < −0.700000 | >−0.700000 |
| Simiduia agarivorans SA1 | <−2.655831 | −2.655831 < and < −1.700000 | >−1.700000 |
| Sinorhizobium americanum | <−3.586451 | −3.586451 < and < −1.600000 | >−1.600000 |
| Sodalis glossinidius str | <−2.669986 | −2.669986 < and < −1.600000 | >−1.600000 |
| Sphingobium baderi | <−3.112818 | −3.112818 < and < −1.000000 | >−1.000000 |
| Sphingopyxis alaskensis RB2256 | <−2.976207 | −2.976207 < and < −1.000000 | >−1.000000 |
| Sphingorhabdus flavimaris | <−2.471862 | −2.471862 < and < −1.000000 | >−1.000000 |
| Spongiibacter sp IMCC21906 | <−2.702126 | −2.702126 < and < −1.000000 | >−1.000000 |
| Stappia sp ES058 | <−3.224489 | −3.224489 < and < −1.000000 | >−1.000000 |
| Starkeya novella DSM | <−3.427923 | −3.427923 < and < −1.200000 | >−1.200000 |
| Stenotrophomonas acidaminiphila | <−5.900000 | −5.900000 < and < −0.800000 | >−0.800000 |
| Steroidobacter denitrificans | <−6.700000 | −6.700000 < and < −0.700000 | >−0.700000 |
| Sulfitobacter donghicola DSW25 | <−3.040483 | −3.040483 < and < −1.500000 | >−1.500000 |
| Sulfurifustis variabilis | <−2.956134 | −2.956134 < and < −1.100000 | >−1.100000 |
| Sulfurospirillum halorespirans DSM | <−3.091358 | −3.091358 < and < −0.500000 | >−0.500000 |
| Tateyamaria omphalii | <−3.116738 | −3.116738 < and < −1.100000 | >−1.100000 |
| Tatlockia micdadei | <−2.465314 | −2.465314 < and < −1.000000 | >−1.000000 |
| Tatumella citrea | <−3.029707 | −3.029707 < and < −1.600000 | >−1.600000 |
| Teredinibacter sp 1162TS0a05 | <−2.400000 | −2.400000 < and < −1.000000 | >−1.000000 |
| Thalassobium sp R2A62 | <−2.760664 | −2.760664 < and < −1.500000 | >−1.500000 |
| Thalassolituus oleivorans | <−2.597518 | −2.597518 < and < −1.000000 | >−1.000000 |
| Thalassospira sp CSC3H3 | <−2.928586 | −2.928586 < and < −1.500000 | >−1.500000 |
| Thalassotalea sp LPB0090 | <−1.849969 | −1.849969 < and < −1.000000 | >−1.000000 |
| Thioalkalivibrio nitratireducens DSM | <−3.300713 | −3.300713 < and < −1.000000 | >−1.000000 |
| Thiobacimonas profunda | <−3.903775 | −3.903775 < and < −1.300000 | >−1.300000 |
| Thioclava nitratireducens | <−3.954070 | −3.954070 < and < −0.600000 | >−0.600000 |
| Thiocystis violascens DSM | <−2.622356 | −2.622356 < and < −1.700000 | >−1.700000 |
| Thioflavicoccus mobilis 8321 | <−2.965535 | −2.965535 < and < −1.000000 | >−1.000000 |
| Thiohalobacter thiocyanaticus | <−2.805036 | −2.805036 < and < −1.500000 | >−1.500000 |
| Thiolapillus brandeum | <−3.400000 | −3.400000 < and < −0.800000 | >−0.800000 |
| Thioploca ingrica | <−2.700000 | −2.700000 < and < −1.000000 | >−1.000000 |
| Thiothrix nivea DSM | <−2.982174 | −2.982174 < and < −1.600000 | >−1.600000 |
| Tistrella mobilis KA081020065 | <−3.658232 | −3.658232 < and < −1.500000 | >−1.500000 |
| Tolumonas auensis DSM | <−3.055160 | −3.055160 < and < −0.800000 | >−0.800000 |
| Variibacter gotj awalensis | <−2.690231 | −2.690231 < and < −1.200000 | >−1 .200000 |
| Vibrio alginolyticus | <−2.571917 | −2.571917 < and < −1.200000 | >−1.200000 |
| Vibro shilonii | <−2.672724 | −2.672724 < and < −0.400000 | >−0.400000 |
| Wenzhouxiangella marina | <−4.500000 | −4.500000 < and < −0.900000 | >−0.900000 |
| Woeseia oceani | <−3.800000 | −3.800000 < and < −0.900000 | >−0.900000 |
| Xanthobacter autotrophicus Py2 | <−3.597229 | −3.597229 < and < −1.200000 | >−1.200000 |
| Xanthobacteraceae bacterium 501b | <−3.345780 | −3.345780 < and < −1.100000 | >−1.100000 |
| Xanthomonas albilineans | <−6.700000 | −6.700000 < and < −0.200000 | >−0.200000 |
| Xenorhabdus bovienii str | <−2.919608 | −2.919608 < and < −1.000000 | >−1.000000 |
| Xuhuaishuia manganoxidans | <−3.447165 | −3.447165 < and < −0.300000 | >−0.300000 |
| Yersinia aldovae 67083 | <−2.856461 | −2.856461 < and < −1.000000 | >−1.000000 |
| Zhongshania aliphaticivorans | <−2.513355 | −2.513355 < and < −1.000000 | >−1.000000 |
| Zobellella denitrificans | <−3.576612 | −3.576612 < and < −1.000000 | >−1.000000 |
| Zooshikella ganghwensis | <−2.600000 | −2.600000 < and < −0.400000 | >−0.400000 |
| Pseudomonas syringae pv | <−3.900000 | −3.900000 < and < −0.500000 | >−0.500000 |
| Salinispira pacifica | <−6.300000 | −6.300000 < and < −0.500000 | >−0.500000 |
| Sediminispirochaeta smaragdinae | <−4.500000 | −4.500000 < and < −1.700000 | >−1.700000 |
| DSM | | | |
| Sphaerochaeta globosa str | <−4.318439 | −4.318439 < and < −1.500000 | >−1.500000 |
| Spirochaeta africana DSM | <−3.800000 | −3.800000 < and < −2.400000 | >−2.400000 |
| Treponema azotonutricium ZAS9 | <−3.400236 | −3.400236 < and < −0.500000 | >−0.500000 |
| Acetobacter aceti | <−2.800000 | −2.800000 < and < −1.600000 | >−1.600000 |
| Acidiphilium cryptum JF5 | <−3.205888 | −3.205888 < and < −1.100000 | >−1.100000 |
| Afipia broomeae | <−2.856849 | −2.856849 < and < −1.100000 | >−1.100000 |
| Agrobacterium genomosp 3 | <−3.182662 | −3.182662 < and < −1.500000 | >−1.500000 |
| Altererythrobacter atlanticus | <−2.822028 | −2.822028 < and < −1.500000 | >−1.500000 |
| Aminobacter aminovorans | <−3.196846 | −3.196846 < and < −1.000000 | >−1.000000 |
| Ancylobacter sp FA202 | <−3.336092 | −3.336092 < and < −1.100000 | >−1.100000 |
| Antarctobacter heliothermus | <−3.430722 | −3.430722 < and < −0.800000 | >−0.800000 |
| Asaia bogorensis NBRC | <−2.577357 | −2.577357 < and < −1.000000 | >−1.000000 |
| Aurantimonas manganoxydans | <−2.983673 | −2.983673 < and < −1.100000 | >−1.100000 |
| SI859A1 | | | |
| Azorhizobium caulinodans ORS | <−3.443215 | −3.443215 < and < −1.200000 | >−1.200000 |
| Azospirillum brasilense | <−3.492505 | −3.492505 < and < −1.200000 | >−1.200000 |
| Beijerinckia indica subsp | <−2.839956 | −2.839956 < and < −1.700000 | >−1.700000 |
| Belnapia sp F41 | <−3.271592 | −3.271592 < and < −1.100000 | >−1.100000 |
| Blastochloris viridis | <−3.098774 | −3.098774 < and < −1.100000 | >−1.100000 |
| Blastomonas sp RAC04 | <−2.634917 | −2.634917 < and < −1.500000 | >−1.500000 |
| Bosea sp AS1 | <−3.123630 | −3.123630 < and < −1.200000 | >−1.200000 |
| Bradyrhizobiaceae bacterium SG6C | <−2.887387 | −2.887387 < and < −1.100000 | >−1.100000 |
| Bradyrhizobium diazoefficiens | <−2.662466 | −2.662466 < and < −1.000000 | >−1.000000 |
| Brevundimonas diminuta | <−2.833427 | −2.833427 < and < −0.400000 | >−0.400000 |
| Brucella abortus 2308 | <−3.038021 | −3.038021 < and < −1.800000 | >−1 .800000 |
| Candidatus Filomicrobium marinum | <−2.997037 | −2.997037 < and < −0.400000 | >−0.400000 |
| Caulobacter crescentus CB15 | <−2.700000 | −2.700000 < and < −0.400000 | >−0.400000 |
| Caulobacteraceae bacterium | <−2.632395 | −2.632395 < and < −1.100000 | >−1.100000 |
| OTSzA272 | | | |
| Celeribacter ethanolicus | <−3.510748 | −3.510748 < and < −1.000000 | >−1.000000 |
| Chelativorans sp BNC1 | <−3.516485 | −3.516485 < and < −1.100000 | >−1.100000 |
| Chelatococcus daeguensis | <−3.512001 | −3.512001 < and < −1.200000 | >−1.200000 |
| Citromicrobium sp JL477 | <−2.790781 | −2.790781 < and < −1.200000 | >−1 .200000 |
| Cohaesibacter sp ES047 | <−3.036928 | −3.036928 < and < −1.000000 | >−1.000000 |
| Confluentimicrobium sp EMB200NS6 | <−3.509900 | −3.509900 < and < −1.000000 | >−1.000000 |
| Croceicoccus marinus | <−2.528371 | −2.528371 < and < −1.000000 | >−1.000000 |
| Defluviimonas alba | <−3.546150 | −3.546150 < and < −1.000000 | >−1.000000 |
| Devosia sp A16 | <−3.125063 | −3.125063 < and < −1.100000 | >−1.100000 |
| Dinoroseobacter shibae DFL | <−3.630722 | −3.630722 < and < −0.700000 | >−0.700000 |
| Ensifer adhaerens | <−3.426882 | −3.426882 < and < −1.500000 | >−1.500000 |
| Erythrobacter atlanticus | <−2.514135 | −2.514135 < and < −1.000000 | >−1.000000 |
| Fulvimarina pelagi HTCC2506 | <−2.836540 | −2.836540 < and < −1.500000 | >−1.500000 |
| Geminicoccus roseus DSM | <−3.102675 | −3.102675 < and < −1.100000 | >−1.100000 |
| Gluconacetobacter diazotrophicus PA1 | <−3.084149 | −3.084149 < and < −1.700000 | >−1.700000 |
| Gluconobacter albidus | <−2.900000 | −2.900000 < and < −0.800000 | >−0.800000 |
| Halocynthiibacter arcticus | <−2.919151 | −2.919151 < and < −1.500000 | >−1.500000 |
| Hartmannibacter diazotrophicus | <−3.273364 | −3.273364 < and < −1.100000 | >−1.100000 |
| Henriciella litoralis | <−2.974939 | −2.974939 < and < −0.400000 | >−0.400000 |
| Hirschia baltica ATCC | <−2.682743 | −2.682743 < and < −0.400000 | >−0.400000 |
| Hoeflea phototrophica DFL43 | <−3.062987 | −3.062987 < and < −1.000000 | >−1.000000 |
| Hyphomicrobium denitrificans 1NES1 | <−2.812979 | −2.812979 < and < −1.100000 | >−1.100000 |
| Hyphomonas neptunium ATCC | <−3.266014 | −3.266014 < and < −1.000000 | >−1.000000 |
| Jannaschia sp CCS1 | <−3.211797 | −3.211797 < and < −0.700000 | >−0.700000 |
| Ketogulonicigenium vulgare | <−3.039662 | −3.039662 < and < −1.000000 | >−1.000000 |
| Komagataeibacter europaeus | <−2.700000 | −2.700000 < and < −1.600000 | >−1.600000 |
| Labrenzia aggregata | <−3.189993 | −3.189993 < and < −0.900000 | >−0.900000 |
| Leisingera aquimarina DSM | <−3.517294 | −3.517294 < and < −1.000000 | >−1.000000 |
| Litoreibacter janthinus | <−3.052386 | −3.052386 < and < −0.600000 | >−0.600000 |
| Loktanella vestfoldensis | <−2.800636 | −2.800636 < and < −0.700000 | >−0.700000 |
| Magnetococcus marinus MC1 | <−3.260016 | −3.260016 < and < −1.500000 | >−1.500000 |
| Magnetospira sp QH2 | <−3.290434 | −3.290434 < and < −0.700000 | >−0.700000 |
| Magnetospirillum gryphiswaldense | <−3.114222 | −3.114222 < and < −1.900000 | >−1.900000 |
| MSR1 | | | |
| Maricaulis mans MC S10 | <−3.184234 | −3.184234 < and < −1.100000 | >−1.100000 |
| Marinovum algicola DG | <−3.581252 | −3.581252 < and < −1.500000 | >−1.500000 |
| Maritimibacter alkaliphilus | <−3.671444 | −3.671444 < and < −0.400000 | >−0.400000 |
| HTCC2654 | | | |
| Martelella endophytica | <−3.447367 | −3.447367 < and < −1.500000 | >−1.500000 |
| Mesorhizobium amorphae | <−3.406805 | −3.406805 < and < −1.000000 | >−1.000000 |
| CCNWGS0123 | | | |
| Methylobacterium aquaticum | <−3.240759 | −3.240759 < and < −1.000000 | >−1.000000 |
| Methylocapsa acidiphila B2 | <−2.596260 | −2.596260 < and < −1.000000 | >−1.000000 |
| Methyloceanibacter caenitepidi | <−3.011276 | −3.011276 < and < −0.400000 | >−0.400000 |
| Methylocella silvestris BL2 | <−2.829478 | −2.829478 < and < −1.000000 | >−1.000000 |
| Methylocystis bryophila | <−2.971689 | −2.971689 < and < −1.200000 | >−1.200000 |
| Methyloferula stellata AR4 | <−2.538231 | −2.538231 < and < −1.000000 | >−1 .000000 |
| Methylopila sp 73B | <−3.147754 | −3.147754 < and < −1.200000 | >−1.200000 |
| Methylosinus sp LW3 | <−3.039350 | −3.039350 < and < −1.100000 | >−1.100000 |
| Microvirga ossetica | <−3.189630 | −3.189630 < and < −1.100000 | >−1.100000 |
| Neoasaia chiangmaiensis | <−2.400000 | −2.400000 < and < −1.800000 | >−1.800000 |
| Neorhizobium galegae complete | <−3.406724 | −3.406724 < and < −1.000000 | >−1.000000 |
| Nitratireductor basaltis | <−2.807240 | −2.807240 < and < −1.100000 | >−1.100000 |
| Nitrobacter hamburgensis X14 | <−2.804284 | −2.804284 < and < −1.100000 | >−1.100000 |
| Novosphingobium aromaticivorans | <−3.020822 | −3.020822 < and < −1.000000 | >−1.000000 |
| DSM | | | |
| Oceanicaulis sp HTCC2633 | <−3.366079 | −3.366079 < and < −0.300000 | >−0.300000 |
| Oceanicola litoreus | <−3.601662 | −3.601662 < and < −1.000000 | >−1.000000 |
| Ochrobactrum pseudogrignonense | <−3.199697 | −3.199697 < and < −1.100000 | >−1.100000 |
| Octadecabacter antarcticus 307 | <−2.598415 | −2.598415 < and < −1.500000 | >−1.500000 |
| Oligotropha carboxidovorans OM4 | <−3.092688 | −3.092688 < and < −1.200000 | >−1.200000 |
| Pacificimonas flava | <−2.968269 | −2.968269 < and < −1.000000 | >−1.000000 |
| Pannonibacter phragmitetus | <−3.476118 | −3.476118 < and < −2.000000 | >−2.000000 |
| Paracoccus aminophilus JCM | <−3.183532 | −3.183532 < and < −1.000000 | >−1.000000 |
| Parvibaculum lavamentivorans DS1 | <−3.406858 | −3.406858 < and < −1.100000 | >−1.100000 |
| Pelagibaca abyssi | <−3.781895 | −3.781895 < and < −1.200000 | >−1.200000 |
| Pelagibacterium halotolerans B2 | <−3.113097 | −3.113097 < and < −1.500000 | >−1.500000 |
| Phaeobacter gallaeciensis | <−3.549024 | −3.549024 < and < −0.700000 | >−0.700000 |
| Phenylobacterium zucineum HLK1 | <−3.402358 | −3.402358 < and < −0.200000 | >−0.200000 |
| Phyllobacterium sp Tri48 | <−3.062057 | −3.062057 < and < −1.100000 | >−1.100000 |
| Planktomarina temperata RCA23 | <−2.913244 | −2.913244 < and < −1.000000 | >−1.000000 |
| Polymorphum gilvum SL003B26A1 | <−3.742394 | −3.742394 < and < −1.000000 | >−1.000000 |
| Porphyrobacter neustonensis | <−2.650815 | −2.650815 < and < −1.000000 | >−1.000000 |
| Pseudolabrys sp Root1462 | <−2.826490 | −2.826490 < and < −1.000000 | >−1.000000 |
| Pseudooceanicola batsensis | <−3.677934 | −3.677934 < and < −1.000000 | >−1.000000 |
| HTCC2597 | | | |
| Pseudophaeobacter arcticus DSM | <−3.326592 | −3.326592 < and < −0.700000 | >−0.700000 |
| Pseudorhodoplanes sinuspersici | <−2.666925 | −2.666925 < and < −1.100000 | >−1.100000 |
| Pseudovibrio sp FOBEG1 | <−3.112755 | −3.112755 < and < −0.400000 | >−0.400000 |
| Puniceibacterium sp IMCC21224 | <−3.291579 | −3.291579 < and < −1.000000 | >−1.000000 |
| Reyranella massiliensis 521 | <−2.991860 | −2.991860 < and < −1.000000 | >−1.000000 |
| Rhizobium etli | <−3.517473 | −3.517473 < and < −1.600000 | >−1.600000 |
| Rhizorhabdus dicambivorans | <−3.092399 | −3.092399 < and < −1.100000 | >−1.100000 |
| Rhodospirillum photometricum DSM | <−3.620754 | −3.620754 < and < −1.700000 | >−1.700000 |
| Ecoli MG1655 | <−3.236830 | −3.236830 < and < −1.600000 | >−1.600000 |

According to some embodiments, the interaction strength of a various aSD sequences with different 6 nt sequences are given in Table 3. Any 6 nt sequence not provided in Table 3 for a specific aSD sequence has an interaction strength of zero.

TABLE 3
Canonical aSD:
−0.3: GGCCGG;
−0.4: ATGAGA, CGTGAG, CGAGAC, GAGTGT, GAGTCT, GAGATT, GAGCCT,
GAGCGA, CCAGAG, GTCGAG, GAGTTT, CCGAGA, GAGACT, ATAGAG, CGAGCA, ACCGAG, CGAGTC,
CGAGCG, TACGAG, GCGAGC, GAGCAG, TGTGAG, ATCGAG, TTGAGC, CGAGTA, GAGAGA, ACGAGC,
ATTGAG, GACGAG, CTCGAG, TGAGCG, AAGAGA, GAGTCG, TGCGAG, CGAGAG, CAAGAG, TGAGAT,
AGAGAT, GAGCAT, CGCGAG, TGAGTG, GAGCGC, GAGCAC, CTGAGC, ACAGAG, CAGAGA, AGAGCC,
GAGTAC, ACGAGT, AGAGAA, TAGAGT, GAGTAG, ATGAGT, GAGTGA, TGAGCT, CCGAGT, ACGAGA,
GAGTTA, GAGAAT, GAGAGC, GAGTAT, TTGAGT, GAGCCG, GAGCGG, AAGAGT, GAGTGC, TGAGCC,
GAGATA, GAGTTG, ACTGAG, GAGCGT, GCCGAG, CTAGAG, GAGTAA, CAGAGC, TAAGAG, GAGACG,
CACGAG, CAGAGT, AGAGCT, TCAGAG, CGAGTT, GAGCAA, AATGAG, GAGTGG, AACGAG, GAGCCA,
AAGAGC, GAGCTG, TGAGAC, GAGATC, CTTGAG, CCTGAG, GAGATG, AGAGCG, TCGAGC, CATGAG,
GCTGAG, GAGAAG, CGAGAT, GTAGAG, CTGAGA, GTTGAG, TCCGAG, TTAGAG, AGAGTT, AGAGTG,
GAGTCA, AGAGCA, GAGCTT, CCGAGC, CCCGAG, TGAGTT, GCGAGA, TAGAGC, CGAGTG, TGAGTA,
TGAGTC, TGAGAA, TTGAGA, GTGAGC, TCGAGA, GCAGAG, AGAGTC, CGAGCT, AGAGTA, GTGAGT,
GAGAAA, CGAGCC, GAGTTC, AAAGAG, GATGAG, GAGCTA, CGAGAA, AGAGAC, TATGAG, TTCGAG,
TAGAGA, GAGAAC, GCGAGT, TGAGCA, GAGAGT, GAGCTC, ATGAGC, TCGAGT, GAGCCC, TGAGAG,
TTTGAG, GAGACC, GAAGAG, GAGTCC, CTGAGT, GAGACA, TCTGAG, GTGAGA;
−0.5: AGTTGG, AGATGG, AGCTGG;
−0.8: GATAGG, ACCGGG, AGGCAC, AATGGG, GGGCAC, AGGTAT, CAGGCT, ACAGGC, GTAGGC,
ACTAGG, GGGTTC, ACCAGG, TTGGGC, TAGGTT, GTAGGT, AAGGCG, GACAGG, AGGCCA, ATCGGG,
CTCAGG, TCTAGG, TGGGTA, AGGTTG, ATAAGG, AGGCTT, AAAAGG, TAGGTC, GCAAGG, CCTGGG,
CTAAGG, TAGGCC, TGTGGG, CCCGGG, GGGCGC, CAGGCA, GTCAGG, AGGCTG, GGGTTA, GGGTCT,
GCAGGC, AGGCGT, GGGTAA, AGGCCT, CCGGGC, CGGGCG, CGTAGG, GGGCCA, CTAGGC, TTTGGG,
TGGGCA, TAAGGC, CAAAGG, TGGGCC, GTCGGG, GCCGGG, AAGGTA, GCTAGG, TGGGCT, TTTAGG,
GGGTCA, GTGGGC, CAGGCG, CGGGCT, ATAGGC, TAAAGG, TCCAGG, CCGGGT, TCGGGC, TAGGTA,
AGGCTA, CAAGGT, GTTGGG, AAAGGT, AGGTAC, GATGGG, CATGGG, CCTAGG, AGGTCT, CCAGGC,
AGGTCA, ATGGGT, AGGCCG, ATAGGT, TTAGGC, TCGGGT, TTCGGG, CGGGTA, CGAAGG, CTCGGG,
CTGGGC, GCAGGT, GGGCAT, ACAGGT, ACGGGC, GTAAGG, CACGGG, CACAGG, AGGCGC, TACAGG,
AGGTTA, AACAGG, AACGGG, GGGCTA, AGGCAA, GGGCAA, TAAGGT, AGGTAA, GGGCTC, AAGGCC,
CGGGCA, AAGGCA, ACAAGG, TCCGGG, AAGGCT, AAAGGC, TCTGGG, TTAGGT, AGGTTT, TGTAGG,
CGCGGG, GGGTTG, TAGGCT, GGGCTG, ATGGGC, CAGGCC, GGGCGT, GTGGGT, AGGCGA, AGGTTC,
TCAGGC, GCGGGT, TTCAGG, CAAGGC, TTAAGG, GGGTTT, GCCAGG, CTTGGG, TGCGGG, TATAGG,
TGCAGG, AGGCTC, AATAGG, GGTCGG, CCCAGG, ATTGGG, ATCAGG, CGGGTT, GAAGGT, TCAAGG,
CAGGTT, AGGTCC, CAGGTC, AGGCAT, TGAAGG, CTGGGT, CGGGTC, AAGGTT, CAGGTA, CCAGGT,
GGGTAT, GTTAGG, TAGGCA, CGGGCC, TGGGTC, TACGGG, ACGGGT, TCAGGT, TATGGG, GGGTCC,
GGGCTT, GCTGGG, GGGCCT, GGGCCG, CTAGGT, CGCAGG, CTTAGG, CATAGG, GGGCGA, TTGGGT,
ATTAGG, AGGCCC, CCAAGG, TGGGTT, GGGTAC, GCGGGC, GACGGG, GGGCCC, GAAAGG, ACTGGG,
CGTGGG, AAGGTC, TAGGCG, TGGGCG, GAAGGC;
−0.9: AGGTGT, TGGGTG, AGGTCG, GGGTGT, GGGTCG, GGGTGA, AGGTGC, CAGGTG, AAGGTG,
GGGTGC, TAGGTG, AGGTGA, CGGGTG;
−1: GGCTGG;
−1.1: GGATGC, GGACAC, CGGATC, ACCGGA, GGATTA, GGAAGC, CTTGGA, GGACAT, ACGGAT, CCGGAC,
GGACCT, TCGGAC, TCCGGA, CGGAAT, CACGGA, GGACTC, AATGGA, GACGGA, CATGGA, GGTTGG,
GATGGA, GGACCA, CGGACT, GGAAAG, CTCGGA, TCGGAA, GGATTT, ATTGGA, GGAACG, TGGACA,
GTGGAC, TCTGGA, GGACAA, GGAATC, TGGATT, GGAAGA, TTCGGA, GCGGAC, GGATCA, GGATGA,
GTGGAT, GGAAAC, GGACCG, GGACGA, GGAAAA, GTGGAA, TGGATC, TTGGAA, GGAACT, TTGGAT,
CTGGAT, GGACTG, GGATGT, GGATAC, ATGGAC, AGCGGA, TGGACC, CGGAAA, GGAACC, CCGGAA,
CCCGGA, CGGATA, GGATAA, GCTGGA, TTTGGA, TGGAAT, AACGGA, CTGGAC, GGACTT, TGGACG,
GGATTG, GGAACA, GGATCT, CCGGAT, GGACGT, GGACGC, TGTGGA, TGGAAC, TGGATG, CGGACC,
ATGGAA, TGGAAA, GGATCC, CGTGGA, TGCGGA, GGACCC, TGGACT, CGGATT, GGATAG, GGATCG,
ATGGAT, TGGATA, TGGAAG, TCGGAT, GTTGGA, CGGATG, CGGACG, GTCGGA, GGAAAT, GGATAT,
GGAATA, GGACTA, GCGGAT, GGACAG, CGGAAC, TACGGA, ACTGGA, GCCGGA, TATGGA, GCGGAA,
TTGGAC, ATCGGA, CTGGAA, GGATTC, CGGACA, ACGGAA, CGGAAG, ACGGAC, GGAATT, CGCGGA,
CCTGGA, GGAATG, AGTGGA, GGAAGT;
−1.5: GGGCAG, GGGTAG, AGGCAG, AGAGAG, AGTGAG,
GGCGAG, AGGTAG, AGCGAG, GGTGAG;
−1.7: AGTAGG, AGCAGG, AGAAGG, AGCGGG, AGTGGG;
−1.8: GAAGGG, AAGGGC, AAAGGG, GCAGGG, AGGGCT, TAGGGT, AGGGCC, GTAGGG, CAAGGG, TAAGGG,
TCAGGG, CAGGGT, CTAGGG, AGGGTA, TTAGGG, AGGGCA, ATAGGG, TAGGGC, ACAGGG, AAGGGT,
AGGGTT, AGGGTC, CCAGGG, CAGGGC, AGGGCG, AGGGTG;
−2.5: TGGCGG, GGCGGA, GGCGGT, CGGCGG, GGCGGG, GGCGGC;
−2.6: GGTGGT, CGGTGG, GGTGGG, GGTGGC, TGGTGG, GGTGGA;
−2.7: AAGGGA, AGGGAA, TGGGAC, ACAGGA, TAGGAT, GGGACA, GCGGGA,
TAGGAA, TGGGAT, AGGACG, GGGATA, GGGAAG, GGGAAT, GAAGGA, AGGACA,
GGGATT, AGGAAG, AGGATC, CAGGAC, CAGGGA,
AGGATG, GGGACG, GTGGGA, AGGATA, AGGAAC, AGGGAT, ATAGGA, TTGGGA, TTAGGA, CCAGGA,
CGGGAC, AAGGAA, GGGACC, TCGGGA, AGGGAC, ACGGGA, AGGACT, TAGGAC, TAAGGA, AGGAAA,
AGGAAT, CGGGAA, CTGGGA, TAGGGA, CAAGGA, AGGACC, GGGAAC, GGGAAA, GGGATC, AGGATT,
AAAGGA, TGGGAA, ATGGGA, CGGGAT, CAGGAA, GGGACT, GTAGGA, GGGATG, TCAGGA, CAGGAT,
GCAGGA, AAGGAC, CCGGGA, CTAGGA, AAGGAT;
−2.8: ATGGGG, TTGGGG, TGGGGA, CGGGGT, CGGGGC,
GCGGGG, GGGGCA, GGGGTT, GGGGAA, GGGGCC, GGGGTG, ACGGGG, CTGGGG, CCGGGG, CGGGGA,
GGGGAT, GTGGGG, TGGGGC, TGGGGT, GGGGCT, GGGGTC, GGGGTA, TCGGGG, GGGGCG, GGGGAC;
−3.2: GGACGG, GGCAGG, GGAAGG, GGATGG, GGTAGG;
−3.7: GGAGTT, TCGAGG, CTGAGG, GAGGCG,
GGAGCC, GGAGAG, AAGAGG, GGAGTG, ACGGAG, GCGAGG, GAGGGA, AGAGGA, GGAGCT, AGAGGC,
AGAGGT, GAGGCC, TGAGGT, TTGGAG, CGAGGA, GAGGAT, CCGGAG, TAGAGG, GTGGAG, TGGAGC,
TGGAGA, ATGGAG, CAGAGG, TTGAGG, CGGAGC, GAGGTG, TGAGGA, GAGGTC, CGAGGC, GAGGTT,
ACGAGG, GGAGCA, GGAGAA, AGAGGG, GGAGTC, GGAGAT, GAGAGG, GGAGTA, TGGAGT, GAGGAA,
GAGGGT, CTGGAG, ATGAGG, CCGAGG, GAGGGC, GAGGTA, TGAGGC, GGAGCG, TCGGAG, GGAGAC,
CGAGGG, GTGAGG, GAGGCT, CGAGGT, CGGAGT, GAGGAC, GAGGCA, TGAGGG, GCGGAG, CGGAGA;
−4.1: AGGCGG, GGGCGG;
−4.2: AGGTGG, GGGTGG;
−4.4: CAGGGG, AGGGGA, AAGGGG, GAGGGG, AGGGGT, AGGGGC, TAGGGG;
−5.3: AGGAGT, AGGAGA, GAGGAG, GGGAGT, AGGAGC, GGGAGA,
GGGGAG, AGGGAG, AAGGAG, CAGGAG, GGGAGC, TGGGAG, TAGGAG, CGGGAG;
−6.1: GGGGGC, GGGGGT, CGGGGG, TGGGGG, GGGGGA;
−7: GGAGGG, GGAGGC, GGAGGT, TGGAGG, GGAGGA, CGGAGG;
−7.7: GGGGGG, AGGGGG;
−8.6: GGGAGG, AGGAGG.
GCCGCG aSD: 10.8: CGCGGC;
−0.1: CATTGG, AATGGG, CAATGG, TGGGAC, CTTGGA, TTCTGG, GCCTGG,
TGTAGT, GCTTGG, TTATGG, GACTGG, CACTGG, CCTGGG, AACTGG, TTGGAG, AATGGA, CATGGA,
TGGGAT, GATGGA, ACATGG, CCTTGG, TTTGGG, ATTGGA, ATATGG, TGGACA, TCTGGA, TGGATT,
TGGAGA, ATGGAG, GTATGG, AAATGG, TAATGG, CTATGG, TGGATC, TTGGAA, GTTGGG, GATGGG,
CATGGG, TTGGAT, CCATGG, CTGGAT, ATGGAC, ATCTGG, TGGAGG, TGGACC, TTGGGA, TATTGG,
TTTGGA, TGGAAT, TTTTGG, GGATGG, AGTTGG, TGGAGT, CTGGAC, GTCTGG, TCCTGG, TGGGAG,
TGGACG, CTGGAG, AGATGG, TCTGGG, ACTTGG, CTGGGA, TGGAAC, TGGATG, GCATGG, GATTGG,
ATGGAA, TGGAAA, TCTTGG, CTTGGG, TCATGG, TGGACT, TGTTGT, ATTGGG, TACTGG, CTTTGG,
TGGGAA, ATGGGA, ATGGAT, TGGATA, CTCTGG, TGGAAG, GTTGGA, GAATGG, TATGGG, GTTTGG,
ACCTGG, ACTGGA, AATTGG, TATGGA, TTGGAC, CTGGAA, CCCTGG, ATTTGG, CCTGGA, ACTGGG;
−0.2: GGATGC, CTGAGG, GTGCAG, TTTTGC, TGCATC, ATGCAC, GAATGC, TTGCTA, TGCTAT,
TGCCCC, AGATGC,
AATGCC, CTGCCG, GTGCAT, ATGCTA, TTTGCC, GTGCTT, GTCTGC, TGCATT, ACCTGC, GATGCT, CTATGC,
CACTGC, TGCACG, TTTGCA, TGCACC, GTGCAA, ATTGCT, TCTGCT, ATTGCA, TGCTCG, TTGCTC, TACTGC,
CATGCA, ATCTGC, CCCTGC, ATGCAT, TGCCCG, CCTGCT, CTGCCT, AATTGC, TGCTCT, TGCTAC, TGCCTG,
ATTGCC, AGTGCA, TTGAGG, ATATGC, CTGCTT, TGAGGA, TGCTTC, TGCACT, GTGCAC, AAATGC, GTGCCA,
TGCACA, TGCCAT, GAGTGC, TGCTAA, TGCCAC, GTGCTG, TTGCAT, GTGCCT, GTGCCG, TGTTGG, TGCTGA,
CTGCTC, TGATGC, TGCAAG, ATGCCT, ATGCTG, CTGCTA, TTATGC, CTTTGC, TTGCAG, TGCCAA, CATTGC,
GTTTGC, TGCAGA, CTGCAT, TGCTTG, TTGCTT, CTTGCA, ACTTGC, CATGCT, ATGCTC, TATGCA, ATGCCC,
GATGCC, TGCTTA, TATGCC, TCTGCC, ACATGC, TAATGC, CAGTGC, ATGCAA, CTTGCT, CTTGCC, TTGCCC,
TGCATG, TCTTGC, TGCAAT, ATGCCA, TATTGC, ATGCAG, ATGAGG, GACTGC, CCATGC, TAGTGC, TGTAGG,
AACTGC, TTGCTG, AGTGCC, TGCCGA, AATGCA, CTGCCC, TGCCTC, GTGCTC, TGCCTA, TTGCCG, ATGCTT,
TTTGCT, ATTTGC, GATGCA, TCATGC, GTGCTA, ACTGCA, TGCAAC, CCTGCC, CTCTGC, TGCCCT, TGCCAG,
ATGCCG, GATTGC, TGCTAG, AAGTGC, CTGCAA, CAATGC, GTGAGG, TGCAAA, GTGCCC, TTGCCT, TATGCT,
TGCCTT, GTATGC, TTCTGC, CTGCAC, TTGCAC, TGCCCA, TTGCAA, ACTGCC, TGCTCA, TGATGG, CCTTGC,
TCCTGC, CTGCCA, TCTGCA, TGAGGG, TGCTTT, CTGCAG, AATGCT, TTGCCA, TGCATA, ACTGCT, AGTGCT,
TGCTCC, CCTGCA, CATGCC, CTGCTG;
−0.3: GACGTC, TCGTTT, TCGTCC, CCGTCG, CACCGT, GCCCGT,
AACCGT, CACGTC, CCGTAT, CGTTCC, ACGTAG, CGTCTG, CGTCAA, AAACGT, CCGTCA, CGTCAC, CCGACG,
TGACGT, TCGTTG, GTCGTT, TTACGT, ACGTCA, TTCGTC, CGTACT, CAACGT, CCCGTT, ACGTAA, TTCGTT,
CCGTTG, CCTCGT, AGACGT, GTCGTC, ATCGTC, CGTTTG, TACGTT, ACGTCT, CGTAAC, ATACGT, CGTAAA,
ACGTAC, TTCCGT, CACGTA, CGTTCA, CATCGT, CGTTCT, TACGTC, TCGTAA, CTACGT, CCCGTC, CGTACG,
CCGTAA, ACGTTG, CGACGT, CCGTCC, CCCGTA, CGTATA, CCGTTA, CGTATT, TGTCGT, AACGTC, GCACGT,
AACGTA, CGTTAA, CGTAGA, CCGTTC, CTCGTC, TACGTA, CGTTGA, ACGTTA, CGTTAT, ACCCGT, CGTTTT,
TTCGTA, CGTATG, CACGTT, TCGTCG, CGTAAG, GACCGT, TCGTAG, TCCGTC, ACGTAT, CGTAAT, ATTCGT,
GGACGT, CGTCCT, GACGTT, TCGTCA, TCGTAC, GCTCGT, CGACGA, TCGTTA, GTCGTA, GATCGT, CGTTCG,
CGTCCG, ACCGTC, CGTTTC, CTTCGT, ATCGTT, CGTCTT, CCGTCT, TCCGTA, TCTCGT, CGTCAT, CCGTAG,
ACACGT, ATCGTA, CGTTAG, CTCGTA, CCACGT, TAACGT, TCACGT, ACGTTC, CGTACC, TCGACG, CCCCGT,
ACGACG, GACGTA, ACTCGT, TATCGT, CCGTTT, CGTTAC, CGTTTA, CGTCCA, CGTCTC, TCCCGT, CGTCGA,
TACCGT, CGTCAG, TCGTAT, GTACGT, CTCCGT, AATCGT, TCGTCT, CGTCTA, CGTATC, CTCGTT, AACGTT,
ACGTCG, GTTCGT, ATCCGT, AGTCGT, ACCGTT, CGTACA, GAACGT, ACGTCC, ACCGTA, ACGTTT, CGTCCC,
GTCCGT, TCGTTC, TCCGTT, TTTCGT, CCGTAC;
−0.4: GCCAGC, GCTTGC, GCTAGC, GCCTGC, GCATGC, GCAAGC;
−0.6: AGTTGC, GTAGCA, GTTGCT, GTAGCT, GTAGCC, TGGAGC, GTTGCA, GTTGCC, AGTAGC;
−0.7: AGTGTG, TGTGAA, TTGTGT, CATGTG, CTGTGA, TGTGTT, TATGTG, ATGTGT, TGTGAG, TGTGTA,
TTGTGA, TCTGTG, TGTGCA, ATGTGA, ATTGTG, ATGTGC, TTGTGC, GATGTG, GTGTGA, CTGTGT, GTTGTG,
AATGTG, TGTGTC, TGTGAT, CCTGTG, TGTGAC, CTTGTG, TGTGCC, TTTGTG, TGTGTG, CTGTGC, TGTGCT,
ACTGTG;
−0.8: GCCGTC, GCTGTG, GCAGTT, GCTGTC, GCCGTA, GCAGTG, GCCGTT, GCAGTC, GCTGTA,
AGCCGT, AGCTGT, GCTGTT, GCAGTA, AGCAGT;
−1: CGAAGC, GGGAGC;
−1.1: CGATGC, CGTCGT;
−1.2: GGTAGT, AGGTAT, GGTCTA, AGGTGT, GGGTTC, TAGGTT, GGTCGA, GGTAAA, CGAGCA, AGGTTG,
GGTGCT, TAGGTC, GGTGAT, GGTTCA, ACGAGC, GGTTGG, GGTGAA, GGTTTA, GGTGCA, GGGTTA,
GGGTCT, GCAGGG, AGGTCG, GGGTAA, GGTTTG, GGGTGT, GGTAAT, TAGGGT, GGTCCT, GGGTCG,
GGTATC, GGGTGA, GGTTCG, AAGGTA, GGTATT, GGGTCA, GGTCCC, GGTACG, GGTTAG, GGTCAT,
TAGGTA, GGGTAG, GGTTCC, CAAGGT, AGGTGC, AAAGGT, AGGTAC, GGTGCC, AGGTCT, AGGTCA,
GGTCTT, ATAGGT, CAGGTG, GGTAGC, AGCAGG, GGTCGT, CAGGGT, ACAGGT, GGTTGA, GGTAAC,
AAGGTG, AGGGTA, GGGTGC, GGTTTC, GGTATA, GGTGTC, GCTGGA, AGGTTA, TAAGGT, AGGTAA,
GAGGGT, GGTGTT, TCGAGC, AAGGGT, TTAGGT, AGGTTT, GGTCCG, GGGTTG, GGTCTC, GGTTGC,
AGGGTT, GGTACT, AGGTTC, TAGGTG, GGTCAG, GGTATG, GGTCAC, GGTCTG, GGGTTT, AGGTGA,
GGTCCA, CCGAGC, GGTTGT, GAAGGT, AGGGTC, GGTTCT, CAGGTT, AGGTCC, CAGGTC, GGTACC,
AAGGTT, CAGGTA, CCAGGT, GGGTAT, CGAGCT, AGGTAG, CGAGCC, TCAGGT, GGGTCC, GGTGTG,
GGTTAT, GCTGGG, GGTAGG, GGTGAC, GGTCAA, CTAGGT, GGTTAA, GCAGGA, GGGTAC, AGCTGG,
GGTTTT, GGTGTA, GGTAAG, GGTTAC, GGTACA, GGTAGA, AGGGTG, GGTGAG, AAGGTC;
−1.3: GTAGGT, AGAGGT, GAGGTG, GAGGTC, GAGGTT, GGAGGT, GAGGTA, GTGAGT, GTGTGT;
−1.4: GGGGGG, AGGGGG, CAGGGG, AGGGGA, GGGGAG, GGGGAA, GGGGAT, AAGGGG, GGGGGA, GAGGGG,
TAGGGG, GGGGAC;
−1.5: TGTTGC, TGTAGC;
−1.6: CGGATC, ACCGGG, ACCGGA, ACGGAG, CAACGG, ACGGAT,
CCGGAC, ATCGGG, TCGGAC, TCCCGG, GGACGG, TCCGGA, GTACGG, TGGGTG, TGGGTA, AATCGG,
ACTCGG, CGGAAT, CCCCGG, GAACGG, ATCCGG, GACGGA, CCCGGG, CGGACT, GTTCGG, CTCGGA,
TCGGAA, CCGGAG, CGTTGT, GTCGGG, GCCGGG, TTCGGA, GCCCGG, TTCCGG, ATTCGG, TTTCGG,
ATGGGT, AAACGG, CGTAGT, TTCGGG, CTCGGG, CGGAAA, CCGGAA, CCCGGA, CGGATA, CGGAGG,
AACGGA, CGGGAC, AGCCGG, AACGGG, CTTCGG, GACCGG, TACCGG, TCGGGA, ACGGGA, TCCGGG,
CCGGAT, CTACGG, CGGGAA, CCTCGG, CGGACC, TAACGG, GATCGG, CACCGG, AACCGG, GGTCGG,
CGGATT, TCGGAG, AGTCGG, CATCGG, CTCCGG, CGGGAT, CTGGGT, TTACGG, TGGGTC, TACGGG,
TCGGAT, CGGAGT, CGGATG, CGGACG, GTCGGA, TATCGG, CGGAAC, TACGGA, GCCGGA, TTGGGT,
TGGGTT, GCTCGG, ACCCGG, ATCGGA, CGGACA, ACGGAA, CCGGGA, GACGGG, CGGAAG, ATACGG,
CGGAGA, ACGGAC, TCTCGG, GTCCGG, CGGGAG, AGACGG;
−1.7; CGCCCA, TCGCAA, TCGCTC, CGCTCA,
CGCATG, GCGACA, TCGAGG, AAGCGA, ACGCTC, ACGCTA, GTCGCA, GCGAGG, TATCGC, CGCAAT,
CGCTAA, GAGCGA, CGCTCC, TGCAGT, GTAGCG, CGCCAT, GCCCGC, TTCGCT, CGCTTA, CGCACA, ACGCAC,
CGCTCG, AGCGAC, ACGCCA, CCAGCG, GCACGC, CTAGCG, GGTCGC, GCGCTT, CGCATA, CAGCGT,
GCGCCA, CGCTAT, CGCCGA, GCGTCA, CGCTAG, GTACGC, CGCCTG, CGAGCG, AAACGC, TTCCGC,
ACGCAG, ATTCGC, CGATGG, CCGCAC, GCGCTC, CGCCCC, CGCCCT, TCGCCC, CTCGCT, CGCCCG, AGCGCC,
TACCGC, AACCGC, GCGTAA, TCGCTG, TGAGCG, CGTTGG, AACGCT, CGCATC, ATCGCA, GCTCGC,
GCGACG, CGAGGA, ACAGCG, TAGCGT, TACGCT, ACGCTG, GCGTCG, CGCCTC, GAGCGC, CGTAGG,
GCGCTG, CCGCCG, TCCGCC, ACTCGC, ACCGCC, TGTCGC, GCGATA, AACGCA, ACCCGC, CAAGCG,
GCGCAT, CCCCGC, AACGCC, AATCGC, GCGTCT, TTCGCC, TCCCGC, GCGACC, CGCTCT, GCGTTC, CCCGCC,
CCGCAA, GACGCT, CGCTGA, GAGCGT, CGCCTA, ACGAGG, GCGCCG, TCGCCG, CACGCC, ACGCAA,
ACGCAT, CTACGC, CGCATT, AAGCGC, CGCAAG, CAGCGA, GCGCAA, GACGCC, GCGATT, ACGCCC,
GCGTAG, GCGCAC, CGCTTT, CCGCTA, CTCCGC, CGCTTC, CGCAAA, CGCTAC, TCGCCT, TAGCGC, GCGAAT,
TACGCA, ACCGCT, CACCGC, CCGCTC, GCGTTT, GAACGC, GCGTTA, TCCGCT, TAACGC, GATCGC, ACACGC,
CTCGCC, AGAGCG, TTTCGC, CCGAGG, CCGCAG, GTCGCC, GCGTAC, GCGATG, CCGCCC, GTTCGC,
GGACGC, TAAGCG, TCGCCA, TCGCAT, CCGCTG, CGACGC, AGCGAA, TCGCTA, ATACGC, CGCACG,
GCGCAG, CCACGC, AGCGAT, CAGCGC, AGACGC, CGCAAC, TCAGCG, CACGCA, GGAGCG, CAACGC,
CGCCAG, TAGCGA, GCGATC, AGCGCT, GCGCCC, CGCAGA, GAAGCG, GCGTTG, GCGTAT, AGCGTT,
CATCGC, GCGAGA, TTCGCA, TGCTGT, CGAGGG, CGCACT, CGCCAC, ATCCGC, GACCGC, CGCTTG, TTACGC,
TGACGC, TGCCGT, TACGCC, GCGCCT, ACGCCT, CCGCCA, GCGTCC, CGCCAA, CCGCCT, CGCCTT, AGTCGC,
ACCGCA, AGCGTC, TCGCAC, GCGACT, ATCGCC, GTCCGC, TCTCGC, ATAGCG, CTTCGC, ATCGCT, CCGCAT,
CCGCTT, ACGCTT, GCGCTA, CCTCGC, AGCGTA, GCGAAG, ACGCCG, TTAGCG, AAAGCG, AGCGAG,
CTCGCA, CGCACC, GACGCA, GCGAAC, TCGCTT, AAGCGT, AGCGCA, TCGCAG, CACGCT, CCCGCA, GTCGCT,
GCGAAA, CCCGCT, TCCGCA, TCACGC;
−2: GCACGG, CACGGA, CCACGG, CACGGG, ACACGG, TCACGG;
−2.1: ATGGTC, ATGGTT, TGGTGA, AATGGT, TTGGTT, TGCCGG, TTGGTA, TGGTTC, TGCTGG, TGGTCA,
TGGTCT, TGGTCG, TGGTGT, CTGGTT, CTGGTG, TGGTAC, TATGGT, CGGAGC, TGGTAT, TGGTTA, ATGGTA,
TTTGGT, CTGGTC, CCTGGT, TGGTAG, TGACGG, CTGGTA, ATGGTG, TGGTTG, GATGGT, GTTGGT, ACTGGT,
TTGGTC, TGGTAA, TCTGGT, TGGTGC, TGCAGG, TGGTTT, CTTGGT, CATGGT, TGGTCC, ATTGGT, TGTCGG,
TTGGTG;
−2.2: CGTGAG, CGTGTG, GCCGCT, ATCGTG, ACCGTG, GACGTG, TGAGGT, CGTGTT, ACGTGT,
CCGTGA, CGTGAC, AGCTGC, CGTGAA, TCCGTG, CGTGAT, ACGTGA, GCTGCA, TACGTG, GCCGCA,
CACGTG, GCAGCC, CCGTGC, CGTGTA, AGCGTG, AACGTG, GCCGTG, CGTGCC, GTCGTG, AGCCGC,
GCAGCA, GCAGCT, AGCAGC, GCGTGA, CTCGTG, CGTGCA, CGTGCT, CCCGTG, TTCGTG, GCAGCG,
TCGTGC, CGTGTC, GCTGCT, CCGTGT, GCTGCC, TCGTGT, ACGTGC, GCCGCC, TCGTGA;
−2.3: ATGGGG, TTGGGG, TGGGGA, CTGGGG, TGGGGG;
−2.5: CGTCGC;
−2.6: GGCTGC, TCTGCG, AGGCAC, TGCGTT,
GGGCAC, GGCTCA, CAGGCT, GGCTTG, ACAGGC, GGCACA, GGCCGG, GGCAGC, TGCGCT, AAGGCG,
TTGCGT, AGGCCA, GGCCTA, GCTGCG, GGCTGG, CTTGCG, AAGGGC, GGCTAA, CTGCGA, TGCGCC,
GGCTAT, ATTGCG, GGCCAA, AGGCTT, GGCTTT, TAGGCC, CATGCG, GGGCGC, CAGGCA, GGCAGG,
GATGCG, GGCTGA, TGCGAG, GGCGTA, GGCCCT, AGGCTG, AGGCGT, AGGCCT, GGCGCC, GGGCCA,
AGGGCT, CTAGGC, TAAGGC, GTTGCG, GGCGAT, GGCCCG, AGGGCC, GGCGTC, TTTGCG, GGGCAG,
GGCACT, CAGGCG, GGCCAG, GGCCAT, ATAGGC, GGTGCG, GGCTTA, GGCACG, GGCGAA, GGCTTC,
AGGCTA, GGCCGC, GGCTCG, CCAGGC, AGGCCG, AGGCAG, CGTGCG, TTAGGC, GGCACC, GGCGCA,
GGCATG, GGGCAT, GGCAAG, GGCATT, AGGCGC, TTGCGA, GGCGAC, AGGGCA, GGGCTA, ATGCGC,
AGGCAA, GGGCAA, AATGCG, TTGCGC, GGCCCA, GGGCTC, AAGGCC, CTGCGT, ACTGCG, AAGGCA,
AGTGCG, TAGGGC, AAGGCT, TGTGCG, GGCCGA, GGCAAT, GAGGGC, AAAGGC, TGCGAT, GGCAAC,
TAGGCT, TATGCG, GGCCTC, GGGCTG, CAGGCC, GGGCGT, AGGCGA, GGCTCC, GGCAGT, GGCAGA,
TCAGGC, GGCGTG, CAAGGC, GGCTAC, AGGCTC, GGCATA, TGCGTC, TGCGTA, GGCTCT, CTGCGC,
AGGCAT, GTGCGT, GGCGAG, TAGGCA, TGCGCA, GGCTGT, TGCGAC, GGCCAC, GGCTAG, GGCCCC,
GGGCTT, GGCCTT, GGGCCT, GGGCCG, GGGCGA, GGCATC, AGGCCC, GGCCGT, GGCCTG, CCTGCG,
GGCAAA, TGCGAA, TGCGTG, ATGCGA, ATGCGT, CAGGGC, GGGCCC, AGGGCG, GGCGCT, GGCGTT,
GTGCGA, TAGGCG, GAAGGC;
−3: CGTAGC, TTGGGC, TGGGCA, TGGGCC, TGGGCT, CTGGGC, CGTTGC, ATGGGC, TGGGCG;
−3.1: AGGTGG, GGGTGG, GGTGGG, AAGTGG, GTGGAG, GTGGAC, GTGGGC,
GTGGAT, TGCTGC, CCGGGT, GTGGAA, GTGGGA, TCGGGT, CGGGTA, TGGTGG, GAGTGG, GTGGGG,
CAGTGG, TAGTGG, GTGGGT, GGTGGA, TGCAGC, CGGGTT, CGGGTC, ACGGGT, CGGGTG, AGTGGG,
TGCCGC, AGTGGA;
−3.2: GCGTGT, CGCCGT, GCAGGT, CGCAGT, CGCTGT, GCTGGT, GCGAGT;
−3.4: GGGGGT, GGGGTT, GGGGTG, AGGGGT, GGGGTC, GGGGTA;
−3.5: GTTGGC, GATGGC, TTGGCA, TGGCGC,
ATGGCG, TTTGGC, ATGGCT, AATGGC, TGGCTA, TATGGC, TGGCTC, TGGCAA, TGGCAG, CTTGGC, TTGGCC,
TTGGCG, TGGCGT, CATGGC, ATTGGC, ACTGGC, ATGGCA, TGGCTT, TGGCGA, CTGGCG, TGGCCT,
TGGCAC, CTGGCC, TCTGGC, CTGGCT, TGGCCA, TGGCAT, TTGGCT, TGGCCG, TGGCTG, ATGGCC, CCTGGC,
CTGGCA, TGGCCC;
−3.6: CCGGTA, CCGGTG, TCGGTT, CGCTGG, TCGGTA, CTCGGT, TCCGGT, CGGTGG,
TCGGTC, CGCCGG, CGGTCG, TACGGT, CGGTAC, ACCGGT, CGGTGC, CGGTGA, ACGGTA, TTCGGT,
CGTCGG, CGGTTG, CCGGTC, CCCGGT, TCGGTG, CGGTTC, CGGTAT, CGGTTA, GACGGT, GTCGGT,
CGACGG, CGGTCC, TGAGGC, CGGTAA, ACGGTT, ACGGTG, CGGTAG, AACGGT, CGGTTT, ATCGGT,
CGCAGG, CCGGTT, CGGTCT, GCCGGT, ACGGTC, CGGTCA, CGGTGT;
−3.7: CGAGGT:
−3.8: CGGGGG,
ACGGGG, CCGGGG, CGGGGA, TCGGGG;
−4: TGTGGG, GTGTGG, CTGTGG, CACGGT, TTGTGG, TGTGGA,
ATGTGG;
−4.1: CGCGCC, GACGCG, CGCGAT, ATCGCG, CGCGCG, GCCGCG, ACGCGA, CCGCGA, CGCGAG,
CCGCGT, AACGCG, TCGCGA, CGCGAC, CGCGTG, TTCGCG, ACGCGC, TCGCGT, TCGCGC, TACGCG,
TGCGCG, CGCGCT, CCGCGC, ACCGCG, CGCGTT, GTCGCG, ACGCGT, CGCGCA, TCCGCG, CGCGTA,
CGCGAA, GCGCGA, GGCGCG, CACGCG, CCCGCG, CTCGCG, CGCGTC, GCGCGT, AGCGCG;
−4.3: TGGGGT;
−4.5: CCGGGC, CGGGCG, CGGGCT, TCGGGC, ACGGGC, CGGGCA, CGGGCC;
−4.6: CGCCGC, GCGAGC,
GCTGGC, GCAGGC, GCGCGC, CGCAGC, GCGTGC, CGCTGC;
−4.8: GGGGGC, GGGGCA, GGGGCC, AGGGGC,
GGGGCT, GGGGCG;
−5: CTCGGC, ACGGCT, CCGGCG, TGGCGG, CCCGGC, CGGCGT, AGGCGG, ACGGCG,
GCGGGA, AACGGC, GCCGGC, TTCGGC, TCGGCG, GCGGGG, GCGGAC, CCGGCC, CCGGCT, GAGCGG,
GGCGGA, CGGCCC, TCCGGC, ACGGCA, CGGCTG, AGCGGA, CGGCGC, CGGCTA, CGGCCT, CGGCAA,
CGGCTT, CAGCGG, CGGCGA, ACCGGC, CGGCGG, ACGGCC, TCGGCC, TAGCGG, GACGGC, GCGGGT,
AGCGGG, GTCGGC, CCGGCA, TCGGCT, CGGCAC, GGCGGG, GGGCGG, CGGCCG, AAGCGG, GCGGAT,
TCGGCA, ATCGGC, CGGCAG, GCGGAA, GCGGAG, CGGCTC, GCGGGC, CGGCCA, TACGGC, CGGCAT;
−5.1: GGTGGT, CGAGGC, GTGGTA, GTGGTC, AGTGGT, GTGGTG, GTGGTT;
−5.4: CACGGC;
−5.5: ACGTGG,
TCGTGG, CGTGGA, GCGTGG, CGTGGG, CCGTGG;
−5.7: TGGGGC;
−5.8: CGGGGT;
−5.9: CTGCGG, GTGCGG,
TTGCGG, TGCGGG, TGCGGA, ATGCGG;
−6: TGTGGT;
−6.5: GTGGCT, GTGGCG, GTGGCA, GGTGGC, GTGGCC, AGTGGC;
−7: AGCGGT, GGCGGT, GCGGTT, GCGGTA, GCGGTG, GCGGTC;
−7.2: CGGGGC;
−7.4: GCGCGG, ACGCGG, TGTGGC, CCGCGG, TCGCGG, CGCGGG, CGCGGA;
−7.5: CGTGGT;
−7.9: TGCGGT;
−8.4: GCGGCT, AGCGGC, GCGGCA, GGCGGC, GCGGCG, GCGGCC;
−8.9: CGTGGC;
−9.3: TGCGGC;
−9.4; CGCGGT.,
CGGCTG aSD:
−0.1: AACAGA, TCACCC, GTCAGA, CAACCT, GTGCAG, ACCAGG, GCAACC, GACAGG,
ACAGGA, TCACAG, CCAGAG, CTCAGG, CAACAG, TTCCAG, CTACAG, ATCCAG, CCAGAA, ACGCAG,
ACAGAA, CAGAAA, GCATCC, CAGGGG, TACCAG, CATGCG, TCTCAG, GTCCAG, GTCAGG, GCAGGG,
AAACAG, CCAGAC, ACAGAG, CAGAGA, ACTCAG, AACCAG, CACAGA, GAACAG, AGATCG, TTACAG,
CCAGAT, CAGAAC, CCATCC, GGTTCG, ACAGAT, AGTTCG, CACCCT, CCCCAG, GGTACG, CTTCAG, CTCCAG,
CACCCA, CAGGAC, CCACAG, CATCCT, GGTGCG, TCATCC, CAGGGA, CAGACA, TCCAGG, TATCAG, GCACCC,
ATCAGA, TGACAG, CAGATT, TCAGAT, CAGAGT, TTGCAG, TCAGAG, CATCAG, TGCAGA, TCAGGG,
CAGGGT, TCAACC, CACAGG, TAACAG, TACAGG, AACAGG, CCAGGA, CATCCC, ACACCC, GCAGAA,
GTACAG, CCAACC, AGTGCG, ACAGGG, ATGCAG, CCCAGA, ACAACC, ACATCC, ACAGAC, ACACAG,
CAGAAT, GCGCAG, ACCCAG, CCTCAG, CACGCG, TCAGAC, TTCAGG, CAGATA, GATCAG, CACCAG,
CATCCA, CGCAGA, TGCAGG, CCCAGG, ATCAGG, TCCAGA, GCACAG, AGTACG, TTCAGA, CGACAG,
AGACAG, GGATCG, GCAGAG, CCACCC, GACCAG, CGTCAG, CAGGAA, ATACAG, AATCAG, CAACCA,
TGTCAG, GCAGAT, TCCCAG, ATTCAG, TCAGAA, GGACAG, CGCAGG, TACAGA, TCAGGA, TTTCAG,
CAGGAT, CTGCAG, GCAGGA, ACCAGA, CCAGGG, CAACCC, CTCAGA, GTTCAG, CACCCC, GACAGA,
TCGCAG, GCAGAC, CAGATG;
−0.3: AGGTGT, TGTTGC, CTGTTG, CACGTC, ATGTTG, TGGGTG, TTGTTG,
TGTTGA, GTTGTA, TCGTTG, CAACTG, CGTTGG, CATCTG, GGGTGT, CGTTGT, GTTGCG, GGGTGA, CATGTC,
GAGGTG, GTTGAT, GTTGAA, GTTGGG, AGGTGC, ACGTTG, GGGGTG, TGTTGG, GTTGTC, TCGGGT,
CGGGTA, CGTTGA, AAGGTG, GGGTGC, CGTTGC, GTTGTT, GTTGTG, GTTGGT, GTTGCA, GTTGAC,
GTTGAG, GTGTTG, AGGTGA, GCGTTG, TGTTGT, CGGGTT, CAGATC, ACGGGT, GTTGGA, CACCTG,
AGGGTG;
−0.4: GGACCT, TAGACG, GGACCA, CAAACG, CAGAGG, AGACCT, AAGACC, CAGGAG, TGGACC,
AGACCC, GGGACC, AGGACC, GGACCC , AGACCA, GAGACC;
−0.5: GGTAGT, TGTAGT, CTTAGT, CTAGTG,
GTAGTG, GTAGTA, ATTAGT, TAGTAC, ATAGTA, ATAGTG, TAGTAT, TAGTGT, AGTAGT, CATAGT, TTAGTG,
CGTAGT, TTTAGT, TAGTGC, TAGTAG, TATAGT, TTAGTA, TAGTGA, CTAGTA, TCTAGT, GATAGT, GTTAGT,
ACTAGT, AATAGT, TAGTAA, CCTAGT;
−0.6: CGGACT, GGACTG, CAGAAG, AGACTG;
−0.7: CTGCGG,
GCGGGA, ACGCGG, GCGGGG, GCGGAC, GTGCGG, TTGCGG, TCGCGG, CGCGGG, GCGGGT, TGCGGG,
TGCGGA, GCGGAT, GCGGAA, GCGGAG, CGCGGA, ATGCGG;
−0.9: GCTAAG, CGTGGC, TCGCTC, CGCTCA,
GTTGGC, GCTATG, AGGCAC, AAGCGA, GCTTTA, ACGCTC, GGGCAC, CAAAGC, ACGCTA, GTAGGC,
GGCACA, CGAAGC, GCTTCG, TTGCTA, TTGGGC, GGAAGC, TGCGCT, CGCTAA, GCTTAC, GCTCAC, TGCTAT,
GAGCGA, CGCTCC, GATGGC, GGGGGC, TTGGCA, TGAAGC, TTCGCT, CGCTTA, AGCGAC, GCTTTG,
GCGCTT, TGGCGC, CGCTAT, AAGGGC, GCTACT, CGAGCA, GCTTGG, ATGCTA, CGCTAG, GTTGCT,
ATGGCG, CGAGCG, GTGCTT, GCGAGC, GGTGCT, GAGCAG, AGAGGC, GCGCTC, TTTGGC, AGCAAG,
GTGGCG, CTCGCT, TTGAGC, CGGGGC, ACGAGC, GATGCT, AGCACA, GAAAGC, AATGGC, TGAGCG,
GGAGGC, GGCAGG, AGGAGC, AACGCT, GCTACC, GGCGTA, GCTAAA, TATGGC, AAGCAT, GCTTAG,
ATTGCT, TACGCT, GCTTTC, TCTGCT, AGCATT, GAGCAT, TTGCTC, TGGCAA, GAGCGC, GTGGCA, AAGCAG,
TGGCAG, CTTGGC, GAGCAC, CTGAGC, CTAGGC, TGGGCA, GCTTGC, TAAGGC, CCTGCT, GGCGAT,
TGCTCT, TGCTAC, TGGAGC, GGCGTC, AAAAGC, CAAGCG, GCTAGG, CGGAGC, GCTTGT, GTGGGC,
GGGCAG, GGCACT, CTGCTT, TTGGCG, GGGGCA, TGCTTC, GCTTGA, GAGAGC, CGCTCT, ATAGGC,
GCTATT, CTAAGC, TGTGGC, TCAAGC, GACGCT, GCTTCC, AGCAAA, CGAGGC, GGCGAA, TGGCGT,
TGCTAA, GAGCGT, CATGGC, GCTCCT, GCTCTC, CTGCTC, CAGAGC, ATAAGC, AGGCAG, AAGCGC,
ATTGGC, TTAGGC, CCAAGC, GGCACC, CTGCTA, GGAGCA, AGCAGG, GGGAGC, GGCGCA, GGCATG,
AAGCAC, CGCTTT, AGCGTG, CTGGGC, CGCTTC, GAGCAA, GGGCAT, GGCAAG, GGCATT, TGCTTG,
CGCTAC, TTGCTT, ACTGGC, ATGCTC, AAGAGC, TGCTTA, GGCGAC, ATGGCA, GCTCTG, GCTATC, AGGGCA,
CTTGCT, CGCGCT, AGGCAA, AGCATA, GGGCAA, ACAAGC, GCTTTT, TGGCGA, TGGGGC, GCTTCT,
AAGGCA, TAGGGC, CTGGCG, AGAGCG, GCTAGT, TCGAGC, GCTCTT, GGCAAT, GAGGGC, AGCAAT,
AAAGGC, GGCAAC, GCTCTA, TAAGCG, AGCGAA, TCGCTA, ATGGGC, GTGCTC, GTAAGC, AGCATG,
ATGCTT, AGAAGC, TTTGCT, TGGCAC, GCTCAG, TGAGGC, AGCGAT, AAAGCA, GGCAGA, GTGCTA,
GGCGTG, AGCACT, GGAGCG, CAAGGC, TCTGGC, AGAGCA, AGCGCT, GCTTAT, GAAGCA, GGCATA,
GCTAAC, GAAGCG, AGCGTT, TGCTAG, GCTACA, TAGAGC, AAGCAA, CGTGCT, CGCTTG, GCTCCA,
AGGGGC, AGGCAT, TAAAGC, GTGAGC, AGCATC, GCTACG, GGCGAG, TAAGCA, TAGGCA, GCTTAA,
TGGCAT, AGCGTC, TATGCT, GCTTCA, TTAAGC, GCAAGC, GAGGCA, GCTAGA, ATCGCT, ACGCTT, TGCTCA,
GCGCTA, GCTATA, AGCAAC, TGCTTT, AGCGTA, CAAGCA, GCTCCC, GGCATC, TGAGCA, AATGCT, AAAGCG,
GCTCAT, AGCGAG, ATGAGC, AGCAGA, CCTGGC, ACTGCT, AGTGCT, AGCACC, TGCTCC, GGCAAA, TCGCTT,
AAGCGT, AGCGCA, CTGGCA, CAGGGC, GGCGCT, TGTGCT, GCTCAA, GGCGTT, GCTAAT, GAAGGC;
−1: CTAGTT, TAGTTT, TAGTTC, GCAGGT, ACAGGT, GTAGTT, TTAGTT, ATAGTT, CAGGTT,
CAGGTA, CCAGGT, TCAGGT, TAGTTA;
−1.2: GCACGG, CGCTCG, GCACGC, CGCGCG, GCGCGG, TGCACG, GCTCGC, TGCTCG,
GCACGA, GCTCGA, GCACGT, TGCGCG, GCGCGC, GCTCGT, GCGCGA, CGCACG, GCGCGT, GCTCGG;
−1.3: CATGCT, TAGGTG, CGGACG, CAGACT, CACGCT;
−1.4: GGTGGT, AGGTGG, TAGACC, TCGGTA, GGGTGG,
CTCGGT, TGCGGT, CGCGGT, GGTGGG, TACGGT, AAGTGG, CGGTAC, GGTGGC, CGGTGC, CGGTGA,
ACGGTA, TTCGGT, CACGGT, TGGTGG, TCGGTG, GAGTGG, CGGTAT, GACGGT, GGTGGA, AGTGGT,
CGGTAA, ACGGTG, CGGTAG, AGTGGC, AACGGT, GCGGTA, ATCGGT, GCGGTG, AGTGGG, CGGTGT,
AGTGGA;
−1.5: ATGGTC, GGTCTA, GAGTCT, TGGTCA, AGTCTG, CGAGTC, AGTCAT, AAGTCT, TGGTCT,
TAGGTC, GGGTCT, GGTCCT, GGGTCA, GGTCCC, AGTCCT, GAGGTC, TAAGTC, AAGTCC, GGTCAT, AAAGTC,
CAAGTC, AGGTCT, AGGTCA, GGTCTT, CTGGTC, AGTCTT, GGAGTC, AGTCAG, AGTCAA, AGTCCA, GTGGTC,
AGTCTC, TTGGTC, GGTCTC, AGTCAC, GGTCAG, GGTCAC, GGTCTG, GAGTCA, GGTCCA, AGTCTA, AGGGTC,
TGAGTC, AGGTCC, CAGGTC, CGGGTC, AGAGTC, TGGGTC, GGGGTC, AGTCCC, AAGTCA, GGGTCC,
TGGTCC, GGTCAA, GAAGTC, GAGTCC, AAGGTC;
−1.6: CCGGTA, CCGGTG, ACCGGG, ACCGGA, GTCCCG,
ACCGAA, CCGAAG, CCGGAC, AACCGT, TCCGAT, CCGTAT, TCCGGT, TCCCCG, TCCCGG, CCGAGA, TCCGGA,
CCGTCA, CCGACG, ACCGAG, TTCCCG, GACCCG, ACCGTG, TTCCGC, ACTCCG, TGACCG, CCCCGG, CCGCAC,
GCTCCG, ATCCGG, GATCCG, TAACCG, TACCGC, CCCGGG, AACCGC, CCGTGA, CCCGTT, CCGCGA, CCGATC,
CCGACA, ATCCGA, TATCCG, CCGCGT, CCGGAG, CCGTTG, TTACCG, CCCGAC, ACCGAT, CTTCCG, CTCCCG,
GACCGA, ACCCGC, ACCGGT, TTCCGT, CCCCGC, CCGAAA, CCGAGT, CCGAAC, TCCGTG, CCCGAT, CCGACT,
TCCGAC, TACCGA, TCCCGC, CCGATG, ACCCGA, CCGCAA, TTTCCG, CCGGGT, TTCCGA, ATTCCG, CCCGTC,
TTCCGG, CCGCGG, TCCGAA, CCGTAA, TACCCG, CCGTCC, CCCGTA, AATCCG, CCGTTA, CCGTGC, CCCGGT,
CCGGGG, CCGCTA, CTCCGC, CCGTTC, CCGGAA, AACCCG, CCCGGA, ACCCGT, ACCGCT, ATACCG, CCGCTC,
GTACCG, TCCGCT, CCGCGC, GACCGG, TACCGG, GACCGT, CTCCGA, TCCGTC, TCTCCG, ACCGCG, TCCGGG,
CCGAGG, CCGGAT, CCGCAG, TCCGCG, ACCGAC, CCCCGA, ACCGTC, TCCGAG, CCGTCT, CCTCCG, TCCGTA,
CCGTAG, CCCGCG, AACCGG, TGTCCG, GTCCGA, CCGAAT, CCGAGC, CCCCGT, CCCGAG, CCGTTT, CCCCCG,
ATCCCG, TCCCGT, ATCCGC, GACCGC, CCCGTG, CTCCGG, CCGACC, TACCGT, CCGATT, CCCGAA, CTCCGT,
ACCGCA, TCCCGA, GTCCGC, CCGATA, CCGCAT, CCGCTT, CCGTGT, ATCCGT, CTACCG, ACCGTT, ACCCGG,
GTTCCG, ACCGTA, CCGGGA, CCCGCA, GTCCGT, AAACCG, GAACCG, TCCGTT, GTCCGG, CCCGCT, TCCGCA,
ACCCCG, AACCGA, CCGTAC, CCGTGG;
−1.7: CCGGGC, TCGGGC, ACGGGC, CGGGCA, GCGGGC;
−1.8: CGACCG, CGTCCG;
−1.9: TCGGTT, TTTAGC, ATTAGC, CGTAGC, AGTTGC, GTAGCA, GTAGCG, AGTTGA,
CTAGCG, GATAGC, AGGTTG, CATAGC, AGTTGT, GGTTGG, TCTAGC, TAGCGT, TAGCAA, AATAGC,
GTTAGC, GAGTTG, GCTAGC, GGTAGC, TGTAGC, TTAGCA, GGTTGA, CGGTTC, CCTAGC, TAGCGC,
ACTAGC, TGGTTG, AGTTGG, GCGGTT, CGGTTA, CTTAGC, TAGCAT, GGGTTG, ATAGCA, TAGCAG,
AAGTTG, GGTTGC, CTAGCA, ACGGTT, TAGCGA, GGTTGT, TAGCAC, TATAGC, CGGTTT, AGTAGC, ATAGCG,
CCGGTT, TTAGCG:
−2: CAGACG;
−2.1: CACCGT, TTCAGT, TGCAGT, CCACCG, ATCAGT, CCAGTA, ACAGTA,
CACCGA, TCAGTG, GCAGTG, TACAGT, CTCAGT, GCACCG, TCAGTA, CAGTAG, CCCAGT, CAGTGT, TCCAGT,
CGCAGT, CACCGC, GTCAGT, CAGTGC, CCAGTG, CACAGT, ACACCG, CAGTGA, GGCAGT, CACCGG,
CAGTAT, GACAGT, GCAGTA, AGCAGT, ACAGTG, AACAGT, CAGTAA, ACCAGT, CAGTAC, TCACCG;
−2.2: GAGGCG, AAGGCG, GGGCGC, AGGCGT, AGGCGC, GGGCGT, AGGCGA, CGGGTG, GGGCGA, GGGGCG,
AGGGCG, TGGGCG;
−2.3: CCGTCG, GTCGCA, GTCGAG, TGGCGG, GTCGTT, AGGCGG, GCGTCG, GTCGTC,
TGTCGC, GTCGAC, GTCGGG, GTGTCG, ATGTCG, GTCGAT, GAGCGG, GGCGGA, CGTCGC, CGTCGG,
TGTCGT, AGCGGA, AGCGGT, GGCGGT, TCGTCG, GTCGTG, GTCGCG, CTGTCG, GTCGGT, GTCGTA,
CGGACC, AGCGGG, TGTCGA, CGTCGT, CGTCGA, GGCGGG, GTCGGA, GGGCGG, GTCGAA, ACGTCG,
AAGCGG, TGTCGG, TTGTCG, GTCGCT;
−2.4: ACAGGC, CAGGCA, GCAGGC, CCAGGC, TAGTGG, TCAGGC;
−2.5: GGCTCA, CAGGCT, GGCTTG, CACCCG, GTGGCT, CAACCG, GGCTAA, GGAGCT, GGCTAT, AGGCTT,
GGCTTT, GAAGCT, ATGGCT, AGCTAG, TGGCTA, TGGCTC, CATCCG, AGCTTT, AGGGCT, AGCTTA, AGCTTG,
TGAGCT, TGGGCT, CGGGCT, ATAGTC, TAAGCT, GGCTTA, GGCTTC, AGGCTA, CAAGCT, AGAGCT, AGCTTC,
AAGCTA, GGGCTA, AGCTAC, AAGCTT, TGGCTT, GGGCTC, AAGGCT, AGCTCA, TAGGCT, AGCTCT, AAGCTC,
TAGTCT, GGCTCC, AGCTAA, AGCTAT, GGCTAC, GAGCTT, CTGGCT, AGGCTC, TAGTCC, GGCTCT, AAAGCT,
TAGTCA, GGGGCT, GAGGCT, CGAGCT, GAGCTA, GGCTAG, TTGGCT, GGGCTT, GTAGTC, CTAGTC,
GAGCTC, TTAGTC, AGCTCC;
−2.6: CGCCCA, CGCGCC, GCCCTC, GCCCGT, GCCCGA, TGCCCC, GCCTAC,
CGCCAT, GCCTGG, GCCCGC, AATGCC, ACGCCA, GCGCCA, GCAGTT, GCCCTG, TGCGCC, GCCAAC,
CGCCTG, GCCAGA, TTTGCC, CGCCCC, CGCCCT, CAGTTC, CAGTTT, GCCATT, TCGCCC, GCCATG, CGCCCG,
AGCGCC, GCCCTA, ACAGTT, GCCACC, GCCTAA, GCCTGT, GGCGCC, CGCCTC, TGCCCG, GCCACG, CTGCCT,
TCCGCC, ACCGCC, TGCCTG, ATTGCC, AACGCC, GCCTCG, GCCCGG, GCCTTG, TTCGCC, GCCTCC, GTGCCA,
CCCGCC, GCCAAG, GCCTCT, TGCCAT, GCCACA, TGCCAC, TCAGTT, CGCCTA, GCCACT, GCCCCC, GTGCCT,
GGTGCC, GCCTGA, CCAGTT, ATGCCT, GACGCC, ACGCCC, GCCAAA, TGCCAA, TCGCCT, ATGCCC, GATGCC,
CGTGCC, GCCCCT, TATGCC, TCTGCC, GCCTTA, GCCTGC, CTTGCC, TTGCCC, ATGCCA, CTCGCC, GCCCAT,
GTCGCC, CCGCCC, AGTGCC, TCGCCA, CTGCCC, TGCCTC, TGCCTA, GCCCAA, CAGTTA, GCCCTT, CGCCAG,
GCCAGG, CCTGCC, GCCCAC, TGCCCT, GCGCCC, GCCTAG, TGCCAG, GCCAAT, GCCTCA, CGCCAC, GCCATC,
GCCAGT, TACGCC, GTGCCC, GCGCCT, ACGCCT, CCGCCA, TTGCCT, GTTGCC, GCCTTC, CGCCAA, CCGCCT,
CGCCTT, GCCTAT, TGCCTT, ATCGCC, TGCCCA, TGTGCC, ACTGCC, GCCTTT, CTGCCA, TTGCCA, GCCCAG,
GCCCCG, GCCATA, GCCCCA;
−2.8: CGCTGG, GCTGTG, CTCGGC, CCGGCG, GCTGCG, TGCTGG, CCCGGC,
AGTCCG, CGGCGT, AGCTCG, ACGGCG, GCTGTC, AACGGC, TCGCTG, GCTGGC, TTCGGC, ACGCTG,
GCTGAC, TCGGCG, GCGCTG, CGCGGC, AGACCG, GCTGCA, TGCTGC, GGACCG, GGCACG, CGCTGA,
TCCGGC, GTGCTG, ACGGCA, GGCTCG, TGCTGA, GCTGTA, ATGCTG, CGGCGC, CGGCAA, GCTGGA,
CGGCGA, ACCGGC, TGCGGC, AGCGGC, GGTCCG, GCTGAG, TTGCTG, CCGCTG, GACGGC, GGCGCG,
CGCTGT, GCTGGT, CACGGC, GTCGGC, CCGGCA, TGCTGT, GCTGTT, CGGCAC, AGCGCG, AGCACG,
GCTGCT, GCTGGG, GCGGCA, TCGGCA, ATCGGC, GGCGGC, GCTGCC, CGGCAG, GCGGCG, CGCTGC,
GCTGAA, TACGGC, CGGCAT, CTGCTG, GCTGAT;
−2.9: TAGTTG, CAGGTG;
−3: CAGACC, CACGCC, CATGCC;
−3.2: TAGGCG;
−3.3: CGGTGG, TAGCGG;
−3.4: TCGGTC, CCGGTC, CGGTCC, CGGTCT, GCGGTC, ACGGTC, CGGTCA;
−3.5: GCCAGC, GGCAGC, ACCAGC, CCAGCG, CAGCGT, CACAGC, CAGCAC, GTAGCT, CCCAGC,
GTCAGC, CTCAGC, ACAGCG, AACAGC, ATAGCT, CAGCAG, TAGCTC, CAGCGA, CCAGCA, ACAGCA,
GCAGCA, TCAGCA, TTCAGC, CGCAGC, CAGCGC, CTAGCT, TAGCTT, TCAGCG, TGCAGC, ATCAGC,
TACAGC, AGCAGC, TCCAGC, GCAGCG, CAGCAT, CAGCAA, TAGCTA, GACAGC, TTAGCT;
−3.8: CGGTTG;
−3.9: GGTCGA, GGTCGC, TGGTCG, GAGTCG, AGGTCG, GGGTCG, AAGTCG, AGTCGA, GGTCGT, GGTCGG,
AGTCGG, AGTCGC, AGTCGT;
−4: CAGTGG;
−4.1: TCAGTC, ACAGTC, CGGGCG, GCAGTC, CCAGTC, CAGTCC,
CAGTCA, CAGTCT;
−4.2: GGAGCC, GAGCCT, AGGCCA, GGCCTA, AGCCCA, GGCCAA, TAAGCC, AAGCCT,
GAGGCC, TAGGCC, GGCCCT, GAAGCC, AAGCCC, AGGCCT, GGGCCA, AGAGCC, TTGGCC, GGCCCG,
AGGGCC, TGGGCC, GTGGCC, AGCCCC, AGCCTC, GGCCAG, GGCCAT, AGCCAC, AAAGCC, AGCCAT,
TGAGCC, CAAGCC, GGGGCC, AGCCAA, AGCCTG, AGCCTA, GAGCCA, AGCCTT, GGCCCA, AAGGCC,
CGGCGG, AGCCCT, TGGCCT, GGCCTC, CAGGCC, CTGGCC, AGCCAG, TGGCCA, AGCCCG, CGGGCC,
CGAGCC, AAGCCA, GGCCAC, GGCCCC, GGCCTT, GGGCCT, AGGCCC, GGCCTG, ATGGCC, GAGCCC,
TGGCCC, GGGCCC;
−4.4: GGCTGC, GCGGCT, ACGGCT, GGCTGG, GGCTGA, AGGCTG, AGCTGC, CCGGCT,
AGCTGA, CGGCTA, CGGCTT, GAGCTG, GGGCTG, AAGCTG, AGCTGT, TCGGCT, GGCTGT, AGCTGG,
TGGCTG, CGGCTC;
−4.5: CAGTTG;
−4.8: CAGGCG;
−4.9: TAGTCG;
−5: GCCGTC, GCCGCT, CGCCGC, CTGCCG,
TGCCGG, CGCCGA, CGCCGG, GCCGCG, GCCGGC, GCCGTA, CCGCCG, GCCGGG, GCCGTT, GCCGCA,
CGCCGT, GCGCCG, GTGCCG, GCCGAG, TCGCCG, GCCGAC, GCCGTG, GCCGAA, TGCCGA, TTGCCG,
GCCGAT, ATGCCG, TGCCGT, GCCGGA, ACGCCG, GCCGGT, GCCGCC, TGCCGC;
−5.1: CAGCTC, CCAGCT,
CAGCTA, GCAGCT, CAGCTT, ACAGCT, TCAGCT;
−5.2: TAGCCC, CTAGCC, ATAGCC, GTAGCC, TAGCCA,
TTAGCC, TAGCCT;
−5.4: TAGCTG;
−5.8: CGGTCG;
−6.1: CCGGCC, CGGCCC, CGGCCT, ACGGCC, TCGGCC,
CGGCCA, GCGGCC;
−6.3: CGGCTG;
−6.5: CAGTCG;
−6.6: AGCCGA, GAGCCG, GGCCGC, AGGCCG, AGCCGT,
AGCCGG, AGCCGC, GGCCGA, GGGCCG, GGCCGT, TGGCCG, AAGCCG;
−6.8: CAGCCA, TCAGCC, GCAGCC,
CCAGCC, CAGCCT, ACAGCC, CAGCCC;
−7: CAGCTG;
−7.6: TAGCCG;
−8.5: CGGCCG;
−9.2: CAGCCG.,
CTCCTT aSG:
−0.4: ATGAGA, CGTGAG, CGAGAC, GAGTGT, GAGTCT, GAGATT, GAGCCT, GAGCGA,
CCAGAG, GTCGAG, GAGTTT, CCGAGA, GAGACT, ATAGAG, CGAGCA, ACCGAG, CGAGTC, CGAGCG,
TACGAG, GCGAGC, GAGCAG, TGTGAG, ATCGAG, TTGAGC, CGAGTA, GAGAGA, ACGAGC, ATTGAG,
GACGAG, CTCGAG, TGAGCG, AAGAGA, GAGTCG, TGCGAG, CGAGAG, CAAGAG, TGAGAT, AGAGAT,
GAGCAT, CGCGAG, TGAGTG, GAGCGC, GAGCAC, CTGAGC, ACAGAG, CAGAGA, AGAGCC, GAGTAC,
ACGAGT, AGAGAA, TAGAGT, GAGTAG, ATGAGT, GAGTGA, TGAGCT, CCGAGT, ACGAGA, GAGTTA,
GAGAAT, GAGAGC, GAGTAT, TTGAGT, GAGCCG, GAGCGG, AAGAGT, GAGTGC, TGAGCC, GAGATA,
GAGTTG, ACTGAG, GAGCGT, GCCGAG, CTAGAG, GAGTAA, CAGAGC, TAAGAG, GAGACG, CACGAG,
CAGAGT, AGAGCT, TCAGAG, CGAGTT, GAGCAA, AATGAG, GAGTGG, AACGAG, GAGCCA, AAGAGC,
GAGCTG, TGAGAC, GAGATC, CTTGAG, CCTGAG, GAGATG, AGAGCG, TCGAGC, CATGAG, GCTGAG,
GAGAAG, CGAGAT, GTAGAG, CTGAGA, GTTGAG, TCCGAG, TTAGAG, AGAGTT, AGAGTG, GAGTCA,
AGAGCA, GAGCTT, CCGAGC, CCCGAG, TGAGTT, GCGAGA, TAGAGC, CGAGTG, TGAGTA, TGAGTC,
TGAGAA, TTGAGA, GTGAGC, TCGAGA, GCAGAG, AGAGTC, CGAGCT, AGAGTA, GTGAGT, GAGAAA,
CGAGCC, GAGTTC, AAAGAG, GATGAG, GAGCTA, CGAGAA, AGAGAC, TATGAG, TTCGAG, TAGAGA,
GAGAAC, GCGAGT, TGAGCA, GAGAGT, GAGCTC, ATGAGC, TCGAGT, GAGCCC, TGAGAG, TTTGAG,
GAGACC, GAAGAG, GAGTCC, CTGAGT, GAGACA, TCTGAG, GTGAGA;
−0.8: GATAGG, ACCGGG, AGGCAC,
AATGGG, GGGCAC, AGGTAT, CAGGCT, ACAGGC, GTAGGC, ACTAGG, GGGTTC, ACCAGG, TTGGGC,
TAGGTT, GTAGGT, GACAGG, AGGCCA, ATCGGG, CTCAGG, TCTAGG, TGGGTA, AGGTTG, AGGCTT,
TAGGTC, AGGCGG, CCTGGG, TAGGCC, TGTGGG, CCCGGG, GGTGGG, GGGCGC, CAGGCA, GGCAGG,
AGTAGG, GTCAGG, AGGCTG, GGGTTA, GGGTCT, GCAGGC, AGGCGT, AGGTCG, GGGTAA, AGGCCT,
CCGGGC, CGGGCG, CGTAGG, GGGCCA, CTAGGC, TTTGGG, TGGGCA, GGGTCG, TGGGCC, GTCGGG,
GCCGGG, GCTAGG, TGGGCT, TTTAGG, GGGTCA, GTGGGC, CAGGCG, CGGGCT, ATAGGC, TCCAGG,
CCGGGT, TCGGGC, TAGGTA, AGGCTA, GTTGGG, AGGTAC, GATGGG, CATGGG, CCTAGG, AGGTCT,
CCAGGC, AGGTCA, ATGGGT, AGGCCG, ATAGGT, TTAGGC, TCGGGT, AGCAGG, TTCGGG, CGGGTA,
CTCGGG, CTGGGC, GCAGGT, GGGCAT, ACAGGT, ACGGGC, CACGGG, CACAGG, AGGCGC, TACAGG,
AGGTTA, AACAGG, AACGGG, GGGCTA, AGGCAA, GGGCAA, AGGTAA, GGGCTC, CGGGCA, TCCGGG,
TCTGGG, TTAGGT, AGGTTT, TGTAGG, CGCGGG, GGGTTG, TAGGCT, GGGCTG, ATGGGC, CAGGCC,
GGGCGT, GTGGGT, AGGCGA, AGGTTC, TCAGGC, GCGGGT, TTCAGG, GGGTTT, AGCGGG, GCCAGG,
CTTGGG, TGCGGG, TATAGG, TGCAGG, AGGCTC, AATAGG, CCCAGG, ATTGGG, ATCAGG, CGGGTT,
CAGGTT, AGGTCC, CAGGTC, AGGCAT, CTGGGT, CGGGTC, CAGGTA, CCAGGT, GGGTAT, GTTAGG,
TAGGCA, CGGGCC, TGGGTC, TACGGG, ACGGGT, TCAGGT, GGCGGG, TATGGG, GGGTCC, GGGCTT,
GGGCGG, GCTGGG, GGTAGG, GGGCCT, GGGCCG, CTAGGT, CGCAGG, CTTAGG, CATAGG, GGGCGA,
AGTGGG, TTGGGT, ATTAGG, AGGCCC, TGGGTT, GGGTAC, GCGGGC, GACGGG, GGGCCC, ACTGGG,
CGTGGG, TAGGCG, TGGGCG;
−0.9: AGGTGG, AGGTGT, GGGTGG, TGGGTG, GGGTGT, GGGTGA, AGGTGC,
CAGGTG, GGGTGC, TAGGTG, AGGTGA, CGGGTG;
−1.1: GGATGC, GGACAC, CGGATC, ACCGGA, GGATTA,
GGAAGC, CTTGGA, GGACAT, ACGGAT, CCGGAC, GGACCT, TCGGAC, GGACGG, TCCGGA, CGGAAT,
CACGGA, GGACTC, AATGGA, GACGGA, CATGGA, GATGGA, GGACCA, CGGACT, GGAAAG, CTCGGA,
TCGGAA, GGATTT, ATTGGA, GGAACG, TGGACA, GTGGAC, TCTGGA, GGACAA, GGAATC, TGGATT,
GGAAGA, TTCGGA, GCGGAC, GGATCA, GGATGA, GTGGAT, GGAAAC, GGACCG, GGCGGA, GGACGA,
GGAAAA, GTGGAA, TGGATC, TTGGAA, GGAACT, TTGGAT, CTGGAT, GGACTG, GGATGT, GGATAC,
ATGGAC, AGCGGA, TGGACC, CGGAAA, GGAACC, CCGGAA, CCCGGA, CGGATA, GGATAA, GCTGGA,
TTTGGA, TGGAAT, AACGGA, GGATGG, CTGGAC, GGACTT, TGGACG, GGATTG, GGAACA, GGATCT,
CCGGAT, GGACGT, GGACGC, TGTGGA, TGGAAC, TGGATG, CGGACC, ATGGAA, TGGAAA, GGTGGA,
GGATCC, CGTGGA, TGCGGA, GGACCC, TGGACT, CGGATT, GGATAG, GGATCG, ATGGAT, TGGATA,
TGGAAG, TCGGAT, GTTGGA, CGGATG, CGGACG, GTCGGA, GGAAAT, GGATAT, GGAATA, GGACTA,
GCGGAT, GGACAG, CGGAAC, TACGGA, ACTGGA, GCCGGA, TATGGA, GCGGAA, TTGGAC, ATCGGA,
CTGGAA, GGATTC, CGGACA, ACGGAA, CGGAAG, ACGGAC, GGAATT, CGCGGA, CCTGGA, GGAATG,
AGTGGA, GGAAGT;
−1.5: GGGCAG, GGGTAG, AGGCAG, AGAGAG, AGTGAG, GGCGAG, AGGTAG,
AGCGAG, GGTGAG;
−1.7: AAGGCG, ATAAGG, AAAAGG, GCAAGG, CTAAGG, TAAGGC, CAAAGG, AAGGTA,
TAAAGG, GGAAGG, CAAGGT, AAAGGT, CGAAGG, GTAAGG, TAAGGT, AAGGCC, AAGGCA, ACAAGG,
AAGGCT, AGAAGG, AAAGGC, CAAGGC, TTAAGG, GAAGGT, TCAAGG, TGAAGG, AAGGTT, CCAAGG,
GAAAGG, AAGGTC, GAAGGC;
−1.8: GCAGGG, AGGGCT, TAGGGT, AGGGCC, GTAGGG, TCAGGG, CAGGGT,
CTAGGG, AAGGTG, AGGGTA, TTAGGG, AGGGCA, ATAGGG, TAGGGC, ACAGGG, AGGGTT, AGGGTC,
CCAGGG, CAGGGC, AGGGCG, AGGGTG;
−2.1: TCGAGG, CTGAGG, GAGGCG, AAGAGG, GCGAGG,
AGAGGC, AGAGGT, GAGGCC, TGAGGT, TAGAGG, CAGAGG, TTGAGG, GAGGTC, CGAGGC, GAGGTT,
ACGAGG, GAGAGG, ATGAGG, CCGAGG, GAGGTA, TGAGGC, GTGAGG, GAGGCT, CGAGGT, GAGGCA;
−2.2: GAGGTG;
−2.7: TGGGAC, GAAGGG, ACAGGA, TAGGAT, AAGGGC, AAAGGG, GGGACA, GCGGGA,
TAGGAA, TGGGAT, AGGACG, GGGATA, GGGAAG, GGGAAT, AGGACA, GGGATT, AGGAAG, AGGATC,
CAGGAC, AGGATG, CAAGGG, GGGACG, GTGGGA, AGGATA, AGGAAC, TAAGGG, ATAGGA, TTGGGA,
TTAGGA, CCAGGA, CGGGAC, GGGACC, TCGGGA, ACGGGA, AGGACT, TAGGAC, AAGGGT, AGGAAA,
AGGAAT, CGGGAA, CTGGGA, AGGACC, GGGAAC, GGGAAA, GGGATC, AGGATT, TGGGAA, ATGGGA,
CGGGAT, CAGGAA, GGGACT, GTAGGA, GGGATG, TCAGGA, CAGGAT, GCAGGA, CCGGGA, CTAGGA;
−2.8: ATGGGG, TTGGGG, CGGGGT, CGGGGC, GCGGGG, GGGGCA, GGGGTT, GGGGCC, GGGGTG, ACGGGG,
CTGGGG, CCGGGG, GTGGGG, TGGGGC, TGGGGT, GGGGCT, GGGGTC, GGGGTA, TCGGGG, GGGGCG;
−3.1: AGAGGG, GAGGGT, GAGGGC, CGAGGG, TGAGGG;
−3.2: TGGGGA, GGGGAA, CGGGGA, GGGGAT,
GGGGAC;
−3.3: AAGGGA, AGGGAA, GAGGGA, CAGGGA, AGGGAT, AGGGAC, TAGGGA;
−3.6: GAAGGA,
AAGGAA, TAAGGA, CAAGGA, AAAGGA, AAGGAC, AAGGAT;
−3.7: GGAGTT, GGAGCC, GGAGAG, GGAGTG,
ACGGAG, GGAGGG, GGAGCT, TTGGAG, GGAGGC, CCGGAG, GTGGAG, TGGAGC, TGGAGA, ATGGAG,
CGGAGC, GGAGGT, GGAGCA, GGAGAA, TGGAGG, CGGAGG, GGAGTC, GGAGAT, GGAGTA, TGGAGT,
CTGGAG, GGAGCG, TCGGAG, GGAGAC, CGGAGT, GCGGAG, CGGAGA;
−4: AGAGGA, CGAGGA, GAGGAT,
TGAGGA, GGAGGA, GAGGAA, GAGGAC;
−4.4: GGGGGC, CAGGGG, AGGGGA, GGGGGT, CGGGGG,
TGGGGG, GGGGGA, AGGGGT, AGGGGC, TAGGGG;
−4.9: GGGGGG;
−5: AGGGGG;
−5.3: AGGAGT,
AGGAGA, GGGAGG, GGGAGT, AGGAGG, AGGAGC, GGGAGA, CAGGAG, GGGAGC, AAGGGG, TGGGAG,
TAGGAG, CGGGAG;
−5.7: GAGGGG;
−5.8: GGGGAG;
−5.9: AGGGAG;
−6.2: AAGGAG;
−6.6: GAGGAG.,
GCCGTA aSD:
−0.1: AAGGGA, CATTGG, AGGGAA, CGCTGG, TGGGAC, CTTGGA, TTCTGG, GCCTGG,
GAAGGG, GAGGGA, GGGGGG, AGGGGG, GGAGGG, AAAGGG, GCTTGG, GACTGG, CACTGG, CAGGGG,
CCTGGG, AACTGG, TTGGAG, TGTGGG, TGGGAT, CGTTGG, AAGTGG, GCAGGG, AGGGGA, GTGTGG,
CCTTGG, TTTGGG, ATTGGA, GTGGAG, TGGACA, TGGAGC, GTGGAC, TCTGGA, ACGTGG, TGGATT,
TGGAGA, CTGTGG, GTGGAT, GGGGAG, AGGGAG, CAGGGA, CAAGGG, GTGGAA, TGGATC, TTGGAA,
GTTGGG, GGGGAA, GTGGGA, TTGGAT, CTGGAT, TGTTGG, TAAGGG, ATCTGG, TGGAGG, TGGACC,
AGGGAT, TCAGGG, AGAGGG, TTGGGA, GAGTGG, TCGTGG, GCTGGA, TATTGG, TTTGGA, TGGAAT,
TTTTGG, GGGGAT, AGTTGG, TGGAGT, CTGGAC, GTCTGG, AAGGGG, TCCTGG, TGGGAG, AGGGAC,
TGGACG, ACAGGG, CAGTGG, CTGGAG, TCTGGG, GGGGGA, TTGTGG, ACTTGG, TGTGGA, CTGGGA,
TGGAAC, TGGATG, TAGTGG, GAGGGG, GATTGG, TGGAAA, TCTTGG, CGTGGA, CTTGGG, TGGACT,
ATTGGG, CTTTGG, TGGGAA, CGAGGG, ATGTGG, TGGATA, CTCTGG, TGGAAG, GTTGGA, GCTGGG,
GTTTGG, ACCTGG, TGAGGG, AGTGGG, ACTGGA, AATTGG, CCAGGG, AGCTGG, TTGGAC, CTGGAA,
CCCTGG, ATTTGG, CCTGGA, ACTGGG, CGTGGG, AGTGGA, GGGGAC, CCGTGG;
−0.3: GCGACA, AAGCGA,
GCGAGG, GAGCGA, GTAGCG, GACGCG, AGCGAC, CCAGCG, CTAGCG, GCGCTT, CAGCGT, GCGCCA,
GCGTCA, CGCGAT, ATCGCG, GCGCTC, AGCGCC, GCGTAA, TGAGCG, ACGCGA, GCGACG, CCGCGA,
TAGCGT, CGCGAG, GCGTCG, GAGCGC, CCGCGT, GCGCTG, GCGATA, AACGCG, CAAGCG, GCGCAT,
GCGTCT, TCGCGA, GCGACC, CGCGAC, GCGTTC, CGCGTG, GAGCGT, GCGCCG, TTCGCG, AAGCGC,
CAGCGA, GCGCAA, GCGATT, GCGTAG, GCGCAC, AGCGTG, TCGCGT, TAGCGC, GCGAAT, GCGTTT,
GCGTTA, TATTGC, AGAGCG, CGCGTT, GTCGCG, TCCGCG, GCGTAC, CGCGTA, GCGATG, TAAGCG,
AGCGAA, CGCGAA, GCGCGA, GCGCAG, AGCGAT, CAGCGC, CACGCG, TCAGCG, GGAGCG, TAGCGA,
GCGATC, AGCGCT, CCCGCG, GCGCCC, GAAGCG, GCGTTG, GCGTAT, AGCGTT, CTCGCG, CGCGTC,
GCGAGA, GCGTGA, GCGCCT, TATAGC, GCGTCC, AGCGCG, AGCGTC, GCGACT, ATAGCG, GCGCTA,
GCGTGG, AGCGTA, GCGAAG, TTAGCG, AAAGCG, AGCGAG, GCGAAC, AAGCGT, AGCGCA, GCGAAA;
−0.4: TGCAGT, TACTGT, TACAGT, TGCTGT, TGCCGT, TACCGT;
−0.5: CACAGC, AACCGC, CACTGC, ACAGCG,
ACCGCC, AACAGC, ACCGCT, CACCGC, ACAGCA, ACCGCG, GACTGC, AACTGC, ACTGCA, ACAGCC,
GACCGC, ACCGCA, ACAGCT, ACTGCC, GACAGC, ACTGCT;
−0.8: GCCGCT, CGCCGC, TGCTGG, GCCGCG,
AGCTGC, GCTGCA, GCCGCA, GCAGCC, TACAGG, AGCCGC, GCAGCA, GCAGCT, CGCAGC, TGCAGG,
TACTGG, AGCAGC, GCAGCG, GCTGCT, GCTGCC, CGCTGC, GCCGCC;
−1.1: TTGGGG, TGGGGA, ATGTGC,
CTGGGG, GTGGGG, TGGGGG, ATGAGC;
−1.2: GGTAGT, CGCGCC, AGGTGG, AGGTAT, GGTCTA, AGGTGT,
GGGTTC, GGGTGG, TAGGTT, GTAGGT, GGTCGA, GGTCGC, GGTAAA, TGGGTG, CGAGCA, CGAGCG,
TGGGTA, AGGTTG, CGCGCG, GGTGCT, TAGGTC, AGAGGT, GGTGAT, GGTTCA, GGTTGG, GGTGAA,
GGTGGG, GGTTTA, GGTGCA, GGGTTA, GGGTCT, AGGTCG, GGGTAA, GGTTTG, GGGTGT, GGTAAT,
GGTCCT, GGGTCG, GGTATC, GGGTGA, GGTTCG, AAGGTA, GGTATT, GAGGTG, GGGTCA, GGTCCC,
GAGGTC, GGTTAG, GGTCAT, TAGGTA, GGGTAG, GGTTCC, CAAGGT, GAGGTT, AGGTGC, AAAGGT,
AGGTAC, GGAGGT, GGTGCC, AGGTCT, AGGTCA, GGTCTT, ATAGGT, CCGTGC, CAGGTG, GGTAGC,
GGTCGT, GGTTGA, GGTAAC, AAGGTG, TCGCGC, GGGTGC, GGTTTC, GGTATA, GGTGTC, CGTGCC,
AGGTTA, CGCGCT, TAAGGT, AGGTAA, CCGCGC, GGTGTT, TCGAGC, CGCGCA, TTAGGT, AGGTTT,
GGTCCG, GAGGTA, GGGTTG, GGTCTC, GTGGGT, GGTTGC, GGTACT, AGGTTC, TAGGTG, GGTCAG,
GGTATG, GGTCAC, GGTGGA, GGTCTG, GGGTTT, AGGTGA, GGTCCA, CCGAGC, GGTTGT, CGTGCA,
GAAGGT, GGTTCT, CAGGTT, CGTGCT, AGGTCC, CAGGTC, CTGGGT, GGTACC, AAGGTT, CAGGTA,
CCAGGT, GGGTAT, CGAGCT, CGAGGT, TCGTGC, TGGGTC, AGGTAG, CGAGCC, TCAGGT, GGGTCC,
GGTGTG, GGTTAT, GGTAGG, GGTGAC, GGTCAA, CTAGGT, TTGGGT, GGTTAA, TGGGTT, GGGTAC,
GGTTTT, GGTGTA, GGTAAG, GGTTAC, GGTACA, GGTAGA, GGTGAG, AAGGTC;
−1.3: TCTGCG, TGCGTT,
TGCGCT, TTGCGT, GCTGCG, GTTACG, CTACGA, CTTGCG, CTGCGA, TGCGCC, TCTACG, GTACGC, ATTGCG,
TACGAG, TTACGT, GATACG, CATGCG, GATGCG, TGCGAG, TACGCT, GTTGCG, TACGTT, ATACGT, TTTGCG,
TACGAT, GGTACG, TACGTC, GGTGCG, TACGTG, CTACGT, CTTACG, TTTACG, CGTACG, TACGAC, ACTACG,
CTACGC, CCTACG, CGTGCG, CATACG, TTACGA, TACGTA, TACGCA, TACGCG, TTGCGA, TGCGCG, ATGCGC,
AATGCG, TTGCGC, CTGCGT, ACTGCG, AGTGCG, TGTGCG, TGCGAT, ATACGA, AATACG, TATGCG,
ATACGC, TACGAA, GTACGA, TATACG, TGCGTC, TGCGTA, AGTACG, CTGCGC, TTACGC, GTGCGT, TACGCC,
GCTACG, GTACGT, TGCGCA, TGTACG, TGCGAC, CCTGCG, ATTACG, TGCGAA, TGCGTG, GTGCGC,
ATGCGA, ATGCGT, GTGCGA;
−1.4: GTAGGG, CTAGGG, TTAGGG, ATAGGG, TAGGGA, TAGGGG;
−1.5: AATGGG, ATGGGG, CAATGG, CGATGG, AATGGA, CATGGA, ACGTGT, GATGGA, ACATGG, ACGAGT,
ATGGAG, AAATGG, TAATGG, GATGGG, CATGGG, CCATGG, ATGGGT, ATGGAC, ACAGGT, GGATGG,
AGATGG, ACGCGT, GCATGG, ATGGAA, TCATGG, ATGGGA, ATGGAT, GAATGG, TGATGG;
−1.6: CGGATC,
ACCGGG, ACCGGA, CCGGAC, TGCCGG, ATCGGG, TCGGAC, TCCCGG, TCCGGA, AATCGG, ACTCGG,
CGGAAT, CCCCGG, ATCCGG, CGCCGG, CCCGGG, CGGACT, GTTCGG, CTCGGA, TCGGAA, CCGGAG,
GTCGGG, GCCGGG, TTCGGA, CGGAGC, GCCCGG, CGGGGG, CCGGGT, TTCCGG, ATTCGG, TTTCGG,
CGTCGG, TCGGGT, TTCGGG, CGGGTA, CCGGGG, CTCGGG, CGGAAA, CCGGAA, CGGGGA, CCCGGA,
CGGATA, CGGAGG, CGGGAC, AGCCGG, CTTCGG, GACCGG, TACCGG, TCGGGA, TCCGGG, CCGGAT,
CGGGAA, CCTCGG, CGGACC, GATCGG, CACCGG, AACCGG, GGTCGG, CGGATT, TCGGAG, CGGGTT,
AGTCGG, CATCGG, CTCCGG, CGGGAT, CGGGTC, TCGGAT, CGGAGT, CGGATG, CGGACG, GTCGGA,
CGGGTG, TCGGGG, TATCGG, CGGAAC, GCCGGA, GCTCGG, ACCCGG, ATCGGA, TGTCGG, CGGACA,
CCGGGA, CGGAAG, CGGAGA, TCTCGG, GTCCGG, CGGGAG;
−1.8: TACCGC, TACTGC, GCGTGT, TGCTGC,
GCAGGT, TGCAGC, TACAGC, GCGCGT, GCGAGT, TGCCGC;
−1.9: TGAGGT;
−2.1: GGTGGT, TGGTGA, TTGGTT, CGGGGT, TTGGTA, TGGTTC, TGGTCA, TGGTCT, CGTGGT,
TGGTCG, TGTGGT, TGGTGT, CTGGTT,
CTGGTG, TGGTAC, TGGTAT, GGGGGT, GGGGTT, TGGTTA, GGGGTG, TTTGGT, CTGGTC, CCTGGT,
GTGGTA, TGGTGG, TGGTAG, CAGGGT, AGGGTA, CTGGTA, TGGTTG, GAGGGT, GTGGTC, AAGGGT,
GTTGGT, ACTGGT, TTGGTC, TGGTAA, AGGGTT, AGGGGT, TCTGGT, AGTGGT, TGGTGC, GCTGGT,
TGGTTT, AGGGTC, GTGGTG, CTTGGT, GGGGTC, GGGGTA, GTGGTT, TGGTCC, ATTGGT, AGGGTG,
TTGGTG;
−2.6: GGCTGC, AGGCAC, GGGCAC, GAGGCG, GGCTCA, CAGGCT, GGCTTG, GTAGGC, GGCACA,
GGCCGG, GGCAGC, TTGGGC, AAGGCG, AGGCCA, GGCCTA, GGCTGG, GGCTAA, GGCTAT, GGCCAA,
AGGCTT, AGAGGC, GGCTTT, GAGGCC, TAGGCC, GGGCGC, CAGGCA, GGAGGC, GGCAGG, GGCTGA,
GGCGTA, GGCCCT, AGGCTG, AGGCGT, AGGCCT, CCGGGC, GGCGCC, CGGGCG, GGGCCA, CTAGGC,
TGGGCA, TAAGGC, GGCGAT, GGCCCG, TGGGCC, GGCGTC, TGGGCT, GTGGGC, GGGCAG, GGCACT,
CAGGCG, GGCCAG, GGCCAT, CGGGCT, ATAGGC, GGCTTA, GGCACG, CGAGGC, TCGGGC, GGCGAA,
GGCTTC, AGGCTA, GGCCGC, GGCTCG, CCAGGC, AGGCCG, AGGCAG, TTAGGC, GGCACC, GGCGCA,
GGCATG, CTGGGC, GGGCAT, GGCAAG, GGCATT, AGGCGC, GGCGAC, GGGCTA, AGGCAA, GGGCAA,
GGCCCA, GGGCTC, AAGGCC, CGGGCA, AAGGCA, AAGGCT, GGCCGA, GGCAAT, AAAGGC, GGCAAC,
TAGGCT, GGCCTC, GGGCTG, ATGGGC, CAGGCC, GGGCGT, AGGCGA, GGCTCC, GGCGCG, GGCAGT,
GGCAGA, TCAGGC, GGCGTG, CAAGGC, GGCTAC, AGGCTC, GGCATA, GGCTCT, AGGCAT, GAGGCT,
GGCGAG, TAGGCA, CGGGCC, GGCTGT, GGCCAC, GGCTAG, GGCCCC, GAGGCA, GGGCTT, GGCCTT,
GGGCCT, GGGCCG, GGGCGA, GGCATC, AGGCCC, GGCCGT, GGCCTG, GGCAAA, GGGCCC, GGCGCT,
GGCGTT, TAGGCG, TGGGCG, GAAGGC;
−2.8: TTATGG, ATATGG, GTATGG, CTATGG, TATGGG, TATGGA;
−2.9: ACAGGC, ACGAGC, ACGCGC, ACGTGC;
−3.1: TGGGGT;
−3.2: GCGAGC, GCAGGC, GCGCGC, GCGTGC;
−3.3: GCACGG, ACGGAG, CAACGG, ACGGAT, GGACGG, GAACGG, CACGGA, GACGGA, CCACGG, ACGGGG,
AAACGG, TGACGG, ACGGGC, CACGGG, ACACGG, AACGGA, AACGGG, TCACGG, ACGGGA, CGACGG,
TGAGGC, TAACGG, ACGGGT, ACGGAA, GACGGG, ACGGAC, AGACGG;
−3.4: TAGGGT;
−3.5: CGTGGC,
GTTGGC, ATGGTC, ATGGTT, GTGGCT, AATGGT, GGGGGC, TTGGCA, TGGCGC, AAGGGC, TTTGGC,
GTGGCG, CGGGGC, TGGCTA, GCTGGC, TGGCTC, TGGCAA, GTGGCA, TGGCAG, CTTGGC, AGGGCT,
GGTGGC, TTGGCC, AGGGCC, GTGGCC, TTGGCG, GGGGCA, TGTGGC, ATGGTA, TGGCGT, GGGGCC,
ATTGGC, ATGGTG, ACTGGC, AGGGCA, TGGCTT, TGGCGA, CTGGCG, GATGGT, GAGGGC, TGGCCT,
TGGCAC, CTGGCC, TCTGGC, CTGGCT, TGGCCA, AGTGGC, AGGGGC, GGGGCT, CATGGT, TGGCAT,
TTGGCT, TGGCCG, TGGCTG, CCTGGC, CTGGCA, TGGCCC, GGGGCG, CAGGGC, AGGGCG;
−3.6: CCGGTA,
CCGGTG, TCGGTT, TCGGTA, CTCGGT, TCCGGT, TGGCGG, CGGTGG, TCGGTC, AGGCGG, GCGGGA,
GCGCGG, CGGTCG, CGGTAC, ACGCGG, ACCGGT, GCGGGG, GCGGAC, CGGTGC, CGGTGA, TTCGGT,
GAGCGG, GGCGGA, CCGCGG, CGGTTG, CCGGTC, AGCGGA, CCCGGT, TCGGTG, CGGTTC, TCGCGG,
CAGCGG, CGGTAT, CGGTTA, GTCGGT, CGCGGG, CGGTCC, TAGCGG, GCGGGT, CGGTAA, AGCGGG,
CGGTAG, CGGTTT, ATCGGT, GGCGGG, GGGCGG, AAGCGG, GCGGAT, CCGGTT, CGGTCT, GCCGGT,
GCGGAA, GCGGAG, GCGGGC, CGGTCA, CGGTGT, CGCGGA;
−4.5: TGGGGC;
−4.6: CTGCGG, GTACGG,
GTGCGG, TTGCGG, CTACGG, TGCGGG, TGCGGA, TTACGG, TACGGG, TACGGA, ATACGG, ATGCGG;
−4.8: TATGGT, TAGGGC;
−4.9: GATGGC, ATGGCG, ATGGCT, AATGGC, CATGGC, ATGGCA, ATGGCC;
−5: CTCGGC,
CCGGCG, CCCGGC, CGGCGT, GCCGGC, TTCGGC, TCGGCG, CCGGCC, CCGGCT, CGGCCC, TCCGGC,
CGGCTG, CGGCGC, CGGCTA, CGGCCT, CGGCAA, CGGCTT, CGGCGA, ACCGGC, CGGCGG, TCGGCC,
GTCGGC, CCGGCA, TCGGCT, CGGCAC, CGGCCG, TCGGCA, ATCGGC, CGGCAG, CGGCTC, CGGCCA,
CGGCAT;
−5.3: ACGGTA, CACGGT, GACGGT, ACGGTT, ACGGTG, AACGGT, ACGGTC;
−5.6: CGCGGT,
AGCGGT, GGCGGT, GCGGTT, GCGGTA, GCGGTG, GCGGTC;
−6.2: TATGGC;
−6.6: TGCGGT, TACGGT;
−6.7;
ACGGCT, ACGGCG, AACGGC, ACGGCA, ACGGCC, GACGGC, CACGGC;
−7: GCGGCT, CGCGGC, AGCGGC,
GCGGCA, GGCGGC, GCGGCG, GCGGCC;
−8: TGCGGC, TACGGC.,
GCGGCT aSD: 10: GGCCGC, AGCCGC;
−0.1: AGATCG, GGTTCG, AGTTCG, GGTACG, AGTACG, GGATCG;
−0.2: GTGCAG, TGCATC, ATGCAC, GAATGC, GCAAGT, CGATGC, GTGCAT, TGCATT, CATGCG, CTATGC,
GATGCG, TGCGAG, TGCACC, GTGCAA, CATGCA, TGTGCA, ATGCAT, ATGTGC, ATATGC, TGCACT, GTGCAC,
AAATGC, TGCACA, TTGTGC, TGATGC, TGCAAG, TTATGC, GCAGGT, TGCAGA, TATGCA, ACATGC, TAATGC,
ATGCAA, AATGCG, TGCATG, TGCAAT, ATGCAG, TGTGCG, CCATGC, TGCGAT, TATGCG, AATGCA,
GATGCA, TCATGC, TGCAAC, TGCAGG, CAATGC, TGCAAA, TGCGAC, GTATGC, GCGAGT, TGCATA,
TGCGAA, ATGCGA, GTGTGC, GTGCGA;
−0.3: GACGTC, CGTGAG, CGTGTG, TGCGTT, GTCACT, CACGTC,
GTCACC, CGTTCC, ACGTAG, CGTCTG, CGTCAA, ATGTTG, AAACGT, TGGGTG, GCGTCA, TTGTTG, CGTCAC,
TGTTGA, GACGTG, TGACGT, TTACGT, ACGTCA, CGTGTT, ACGTGT, GCGTAA, CGTACT, CGTTGG, CAACGT,
ACGTAA, CGTAGG, CGTGAC, GGGTGA, CGTTTG, TACGTT, ACGTGG, ACGTCT, CGTAAC, ATACGT,
CGTAAA, ACGTAC, CGTGAA, GAGGTG, GTTGAT, CACGTA, CGTTCA, GCGTCT, CGTTCT, CGTGAT, TACGTC,
ACGTGA, GCGTTC, TACGTG, CTACGT, CGTACG, GTTGAA, CACGTG, GTTGGG, ACGTTG, CGACGT,
GGGGTG, TGTTGG, CGTATA, CGTATT, CGTGCG, CGTGTA, AACGTC, CAGGTG, CGTAGT, AACGTA,
CGTTAA, GCGTAG, TGTCAC, CGTAGA, AACGTG, TACGTA, CGTTGA, ACGTTA, AAGGTG, CGTTAT, GCGTTT,
CGTTTT, GCGTTA, CGTATG, CACGTT, CGTAAG, ACGTAT, CGTAAT, GCGTAC, GTTGGT, CGTCCT, GACGTT,
GTTGAC, CGTTCG, GTTGAG, CGTTTC, GTGTTG, TAGGTG, CGTCTT, AGGTGA, CGTCAT, ACACGT, CGTGGA,
CGTTAG, TGCGTC, CCACGT, TAACGT, TCACGT, GCGTTG, ACGTTC, CGTACC, GCGTAT, GACGTA, TGCGTA,
CGTTAC, CGTTTA, GCGTGA, CGTGCA, CGTCCA, CGTCTC, GTCACG, GCGTCC, CGTCAG, GTCACA, GTTGGA,
CGTGTC, CGTCTA, CGTATC, CGGGTG, AACGTT, GCGTGG, CGTACA, GAACGT, ACGTCC, TGCGTG, ACGTTT,
ACGTGC, ATGCGT, CGTCCC, AGGGTG, CGTGGG;
−0.4: TAGACC, GGACCT, CAGACC, GGACCA, AGACCT,
AAGACC, TGGACC, AGACCC, GGGACC, AGGACC, CGGACC, GGACCC, AGACCA, GAGACC;
−0.5: GTCAGT,
GTGCGT, GTACGT;
−0.6: GGACTG, AGACTG;
−0.7: TTTTGC, TTGCGT, CTTGCG, ATTGCG, GCGGGA,
TTTGCA, ATTGCA, AATTGC, TGGTGT, TTTGCG, GCGGGG, GCGGAC, GTGCGG, TTGCAT, TTGCGG, CTTTGC,
TTGCAG, CATTGC, GTTTGC, CTTGCA, ACTTGC, GGTGTC, TTGCGA, TCTTGC, TATTGC, GGTGTT, ATTTGC,
GCGGGT, TGCGGG, TGCGGA, GATTGC, GGTGTG, TTGCAC, GCGGAT, TTGCAA, CCTTGC, GCGGAA,
GCGGAG, GGTGTA, CGGTGT, ATGCGG;
−0.8: GGTAGT, GGATGC, TGCAGT, AGATGC, GCAGTT, GCAGTG,
AGTAGT, GCAGTA;
−0.9: GCTAAG, GTTGGC, GCTATG, AGGCAC, AAGCGA, GCTTTA, GGGCAC, CAAAGC,
TTTAGC, ACAGGC, GTAGGC, GGCACA, ATTAGC, CGAAGC, CTCGGC, GCTTCG, TTGCTA, CGTAGC,
TTGGGC, GCTTAC, GCTCAC, TGCTAT, GAGCGA, GTAGCA, GTAGCG, GATGGC, GGGGGC, TTGGCA,
TGAAGC, GCTTTG, CTAGCG, AAGGGC, GATAGC, GCTACT, CACAGC, CGAGCA, GCTTGG, ATGCTA,
ATGGCG, CGAGCG, GTGCTT, CATAGC, GAGCAG, TTTGGC, ACGGCG, CAGCAC, AGCAAG, TTGAGC,
CGGGGC, ACGAGC, GATGCT, AGCACA, GAAAGC, AATGGC, AACGGC, CAGGCA, TGAGCG, GGCAGG,
CTCAGC, GCTACC, GCTAAA, TATGGC, ACAGCG, AAGCAT, TCTAGC, GCTTAG, ATTGCT, TTCGGC, GCTTTC,
AGCATT, TAGCAA, GAGCAT, TCGGCG, TTGCTC, TGGCAA, AAGCAG, TGGCAG, CTTGGC, GAGCAC,
CTGAGC, CTAGGC, TGGGCA, TAAGGC, GGCGAT, TGCTCT, TGCTAC, TGGAGC, AACAGC, AAAAGC,
CAAGCG, GCTAGG, CGGAGC, GTGGGC, GGGCAG, GGCACT, TTGGCG, GGGGCA, TGCTTC, GCTTGA,
GAGAGC, ATAGGC, GCTATT, CAGCAG, CTAAGC, TCAAGC, GCTTCC, AGCAAA, CGAGGC, AATAGC,
TCGGGC, GGCGAA, GTTAGC, TGCTAA, CATGGC, ACGGCA, GCTCCT, GCTCTC, CCAGGC, CAGAGC,
ATAAGC, AGGCAG, ATTGGC, TTAGGC, CCAAGC, GGAGCA, AGCAGG, CAGCGA, GGCATG, TGTAGC,
TTAGCA, AAGCAC, CTGGGC, GAGCAA, GGGCAT, GGCAAG, GGCATT, CGGCAA, TGCTTG, ACGGGC,
TTGCTT, CCTAGC, CATGCT, ACTAGC, ACTGGC, ATGCTC, AAGAGC, ACAGCA, TGCTTA, ATGGCA, GCTCTG,
GCTATC, AGGGCA, CTTGCT, AGGCAA, AGCATA, GGGCAA, ACAAGC, GCTTTT, TGGCGA, CGGCGA,
TGGGGC, GCTTCT, CGGGCA, AAGGCA, TAGGGC, CTGGCG, AGAGCG, TCGAGC, GCTCTT, GGCAAT,
GAGGGC, AGCAAT, AAAGGC, CTTAGC, TAGCAT, TCAGCA, GCTCTA, TAAGCG, TTCAGC, AGCGAA,
ATAGCA, ATGGGC, TAGCAG, GTGCTC, GTAAGC, AGCATG, ATGCTT, TTTGCT, TGGCAC, GCTCAG,
GACGGC, TGAGGC, AGCGAT, AAAGCA, GGCAGA, GTGCTA, CTAGCA, TCAGGC, AGCACT, TCAGCG,
GGAGCG, CAAGGC, TCTGGC, TAGCGA, AGAGCA, GCTTAT, GAAGCA, GGCATA, GCTAAC, GAAGCG,
CACGGC, TAGCAC, ATCAGC, TACAGC, TGCTAG, GCTACA, TAGAGC, AAGCAA, CGTGCT, GCTCCA,
AGGGGC, AGGCAT, TAAAGC, GTGAGC, AGCATC, GCTACG, TATAGC, GGCGAG, TAAGCA, TAGGCA,
GCTTAA, CGGCAC, TGGCAT, TATGCT, GCTTCA, TTAAGC, GAGGCA, GCTAGA, CAGCAT, ATAGCG, CAGCAA,
TGCTCA, GCTATA, TCGGCA, ATCGGC, TGCTTT, CAAGCA, GCTCCC, GGCATC, TGAGCA, AATGCT, TTAGCG,
AAAGCG, GCTCAT, AGCGAG, ATGAGC, CGGCAG, AGCAGA, GACAGC, CCTGGC, TGCTCC, GGCAAA,
CTGGCA, TACGGC, CAGGGC, CGGCAT, TGTGCT, GCTCAA, GCTAAT, GAAGGC;
−1: AGTGCA, GCTTGT,
GCGTGT, GAGTGC, CAGTGC, AGTGCG, GCTAGT, TAGTGC, GCATGT, AAGTGC, AGTGCT;
−1.2: GCACGG,
ACCAGC, CCAGCG, AGAGGC, CCCAGC, GGAGGC, AGGAGC, TGCACG, TGCTCG, GCACGA, GCTCGA,
GGGAGC, CCAGCA, TCCAGC, GCTCGG;
−1.3: TCGTTT, TCGTCC, ATCGTG, AGCGAC, TCGTTG, TTCGTC,
TTCGTT, CCTCGT, ATCGTC, CATCGT, TCGTAA, CTCGTC, TCGTGG, GGCGAC, TTCGTA, TCGTAG, ATTCGT,
TCGTCA, TCGTAC, TCGTTA, GATCGT, CTTCGT, ATCGTT, TCTCGT, ATCGTA, CTCGTA, ACTCGT, TATCGT,
CTCGTG, TTCGTG, TCGTAT, TCGTGC, AATCGT, TCGTCT, CTCGTT, GTTCGT, TCGTGT, TCGTTC, TCGTGA,
TTTCGT;
−1.4: AGGTGG, GTCTGT, ACTGTC, CTGTGA, GGAAGC, GGGTGG, CTCTGT, CTGTTG, ACCTGT,
CTGTAA, CCTGTT, CGGTGG, ACTGTT, CCCTGT, GGTGGG, AAGTGG, TACTGT, TCTGTG, GACTGT, AGACGT,
CTGTAT, CTGTGG, CCTGTA, CTGTCT, TCTGTC, TCTGTT, CTGTTA, TGGTGG, TCCTGT, GAGTGG, CTGTCA,
ATCTGT, CTGTGT, TTCTGT, CAGTGG, GGACGT, CTGTAG, CTGTAC, TAGTGG, TCTGTA, AGAAGC, AACTGT,
GGTGGA, ACTGTA, CTGTCC, CTGTTC, CCTGTG, CTGTTT, CACTGT, AGTGGG, CTGTGC, CCTGTC, ACTGTG,
AGTGGA;
−1.5: ATGGTC, GGTCTA, GAGTCT, TCAGTC, CAGCGT, TGGTCA, AGTCTG, CGAGTC, AGTCAT,
ACAGTC, AAGTCT, TGGTCT, TAGGTC, TCGGTC, GGGTCT, TAGCGT, GGTCCT, GGGTCA, GGTCCC, AGTCCT,
ATAGTC, GAGGTC, TAAGTC, AAGTCC, GGTCAT, AAAGTC, CAAGTC, GCAGTC, GAGCGT, AGGTCT,
AGGTCA, GGTCTT, CTGGTC, GGCACC, AGCGTG, AGTCTT, GGAGTC, AGTCAG, AGTCAA, AGTCCA, AGTCTC,
CCAGTC, TTGGTC, GGTCTC, TAGTCT, CGGTCC, CAGTCC, GGTCAG, GGTCTG, GAGTCA, GGTCCA, AGTCTA,
AGCGTT, TAGTCC, AGGGTC, TGAGTC, TAGTCA, AGGTCC, CAGGTC, CGGGTC, AGAGTC, TGGGTC,
GGGGTC, AGTCCC, AGCGTC, AAGTCA, GGGTCC, TGGTCC, GGTCAA, GTAGTC, CAGTCA, CAGTCT,
AGCGTA, CTAGTC, CGGTCT, GAAGTC, ACGGTC, AGCACC, TTAGTC, AAGCGT, CGGTCA, GAGTCC,
AAGGTC;
−1.6: CCGGTA, CCGGTG, ACCGGG, ACCGGA, CGACCG, CACCCG, GTCCCG, CCGGCG, ACCGAA,
CCGAAG, CAACCG, CCGGAC, TCCGAT, TCCGGT, TCCCCG, TCCCGG, CCGAGA, TCCGGA, CCGACG,
ACCGAG, TTCCCG, CCCGGC, GACCCG, CCACCG, ACTCCG, TGACCG, GCGAGC, CCCCGG, GCTCCG,
ATCCGG, GATCCG, TAACCG, CCCGGG, CACCGA, CCGATC, GCAGGC, CCGACA, CATCCG, ATCCGA,
TATCCG, CCGGGC, CCGGAG, TTACCG, CCCGAC, ACCGAT, CTTCCG, CTCCCG, GACCGA, ACCGGT, CCGAAA,
CCGAGT, CCGAAC, CCCGAT, CCGACT, TCCGAC, TACCGA, GCACCG, CCGATG, ACCCGA, TTTCCG, CCGGGT,
TTCCGA, ATTCCG, TTCCGG, TCCGGC, TCCGAA, TACCCG, AATCCG, CCGGTC, CCCGGT, CCGGGG, CCGGAA,
AACCCG, CCCGGA, ATACCG, GTACCG, GACCGG, TACCGG, CTCCGA, TCTCCG, ACCGGC, TCCGGG,
CCGAGG, CCGGAT, GGCAAC, ACCGAC, ACACCG, CCCCGA, CGTCCG, TCCGAG, CACCGG, CCTCCG,
AACCGG, TGTCCG, GTCCGA, CCGAAT, CCGAGC, CCCGAG, CCGGCA, CCCCCG, ATCCCG, CTCCGG,
CCGACC, CCGATT, CCCGAA, TCCCGA, GCAAGC, CCGATA, AGCAAC, CCGGTT, CTACCG, ACCCGG, GTTCCG,
GCGGGC, CCGGGA, TCACCG, AAACCG, GAACCG, GTCCGG, ACCCCG, AACCGA;
−1.7: CGCTCA, CGCATG,
ACGCTC, ACGCTA, TGCGCT, CGCAAT, CGCTAA, CGCTCC, CGCTTA, GACGCG, CGCACA, ACGCAC, CGCTCG,
GCGCTT, CGCATA, CGCTAT, CGCGAT, CGCTAG, AAACGC, ACGCAG, CGCGCG, GCGCTC, GCGCGG,
AACGCT, CGCATC, ACGCGA, TACGCT, CGCGAG, ACGCGG, AACGCA, AACGCG, GCGCAT, CGCTCT,
CGCGAC, CGCGTG, GACGCT, ACGCAA, ACGCAT, CTACGC, CGCATT, CGCAAG, GCGCAA, GCGCAC,
ACGCGC, CGCTTT, CGCTTC, CGCAAA, CGCTAC, CGCAGT, TACGCA, TACGCG, TGCGCG, GAACGC, ATGCGC,
CGCGCT, TAACGC, TTGCGC, ACACGC, CGCGTT, ACGCGT, CGCGCA, CGCGTA, CGCGGG, CGACGC,
CGCGAA, GCGCGA, ATACGC, CGCACG, GCGCAG, CCACGC, CACGCG, CGCAAC, CACGCA, CAACGC,
CGCAGA, CGCGTC, CGCACT, CGCTTG, TTACGC, TGACGC, TGCGCA, ACGCTT, GCGCTA, CGCAGG, CGCACC,
GACGCA, CACGCT, CGCGGA, TCACGC;
−1.8: CGTGGT, TGTGGT, GTGGTA, GTGGTC, GTGGTG, GTGGTT;
−1.9: AGTTGA, GTACGC, AGGTTG, GGTTGG, GTCAGC, TAGTTG, GAGTTG, CGGTTG, GGTTGA, TGGTTG,
AGTTGG, GGGTTG, AAGTTG, AGTCAC, GGTCAC, CAGTTG, GTGCGC;
−2.1: GGTGCT, GGTGCA, CGGTGC,
GGTGCG, TGGTGC;
−2.2: GAGGCG, AAGGCG, CGGGCG, CAGGCG, GGTAGC, GCAGCA, CGCAGC, AGGCGA,
TGCAGC, GCAGCG, AGTAGC, GGGCGA, GGGGCG, AGGGCG, TAGGCG, TGGGCG;
−2.3: AGGTGT, GTCGAG,
TGGCGG, GTTGTA, AGGCGG, GCGTCG, GGGTGT, CGTTGT, GTCGAC, GTCGGG, GTGTCG, ATGTCG,
GTCGAT, GAGCGG, GGCGGA, CGTCGG, GTTGTC, AGCGGA, GTTGTT, CAGCGG, TCGTCG, GTTGTG,
CGGCGG, CTGTCG, GTCGGT, TAGCGG, AGCGGG, TGTTGT, TGTCGA, GTCGGC, CGTCGA, GGCGGG,
GTCGGA, GGGCGG, GTCGAA, ACGTCG, AAGCGG, TGTCGG, TTGTCG;
−2.4: GCTTGC, GCTAGC, GGCAGT,
GCATGC, GCGTGC, AGCAGT;
−2.5: GGCTCA, CAGGCT, GGCTTG, ACGGCT, GGCTAA, GGAGCT, GGCTAT,
AGGCTT, GGCTTT, GAAGCT, ATGGCT, GTAGCT, AGCTAG, TGGCTA, TGGCTC, AGCTTT, AGGGCT, AGCTTA,
AGCTTG, TGAGCT, TGGGCT, ATAGCT, CGGGCT, TAAGCT, CCGGCT, GGCTTA, TAGCTC, GGCTTC, AGGCTA,
CAGCTC, CAAGCT, CGGCTA, AGAGCT, AGCTTC, AAGCTA, CGGCTT, CCAGCT, CAGCTA, GGGCTA, AGCTAC,
AAGCTT, TGGCTT, GGGCTC, AAGGCT, GCAGCT, AGCTCA, TAGGCT, AGCTCT, AAGCTC, GGCTCC, AGCTAA,
AGCTAT, CTAGCT, TAGCTT, GGCTAC, GAGCTT, CTGGCT, AGGCTC, CAGCTT, GGCTCT, AAAGCT, TCGGCT,
GGGGCT, GAGGCT, CGAGCT, GAGCTA, GGCTAG, TTGGCT, GGGCTT, ACAGCT, TAGCTA, GAGCTC,
CGGCTC, TCAGCT, AGCTCC, TTAGCT;
−2.6: CGCCCA, CGCGCC, GCCCTC, GCCCGA, TGCCCC, GCCTAC,
CGCCAT, GCCTGG, AATGCC, ACGCCA, GCGCCA, GCCCTG, TGCGCC, GCCAAC, CGCCTG, GCCAGA, TTTGCC,
CGGCGT, CGCCCC, CGCCCT, GCCATT, GCCATG, CGCCCG, GCCCTA, GGCGTA, GCCTAA, CGCCTC, TGCCCG,
TGCCTG, GGCGTC, ATTGCC, AACGCC, GCCTCG, GCCCGG, GCCTTG, GCCTCC, GTGCCA, GCCAAG, GCCTCT,
TGCCAT, TGGCGT, CGCCTA, GCCCCC, GTGCCT, GGTGCC, GCCTGA, CACGCC, ATGCCT, GACGCC, ACGCCC,
GCCAAA, TGCCAA, ATGCCC, GATGCC, CGTGCC, GCCCCT, TATGCC, GCCTTA, CTTGCC, TTGCCC, ATGCCA,
GCCCAT, AGTGCC, TGCCTC, TGCCTA, GCCCAA, GGCGTG, GCCCTT, CGCCAG, GCCAGG, GCCCAC, TGCCCT,
GCGCCC, GCCTAG, TGCCAG, GCCAAT, GCCTCA, GCCATC, TACGCC, GTGCCC, GCGCCT, ACGCCT, TTGCCT,
GCCTTC, CGCCAA, CGCCTT, GCCTAT, TGCCTT, TGCCCA, TGTGCC, GCCTTT, TTGCCA, GCCCAG, GCCCCG,
GCCATA, GCCCCA, CATGCC, GGCGTT;
−2.7: TCGCAA, TCGCTC, TATCGC, TTCGCT, TGCGGT, ATCGCG,
CGCGGT, ATTCGC, TCGCCC, CTCGCT, ATCGCA, ACTCGC, AATCGC, TCGCGA, TTCGCC, TTCGCG, TCGCGT,
TCGCCT, TCGCGC, TCGCGG, GATCGC, CTCGCC, GCGGTT, TTTCGC, GTTCGC, TCGCCA, TCGCAT, TCGCTA,
CATCGC, CTCGCG, TTCGCA, GCGGTA, TCGCAC, ATCGCC, TCTCGC, CTTCGC, GCGGTG, ATCGCT, CCTCGC,
GCGGTC, CTCGCA, TCGCTT, TCGCAG;
−2.8: TCTGCG, CGCTGG, CTGCGG, CTGCGA, TGCTGG, AGTCCG,
GTCTGC, AGCTCG, ACCTGC, TCGCTG, CACTGC, GCTGGC, ACGCTG, TCTGCT, GCTGAC, TACTGC, GCGCTG,
ATCTGC, CCCTGC, CCTGCT, CTGCCT, AGACCG, CTGCTT, GGACCG, GGCACG, CGCTGA, GTGCTG, GGCTCG,
TGCTGA, CTGCTC, ATGCTG, CTGCTA, CTGCAT, GCTGGA, TCTGCC, CTGCGT, ACTGCG, GACTGC, GGTCCG,
AACTGC, GCTGAG, GGACGC, TTGCTG, CTGCCC, ACTGCA, AGACGC, CCTGCC, GCTGGT, CTCTGC, CTGCAA,
CTGCGC, AGCACG, TTCTGC, GCTGGG, CTGCAC, ACTGCC, TCCTGC, CTGCCA, TCTGCA, CTGCAG, CCTGCG,
ACTGCT, CCTGCA, GCTGAA, CTGCTG, GCTGAT;
−2.9: AGCGCC, GAGCGC, AAGCGC, TAGCGC, CAGCGC,
AGCGCT, AGCGCG, AGCGCA;
−3: GCCACC, GCCACG, GCCACA, TGCCAC, GCCACT, CGCCAC;
−3.2: CGTGGC,
GTGGCT, GTGGCG, GCCTGT, GTGGCA, TGTGGC, GCACGT, GCTCGT, GCCAGT, GCGCGT;
−3.4: GGTGGT,
AGTGGT;
−3.6: CCGTCG, CACCGT, GCCCGT, AACCGT, CCGTAT, CCGTCA, ACCGTG, CCGTGA, CCCGTT,
CCGTTG, TTCCGT, TCCGTG, CCCGTC, CCGTAA, CCGTCC, CCCGTA, CCGTTA, CCGTGC, CCGTTC, ACCCGT,
GACCGT, TCCGTC, ACCGTC, CCGTCT, TCCGTA, CCGTAG, CCCCGT, CCGTTT, TCCCGT, CCCGTG, TACCGT,
CTCCGT, CCGTGT, ATCCGT, ACCGTT, ACCGTA, GTCCGT, TCCGTT, CCGTAC, CCGTGG;
−3.7: TGTTGC,
GTTGCT, GTTGCG, AGGTGC, GGGTGC, CGTTGC, GTTGCA, GTTGCC;
−3.8: GGCAGC, AGCAGC;
−3.9: GGTCGA, AGTTGT, TGGTCG, CGGTCG, GAGTCG, AGGTCG, GGGTCG, AAGTCG, CAGTCG, AGTCGA,
GGTCGG, GGTTGT, AGTCGG, TAGTCG;
−4: TGGCGC, GGCGCC, CGGCGC, GGCGCA, GGCGCG, GGCGCT;
−4.1: GCGGCT, CGCGGC, TGCGGC, GCGGCA, GCGGCG;
−4.2: GGAGCC, TAGCCC, GAGCCT, AGGCCA,
CTAGCC, GGCCTA, AGCCCA, GGCCAA, TAAGCC, AAGCCT, GAGGCC, TAGGCC, CAGCCA, ATAGCC,
GGCCCT, GTAGCC, GAAGCC, AAGCCC, AGGCGT, AGGCCT, GGGCCA, AGAGCC, TTGGCC, TCAGCC,
GGCCCG, AGGGCC, TGGGCC, GTGGCC, CCGGCC, AGCCCC, AGCCTC, GGCCAG, GGCCAT, AAAGCC,
AGCCAT, CGGCCC, TGAGCC, CAAGCC, GCAGCC, TAGCCA, GGGGCC, CCAGCC, AGCCAA, AGCCTG,
CGGCCT, AGCCTA, GAGCCA, AGCCTT, GGCCCA, AAGGCC, AGCCCT, TTAGCC, TGGCCT, GGCCTC, ACGGCC,
TCGGCC, CAGGCC, GGGCGT, CAGCCT, CTGGCC, AGCCAG, TAGCCT, TGGCCA, ACAGCC, AGCCCG,
CGGGCC, CGAGCC, AAGCCA, GGCCCC, GGCCTT, GGGCCT, AGGCCC, CAGCCC, GGCCTG, ATGGCC,
GAGCCC, CGGCCA, TGGCCC, GGGCCC, GCGGCC;
−4.3: GTCGTT, GTCGTC, TGTCGT, AGCGGT, GGCGGT,
GTCGTG, GTCGTA, CGTCGT;
−4.4: CAGCTG, GGCTGG, GGCTGA, AGGCTG, AGCTGA, CGGCTG, GAGCTG,
GGGCTG, AAGCTG, TAGCTG, AGCTGG, TGGCTG;
−4.6: GCCAGC, GCACGC, GCTCGC, AGCCAC, GCCTGC,
GCGCGC, GGCCAC;
−4.8: GCTGTG, GCTGTC, GGTGGC, GCTGTA, CGCTGT, TGCTGT, AGTGGC, GCTGTT;
−5: GCCCGC, CTGCCG, TGCCGG, CGCCGA, TTCCGC, CCGCAC, CGCCGG, TACCGC, AACCGC, CCGCGA,
GCCGGC, CCGCGT, CCGCCG, TCCGCC, ACCGCC, ACCCGC, GCCGGG, CCCCGC, TCCCGC, CCCGCC, CCGCAA,
CCGCGG, GCGCCG, GTGCCG, GCCGAG, TCGCCG, GCCGAC, CCGCTA, CTCCGC, ACCGCT, CACCGC,
CCGCTC, TCCGCT, CCGCGC, GCCGAA, ACCGCG, CCGCAG, TCCGCG, CCGCCC, TGCCGA, CCGCTG, TTGCCG,
GCCGAT, CCCGCG, ATGCCG, ATCCGC, GACCGC, CCGCCA, CCGCCT, ACCGCA, GTCCGC, CCGCAT, CCGCTT,
GCCGGA, ACGCCG, GCCGGT, CCCGCA, CCCGCT, TCCGCA;
−5.3: AGTTGC, GGTTGC;
−5.6: AGGCGC;
−5.7: GTCGCA, TGTCGC, CGTCGC, GTCGCG, GTCGCC, AGCGGC, GGCGGC, GTCGCT; 5.9: GGTCGT, AGTCGT;
−6.2: GCTGCG, GCTGCA, TGCTGC, GCTGCT, GCTGCC, CGCTGC;
−6.4: AGCTGT, GGCTGT;
−6.6: GGCCGG,
AGCCGA, GAGCCG, AGGCCG, CAGCCG, AGCCGG, TAGCCG, GGCCGA, CGGCCG, GGGCCG, TGGCCG,
AAGCCG;
−7: GCCGTC, GCCGTA, GCCGTT, CGCCGT, GCCGTG, TGCCGT;
−7.3: GGTCGC, AGTCGC;
−7.8: GGCTGC, AGCTGC;
−8.4: GCCGCT, CGCCGC, GCCGCG, GCCGCA, GCCGCC, TGCCGC;
−8.6: AGCCGT,
GGCCGT.,
GTGGCT aSD:
−0.1: CCGGTA, CCGGTG, ACCGGG, ACCGGA, CGACCG, GTCCCG, ACCGAA, CCGAAG,
CAACCG, CCGGAC, AACCGT, TCCGAT, CCGTAT, TCCGGT, TCCCCG, TCCCGG, CCGAGA, TCCGGA, CCGACG,
ACCGAG, TTCCCG, GACCCG, ACCGTG, ACTCCG, TGACCG, CCCCGG, ATCCGG, GATCCG, TAACCG,
CCCGGG, CCGTGA, CCCGTT, CCGATC, CCGACA, CATCCG, ATCCGA, TATCCG, CCGGAG, CCGTTG, TTACCG,
CCCGAC, ACCGAT, CTTCCG, CTCCCG, GACCGA, ACCGGT, TTCCGT, CCGAAA, CCGAGT, CCGAAC, TCCGTG,
CCCGAT, CCGACT, TCCGAC, TACCGA, CCGATG, ACCCGA, TTTCCG, CCGGGT, TTCCGA, ATTCCG, CCCGTC,
TTCCGG, TCCGAA, CCGTAA, TACCCG, CCGTCC, CCCGTA, AATCCG, CCGTTA, CCGTGC, CCCGGT, CCGGGG,
CCGTTC, CCGGAA, AACCCG, CCCGGA, ACCCGT, ATACCG, GTACCG, GACCGG, TACCGG, GACCGT,
CTCCGA, TCCGTC, TCTCCG, TCCGGG, CCGAGG, CCGGAT, ACCGAC, CCCCGA, CGTCCG, ACCGTC, TCCGAG,
CCGTCT, CCTCCG, TCCGTA, CCGTAG, AACCGG, TGTCCG, GTCCGA, CCGAAT, CCCCGT, CCCGAG, CCGTTT,
CCCCCG, ATCCCG, TCCCGT, CCCGTG, CTCCGG, CCGACC, TACCGT, CCGATT, CCCGAA, CTCCGT, TCCCGA,
CCGATA, CCGTGT, CCGGTT, ATCCGT, ACCGTT, ACCCGG, GTTCCG, ACCGTA, CCGGGA, GTCCGT, AAACCG,
GAACCG, TCCGTT, GTCCGG, ACCCCG, AACCGA, CCGTAC, CCGTGG;
−0.2: ACACTA, GCACGG, GGTGGT,
ACACTT, TCTGCG, AGGTGG, CACACA, CACCGT, CACGAA, CACCCG, ATGCAC, CTGCGG, GCGAGG,
GAACAC, TGGTGA, CACAAA, GGGTGG, GACACT, GACACC, TACACC, CACGTC, CACAAG, ACGCAC,
AAGTGA, TGCGGT, CTGCGA, CACAAT, CGGTGG, GTCTGC, CACTGG, CGCGGT, CACGGA, ACCTGC,
AAACAC, CACATA, GCGGGA, GGTGAA, GCGCGG, GGTGGG, CACTGC, GCACTC, TGCACG, ACGCGA,
CACCGA, AAGTGG, TGCGAG, TGCACC, CGCGAG, AACACC, TACTGC, ACGCGG, ATCTGC, CCCTGC,
AGTGAA, CACAGA, CACACG, CACTTA, GGGTGA, GAGTGA, GCACGA, GCGGGG, ACACAC, CACGTA,
CACCCT, GCGGAC, AACACG, CGGTGA, TTACAC, TGCACT, GCACCG, GACACG, GCACCT, CACATT, CACTAA,
ACACTC, CACTCC, CACACC, GCACCC, GCACTG, GTGCGG, CACGTG, TACACT, GCACTT, TTGCGG, CACGGT,
GCACGT, CACGAG, ATACAC, TGGTGG, CACTTG, CTGCAT, CACGAT, GCGAAT, GAGTGG, CACGGG,
CACAGG, ACACGG, TTGCGA, CACATG, TACACA, TACACG, CACATC, ACACCC, ACACGC, CACGTT, CTGCGT,
ACTGCG, GACACA, CACCTC, CGACAC, CACAGT, CAGTGG, CACACT, GCGGTT, TAACAC, GACTGC, CACCTA,
ACACCT, AACTGC, CGCGGG, ACACAA, CGCGAA, GCGCGA, TAGTGG, ACACAG, ACACCG, ACACTG,
AACACT, AGTGAG, CGCACG, CACTTC, CAGTGA, CACGCG, ACTGCA, CACCGG, GGTGGA, CACGCA,
GCGGGT, AGTGGT, CACAAC, CACTCT, AGGTGA, ACACGT, CTCTGC, TGCGGG, TGCGGA, CACTCG, CACCTT,
GCACTA, GCGAGA, CGCACT, CTGCAA, CTGCGC, GCGGTA, ACACGA, CAACAC, TAGTGA, TGACAC,
CACTAT, TTCTGC, CACTGT, CTGCAC, TTGCAC, GCGAGT, GCGGTG, GCGGAT, TCCTGC, TCTGCA, CACTCA,
AGTGGG, CACTTT, GCGAAG, CTGCAG, CACGAC, CGCACC, GCGGAA, CACCTG, AACACA, GCGGAG,
CACTAG, CCTGCG, CACCCC, ACACAT, CCTGCA, GCGAAC, TGCGAA, ATGCGA, CACTGA, CGCGGA,
GCGAAA, GTGCGA, GGTGAG, AGTGGA, ATGCGG;
−0.3: GCAACC, GCAACG, AGTAAC, GCAACT, GGTAAC,
CGCAAC, TGCAAC, GCAACA;
−0.4: TAGACC, GGACCT, CGCACA, GCACAA, CAGACC, GTACAC, AGACCT,
GTGCAC, TGCACA, AAGACC, GCGCAA, TGGACC, AGACCC, GGGACC, CGCGCA, AGGACC, GCGCAG,
CGGACC, GGACCC, GCACAG, TGCGCA, GAGACC;
−0.5: CGGTAC, TGGTAC, GGTACG, GGTACT, GGTACC,
GGTACA;
−0.8: TCGCAA, CCAACA, GTACCA, CCGTCG, AGGTAT, ACCAGG, TATCGC, CCCAAT, CTCCAA,
CCAGAG, TCCCCA, GTCGAG, TTCCAG, GAACCA, ATCCAG, CCAGAA, ACCAAA, ATCGCG, GTTATG, GTCGTT,
ATTCGC, AATCCA, GATCCA, TCTCCA, TACCAG, CCAGTA, AACCAA, ACACCA, ATCGCA, GTCCAG, ATCCAA,
CCAAAG, GCGTCG, CCAGAC, CCAAAT, ACCAAC, AACCAG, AAACCA, GTCGTC, ACTCGC, GACCCA, TTACCA,
CCAGAT, GTCGAC, GTCGGG, AATCGC, GTTATT, GTGTCG, TCGCGA, CCCCAG, ATGTCG, CTCCCA, CTCCAG,
CACCCA, GTCGAT, TAACCA, CCAAGA, CCCAAC, CCAATC, CCAACT, ACCCAA, TCCAGG, CCAATG, CGTCGG,
TATCCA, GTTCCA, TTCGCG, ACTCCA, TCCAAT, CCAGTT, TGTCGT, ACCAAT, CCCAGT, CCAAAC, CCCCAA,
TCCAGT, TCGCGT, CCCAAG, TCGCGC, TACCCA, ATACCA, CGTTAT, TACCAA, TGTCCA, GACCAA, CCAGGA,
TCGCGG, GATCGC, CCTCCA, TCGTCG, GTCGTG, CCAGTG, CCAACC, ATTCCA, ACCCCA, CCCAGA, TTTCGC,
TGTTAT, GCGTAC, TTTCCA, CTGTCG, GTCGGT, GTTCGC, TCCAAA, CCAAGT, TCGCAT, GTCGTA, ACCCAG,
CCAACG, TCCAAC, CCAATA, CCAAAA, TTCCAA, CGACCA, CACCAG, CATCCA, CATCGC, GTCCAA, TGTCGA,
CCCAGG, CTCGCG, GTTATA, TCCAGA, TTCGCA, ATCCCA, CCAATT, CGTCCA, CGTCGT, CGTCGA, CCAGGT,
GGGTAT, CTTCCA, GACCAG, ACCAAG, TCCAAG, GCATAC, TCCCAA, CAACCA, TCGCAC, GTCGGA, TCCCAG,
TCTCGC, CCCAAA, GTCGAA, ACGTCG, CTTCGC, GTTATC, TTCCCA, TGACCA, CCTCGC, CCAAGG, AACCCA,
ACCAGA, CCAGGG, CTCGCA, GTCCCA, ACCAGT, TGTCGG, TCGCAG, GCACCA, TTGTCG, CACCAA, CCCCCA;
−0.9: TCGCTC, CGCTCA, GTTGGC, GCTTTA, ACGCTC, CAAAGC, GAGGCG, TTTAGC, CGCTGG, GCTGTG,
ACAGGC, GTAGGC, ATTAGC, CGAAGC, CTCGGC, GCTTCG, CGTAGC, TTGGGC, GGAAGC, TGCGCT,
CCGGCG, AAGGCG, GCTTAC, CGCTCC, GTAGCA, GTAGCG, GATGGC, GGGGGC, TTGGCA, TGAAGC,
TTCGCT, ACCAGC, CGCTTA, CGCTCG, CCAGCG, GCTTTG, CTAGCG, GCGCTT, CAGCGT, AAGGGC, GATAGC,
CACAGC, CGAGCA, GCTTGG, TGCTGG, CCCGGC, ATGGCG, CGAGCG, GTGCTT, CGGCGT, GCGAGC,
CATAGC, GGTGCT, GAGCAG, AGAGGC, GCGCTC, TTTGGC, GCTCCG, ACGGCG, AGCAAG, CTCGCT,
TTGAGC, CGGGGC, ACGAGC, GATGCT, GCTGTC, GAAAGC, AATGGC, AACGGC, CCCAGC, TCGCTG,
TGAGCG, GGAGGC, GGCAGG, AGGAGC, AACGCT, CTCAGC, GGCGTA, GCTGGC, TATGGC, ACAGCG,
GCAGGC, AAGCAT, TCTAGC, GCTTAG, ATTGCT, TAGCGT, TACGCT, TTCGGC, ACGCTG, GCTTTC, AGGCGT,
TCTGCT, AGCATT, TAGCAA, GCTGAC, GAGCAT, TCGGCG, CCGGGC, TGCTCG, TTGCTC, TGGCAA,
CGGGCG, AAGCAG, TGGCAG, CTTGGC, CTGAGC, GCGCTG, CTAGGC, GCTTGC, TAAGGC, CCTGCT,
TGCTCT, TGGAGC, AACAGC, GGCGTC, AAAAGC, CAAGCG, CGGAGC, GCTTGT, GTGGGC, GCTCGA,
CAGGCG, CTGCTT, TTGGCG, TGCTTC, GCTTGA, GAGAGC, CGCTCT, ATAGGC, CAGCAG, CTAAGC, TCAAGC,
GACGCT, GCTTCC, AGCAAA, CGAGGC, AATAGC, TCGGGC, CGCTGA, TGGCGT, GTTAGC, TCCGGC,
GAGCGT, GTGCTG, CATGGC, ACGGCA, GCTCCT, GCTCTC, TGCTGA, CCAGGC, CTGCTC, CAGAGC, ATAAGC,
ATTGGC, TTAGGC, GCTGTA, CCAAGC, GGTAGC, ATGCTG, GGAGCA, AGCAGG, GGGAGC, TGTAGC,
TTAGCA, CGCTTT, AGCGTG, CTGGGC, CGCTTC, GAGCAA, GGCAAG, CGGCAA, TGCTTG, ACGGGC,
TTGCTT, CCTAGC, CCAGCA, CATGCT, ACTAGC, ACTGGC, ATGCTC, AAGAGC, ACAGCA, GCTGGA, TGCTTA,
ATGGCA, GCTCTG, CTTGCT, CGCGCT, AGCATA, ACAAGC, GCTTTT, TGGGGC, GCTTCT, TAGGGC, CTGGCG,
ACCGGC, AGAGCG, TCGAGC, GCTCTT, GCAGCA, GGCAAT, GAGGGC, AGCAAT, AAAGGC, CTTAGC,
TAGCAT, GCTGAG, GGACGC, TCAGCA, TTGCTG, GCTCTA, TAAGCG, GCTCGT, TTCAGC, CGCAGC, ATAGCA,
ATGGGC, TAGCAG, GTGCTC, GTAAGC, GGGCGT, AGCATG, ATGCTT, AGAAGC, TTTGCT, GCTCAG,
GACGGC, TGAGGC, AAAGCA, GGCAGT, GGCAGA, CTAGCA, TCAGGC, CGCTGT, GGCGTG, AGACGC,
TCAGCG, GGAGCG, CAAGGC, TCTGGC, AGAGCA, GCTGGT, GCTTAT, GAAGCA, TGCAGC, CCGAGC,
GAAGCG, CACGGC, AGCGTT, ATCAGC, TACAGC, GTCGGC, CCGGCA, TGCTGT, TAGAGC, AAGCAA,
CGTGCT, CGCTTG, GCTGTT, GCTCCA, AGGGGC, TAAAGC, GTGAGC, AGCATC, TATAGC, TAAGCA, GCTTAA,
TCCAGC, GCAGCG, AGCAGT, AGCGTC, TATGCT, GCTTCA, TTAAGC, GCAAGC, AGTAGC, GCTGGG,
CAGCAT, ATAGCG, ATCGCT, CAGCAA, ACGCTT, TGCTCA, TCGGCA, ATCGGC, TGCTTT, AGCGTA, CAAGCA,
GCTCCC, TGAGCA, AATGCT, TTAGCG, AAAGCG, GCTCGG, ATGAGC, CGGCAG, AGCAGA, GACAGC,
CCTGGC, ACTGCT, AGTGCT, GCGGGC, TGCTCC, GGCAAA, TCGCTT, AAGCGT, CTGGCA, GCTGAA, CACGCT,
TACGGC, GGGGCG, CAGGGC, AGGGCG, TGTGCT, GCTCAA, CTGCTG, GGCGTT, GCTGAT, TAGGCG,
TGGGCG, GAAGGC;
−1: AGTTAG, GGGTTA, GAGCGC, GAGTTA, GGTTAG, TGGTTA, AAGCGC, AAGTTA,
TAGCGC, AGGTTA, AGTTAA, CGGTTA, CAGCGC, CAGTTA, AGCGCT, TAGTTA, GGTTAA;
−1.1: TGTTGC,
GTTGCT, GTTGCG, AGGTGC, GGGTGC, CGTTGC, GTTGCA;
−1.2: TCACCC, CTACTT, ATCACT, TCTACC,
TCACGA, TCACAG, CTACAA, CTCACG, CTACGA, TACTAC, TCTCAC, TATCAC, CTACAG, TCTACG, TCACCT,
CTACTA, GACTAC, ATCACC, CTACAT, CCTACT, CCTACA, CTCACA, CTACTC, TTCACC, CTACCT, TCACAT,
CTACTG, CTACCA, CTACCC, GTTCAC, GATCAC, ATCACA, CTCACT, TTCTAC, CTACGT, ATTCAC, ACTACG,
CTACGC, CCTACG, AACTAC, TCACTG, GGCATG, ATCTAC, GGCATT, TCACTC, TCCTAC, CACTAC, ACTACT,
CTACAC, TCACAC, TCACGG, ACTACC, TTTCAC, TTCACT, AATCAC, TCTACT, TCACAA, CTACGG, TCTACA,
TCACTA, ACTACA, ATCACG, ACTCAC, CCTACC, TTCACA, TCACTT, GGCATA, TCACGT, CTCACC, CTCTAC,
ACCTAC, TGGCAT, TTCACG, TCACCA, CATCAC, GTCTAC, CTACCG , GGCATC, CTTCAC, CCCTAC, TCACCG,
CCTCAC, CGGCAT, TCACGC; 1.3: GGACAC, AGACAC, CGTGAC, AGACCG, GTGACA, GGACCG, GTGACT,
GTGACC, AGCGCG, TGTGAC, GTGACG;
−1.4: CAGCAC, CAGGCA, GAGCAC, TGGGCA, GGGCAG, GGGGCA,
AGGCAG, AAGCAC, AGGGCA, AGGCAA, GGGCAA, CGGGCA, AAGGCA, AGCACT, TAGCAC, TAGGCA,
AGCACG, GAGGCA, AGCACC;
−1.5: GTCAGA, ATGGTC, GGTCTA, GAGTCT, TCAGTC, GTCAAG, CGTCAA,
CCGTCA, GCGTCA, AGTCTG, TGTCAA, AGTCCG, CGAGTC, ACAGTC, AAGTCT, TGGTCT, TAGGTC, TCGGTC,
ACGTCA, GTCAGC, GTCAGG, GGGTCT, GTCAAC, GGTCCT, GTCAAA, GGTCCC, AGTCCT, ATAGTC, GAGGTC,
TAAGTC, AAGTCC, GTGTCA, AAAGTC, CAAGTC, GCAGTC, AGGTCT, CCGGTC, GGTCTT, CTGGTC, AGTCTT,
GGAGTC, CTGTCA, GTCAGT, GTGGTC, CCAGTC, GGTCCG, TCGTCA, TTGGTC, TAGTCT, CGGTCC, CAGTCC,
GGTCTG, AGTCTA, TAGTCC, AGGGTC, TGAGTC, AGGTCC, CAGGTC, CGGGTC, AGAGTC, CGTCAG,
TGGGTC, GGGGTC, AGTCCC, TGTCAG, GGGTCC, TGGTCC, GTAGTC, CAGTCT, CTAGTC, CGGTCT, GCGGTC,
GAAGTC, ACGGTC, TTAGTC, GTCAAT, ATGTCA, GAGTCC, TTGTCA, AAGGTC;
−1.6: CGTGGC, CGCGAT,
GGTGAT, GTGGCG, AGTGAT, GTGGCA, GCGATA, TGTGGC, GCGATT, AGTCTC, TGCGAT, GCGATG,
GGTCTC, GCGATC;
−1.8: AAGCGA, GAGCGA, TGGCGG, AGGCGG, GCGCAT, GAGCGG, GGCGGA, GGCGAA,
AGCGGA, CAGCGA, AGCGGT, CAGCGG, GGCGGT, TGGCGA, CGGCGA, CGGCGG, AGCGAA, AGGCGA,
TAGCGG, AGCGGG, TAGCGA, GGCGAG, GGCGGG, GCACAT, GGGCGG, AAGCGG, GGGCGA, GCTCAT,
AGCGAG;
−1.9: GCTAAG, ACGCTA, TTGCTA, CGCTAA, ATGCTA, CGCTAG, GCTAAA, GCTAGG, TGCTAA,
GCTAGC, CTGCTA, GCTAGT, GGCAAC, TCGCTA, GTGCTA, GCTAAC, TGCTAG, GCTAGA, GCGCTA, AGCAAC,
GCTAAT;
−2: AGCACA, GGACCA, AGTCCA, GGTCCA, AGACCA, AGCGCA;
−2.1: GTTACC, GTTACG, TGGCGC,
TGTTAC, GTTACT, AGGTAC, CGGCGC, GGCGCA, GTTACA, GGCGCG, CGTTAC, GGGTAC, GGCGCT;
−2.2: CCATAA, ACCATC, GGCAGC, CCCATC, GACCAT, CCATGT, AACCAT, CCATAT, CCATCG, CCATCC, TCCATT,
CCATTC, CCATTG, CCATTA, CCCCAT, TCCATC, CACCAT, CCATGG, TCCATG, CCATAC, CCATTT, ACCCAT,
ACCATG, ATCCAT, CCATGC, CCATAG, ACCATT, TTCCAT, CCATCA, TACCAT, TCCCAT, CCCATG, CCATCT,
CTCCAT, AGCAGC, CCCATT, CCCATA, GTCCAT, CCATGA, TCCATA, ACCATA;
−2.4: GGTCGA, TGGTCG,
CGGTCG, GAGTCG, AGGTCG, GGGTCG, AGTTAT, AAGTCG, CAGTCG, AGTCGA, GGTCGT, GGTCGG,
AGTCGG, TAGTCG, GGTTAT, AGTCGT;
−2.5: CAGCTG, GGCTCA, CAGGCT, GGCTTG, GTGGCT, GGCACA,
ACGGCT, GGCTGG, GGAGCT, AGGCTT, AGCTCG, GGCTTT, GAAGCT, ATGGCT, GTAGCT, GGCTGA,
AGGCTG, TGGCTC, AGCTTT, AGGGCT, AGCTTA, AGCTTG, TGAGCT, TGGGCT, GGCACT, ATAGCT, CGGGCT,
TAAGCT, CCGGCT, GGCTTA, TAGCTC, GGCACG, AGCTGA, GGCTTC, CAGCTC, GGCTCG, CAAGCT, CGGCTG,
GGCACC, AGAGCT, AGCTTC, CGGCTT, CCAGCT, GAGCTG, AAGCTT, TGGCTT, GGGCTC, AAGGCT, GCAGCT,
AGCTCA, TAGGCT, AGCTCT, GGGCTG, AAGCTC, TGGCAC, GGCTCC, AAGCTG, CTAGCT, TAGCTT, AGCTGT,
GAGCTT, CTGGCT, AGGCTC, CAGCTT, GGCTCT, AAAGCT, TCGGCT, GGGGCT, GAGGCT, CGAGCT,
CGGCAC, GGCTGT, TAGCTG, TTGGCT, GGGCTT, ACAGCT, GAGCTC, AGCTGG, TGGCTG, CGGCTC, TCAGCT,
AGCTCC, TTAGCT;
−2.6: CGCCCA, CGCGCC, GCCCTC, GCCCGT, GCCCGA, TGCCCC, GCCTGG, AATGCC,
GCCCTG, TGCGCC, CGCCTG, TTTGCC, CGCCCC, CGCCCT, TCGCCC, CGCCCG, AGCGCC, GCCCTA, GCCTAA,
GCCTGT, GGCGCC, TGCCCG, CTGCCT, TGCCTG, ATTGCC, AACGCC, GCCCGG, GCCTTG, TTCGCC, CGCCTA,
GCCCCC, GTGCCT, GGTGCC, GCCTGA, CACGCC, ATGCCT, GACGCC, ACGCCC, TCGCCT, ATGCCC, GATGCC,
CGTGCC, GCCCCT, TATGCC, TCTGCC, GCCTTA, CTTGCC, TTGCCC, CTCGCC, GCCCAT, AGTGCC, CTGCCC,
TGCCTA, GCCCAA, GCCCTT, CCTGCC, TGCCCT, GCGCCC, GCCTAG, TACGCC, GTGCCC, GCGCCT, ACGCCT,
TTGCCT, GTTGCC, GCCTTC, CGCCTT, GCCTAT, TGCCTT, ATCGCC, TGCCCA, TGTGCC, ACTGCC, GCCTTT,
GCCCAG, GCCCCG, GCCCCA, CATGCC;
−2.7: AGTTGC, GCACGC, GCTCGC, CGCCTC, GCCTCG, GCCTCC,
GCCTCT, GCCTGC, GCGCGC, TGCCTC, GGTTGC, GCCTCA;
−2.8: GGGCAT, AGGCAT;
−2.9: GCGACA,
GCGACG, GCGACC, CGCGAC, GTCATA, GTCATT, CGTCAT, GTCATC, AGTGAC, TGCGAC, GCGACT,
GGTGAC, TGTCAT, GTCATG;
−3.1: GCTCAC, GCCTAC, GCCCGC, TGGTCA, TTCCGC, CCGCAC, TACCGC,
AACCGC, CCGCGA, CCGCGT, TCCGCC, ACCGCC, ACCCGC, CCCCGC, GGGTCA, TCCCGC, CCCGCC, CCGCAA,
CCGCGG, AGGTCA, GCGCAC, CCGCTA, CTCCGC, ACCGCT, CACCGC, CCGCTC, AGTCAG, AGTCAA, TCCGCT,
CCGCGC, ACCGCG, CCGCAG, TCCGCG, CCGCCC, CCGCTG, GCACAC, GGTCAG, GAGTCA, CCCGCG,
ATCCGC, GACCGC, TAGTCA, CCGCCT, ACCGCA, AAGTCA, GTCCGC, CCGCAT, GGTCAA, CCGCTT, CAGTCA,
CGGTCA, CCCGCA, CCCGCT, TCCGCA;
−3.2: GCGGCT, GGTGGC, CGCGGC, GGCGAT, TGCGGC, AGCGAT,
AGTGGC, GCGGCA, GCGGCG;
−3.3: GCTATG, TGCTAT, CGCTAT, GCTATT, GCTATC, GCTATA;
−3.5: GCCGTC, CACCAC, CCCACT, CTGCCG, TGCCGG, CGCCGA, GGCTAA, CCACCG, CCACTG, CGCCGG,
CCACGG, AGCTAG, TGGCTA, CCCACG, GCCGGC, GCCGTA, TTCCAC, CCGCCG, CCCCAC, ACCACA,
GCCGGG, CTCCAC, CCACAA, CCACAG, CCCACC, TCCACA, GCCGTT, AGGCTA, CGCCGT, GCGCCG, GTGCCG,
CCCACA, GCCGAG, TCGCCG, CCACGA, CGGCTA, CCACAC, GCCGAC, TCCACT, AAGCTA, GCCGTG, ACCACC,
CAGCTA, GGGCTA, AACCAC, GCCGAA, CCACTT, ACCACT, TGCCGA, GACCAC, CCACGC, TTGCCG, AGCTAA,
CCACTC, GCCGAT, GCCCAC, CCACGT, CCACCA, CCACCT, ATGCCG, TGCCGT, TCCACG, CCACCC, ACCACG,
TCCACC, GAGCTA, GGCTAG, TACCAC, GTCCAC, ACCCAC, ATCCAC, TAGCTA, GCCGGA, ACGCCG, GCCGGT,
CCACAT, TCCCAC, CCACTA;
−3.6: GCTGCG, GCTGCA, TGCTGC, GCTGCT, GCTGCC, CGCTGC;
−3.7: GGGCGC,
AGTTAC, AGGCGC, GGTTAC;
−3.8: GTCGCA, TGTCGC, CGTCGC, GTCGCG, GTCGCC, GTCGCT;
−4.1: AGGCAC, GGGCAC;
−4.2: GCCAGC, GGAGCC, TAGCCC, GTCACT, GAGCCT, CTAGCC, GGCCTA, GTCACC,
ACGCCA, AGCCCA, GCGCCA, CGTCAC, GCCAAC, GCCAGA, TAAGCC, AAGCCT, GAGGCC, TAGGCC,
ATAGCC, GGCCCT, GTAGCC, GAAGCC, AAGCCC, AGGCCT, AGAGCC, TTGGCC, TCAGCC, GGCCCG,
AGGGCC, TGGGCC, GTGGCC, CCGGCC, AGCCCC, AAAGCC, GTGCCA, GCCAAG, CGGCCC, TGAGCC,
CAAGCC, GCAGCC, GGGGCC, CCAGCC, AGCCTG, TGTCAC, GCCAAA, TGCCAA, CGGCCT, AGCCTA,
AGCCTT, GGCCCA, AAGGCC, ATGCCA, AGCCCT, TTAGCC, TCGCCA, TGGCCT, ACGGCC, TCGGCC, CAGGCC,
CAGCCT, CTGGCC, CGCCAG, GCCAGG, TAGCCT, TGCCAG, GCCAAT, ACAGCC, GCCAGT, GTCACG,
AGCCCG, CCGCCA, CGCCAA, CGGGCC, CGAGCC, GTCACA, GGCCCC, GGCCTT, GGGCCT, CTGCCA,
AGGCCC, CAGCCC, TTGCCA, GGCCTG, ATGGCC, GAGCCC, TGGCCC, GGGCCC, GCGGCC;
−4.3: AGCCTC,
GGCCTC;
−4.5: AGCGAC, AGTCAT, GGTCAT, GGCGAC;
−4.6: GCTACT, GCTACC, TGCTAC, CGCTAC,
GCTACA, GCTACG;
−4.8: AGCGGC, GGCGGC;
−4.9: GGCTAT, AGCTAT;
−5.1: GGCCGG, AGCCGA, GAGCCG,
AGGCCG, CAGCCG, AGCCGT, AGCCGG, TAGCCG, GGCCGA, CGGCCG, GGGCCG, GGCCGT, TGGCCG,
AAGCCG;
−5.2: GGCTGC, AGCTGC;
−5.4: GGTCGC, AGTCGC;
−5.6: CGCCAT, GCCATT, GCCATG, TGCCAT,
GCCATC, GCCATA;
−5.8: AGGCCA, GGCCAA, CAGCCA, GGGCCA, GGCCAG, TAGCCA, AGCCAA, GAGCCA,
AGTCAC, GGTCAC, AGCCAG, TGGCCA, AAGCCA, CGGCCA;
−6.2: AGCTAC, GGCTAC;
−6.5: GCCGCT,
CGCCGC, GCCGCG, GCCGCA, GCCGCC, TGCCGC;
−6.9: GCCACC, GCCACG, GCCACA, TGCCAC, GCCACT,
CGCCAC;
−7.2: GGCCAT, AGCCAT;
−8.1: GGCCGC, AGCCGC;
−8.5: AGCCAC, GGCCAC.,
GGCTGG aSD: 10.1: CCAGCC;
−0.1: AACAGA, CAACCT, GTGCAG, CACCGT, CGACCG, CACCCG, GTCCCG,
GCAACC, ACCGAA, CCGAAG, GACAGG, CAACCG, AACCGT, ACAGGA, TCACAG, TCCGAT, CCGTAT,
TCCCCG, CAACAG, CCGAGA, CTACAG, CCGACG, ACCGAG, TTCCCG, GACCCG, ACCGTG, ACGCAG,
ACTCCG, TGACCG, ACAGAA, CAGAAA, GCATCC, CAGGGG, GATCCG, TAACCG, CCGTGA, CACCGA,
GCAGGG, CCGACA, CATCCG, ATCCGA, AAACAG, TATCCG, ACAGAG, CAGAGA, TTACCG, CCCGAC,
CACAGA, ACCGAT, GAACAG, TTACAG, CTTCCG, CTCCCG, GACCGA, CAGAAC, CAGAGG, TTCCGT,
ACAGAT, CACCCT, CCGAAA, TCCGTG, CCCGAT, TCCGAC, TACCGA, GCACCG, CCGATG, CAGGAC, CATCCT,
CAGGGA, CAGACA, ACCCGA, TTTCCG, TTCCGA, ATTCCG, GCACCC, TGACAG, TCCGAA, CCGTAA, TACCCG,
CAGATT, AATCCG, CAGGAG, CAGACG, CAGAGT, TTGCAG, TGCAGA, CAGGGT, AACCCG, CACAGG,
TAACAG, TACAGG, ATACCG, AACAGG, GTACCG, CATCCC, ACACCC, CAGAAG, GCAGAA, GTACAG,
GACCGT, CTCCGA, ACAGGG, TCTCCG, ATGCAG, ACAACC, CCGAGG, ACATCC, ACCGAC, ACAGAC,
ACACAG, ACACCG, CAGAAT, GCGCAG, CCCCGA, CGTCCG, TCCGAG, TCCGTA, CAGATA, CCGTAG,
TGTCCG, GTCCGA, CGCAGA, CCGAAT, TGCAGG, CCCGAG, CGCGTC, GCACAG, ATCCCG, CGACAG,
AGACAG, TACCGT, GCAGAG, CCGATT, CAGGAA, CCCGAA, CTCCGT, ATACAG, TCCCGA, GCAGAT,
CCGATA, GGACAG, CGCAGG, TACAGA, CAGGAT, ATCCGT, CTGCAG, GCAGGA, CAACCC, GTTCCG,
CACCCC, GACAGA, ACCGTA, TCGCAG, GTCCGT, GCAGAC, AAACCG, GAACCG, CAGATG, ACCCCG,
AACCGA, CCGTAC, CCGTGG;
−0.2: TTGGTT, TGGGTG, TGGGTA, CCGATC, TGAGTG, ACTCGC, ATGAGT,
CCGAAC, TTGAGT, CCCCCT, ATGGGT, ACCCCC, GTGGGT, CTCGCG, CCCCCG, TGAGTA, GTGAGT, TCTCGC,
TTGGGT, TCCCCC, CTCGCA, CTGAGT;
−0.3: CACGTC, CGGGGT, CTTGCG, CCGTTG, CCGTTA, CCGTTC,
CTTGCA, ACTTGC, TCTTGC, CCGTTT, CAGATC, CGAGGT, ACCGTT, TCCGTT;
−0.4: TCGTCC, GGACCT,
ATCGGG, TCGGAC, AATCGG, ACTCGG, GTTCGG, CTCGGA, TCGGAA, CATGTC, GTCGGG, AGACCG,
TTCGGA, AGACCT, GGACCG, ATTCGG, TTTCGG, CGTCGG, AAGACC, TTCGGG, TGGACC, CTCGGG,
CTTCGG, AGACCC, GGGACC, TCGGGA, AGGACC, CCTCGG, GATCGG, GGACCC, TCGGAG, CATCGG,
TCGGAT, GTCGGA, TCGACC, TCGGGG, TATCGG, ATCGGA, TGTCGG, GAGACC, TCTCGG;
−0.5: GGTAGT,
TGTAGT, GTAGTG, GTAGTA, CCTACT, TAGTAC, ATAGTA, TCTGTC, ATAGTG, TAGTAT, TAGTGT, AGTAGT,
CATAGT, CGTAGT, TAGTGC, TAGTGG, TAGTAG, TATAGT, TAGTGA, GATAGT, AATAGT, TAGTAA, CCTTCT;
−0.6: CGGACT;
−0.7: TCTACC, GTCACC, TCACCT, ATCACC, CTATGC, TTCACC, CTACCT, CCCGTA, CTACGC,
ACCCGT, ACTACC, TCATGC, CCCCGT, CTCACC, TGAGTT, TCCCGT, CCCGTG, CTGGGT, CTACCG, TGGGTT,
TCACCG, TCACGC;
−0.8: CCAACA, GTACCA, CACCAC, CCATAA, ACCATC, CCCATC, CCCAAT, ACCTGT,
CTCCAA, TCCCCA, GACCAT, GAACCA, ACCAAA, CCCTGT, AATCCA, GATCCA, AACCAT, TCTCCA, CCACGG,
CCATAT, AACCAA, ACACCA, GGACCA, CCCACG, ATCCAA, CCAAAG, CCAAAT, TTCCAC, ACCAAC, AAACCA,
CCCCAC, CCATCG, GACCCA, TTACCA, ACCACA, TCCATT, CTACCA, CCATTG, CCTGTA, CTCCAC, CTCCCA,
CACCCA, TAACCA, CCAAGA, CCACAA, CCCAAC, CCACAG, ACCCAA, CCATTA, CCCCAT, TCCACA, CCAATG,
TATCCA, TCCATC, GTTCCA, CACCAT, CCCACA, ACTCCA, CCATGG, TCCAAT, CCACGA, ACCAAT, TCCATG,
CCACAC, CCCCAA, TCCTGT, CTCGTC, CCCAAG, CCATAC, TACCCA, ATACCA, TACCAA, TGTCCA, CCATTT,
GACCAA, ACCCAT, AACCAC, ACCATG, ATCCAT, ATTCCA, ACCCCA, TTTCCA, TCCAAA, CCATAG, GACCAC,
CCAACG, TCCAAC, ACCATT, TTCCAT, CCATCA, CCAATA, CCAAAA, TTCCAA, TACCAT, CGACCA, CATCCA,
TCCCAT, CCCATG, GTCCAA, CTCCAT, ATCCCA, CCCATT, CCAATT, CGTCCA, CCCATA, CCTGTG, TCCACG,
CTTCCA, ACCACG, ACCAAG, TCCAAG, TCCCAA, CAACCA, CCCAAA, TACCAC, GTCCAC, GTCCAT, TCACCA,
TTCCCA, ACCCAC, ATCCAC, TGACCA, AGACCA, CCAAGG, AACCCA, CCATGA, CCACAT, GTCCCA, TCCATA,
TCCCAC, GCACCA, ACCATA, CACCAA, CCCCCA;
−0.9: GCTAAG, CGTGGC, TCGCTC, CGCTCA, GCTATG,
AGGCAC, AAGCGA, GCTTTA, ACGCTC, GGGCAC, CAAAGC, GAGGCG, CGCTGG, ACGCTA, GCTGTG,
GTAGGC, GGCACA, CGAAGC, GCTTCG, TTGCTA, GGAAGC, TGCGCT, CGCTAA, AAGGCG, GCTTAC,
GCTCAC, TGCTAT, GAGCGA, CGCTCC, GATGGC, GGGGGC, TGAAGC, TTCGCT, CGCTTA, CGCTCG,
GCTGCG, AGCGAC, GCTTTG, GCGCTT, TGGCGC, CGCTAT, AAGGGC, GCTACT, TGGCGG, GCTTGG,
ATGCTA, TGCTGG, GTTGCT, ATGGCG, GTGCTT, GGTGCT, GAGCAG, AGAGGC, GCGCTC, GCTCCG,
AGGCGG, AGCAAG, GTGGCG, GATGCT, AGCACA, GCTGTC, GAAAGC, AATGGC, GGGCGC, TCGCTG,
GGAGGC, GGCAGG, AGGAGC, AACGCT, GCTCGC, GCTACC, GGCGTA, GCTAAA, TATGGC, AAGCAT,
GCTTAG, ATTGCT, TACGCT, ACGCTG, GCTTTC, AGGCGT, AGCATT, GCTGAC, GAGCAT, TGCTCG, TTGCTC,
TGGCAA, GAGCGC, GTGGCA, AAGCAG, TGGCAG, GAGCAC, GCGCTG, GGTGGC, GCTTGC, TAAGGC,
GGCGAT, TGCTCT, TGCTAC, TGGAGC, GGCGTC, AAAAGC, CAAGCG, CCATTC, CGGAGC, GCTTGT,
GGGCAG, GCTCGA, GGCACT, CTGCTT, GGGGCA, TGCTTC, GCTTGA, GAGAGC, CGCTCT, ATAGGC,
CCAATC, GCTATT, CTAAGC, GCTGCA, TGCTGC, TGTGGC, TCAAGC, GAGCGG, GACGCT, GGCGGA,
GCTTCC, AGCAAA, GGCACG, CGCTGA, GGCGAA, TGGCGT, TGCTAA, GAGCGT, GTGCTG, CATGGC,
GCTCCT, GCTCTC, TGCTGA, CTGCTC, CAGAGC, ATAAGC, AGGCAG, AAGCGC, GCTGTA, ATGCTG,
AGCGGA, GGCACC, CTGCTA, GGAGCA, AGCAGG, GGGAGC, GGCGCA, GGCATG, AAGCAC, CGCTTT,
AGCGTG, CGCTTC, GAGCAA, GGGCAT, GGCAAG, GGCATT, TGCTTG, CGCTAC, TTGCTT, AGGCGC,
ATGCTC, AAGAGC, GCTGGA, TGCTTA, GGCGAC, ATGGCA, GCTCTG, GCTATC, AGGGCA, AGGCAA,
AGCATA, GGGCAA, ACAAGC, GCTTTT, TGGCGA, TGGGGC, GCTTCT, AAGGCA, TAGGGC, AGAGCG,
GCTCTT, GGCAAT, GAGGGC, AGCAAT, AAAGGC, GGCAAC, GCTGAG, TTGCTG, GCTCTA, TAAGCG,
GCTCGT, AGCGAA, TCGCTA, GTGCTC, GTAAGC, GGGCGT, AGCATG, ATGCTT, AGAAGC, TTTGCT, TGGCAC,
AGGCGA, TGAGGC, GGCGCG, AGCGAT, AAAGCA, GGCAGA, GTGCTA, CGCTGT, GGCGTG, AGCACT,
GGAGCG, CAAGGC, AGCGGG, AGAGCA, AGCGCT, GCTTAT, GAAGCA, GGCATA, GCTAAC, GAAGCG,
AGCGTT, GCTACA, TGCTGT, TAGAGC, AAGCAA, CTTGTC, CGTGCT, CGCTTG, AGTGGC, GCTGTT, GCTCCA,
AGGGGC, AGGCAT, TAAAGC, AGCATC, GCTACG, GGCGAG, TAAGCA, TAGGCA, GCTTAA, AGCGCG,
AGCACG, TGGCAT, GGCGGG, AGCGTC, TATGCT, GCTTCA, TTAAGC, GCTGCT, GCAAGC, GAGGCA,
GGGCGG, GCTGGG, AAGCGG, ATCGCT, ACGCTT, TGCTCA, GCGCTA, GCTATA, CCGTGT, AGCAAC,
GGGCGA, TGCTTT, AGCGTA, CAAGCA, GCTCCC, GGCATC, AATGCT, AAAGCG, GCTCAT, GCTCGG,
AGCGAG, AGCAGA, ACTGCT, AGTGCT, AGCACC, TGCTCC, GGCAAA, CGCTGC, TCGCTT, AAGCGT,
AGCGCA, GCTGAA, GGGGCG, CAGGGC, AGGGCG, GGCGCT, TGTGCT, GCTCAA, CTGCTG, GGCGTT,
GTCGCT, GCTGAT, GCTAAT, TAGGCG, GAAGGC;
−1: TAGTTT, CTTAGT, ATTAGT, CCTCCT, TAGTTC,
CCGACT, TAGTTG, TTAGTG, CAGGTG, TTTAGT, GCAGGT, ACAGGT, CCTCCA, TCCTCC, CCTCCG, GTAGTT,
ATAGTT, TTAGTA, CAGGTT, CAGGTA, GTTAGT, TAGTTA, ACCTCC;
−1.1: TCACCC, GTTGGC, GTCAGA,
TACTAG, ACTAGG, TTGGCA, CTCAGG, CGCTAG, TCTAGG, TTTGGC, AACTAG, TCTAGA, CTCTAG, GTCTAG,
TCTCAG, GTCAGG, CTTGGC, ACTCAG, GCTAGG, CTACCC, TTGGCG, CTTCAG, ATCTAG, TCATCC, CCTAGA,
TATCAG, ATCAGA, CCTAGG, CTAGAG, ACTAGA, CTAGAT, ATTGGC, TCAGAT, CTAACC, TCAGAG, CATCAG,
TCAGGG, CTAGGG, TCAACC, CGCGCT, CTAGAA, GCTCAG, TCCTAG, CCTCAG, TCAGAC, TTCAGG, ACCTAG,
GATCAG, ATCAGG, TGCTAG, TTCAGA, CGTCAG, AATCAG, TGTCAG, GACTAG, GCTAGA, ATTCAG,
TCAGAA, CTATCC, CCTTGC, TCAGGA, TTTCAG, CCTCGC, CACTAG, CTCAGA, GTTCAG, TTCTAG, CCCTAG,
CTAGGA, CTAGAC;
−1.2: TTCCGC, CCGCAC, TACCGC, AACCGC, CCCGTT, CCGCGA, CCGCGT, CCGCAA,
CCGCGG, CTCCGC, CACCGC, ACCGCG, CCGCAG, TCCGCG, ATCCGC, GACCGC, ACCGCA, GTCCGC,
CCGCAT, TCCGCA;
−1.3: CCCACT, CCTGTT, CCACTG, TTAGGC, CCAAAC, TCCACT, CCCTCC, CCACTT,
ACCACT, CCACTC, CCCCCC, CAGACT, CCACTA, CACGCT:
−1.4: TAGACC, TGCGGT, CGGTGG, CGCGGT,
CGAGTA, TACGGT, CGGTAC, ACGAGT, CGGTGC, CGGTGA, ACGGTA, CACGGT, TCGGGT, CGGGTA,
CATGCT, AGCGGT, GGCGGT, CGGTAT, GACGGT, GCGGGT, CGGTAA, ACGGTG, CGAGTG, CGGTAG,
AACGGT, GCGGTA, ACGGGT, CGGGTG, GCGAGT, GCGGTG, TCGAGT, CGGTGT;
−1.5: ATGGTC, GGTCTA,
GAGTCT, GGTCGA, GGTCGC, TGGTCA, AGTCTG, AGTCCG, AGTCAT, AAGTCT, TGGTCT, TAGGTC, TGGTCG,
GAGTCG, GGGTCT, AGGTCG, TCTGCT, GGTCCT, GGGTCG, GGGTCA, AAGTCG, GGTCCC, AGTCCT,
GAGGTC, TAAGTC, AAGTCC, GGTCAT, AAAGTC, CAAGTC, AGTCGA, AGGTCT, AGGTCA, GGTCTT,
GGTCGT, AGTCTT, GGAGTC, AGTCAG, AGTCAA, AGTCCA, GTGGTC, AGTCTC, GGTCCG, GGTCTC, AGTCAC,
GGTCAG, GGTCAC, GGTCTG, GAGTCA, GGTCCA, AGTCTA, GGTCGG, TTAGTT, AGGGTC, AGTCGG,
AGGTCC, CAGGTC, AGAGTC, GGGGTC, AGTCCC, AGTCGC, AAGTCA, GGGTCC, TGGTCC, GGTCAA,
AGTCGT, GAAGTC, GAGTCC, AAGGTC;
−1.6: TTGGGC, CCATGT, TTGAGC, TGAGCG, CTGAGC, TGGGCA,
CCGAGT, GTGGGC, CCAAGT, ATGGGC, CCACGT, GTGAGC, TGAGCA, ATGAGC, TGGGCG;
−1.7: CGGGGC,
CCAACT, CGAGGC, TTGGTC, CCATCT;
−1.8: CCGTCG, CCGTCA, CTCGCT, CTGGTG, CCTGGT, CTGGTA,
TCCGTC, ACTGGT, ACCGTC, TCTGGT, CCGTCT, GCTGGT;
−1.9: CGTAGC, GTAGCA, GTAGCG, GATAGC,
CATAGC, TAGCGT, TAGCAA, AATAGC, CGGTTG, GGTAGC, TGTAGC, CGAGTT, CGGTTC, TAGCGC, CTTGCT,
GCGGTT, CGGTTA, TAGCAT, ATAGCA, TAGCAG, TAGCGG, ACGGTT, TAGCGA, TAGCAC, CGGGTT,
TATAGC, CGGTTT, AGTAGC, ATAGCG;
−2: TCAGGT, CTAGGT;
−2.1: TGCAGT, ACAGTA, ACCCGC, GCAGTG,
CCCCGC, TACAGT, TCCCGC, CAGTAG, CAGTGT, CTGGGC, CGCAGT, CAGTGC, CACAGT, CAGTGG,
CAGTGA, GGCAGT, CAGTAT, CCCGCG, GACAGT, GCAGTA, AGCAGT, ACAGTG, AACAGT, CAGTAA,
CAGTAC, CCCGCA;
−2.2: ACCTGC, CCCTGC, CCTCCC, CCTTCC, CCTACC, TGAGTC, TGGGTC, TCCTGC,
CCTGCG, CCTGCA;
−2.3: CTGGTT, CCGTGC, CCGCGC, CGGACC;
−2.4: TTTAGC, TCGGTA, ACAGGC, ATTAGC,
CTCGGT, CAGGCA, GCAGGC, CAGGCG, TTCGGT, GTTAGC, TTAGCA, TCGGTG, CTTAGC, GTCGGT,
ATCGGT, TTAGCG;
−2.5: GGCTGC, GGCTCA, CAGGCT, GGCTTG, GTGGCT, GGCTGG, GGCTAA, GGAGCT,
GGCTAT, AGGCTT, AGCTCG, GGCTTT, GAAGCT, ATGGCT, GGCTGA, AGCTAG, AGGCTG, TGGCTA,
TGGCTC, AGCTTT, AGGGCT, AGCTTA, AGCTGC, AGCTTG, ATAGTC, TAAGCT, GGCTTA, AGCTGA, GGCTTC,
AGGCTA, GGCTCG, CAAGCT, AGAGCT, AGCTTC, AAGCTA, GAGCTG, GGGCTA, AGCTAC, AAGCTT,
TGGCTT, GGGCTC, AAGGCT, AGCTCA, TAGGCT, AGCTCT, GGGCTG, AAGCTC, TAGTCT, GGCTCC, AAGCTG,
AGCTAA, AGCTAT, AGCTGT, GGCTAC, GAGCTT, AGGCTC, TAGTCC, GGCTCT, AAAGCT, TAGTCA, TAGTCG,
GGGGCT, GAGGCT, GGCTGT, GAGCTA, GGCTAG, GGGCTT, GTAGTC, GAGCTC, AGCTGG, TGGCTG,
AGCTCC;
−2.6: CGCCCA, GCCGTC, GCCCTC, GCCCGT, GCCCGA, TGCCCC, GCCTAC, CGCCAT, GCCTGG,
CGCCGC, GCCCGC, AATGCC, CTGCCG, ACGCCA, GCGCCA, GCAGTT, CGCCGA, GCCCTG, TGCGCC,
GCCAAC, CGCCTG, TTTGCC, CGCCCC, CGCCCT, CAGTTC, CAGTTT, GCCATT, TCGCCC, GCCATG, CGCCCG,
AGCGCC, GCCCTA, GCCGCG, ACAGTT, GCCGTA, GCCTAA, GCCTGT, GGCGCC, CGCCTC, TGCCCG,
GCCACG, CTGCCT, TGCCTG, ATTGCC, AACGCC, GCCTCG, GCCTTG, TTCGCC, GCCTCC, GTGCCA, GCCAAG,
GCCTCT, TGCCAT, GCCGTT, GCCACA, TGCCAC, GCCGCA, CGCCTA, GCCACT, CGCCGT, GCCCCC, GTGCCT,
GCGCCG, GTGCCG, GGTGCC, GCCGAG, GCCTGA, TCGCCG, ATGCCT, GACGCC, ACGCCC, GCCGAC,
GCCAAA, TGCCAA, TCGCCT, GCCGTG, ATGCCC, GATGCC, CGTGCC, GCCCCT, TATGCC, GCCTTA, GCCTGC,
GCCGAA, TTGCCC, ATGCCA, GCCCAT, GTCGCC, AGTGCC, TGCCGA, TCGCCA, CTGCCC, TGCCTC, TGCCTA,
TTGCCG, GCCCAA, CAGTTA, CAGTTG, GCCGAT, GCCCTT, GCCCAC, TGCCCT, GCGCCC, GCCTAG, ATGCCG,
GCCAAT, GCCTCA, CGCCAC, GCCATC, TGCCGT, TACGCC, GTGCCC, GCGCCT, ACGCCT, TTGCCT, GTTGCC,
GCCTTC, CGCCAA, CGCCTT, GCCTAT, TGCCTT, ATCGCC, TGCCCA, TGTGCC, ACTGCC, GCCTTT, CTGCCA,
ACGCCG, TTGCCA, GCTGCC, GCCCCG, GCCATA, GCCCCA, TGCCGC;
−2.7: ACCGGG, ACCGGA, CCGGAC,
TGCCGG, TCCCGG, TCCGGA, CCCCGG, ATCCGG, CGCCGG, CCCGGG, CCGGAG, GCCGGG, GCCCGG,
CCCGTC, TTCCGG, CCGTCC, CCGGGG, CCGGAA, CCCGGA, GACCGG, TACCGG, TCCGGG, CCGGAT,
CACCGG, AACCGG, CTCCGG, CCGACC, TTGGCT, GCCGGA, ACCCGG, CCGGGA, GTCCGG;
−2.8: CGCGCC,
GCCGCT, CGAGCA, CGAGCG, CGGCGT, GCGAGC, ACGGCG, ACGAGC, AACGGC, CGGGCG, CGCGGC,
TCGGGC, ACGGCA, CGGCGC, CCGCTA, CGGCAA, ACGGGC, ACCGCT, CCGCTC, TCCGCT, CGGCGA,
CGGGCA, TGCGGC, TCGAGC, CGGCGG, AGCGGC, CCGCTG, GACGGC, CACGGC, CGGCAC, GCGGCA,
CCGCTT, GGCGGC, CGGCAG, GCGGCG, GCGGGC, CCTGTC, TACGGC, CGGCAT;
−2.9: TCGGTT:
−3: CCACCG,
CAGACC, GCCACC, CCCACC, CACGCC, CCAAGC, ACCACC, CCATGC, CCACGC, CCGAGC, CCACCA, CCACCT,
TCCACC, TTAGTC;
−3.1: TTCAGT, CTAGTG, ATCAGT, TCAGTG, CTCAGT, TCAGTA, GTCAGT, GCTAGT,
CTAGTA, TCTAGT, ACTAGT, CATGCC, CCTAGT;
−3.2: GCTGGC, TGAGCT, TGGGCT, ACTGGC, TCTGCC,
CTGGCG, TCTGGC, CCTGGC, CTGGCA;
−3.4: ACCAGG, CCAGAG, TTCCAG, ATCCAG, CCAGAA, GCCAGA,
CGAGTC, TACCAG, CGGTCG, GTCCAG, CCAGAC, CTAGGC, AACCAG, CCAGAT, CCATCC, CCCCAG, CTCCAG,
TCCAGG, CCAGGA, CCAACC, CCCAGA, ACCCAG, CGGTCC, TCAGGC, CGCCAG, GCCAGG, CACCAG,
CCCAGG, TGCCAG, TCCAGA, CGGGTC, CCACCC, GACCAG, TCCCAG, CGGTCT, GCGGTC, ACCAGA,
GCCCAG, CCAGGG, ACGGTC, CGGTCA;
−3.5: GGCAGC, CAGCGT, CACAGC, CAGCAC, GTAGCT, ACAGCG,
AACAGC, ATAGCT, CAGCAG, TAGCTC, CAGCGA, ACAGCA, CAGCGG, CTCGCC, GCAGCA, CGCAGC,
CAGCGC, TAGCTT, TGCAGC, TACAGC, AGCAGC, GCAGCG, TAGCTG, CAGCAT, CAGCAA, TAGCTA,
GACAGC;
−3.6: CTAGTT, CCGGGT, TCAGTT, CTTGCC;
−3.7: CCCGCT;
−3.8: CTCGGC, TTCGGC, TCGGCG,
CCTGCT, CTGGTC, GTCGGC, TCGGCA, ATCGGC;
−4: TTAGCT;
−4.1: ACAGTC, CAGTCG, GCAGTC, CAGTCC,
CAGTCA, CAGTCT;
−4.2: GGAGCC, GGCCGG, GAGCCT, AGGCCA, GGCCTA, AGCCCA, GGCCAA, TAAGCC,
AAGCCT, GAGGCC, TAGGCC, GGCCCT, GAAGCC, AAGCCC, AGGCCT, GGGCCA, AGCCGA, AGAGCC,
GGCCCG, AGGGCC, GTGGCC, AGCCCC, AGCCTC, GGCCAG, GGCCAT, AGCCAC, AAAGCC, GAGCCG,
AGCCAT, CAAGCC, GGCCGC, GGGGCC, AGGCCG, AGCCAA, AGCCTG, AGCCGT, AGCCTA, GAGCCA,
AGCCGG, AGCCTT, GGCCCA, AAGGCC, AGCCGC, GGCCGA, AGCCCT, TGGCCT, GGCCTC, CAGGCC,
AGCCAG, TGGCCA, AGCCCG, AAGCCA, GGCCAC, GGCCCC, GGCCTT, GGGCCT, GGGCCG, AGGCCC,
GGCCGT, TGGCCG, GGCCTG, ATGGCC, GAGCCC, TGGCCC, GGGCCC, AAGCCG;
−4.3: CCAGGT;
−4.4: GCGGCT, ACGGCT, TCGGTC, TTGGCC, CGGGCT, CGGCTG, CGGCTA, CGGCTT, CGAGCT, CGGCTC;
−4.5: CTAGCG, GTCAGC, CTCAGC, TCTAGC, CCGCCG, TCCGCC, ACCGCC, GCTAGC, CCTAGC, ACTAGC,
CCGCCC, TCAGCA, TTCAGC, CTAGCA, TCAGCG, ATCAGC, CCGCCA, CCGCCT, GCCGCC;
−4.7: CCGGTA, CCGGTG,
TCCGGT, ACCGGT, CCCGGT, GCCGGT;
−4.8: CTGGCT;
−4.9: TGGGCC, TGAGCC;
−5: CCGGGC;
−5.1: CAGCTG,
TCAGTC, CAGCTC, CAGCTA, GCAGCT, CAGCTT, ACAGCT, CTAGTC;
−5.2: TAGCCC, ATAGCC, GTAGCC,
TAGCCA, TAGCCG, TAGCCT, CCGGTT;
−5.4: CCAGTA, CCCGCC, CCCAGT, TCCAGT, CCAGTG, TCGGCT,
GCCAGT, ACCAGT;
−5.5: CCTGCC; 5.7: CCAGGC, TTAGCC;
−5.9: CCAGTT;
−6.1: CCGGCG, CCCGGC,
GCCGGC, CGGCCC, TCCGGC, CGGCCT, ACCGGC, ACGGCC, CTAGCT, CCGGCA, CGGGCC, CGAGCC,
CGGCCG, CGGCCA, TCAGCT, GCGGCC;
−6.5: CTGGCC;
−6.7: CCGGTC:
−6.8: GCCAGC, ACCAGC, CCAGCG,
CAGCCA, CCCAGC, GCAGCC, CAGCCG, CCAGCA, CAGCCT, ACAGCC, TCCAGC, CAGCCC;
−7.1: TCGGCC;
−7.4: CCAGTC;
−7.7: CCGGCT;
−7.8: CTAGCC, TCAGCC;
−8.4: CCAGCT;
−9.4: CCGGCC.

According to some embodiments, Table 3 includes the interaction strength of the canonical aSD sequence and non-canonical aSD sequences GCCGCG, CGGCTG, CTCCTT, GCCGTA, GCGGCT, GTGGCT and GGCTGG. The interaction strengths that appear in Table 3 are sorted by increasing interaction strength. The interactions gradually increase from weak, to intermediate, to strong interaction strengths. According to some embodiments, interaction strength classification as weak, intermediate or strong is organism specific. In some embodiments, organism specific interaction strength classifications as weak, intermediate and strong are provided in Table 1. According to some embodiments, the interaction strength classifications for a bacterium that is not listed in Table 1 can be deduced based on the interaction strength classification of a bacteria that is disclosed in Table 1 and has the closest evolutionary distance to it. In some embodiments, the interaction strength classification for a bacterium that is not listed in Table 1 can be deduced by using the strengths for a bacterium with the same aSD or aSD subregion sequence.

In some embodiments, the interaction strength is decreased by at least 1%, 5%, 10%, 15%, 20%, 25%, 30%, 35%, 40%, 45%, 50%, 55%, 60%, 65%, 70%, 75%, 80%, 85%, 90%, 95%, 97%, 99% or 100%, relative to the interaction strength between an unmodified region of a nucleic acid molecule and a ribosomal RNA. Each possibility represents a separate embodiment of the invention.

In some embodiments, a weak interaction is an interaction of at most 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.1, 2.2, 2.3, 2.4, 2.5, 2.6, 2.7 or 2.8 kcal/mol. Each possibility represents a separate embodiment of the invention. According to some embodiments, the interaction strength is decreased to a weak interaction strength. Organism specific interaction strengths are provided in Table 1. In some embodiments, the interaction strength of canonical aSD sequence and non-canonical aSD sequences are as provided in Table 3. Organisms specific aSD sequences are known in the art, and can be found, for example is Ruhul Amin, et al., “Re-annotation of 12,495 prokaryotic 16S rRNA 3′ ends and analysis of Shine-Dalgarno and anti-Shine-Dalgarno sequences”, PLoS One, 2018; 13(8).

In some embodiments, an intermediate interaction is an interaction between a weak and a strong interaction. According to some embodiments, the interaction strength is modulated to an intermediate interaction strength. In some embodiments, the interaction strength is decreased to an intermediate reaction strength. In some embodiments, the interaction strength is increased to an intermediate reaction strength. It will be appreciated by a skilled artisan that weak, strong and intermediate interactions are distinct to each prokaryote and what may numerically be a strong interaction for one organism may be weak for another. Organism specific interaction strengths are provided in Table 1. In some embodiments, the interaction strength of canonical aSD sequence and non-canonical aSD sequences are as provided in Table 3.

In some embodiments, the interaction strength is the interaction strength of a subregion of the nucleic acid molecule. In some embodiments, the subregion is at least 1, 2, 3, 4, 5, 6, 7, or 8 nucleotides long. Each possibility represents a separate embodiment of the invention. In some embodiments, the subregion is at most 5, 6, 7, 8, 9, 10, 11 or 12 nucleotides long. Each possibility represents a separate embodiment of the invention. In some embodiments, the subregion is between 4-12, 5-12, 6-12, 7-12, 8-12, 4-11, 5-11, 6-11, 7-11, 8-11, 4-10, 5-10, 6-10, 7-10, 8-10, 4-9, 5-9, 6-9, 7-9, 4-8, 5-8, 6-8 or 7-8 nucleotides long. Each possibility represents a separate embodiment of the invention. In some embodiments, the subregion is the size of a SD sequence. In some embodiments, the subregion is the size of an aSD sequence. In some embodiments, the subregion is 6-nucleotides in length. According to some embodiments, organisms specific 6-nucleotides subregions are provided in Table 3.

In some embodiments, the mutation is within more than one subregion. In some embodiments, the mutation modulates the interaction strength of each subregion differently. In some embodiments, increasing interaction is increasing the cumulative interaction of all the subregions comprising the mutation. In some embodiments, decreasing interaction is decreasing the cumulative interaction of all the subregions comprising the mutation.

In some embodiments, the mutation it is a silent mutation. In some embodiments, the mutation results in the alteration of an amino acid of the sequence encoded by the nuclei acid of the invention to an amino acid with a similar function characteristic. In some embodiments, a characteristic is selected from size, charge, isoelectric point, shape, hydrophobicity and structure. In some embodiments of the methods of the invention, the mutation results in a synonymous codon (Synonymous codons are provided in Table 4). In some embodiments, the mutation does not alter protein function. In some embodiments, the mutation alters protein function. As used herein, the term “silent mutation” refers to a mutation that does not affect or has little effect on protein functionality. A silent mutation can be a synonymous mutation and therefore not change the amino acids at all, or a silent mutation can change an amino acid to another amino acid with the same functionality or structure, thereby having no or a limited effect on protein functionality.

In some embodiments, the nucleic acid molecule comprises at least 1, 2, 3, 4, 5, 7 10, 20, 30, 40, 50, 60, 70, 80, 100, 200, 300, 400, 500, 1000 or 10000 mutations. Each possibility represents a separate embodiment of the invention. According to some embodiments, the nucleic acid molecule comprises mutations at least 1%, 2%, 3%, 4%, 5%, 6%, 7%, 8%, 9%, 10%, 15%, 20%, 25%, 30%, 35%, 40%, 45% 50%, 75% or 100% of positions of the nucleic acid molecule. Each possibility represents a separate embodiment of the invention. In some embodiments, more than one mutation is in the same region. In some embodiments, more than one interaction is in the same subregion. In some embodiments, the nucleic acid molecule comprises at least two mutations and wherein the two mutation are in different regions. In some embodiments, the nucleic acid molecule comprises at least two mutations and wherein the two mutation are in different subregions.

In some embodiments, the nucleic acid molecule comprises a second mutation in a different region than the at least one mutation. In some embodiments, the second mutation modulates interaction strength of the nucleic acid molecule to a 16S ribosomal RNA (rRNA). In some embodiments, the second mutation and at least one mutation modulate synergistically. It will be understood by a skilled artisan that a synergistic modulation will both effect translation in the same way. Thus, if the at least one mutation improves translation potential, then the second mutation also improves translation potential. Similarly, if the at least one mutation decreases translation potential, then the second mutation also decreases translation potential. The two mutations need to create this effect in the same way. For a non-limiting example, the at least one mutation could increase translation initiation efficiency, while the second mutation optimizes ribosomal allocation. Similarly, for example, the at least one mutation may affect early elongation and the second mutation may affect translation termination. In some embodiments, the at least one mutation and the second mutation both improve translation efficiency. In some embodiments, the at least one mutation and the second mutation both decrease translation efficiency. In some embodiments, improving translation efficiency is increasing translation efficiency.

Introduction of a mutation into the genome of a cell is well known in the art. Any known genome editing method may be employed, so long as the mutation is specific to the location and change that is desired. Non-limiting examples of mutation methods include, site-directed mutagenesis, CRISPR/Cas9 and TALEN.

TABLE 4
synonymous codons

| | | | |
|---|---|---|---|
| F | UUC/UUU | P | CCC/CCU/CCA/CCG |
| L | CUC/UUG/CUU/CUG/ | T | ACC/ACU/ACA/ACG |
| | CUA/UUA | | |
| I | AUC/AUU/AUA | A | GCC/GCU/GCG/GCA |
| M | AUG | S | USS/UCU/UCA/UCG/ |
| | | | AGU/AGC |
| V | GUC/GUG/GUU/GUA | Q | CAA/CAG |
| Y | UAC/UAU | N | AAC/AAU |
| STOP | UAA/UAG/UGA | K | AAG/AAA |
| D | GAC/GAU | E | GAG/GAA |
| C | UGU/UGC | W | UGG |
| R | CGU/CGC/CGA/CGG/ | H | CAC/CAU |
| | AGG/AGA | | |
| G | GGU/GGC/GGG/GGA | | |

In some embodiments, the nucleic acid molecule of the invention is part of a vector. In some embodiments, the vector is an expression vector. In some embodiments, the expression vector is a prokaryotic expression vector. In some embodiments, the prokaryotic expression vector comprises any sequences necessary for expression of the protein encoded by the nucleic acid molecule of the invention in a prokaryotic cell. In some embodiments, the expression vector is a eukaryotic expression vector.

### Cells

According to another aspect, there is provided a biological compartment, comprising a nucleic acid molecule of the invention.

According to another aspect, there is provided, a cell comprising a nucleic acid molecule of the invention.

In some embodiments, the biological compartment is a cell. In some embodiments, the biological compartment is a virion. In some embodiments, the biological compartment is a virus. In some embodiments, the biological compartment is a bacteriophage. In some embodiments, the biological compartment is an organelle. Organelles are well known in the art and include, but are not limited to, mitochondria, chloroplasts, rough endoplasmic reticulum, and nuclei.

In some embodiments, the cell is a genetically modified cell. In some embodiments, the cell is prokaryotic cell. In some embodiments, the cell is a eukaryotic cell. In some embodiments, the cell is a mammalian cell. In some embodiments, the cell is a bacterial cell. In some embodiments, the cell is in culture. In some embodiments, the cell is in vivo. In some embodiments, the cell is a pathogen. In some embodiments, the nucleic acid molecule of the invention is an endogenous molecule of the cell that has been mutated. In some embodiments, the nucleic acid molecule of the invention is a heterologous transgene or a heterologous gene that has been added to the cell. In some embodiments, the cell is a virally infected cell.

The bacteria may be selected from a phyla or classes including but not limited to Alphaprobacteria, Betaprotobacteria, Cyanobacteria, Delataprotobacteria, Gammaprtobacteria, Gram positive bacteria, Purple bacteria and Spirochaetes bacteria. According to some embodiments, the bacteria is selected from a phyla or classes selected from Alphaprobacteria, Betaprotobacteria, Cyanobacteria, Delataprotobacteria, Gammaprtobacteria, Gram positive bacteria, Purple bacteria and Spirochaetes bacteria. According to some embodiments the bacteria is selected from the list provided in Table 1. According to some embodiments, the bacterial cell is not Cyanobacteria or Gram-positive bacteria.

In some embodiments, the cell comprises increased fitness. In some embodiments, the cell comprises decreased fitness. In some embodiments, the cell produces increased amounts of the protein encoded by the nucleic acid of the invention as compared to the amount of protein produced by an unmutated nucleic acid.

In some embodiments, a cell comprises a nucleic acid molecule comprising at least one mutation at least one region of the nucleic acid molecule, the region is selected from the group consisting of:
a. positions −8 through −17 upstream of a translational start site;
b. positions −1 upstream of a translational start site through position 5 downstream of the translational start site;
c. positions 6 through 25 downstream of a translational start site;
d. positions 25 downstream of a translational start site through position −13 upstream of a translational termination site;
e. positions −8 through −17 upstream of a translational termination site; and
f. a position downstream of a translational termination site.

According to some embodiments, the nucleic acid molecule comprises a mutation at positions −8 through −17 upstream of a translational start site is introduced into a cell. According to some embodiments, the mutation increases the interaction strength between a nucleic acid molecule region and the 16S ribosomal RNA thereby improving the translation initiation stage.

According to some embodiments, the nucleic acid molecule comprises a mutation at positions −1 upstream of a translational start site through position 5 downstream of the translational start site is introduced into a cell. According to some embodiments, the mutation increases the interaction strength between a nucleic acid molecule region and the 16S ribosomal RNA thereby optimizing ribosomal allocation and chaperon recruitment in the cell.

According to some embodiments, the nucleic acid molecule comprises a mutation at positions 6 through 25 downstream of a translational start site is introduced into a cell. According to some embodiments, the mutation decreases the interaction strength between a nucleic acid molecule region and the 16S ribosomal RNA thereby increasing translation elongation efficiency and avoiding errant translation initiation.

According to some embodiments, the nucleic acid molecule comprises a mutation at positions 25 downstream of a translational start site through position −13 upstream of a translational termination site is introduced into a cell. According to some embodiments, the mutation modulated the interaction strength between a nucleic acid molecule region and the 16S ribosomal RNA thereby increasing the ribosome diffusion efficiency towards the regions surrounding the start codon and/or improving translation initiation efficiency. In some embodiments, the modulation is to an intermediate interaction strength.

According to some embodiments, the nucleic acid molecule comprises a mutation at positions −8 through −17 upstream of a translational termination site is introduced into a cell. According to some embodiments, the mutation increases the interaction strength between a nucleic acid molecule region and the 16S ribosomal RNA improving translation termination fidelity and/or efficiency.

According to some embodiments, the nucleic acid molecule comprises a mutation at a position downstream of a translational termination site is introduced into a cell. According to some embodiments, the mutation decreases the interaction strength between a nucleic acid molecule region and the 16S ribosomal RNA thereby keeping the small sub-unit of the ribosome attached to the transcript after finishing the translation cycle, improving the recycling of ribosomes and thus the translation process. According to some embodiments, the mutation increases the interaction strength between a nucleic acid molecule region and the 16S ribosomal RNA thereby keeping the small sub-unit of the ribosome attached to the transcript after finishing the translation cycle, improving the recycling of ribosomes and thus the translation process.

### Methods

By another aspect, there is provided, a method for improving or impairing the translation process of a nucleic acid molecule, the method comprising introducing a mutation into the nucleic acid molecule, wherein the mutation modulates the interaction strength of the nucleic acid molecule to a 16S ribosomal RNA, thereby improving the translation process of a nucleic acid molecule.

In some embodiments, the mutation is a mutation described hereinabove. In some embodiments, method improves the translation process. In some embodiments, the method impairs the translation process. In some embodiments, the translation process comprises translation potential. In some embodiments, translation process in a cell is improved or impaired. In some embodiments, the translation process comprises translation pre-initiation. In some embodiments, the translation process comprises translation initiation. In some embodiments, the translation process comprises early elongation. In some embodiments, the translation process comprises elongation. In some embodiments, the translation process comprises translation termination.

The term “expression” as used herein refers to the biosynthesis of a gene product, including the transcription and/or translation of the gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of a gene within a cell is well known to one skilled in the art. It can be carried out by, among many methods, transfection, transformation, viral infection, or direct alteration of the cell's genome. In some embodiments, the gene is in an expression vector such as plasmid or viral vector.

Recombinant expression vectors generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence that allows for expression of the nucleotide sequence (e.g. in an in vitro transcription/translation system or in a host cell when the vector is introduced into the host cell).

As used herein the term “in vitro” refers to any process that occurs outside a living organism. As used herein the term “in-vivo” refers to any process that occurs inside a living organism. In one embodiment, “in-vivo” as used herein is a cell within an intact tissue or an intact organ.

In some embodiments, the gene is operably linked to a promoter. The term “operably linked” is intended to mean that the nucleotide sequence of interest is linked to the regulatory element or elements in a manner that allows for expression of the nucleotide sequence.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

General methods in molecular and cellular biochemistry, such as methods useful for carrying out DNA and protein recombination, as well as other techniques described herein, can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998).

As used herein, the term “recombinant protein” refers to protein which is coded for by a recombinant DNA and is thus not naturally occurring. The term “recombinant DNA” refers to DNA molecules formed by laboratory methods of genetic recombination. Generally, this recombinant DNA is in the form of a vector, plasmid or virus used to express the recombinant protein in a cell.

Purification of a recombinant protein involves standard laboratory techniques for extracting a recombinant protein that is essentially free from contaminating cellular components, such as carbohydrate, lipid, or other proteinaceous impurities associated with the peptide in nature. Purification can be carried out using a tag that is part of the recombinant protein or thought immuno-purification with antibodies directed to the recombinant protein. Kits are commercially available for such purifications and will be familiar to one skilled in the art. Typically, a preparation of purified peptide contains the peptide in a highly-purified form, i.e., at least about 80% pure, at least about 90% pure, at least about 95% pure, greater than 95% pure, or greater than 99% pure. Each possibility represents a separate embodiment of the invention.

According to some embodiments, the invention concerns an isolated genetically modified organism, wherein at least one position of a nucleic acid molecule comprising a coding sequence comprises a sequence mutation wherein the genetically modified organism has a modified translation process as compared to an unmodified form of the same organism.

In some embodiments, improving comprises at least one of: increasing translation initiation efficiency, increasing translation initiation rate, increasing diffusion of the small subunit to the initiation site, increasing elongation rate, optimization of ribosomal allocation, increasing chaperon recruitment, increasing termination accuracy, decreasing translational read-through and increasing protein yield. In some embodiments, impairing comprises at least one of: decreasing translation initiation efficiency, decreasing translation initiation rate, decreasing diffusion of the small subunit to the initiation site, decreasing elongation rate, deoptimization of ribosomal allocation, decreasing chaperon recruitment, decreasing termination accuracy, increasing translational read-through and decreasing protein level.

By another aspect, there is provided a method of improving the translation process, the method comprising introducing a sequence mutation to a nucleic acid molecule comprising a coding sequence, thereby modulating the interaction strength of the nucleic acid molecule to a 16S ribosomal RNA and modifying the translation process of a nucleic acid molecule.

By another aspect, there is provided a method of modifying a biological compartment, the method comprising performing a method of the invention on a nucleic acid molecule, thereby modifying the translation potential of the nucleic acid molecule, expression the modulated nucleic acid molecule within the cell, thereby modifying a cell.

By another aspect, there is provided a method of modifying a biological compartment, the method comprising performing a method of the invention on a nucleic acid molecule within the cell, thereby modifying a cell.

According to another aspect, there is provided a method for producing a nucleic acid molecule having an optimized or deoptimized translation process, the method comprising:
a. selecting a nucleic acid molecule comprising a coding sequence, wherein the nucleic acid molecule interacts with a 16S ribosomal RNA,
b. profiling the interaction strength of each position of the nucleic acid molecule to the 16S ribosomal RNA;
c. profiling the interaction strength of each sequence mutation at each position of the nucleic acid molecule; and
d. introducing to the nucleic acid molecule a mutation that modulates the interaction strength to the 16S ribosomal RNA,
   thereby producing a nucleic acid molecule that is optimized or deoptimized for translation.

By another aspect, there is provided a method for producing a nucleic acid molecule having decreased or increased translation potential, comprising:
a. providing a sequence of the nucleic acid molecule;
b. calculating the interaction strength of every 6-nucleotide long subregion of the nucleic acid molecule to a 6-nucleotide long subregion of an aSD of a 16S rRNA of a target bacterium;
c. calculating the cumulative alteration to interaction strength caused by every possible mutation to the nucleic acid molecule; and
d. introducing at least 1 mutation to the nucleic acid molecule, wherein the mutations comprising at least the top 1 mutation that increase or decrease translation potential.
   thereby producing a nucleic acid molecule having decreases or increased translation potential.

In some embodiments, the biological compartment is a cell. In some embodiments, the biological compartment is an organelle. In some embodiments, the biological compartment is a virion. In some embodiments, the biological compartment is a bacteriophage.

In some embodiments, at least the top 1, 2, 3, 5, 10, 15, 20, 25, 30, 35, 40, 45, or 50 mutations are introduced. Each possibility represents a separate embodiment of the invention. In some embodiments, all introduced mutations increase the translation potential. In some embodiments, all introduced mutations decrease the translation potential. In some embodiments, the mutations are selected from the mutations described hereinabove. It will be understood that the mutations are region specific and increasing interaction strength in a particular region will either increase or decrease translation potential, which increasing interaction strength in a different region might have a different effect on translation potential. In some embodiments, the method produces nucleic acid molecules optimized or deoptimized for translation in a target bacterium. In some embodiments, the target bacterium is a bacterium described hereinabove.

According to some embodiments, profiling the interaction strength of a sequence mutation on the interaction strength between a nucleic acid molecule and a ribosomal RNA, comprises comparing the interaction strength of a mutated sequence to a ribosomal RNA to the interaction strength of an unmodified sequence to a ribosomal RNA.

### Computer Program Products

By another aspect, there is provided a computer program product for improving the translation process of a nucleic acid molecule, comprising a non-transitory computer-readable storage medium having program code embodied thereon, the program code executable by at least one hardware processor to:
a. sequence or access sequencing of a nucleic acid molecule that bind a 16S ribosomal RNA;
b. provide the interaction strength of the nucleic acid molecule to a 16S ribosomal RNA;
c. assign a mutation to the nucleic acid sequence; and
d. provide an output regarding the nucleic acid sequence assigned mutation.

By another aspect, there is provided a system for improving the translation process of a nucleic acid molecule, comprising:
a. one or more devices for providing the interaction strength of the nucleic acid molecule to a 16S ribosomal RNA;
b. a processor; and
c. storage medium comprising a computer application that, when executed by the processor, is configured to:
   i. sequence or access sequencing of a nucleic acid molecule that bind a 16S ribosomal RNA;
   ii. provide the interaction strength of the nucleic acid molecule to a 16S ribosomal RNA;
   iii. assign a mutation to the nucleic acid sequence; and
   iv. provide an output regarding the nucleic acid sequence assigned mutation.

By another aspect, there is provided a computer program product for profiling the interaction strength between a nucleic acid molecule and a 16S ribosomal RNA, comprising a non-transitory computer-readable storage medium having program code embodied thereon, the program code executable by at least one hardware processor to:
a. sequence or access sequencing of a nucleic acid molecule that binds a 16S ribosomal RNA;
b. create a null model for the nucleic acid molecule;
c. calculate the interaction strength of positions in the nucleic acid molecule that interacts with the 16S ribosomal RNA;
d. classify the position according to a trinary interaction strength of strong, intermediate, or weak;
e. provide an output regarding the interaction strength of the interacting positions in the nucleic acid molecule.

By another aspect, there is provided a computer program product for modulating translation potential of a nucleic acid molecule comprising a coding sequence, comprising a non-transitory computer-readable storage medium having program code embodied thereon, the program code executable by at least one hardware processor to:
a. measure or access a sequence of the nucleic acid molecule;
b. calculate the interaction strength of every 6-nucleotide long subregion of the nucleic acid molecule to a 6-nucleotide long subregion of an aSD of a 16S rRNA of a target bacterium;
c. calculate the cumulative alteration to interaction strength caused by every possible mutation to the nucleic acid molecule; and
d. provide an output modified sequence of the nucleic acid molecule comprising at least the top 5 mutations that increase or decrease translation potential.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement one or more of the disclosed embodiments described herein. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

By device for sequencing it is meant a combination of components that allows the sequence of a piece of DNA to be determined. In some embodiments, the testing device allows for the high-throughput sequencing of DNA. In some embodiments, the testing device allows for massively parallel sequencing of DNA. The components may include any of those described above with respect to the methods for sequencing.

In certain embodiments the system further comprises a display for the output from the processor.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### EXAMPLES

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998).

### Material and Methods

The analyzed organisms. We analyzed 551 bacteria from the following phyla or classes: Alphaprobacteria, Betaprotobacteria, Cyanobacteria, Delataprotobacteria, Gammaprtobacteria, Gram positive bacteria, Purple bacteria, Spirochaetes bacteria. We analyzed an additional 76 bacteria across the tree of life that do not have a canonical aSD sequence in their 16S rRNA. Additionally, we analyzed 207 bacteria with known growth rates. The full lists can be found in Table 1. All of the bacterial genomes were downloaded from the NCBI database (ncbi.nlm.nih.gov/) on October 2017. For each gene, aside from the annotated coding regions, we also analyzed the 50 nt upstream of the translational start site and the 50 nt downstream of the translational termination site (approximating the end of the 5′UTR, and the beginning of the 3′UTR respectively).

The rRNA-mRNA interaction strength prediction and profile. The prediction of rRNA-mRNA interaction strength is based on the hybridization free energy between two sub-sequences: The first sequence is a 6 nt sequence from the mRNA and the second sequence is the aSD from the rRNA. This energy was computed based on the Vienna package RNAcoFold35, which computes a common secondary structure of two RNA molecules. Lower, more negative free energy is related to stronger hybridization (See below).

The rRNA-mRNA interaction strength profiles include the predicted rRNA-mRNA hybridization strength for each position in each transcript (UTRs and coding regions), and in each bacterium. We calculated the interaction strength between all 6 nucleotide sequences along each transcript (UTR's and coding sequences) with the 16S rRNA aSD. For each possible genomic position along the transcripts we performed a statistical test to decide if the potential rRNA-mRNA interaction in this position is significantly strong, intermediate, or weak. For more details, see below. We also created Z-score maps of the strength of interactions, see below.

The null model. We designed for each bacterial genome 100 randomizations according to the following null model: UTR randomized versions were generated based on nucleotide permutation which preserves the nucleotide distribution, and specifically the GC content. The coding region randomized versions were generated by permuting synonymous codons, thus preserving the codon frequencies, the amino acid order and content, and the GC content of the original protein.

Similar rRNA-mRNA interaction strength profiles as the ones described above were computed for the randomized versions of the transcripts, to compute p-values related to possible selection for strong/intermediate/weak rRNA-mRNA interactions.

We computed an empirical p-value for every position in the transcriptome of a certain organism. To this end, the average rRNA-mRNA interaction strength in the position was compared to the average obtained in all of the randomized genomes. The p-value was computed based on the number of times the real genome average was higher or lower (depend on the hypothesis we checked) than the null model average. A significant position is a position with a p-value smaller than 0.05.

Protein levels. E. coli Endogenous protein abundance data was downloaded from PaxDB (pax-db.org/download), we used “E. coli—whole organism, EmPAI” published in 2012.

The rRNA-mRNA strength prediction. The definition of rRNA-mRNA interaction strength is based on the hybridization free energy between two sub-sequences. The first sequence is a 6 nt sequence from the mRNA and the second sequence is the aSD from the rRNA. The energy value was computed based on the Vienna package RNAcoFold, which computes a common secondary structure of two RNA molecules. The RNAcofold parameters were the default ones to correspond to all of the analyzed bacteria.

Lower and more negative free energy is related to stronger hybridization. We assumed that the interacting sub-sequence at the 16S rRNA 3′ end is TCCTCC (3′ to 5′). However, when we remove this assumption and infer it in an unsupervised manner, the results remain similar.

The rRNA-mRNA interaction strength profiles and selection strength. rRNA-mRNA interaction strength profiles are based on the predicted rRNA-mRNA hybridization strength for each position, in each transcript (UTRs and coding regions), and in each bacterium. We report the average profile of each bacterium.

The Vienna program RNAcoFold (see definition in the section above) was employed to calculate the free energy related to rRNA-mRNA hybridization strength (i.e. the energy which is released when two sequences “bind”). We calculated the interaction strength between all 6 nucleotide sub-sequences that begin in a specific position in the transcript (UTR's and coding sequence) with the 16S ribosomal RNA aSD. By calculating the interaction between the aSD and all possible 6 nt sub-sequences along the mRNA, we achieved the hybridization strength (interaction strength) profile at a resolution of single nucleotides. In order to decide if a position (across the entire transcriptome) tends to include sub-sequences with certain rRNA-mRNA interaction strength (strong, intermediate or weak) we compared it to the properties of sub-sequences observed in a null model in the same position (see further details regarding the null model below).

The intermediate rRNA-mRNA interaction definition. In order to define intermediate interaction strength, we devised an unsupervised adaptive optimization model that defines intermediate interaction strength thresholds. Our goal function in the algorithm was the number of significant positions for intermediate interactions. The algorithm selects thresholds (interaction strength values) and calculates significant positions for intermediate interactions compared to the null model. At each iteration, the thresholds are chosen greedily to improve the number of significant intermediate positions (as compared to the null model). This procedure was also computed for the null model sequences to demonstrate selection.

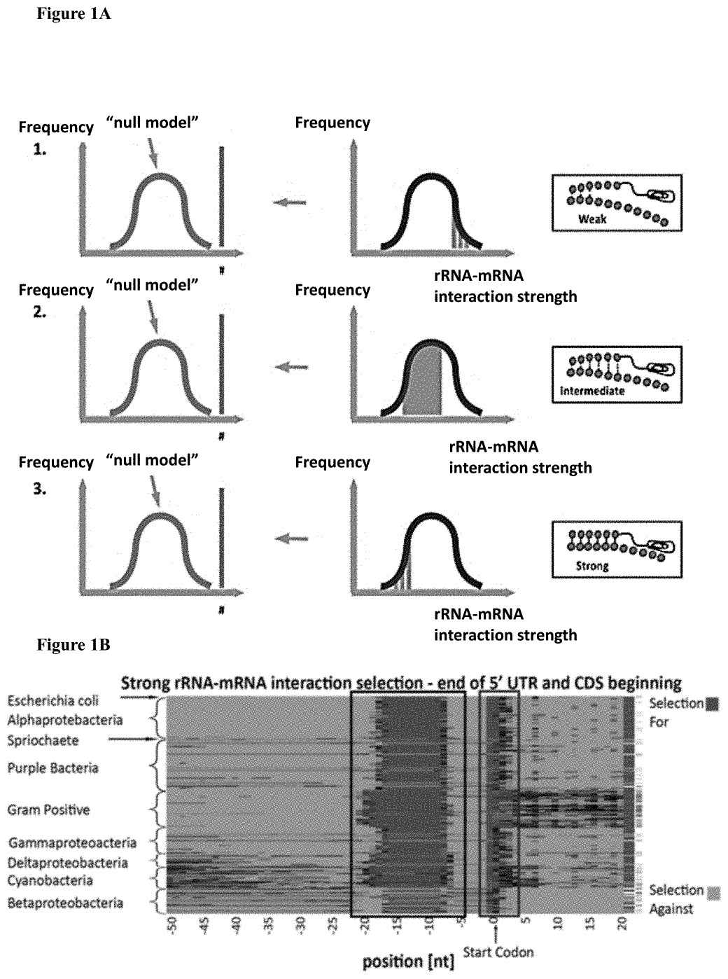

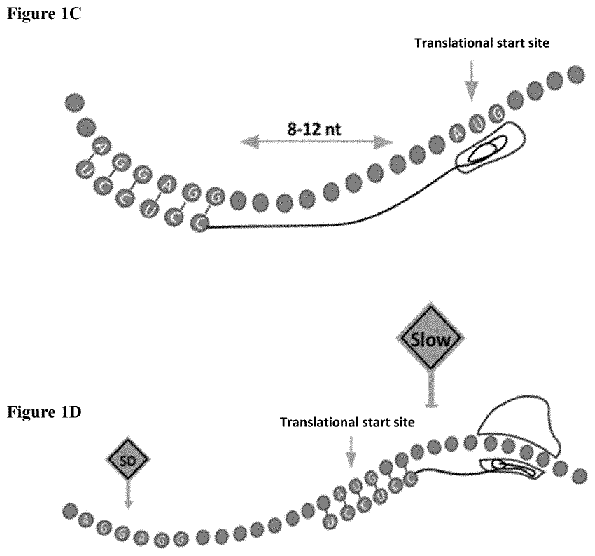

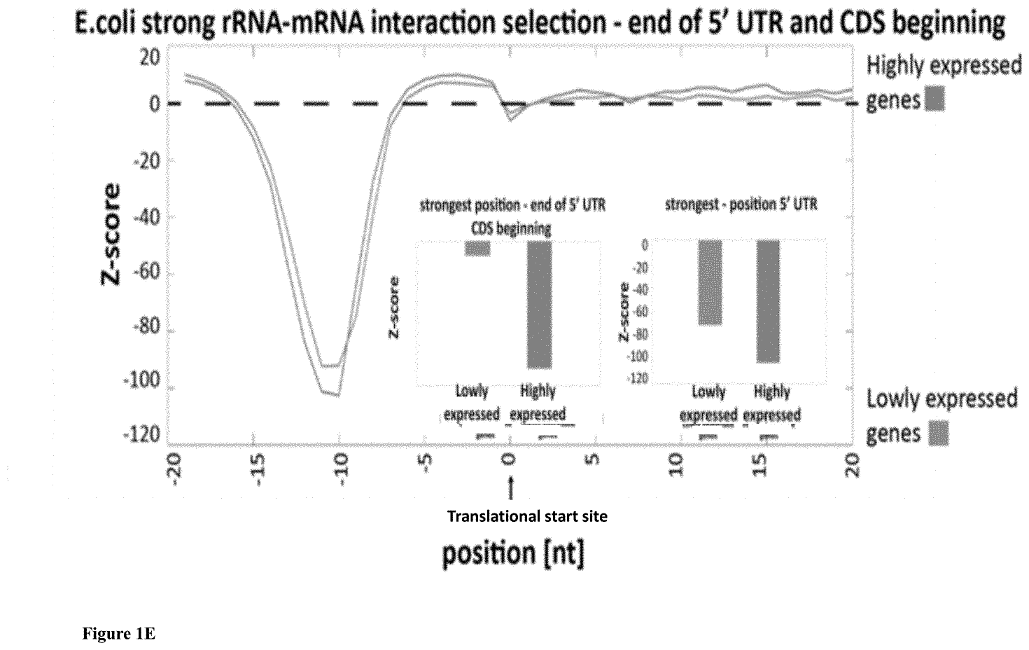

The first iteration thresholds were selected as follows; we created a distribution histogram of interaction strength in the region with the strong canonical SD interaction in the 5′UTR of each bacterium (positions −8 through −17, FIG. 1B). We calculated the area under the strong interaction distribution. We initially chose the ‘high’ (strongest interaction strength—more negative free energy) and ‘low’ (weakest interaction strength—less negative free energy) thresholds to be the interaction strength such that the area up to the chosen threshold interaction value was 5% of the total distribution area from each side of the curve.

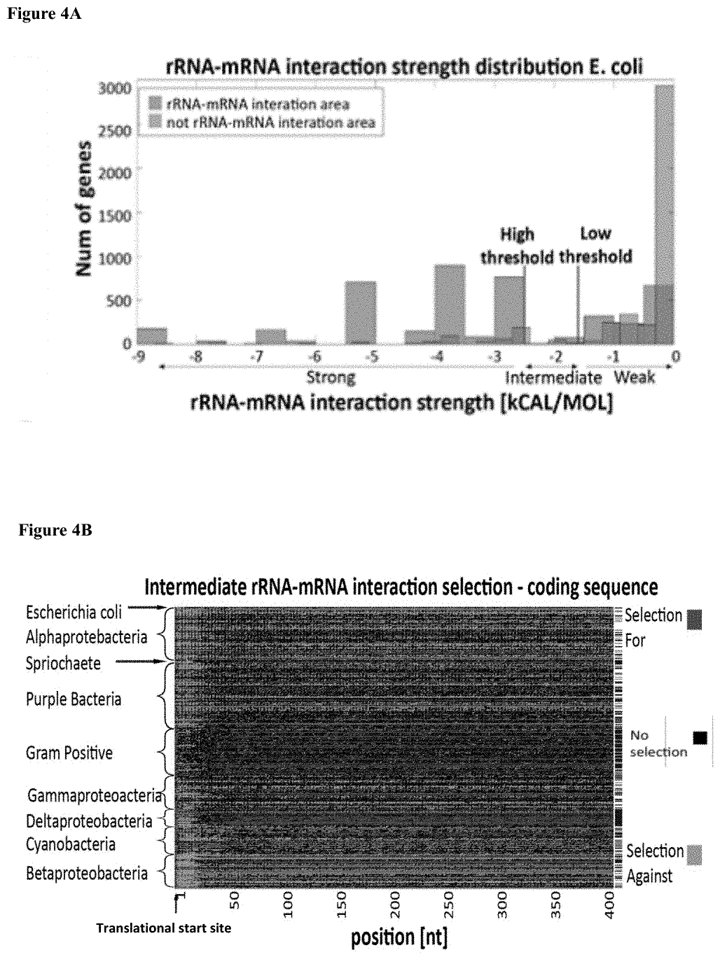

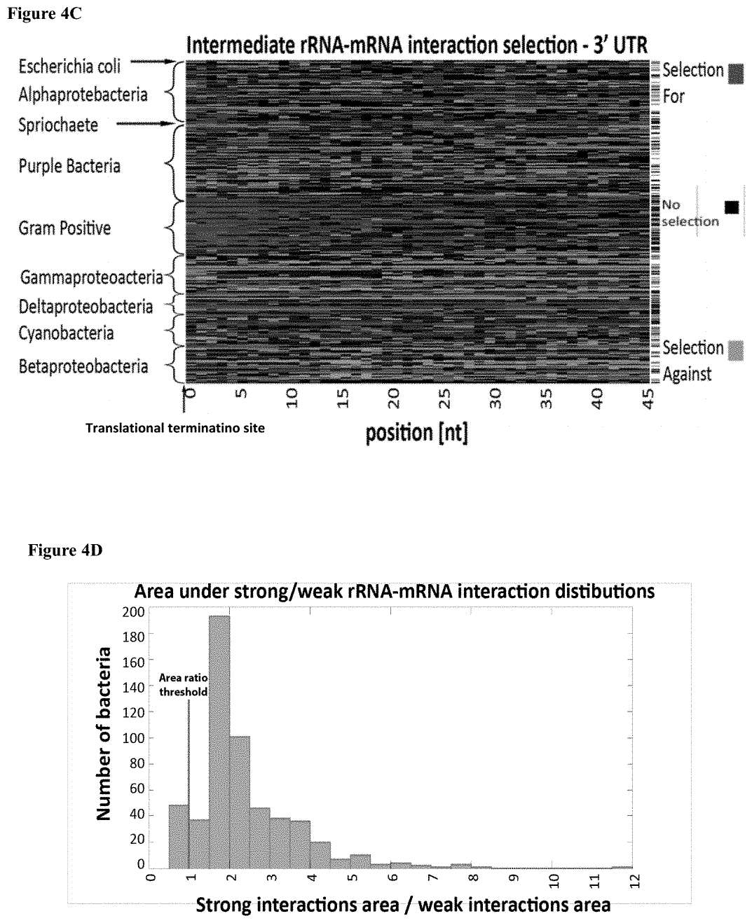

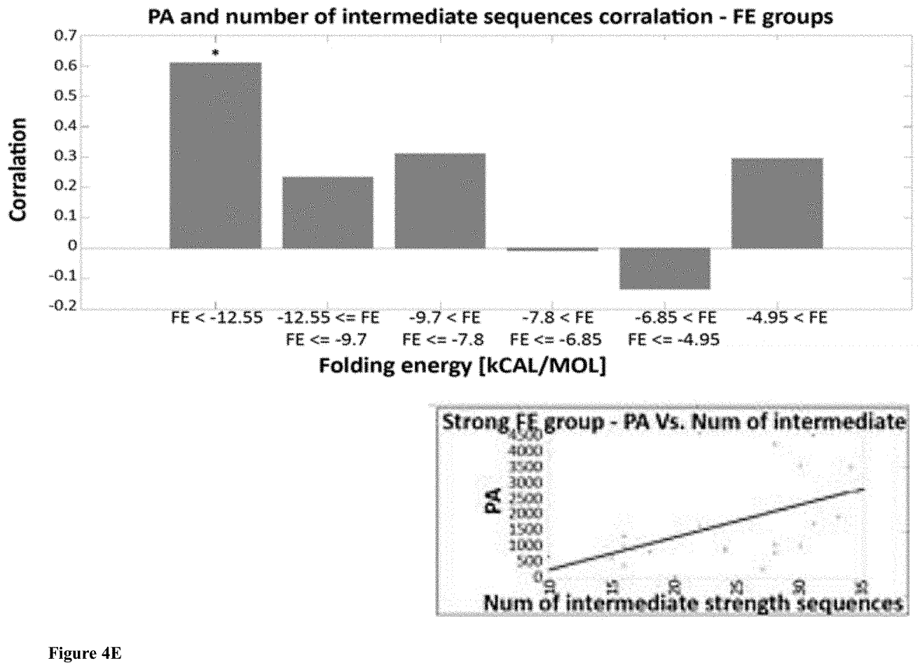

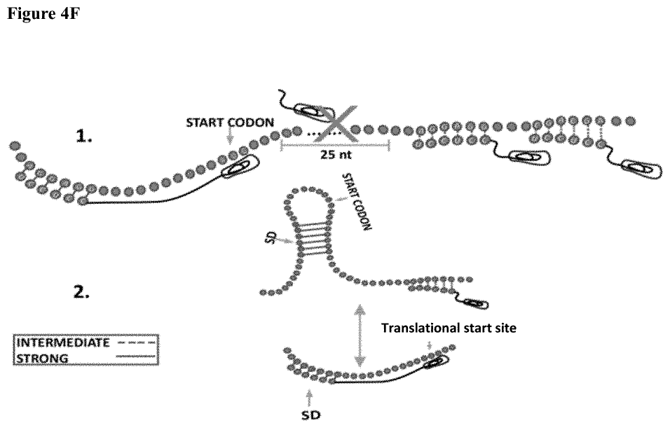

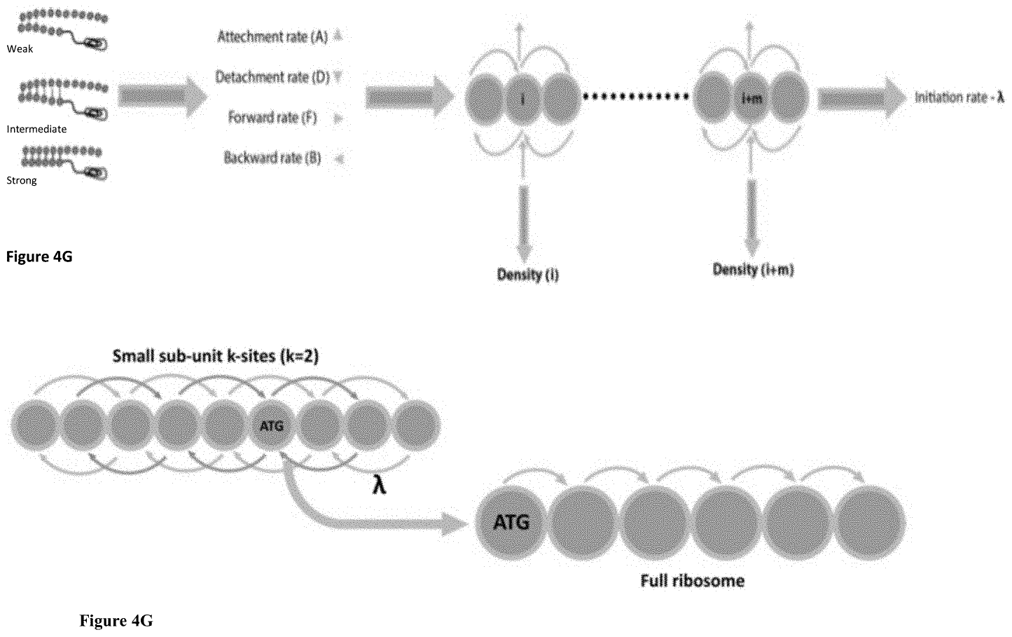

To study the properties of the selected thresholds, we created the interaction strength histograms for two regions in the 5′UTR (FIG. 4A): 1) The distribution of strong interaction strength as mentioned above. 2) The distribution of interaction strength in positions −40 to −50 at the 5′UTR upstream of the STRAT codon (where we do not expect to see strong rRNA-mRNA interaction, as this region doesn't have a known role in translation initiation).

Next, we looked at the positions of the two inferred thresholds in comparison to these two histograms; as can be seen in FIG. 4A, they tend to appear in the region between the two histograms supporting the hypothesis that these are indeed intermediate interaction strength.

To further quantitatively validate the inferred thresholds, we calculated the area under the two histograms mentioned above induced by the two inferred thresholds. The ratio between these two areas (the first one divided by the second one) was computed: A ratio larger than one suggests that it is more probable that the inferred thresholds are related to (intermediate) interactions between the rRNA and mRNA than to lack of interactions; indeed, in most bacteria (503/551) the ratio was larger than one (FIG. 4D).

Relation between the number of intermediate rRNA-mRNA interactions in the coding regions and heterologous protein levels. We aimed at showing that intermediate sequences in the coding region of a gene directly improve its translation initiation efficiency, and thus its protein levels. Hence, we calculated the partial Spearman correlations between the number of intermediate interaction sequences in the GFP variant and the heterologous protein levels (PA), based on 146 synonymous GFP variants that were expressed from the same promoter and the same UTR.

The control variables were the CAI and folding energy (FE) near the start codon. We defined an area of intermediate interactions according to the thresholds received by our model in E. coli and we expanded it by 20% to allow maximum intermediate interactions in this synthetic system (which is expected to differ from endogenous genes). The correlation was indeed positive and significant (r=0.35; P=2·10-5), suggesting that variants with more sub-sequences in the coding region that bind to the rRNA with an intermediate interaction strength tend to have higher PA.

Ribosome Profiling. E. coli Ribosome footprint reads were obtained from (SRR2340141,3-4). E. coli transcript sequences were obtained from NCBI (NC_000913.3). Sequenced reads were mapped as described in Diament, A. & Tuller, T. Estimation of ribosome profiling performance and reproducibility at various levels of resolution. Biol. Direct 11, 24 (2016) herein incorpatered by reference in its interity, with the following minor modifications. We trimmed 3′ adaptors from the reads using Cutadapt (version 1.17, described in Martin, M. Cutadapt removes adapter sequences from high-throughput sequencing reads. EMBnet. journal 17, 10-12 (2011), herein incorpatered by reference in its interity), and utilized Bowtie (version 1.2.1, described in Langmead, B., Trapnell, C., Pop, M. & Salzberg, S. L. Ultrafast and memory-efficient alignment of short DNA sequences to the human genome. Genome Biol. 10, R25 (2009), herein incorpatered by reference in its interity) to map them to the E. coli transcriptome. In the first phase, we discarded reads that mapped to rRNA and tRNA sequences with Bowtie parameters ‘-n 2-seedlen 21-k 1-norc’. In the second phase, we mapped the remaining reads to the transcriptome with Bowtie parameters ‘-v 2-a-strata-best-norc-m 200’. We filtered out reads longer than 30 nt and shorter than 23 nt. Unique alignments were first assigned to the ribosome occupancy profiles. For multiple alignments, the best alignments in terms of number of mismatches were kept. Then, multiple aligned reads were distributed between locations according to the distribution of unique ribosomal reads in the respective surrounding regions. To this end, a 100 nt window was used to compute the read count density RCDᵢ (total read counts in the window divided by length, based on unique reads) in vicinity of the M multiple aligned positions in the transcriptome, and the fraction of a read assigned to each position was RCDᵢ/Σⱼ₌₁^{M} RCDⱼ. The location of the A-site was set for each read length by the peak of read distribution upstream of the translational termination site for that length.

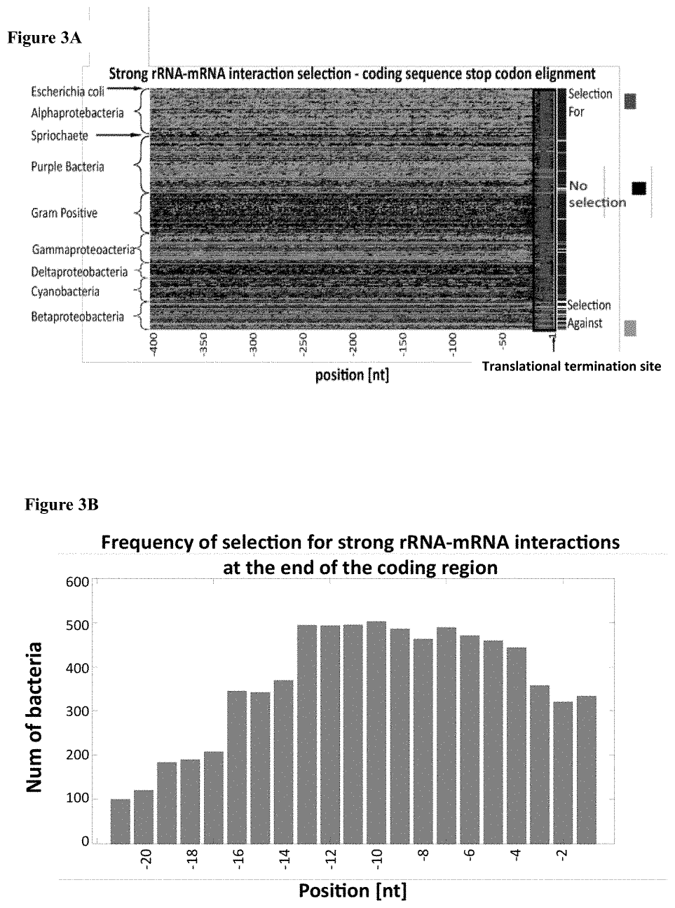

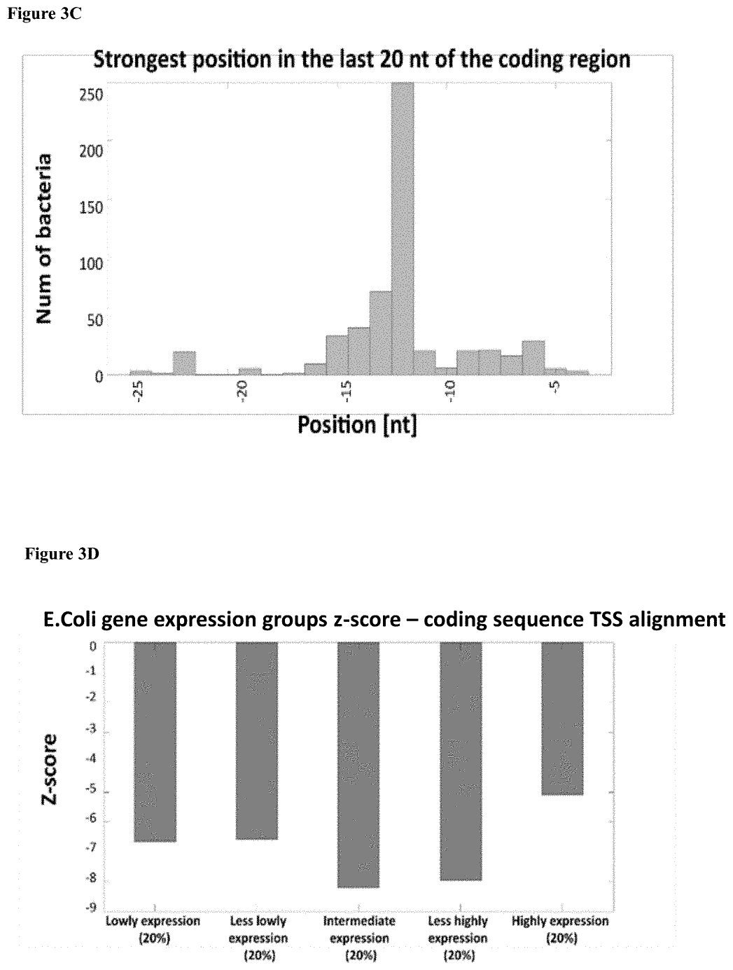

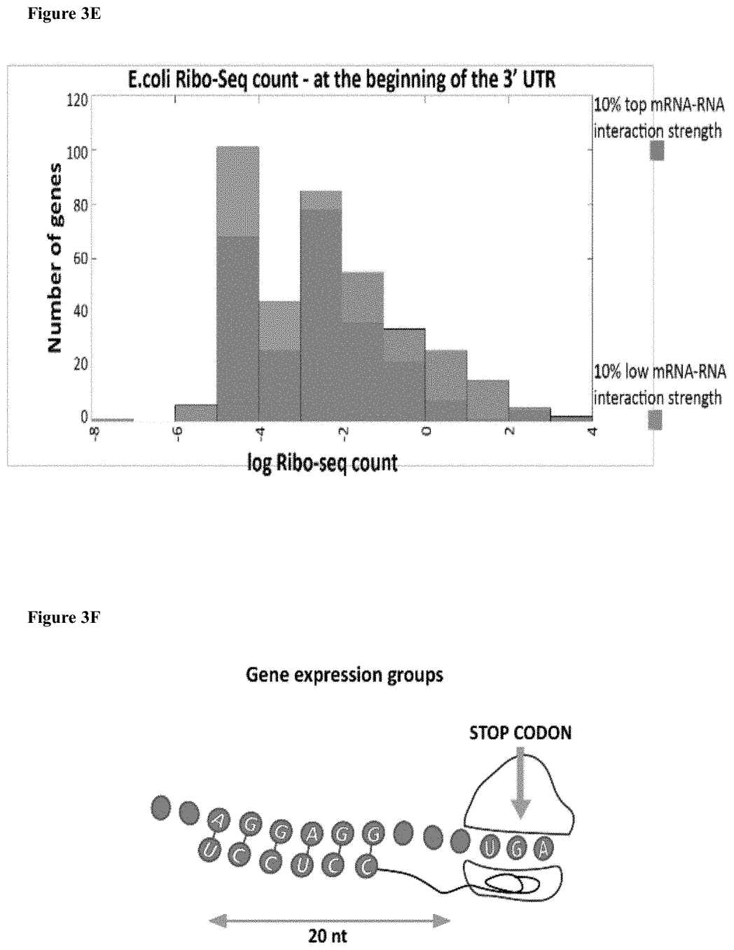

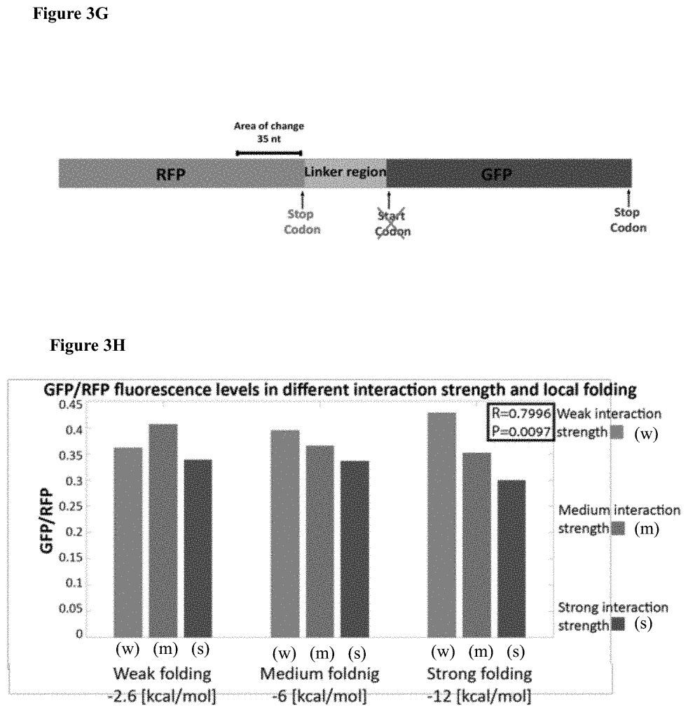

After creating the ribosome profiling distributions, for each gene, we calculated the number of positions with strong rRNA-mRNA interaction in the last 20 nucleotides of the coding region (the location of the reported signal, FIG. 3A). We ranked the genes according to their ‘number of strong positions’ and defined the 10% highest/lowest ranking genes. For the highest and lowest ranking genes, we calculated the average Ribo-seq read count in the first 20 nucleotides of the 3′ UTR (the closest region to the translational termination site), FIG. 3E.

Z-score calculation in highly and lowly expressed genes. To validate the reported signals, we performed all of our analyses on highly and lowly expressed genes of E. coli. We chose the highly and lowly expressed genes according to their PA (20% highest and lowest PA values), and computed Z-scores as explained in the next sub-sections.

Highly Vs. Lowly: Selection for Strong rRNA-mRNA Interactions at the 5′UTR End and at the Beginning of the Coding Region

We calculated the Z score based on the rRNA-mRNA interaction strength in all possible positions in the 5′UTR and coding region in the highly and lowly expressed genes.

$${Z}_{i} = \frac{real\_value ( i ) - mean\_rand \_value ( i )}{std\_rand \_value ( i )} \qquad ( 1 )$$

Zᵢ—Z-score in position i.
real_value(i)—rRNA-mRNA interaction strength in position i.
mean_rand_value(i)—Average rRNA-mRNA interaction strength in position i in all of the randomizations.
std_rand_value(i—Standard deviation of rRNA-mRNA interaction strength in position j in all of the randomizations.

The results of the Z-score analysis can be seen in FIG. 1E.

From a statistical point of view, we defined each gene by two values according to the reported signal: 1) Minimum Z-score value in position −8 through −17 in the 5′UTR. 2) Minimum Z-score value in position 1 through 5 at the beginning of the coding region. The regions were selected according to the reported signal in FIG. 1B.

We performed two Wilcoxon rank sum tests to estimate the p-values for the two reported signals in highly vs. lowly expressed genes.

Highly Vs. Lowly: Selection Against Strong rRNA-mRNA Interactions at the Beginning of the Coding Sequence

We calculated the Z-score (as described above) based on the rRNA-mRNA interaction strength of each position in the first 400 nt of the coding region in the highly and lowly expressed genes.

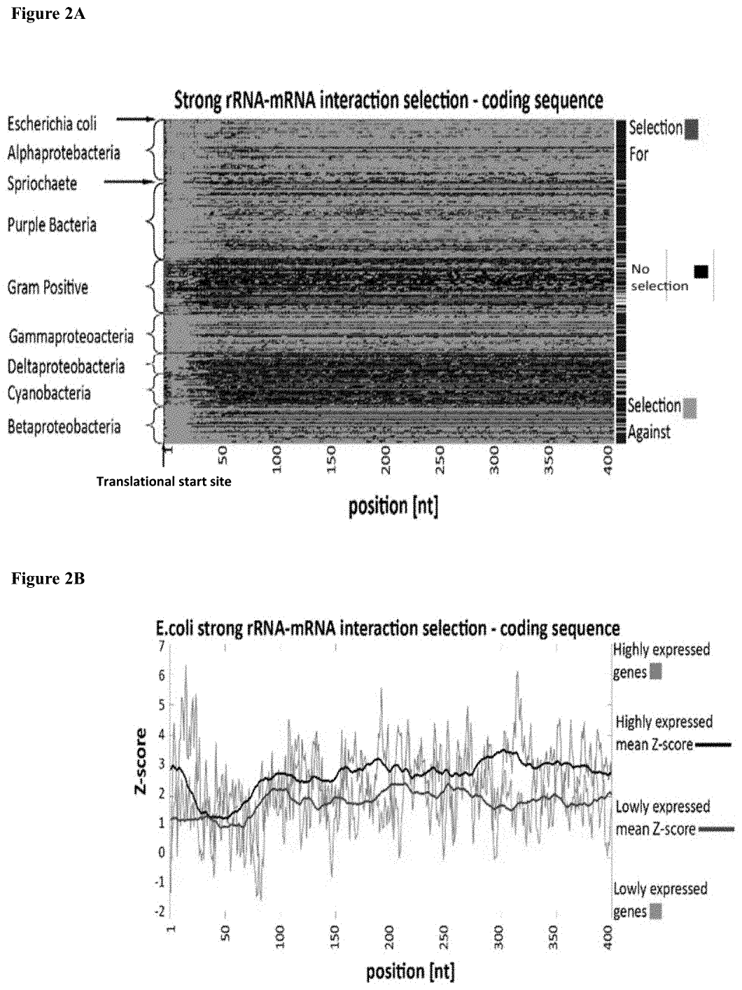

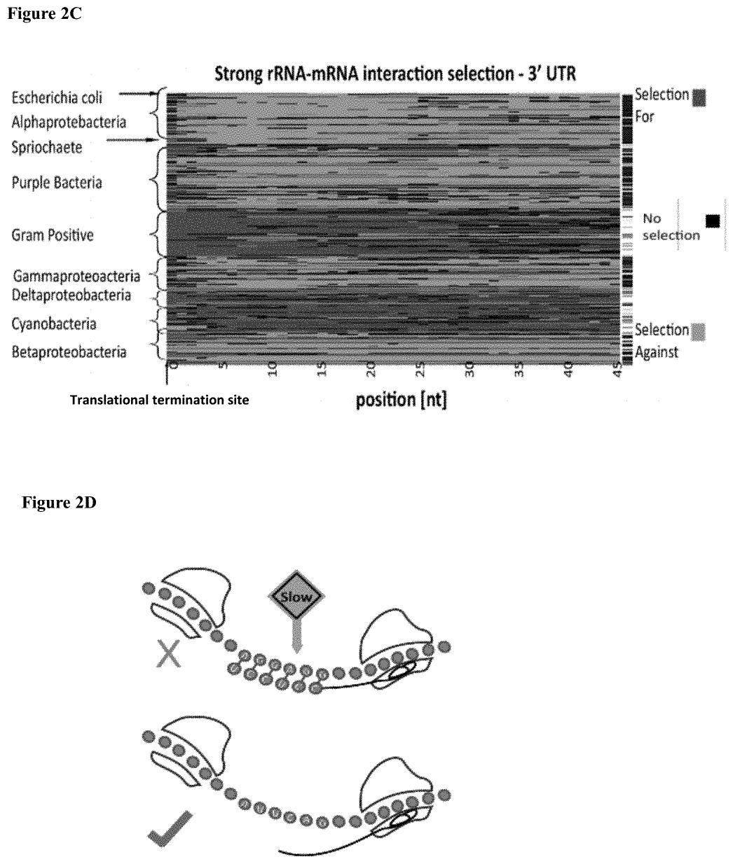

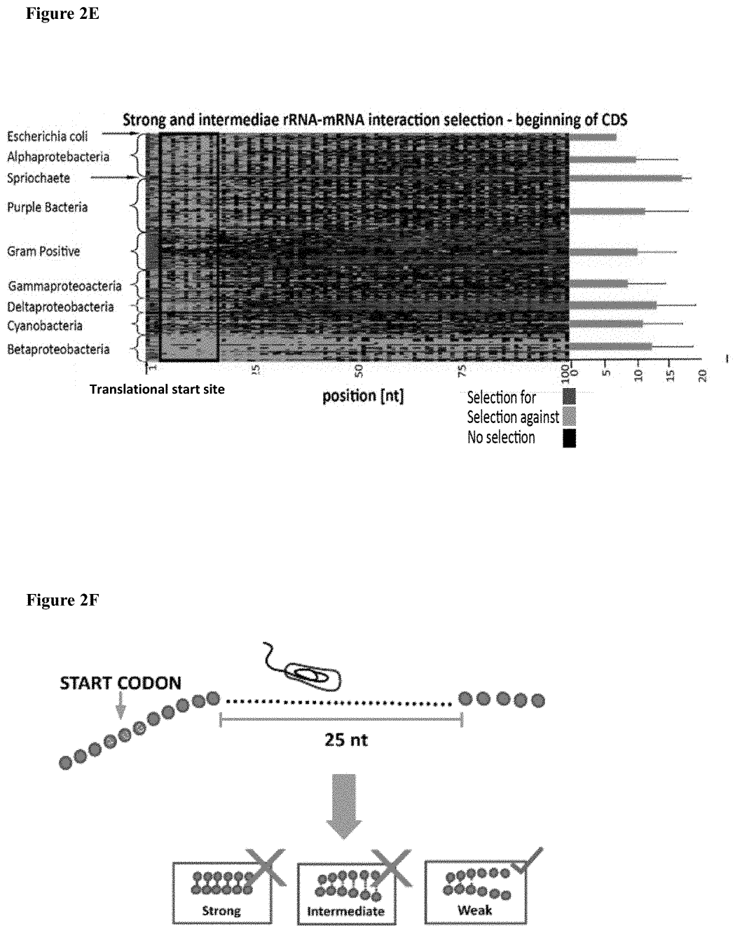

The results of the Z-score analysis can be seen in FIG. 2B. We performed Wilcoxon rank sum tests to estimate the p-values of the reported signals.

Highly Vs. Lowly: Z-Score Calculation of Selection for Strong mRNA-rRNA Interactions at the End of the Coding Sequence

In this case, we calculated the Z score (as described above) based on the rRNA-mRNA interaction strength of each position in the last 20 nt of the coding region in each bacterium.

For each bacterium, we found the position with a minimum Z-score value (strongest interaction compared to the null model). We created a histogram of the positions of strongest z-scores in the last 20 nt of the coding region distribution (FIG. 3C), and a histogram based on gene expression levels (FIG. 3D).

Selection against strong interaction in the coding region in positions that are not upstream to a close AUG codon. To detect signal of selection for/against strong interaction in the coding region after excluding positions that are upstream to a close start codon, we preformed the following analysis. We considered the E. coli genomes (both real and randomized versions) and in each gene we “marked”, position that are up to 14 positions upstream of an AUG (in all frames). We then computed p-value related to selection for strong rRNA-mRNA interactions (as mentioned before) but when we consider only the non-marked positions (both in the real and the randomized genomes). The result can be seen in FIGS. 12A-B.

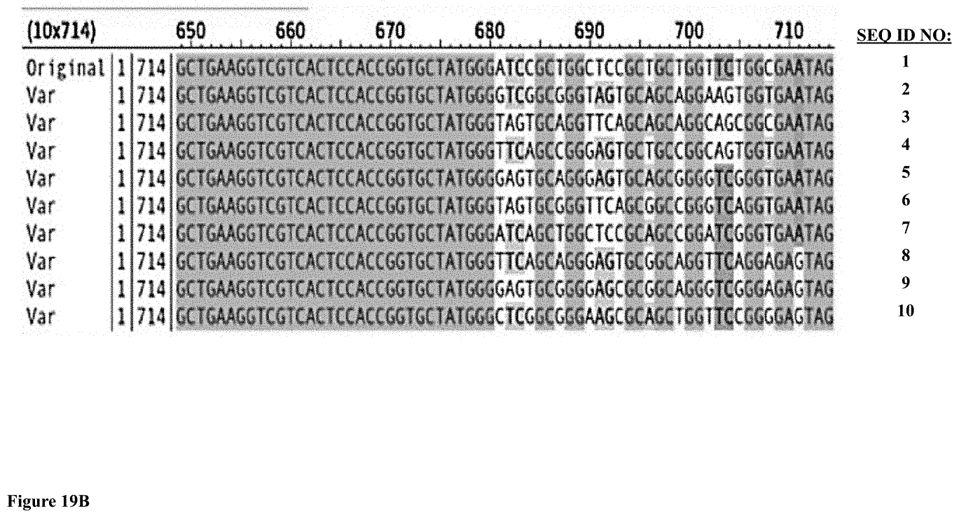

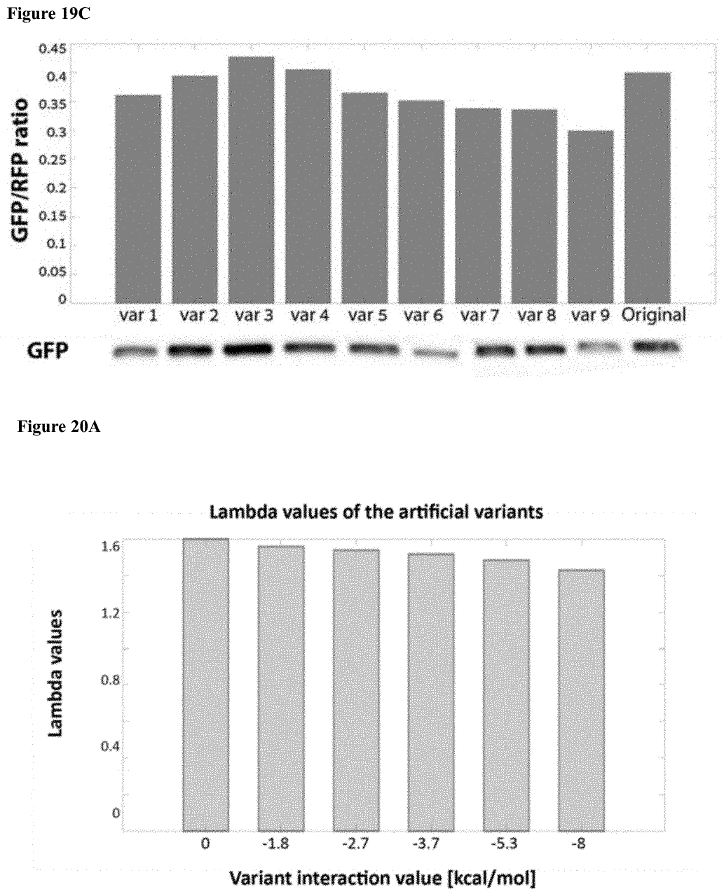

Read-through experiment to evaluate the effect of rRNA-mRNA interaction at the end of the coding region. To investigate the selection for strong rRNA-mRNA interaction at the end of the coding region (alignment to the STOP codon) we used a construct of RFP linked to a GFP (FIG. 3G). We designed nine variants with modifications at the end of the RFP with different levels of predicted rRNA-mRNA hybridization strength and local mRNA folding strength at the last 40 nt (FIG. 19A; Methods).

To investigate the selection for strong rRNA-mRNA interaction at the end of the coding region (alignment to the stop codon) we used a construct of RFP linked to a GFP (FIG. 3G). We created 9 variants with modifications at the end of the RFP with different levels of predicted rRNA-mRNA hybridization strength and local mRNA folding strength at the last 40 nt (FIG. 19A). We specifically checked 3 levels of predicted rRNA-mRNA hybridization strength (0, −0.9, −5.3) and 3 levels of predicted mRNA folding strength (2.3/3.3, −6, −12). The local mRNA folding energy in the last 40 nt of the coding region was calculated by the Vienna program RNAfold.

Unified biophysical translation model of the reported signals. We developed a computational simulative model of translation that includes the pre-initiation, initiation and elongation phases. Our model is based on a mean field approximation of the TASEP model. All of the model parameters are based on rRNA-mRNA interaction strength.

The model consists of two types of ‘particles’: 1. Small sub-units of the ribosome (pre-initiation): in this case, detachment/attachment and bi-direction movement of the particles is possible along the entire transcript. 2. Ribosome (elongation): the movement is unidirectional (from the 5′ to the 3′ of the mRNA) and possible only in the coding region; the initiation rate is affected by the density of the small sub-units of the ribosome at the ribosomal binding site (RBS).

Unified Biophysical Translation Model of the Reported Signals.

To validate that intermediate sequences in the coding region can improve the translation process by improving the pre-initiation diffusion of the small subunit to the initiation site and thus enhance the initiation phase of translation, we constricted a computational model of translation that includes the pre-initiation/initiation, and elongation phases. Our model is based on a mean field approximation of the TASEP model.

All of the model parameters are based on rRNA-mRNA interaction strength. The model consists of two types of ‘particles’: 1. Small sub-units of the ribosome (pre-initiation): their movement is possible through all of the transcript. 2. Ribosome (elongation): the movement is possible only in the coding region.

The model equations: Small sub-unit basic model. In this model there are several parameters that describe the movement of the small sub-unit in each site of the transcript. The small sub-unit can attach to the relevant site in the mRNA at a certain rate (depends on the rRNA-mRNA interaction value at that site). The small sub-unit can detach from a site at a certain rate (depends on the complementary interaction to the rRNA-mRNA interaction).

$$Attachmentn ( i ) = tanh ( \frac{interaction value ( i )}{epsilon} )$$

$$Detachmentn ( i ) = 1 - tanh ( \frac{interaction value ( i )}{epsilon} ) > 0$$

Attachment(i)=c1*Attachmentn(i) 3.

Detachment(i)=c1*Detachmentn(i) 4.

The movement forward of the small sub-unit to the next site depends on the detachment rate from the current site and the attachment rate of the next site.

Flow from cell i to cell i+1

Forward(i)=c2+(Detachment(i)*Attachment(i+1)) 5.

The movement backwards of the small sub-unit to the previous site depends on the detachment rate from the current site and the attachment rate of the previous site.

Flow from cell i+1 to cell i

Backward(i)=c2+(Detachment(i+1)*Attachment(i)) 6.

The start and end terms of the equations depends on the attachment or detachment of the first/last site.

“initiation” of the small sub-unit into the first site:

Forward(0)=c2+Attachment(1)

Backward(0)=c2+Detachment(1)

“termination” of the small sub-unit from the last site:

Forward(end)=c2+Detachment(end)

Backward(end)=c2+Attachment(end)

This is an example of the simple model equations that is based on the RFM. The density of ribosomes of site i depends on the flow to the site (from the site before and the next site), depends on the flow from site i (to the previous site and the next site) and the detachment and attachment rates of site i.

For example, i=2:

\dot{x}₂=Flow(1,2)x₁(1−x₂)−Flow(2,1)x₂(1−x₁)+Flow(3,2)x₃(1−x₂)−Flow(2,3)x₂(1−x₃)+Attachment(2)(1−x₂)−Detachment(2)x₂

Small sub-unit k-sites model. To fully grasp the intermediate interaction effect we extended the small sub-unit model in a way that the i'th site is affected by k sites before it and k sites after it.
1. The density of site i is depended on the flow to the i'th site from i−k:i−l and the flow from the i'th site to i+l:i+k sites.
2. If k is larger than the number of sites before/after the I'th site, k=maximal possible k.

Attachment, Detachment equations are the same as in the basic model.

The movement between sites of the small sub-unit depends on the detachment rate from the i'th site and the attachment rate of the k'th site.

Flow from Cell i to Cell k:

Flow(i,k)=c2+(Detachment(i)*Attachment(k))

Flow_{F}—Flow forward to the first site (initiation)

Flow_{B}—Flow backward from the first site (initiation)

The Model Equations from an mRNA in the Length of n Sites:

Initiation: \dot{x}₁=Flow_{F}(1−x₁)+Attachment(1)(1−x₁)−Flow(1,2)x₁(1−x₂)−Flow_{B}x₁−Detachment(1)x₁+Σⱼ₌₂^{k+1}Flow(j,1)xⱼ(1−x₁)−Flow(1,j)x₁(1−xⱼ) a.

Elongation (k<i<n−k): b.

In this case we have k sites before the i'th site and k sites after the i'th site.

Therefore, we sum all contribution of all k sites (in both sides of site i) to calculate the density of site i.

\dot{x}ₗ=[Σⱼ₌ᵢ₋ₖⁱ⁻¹(Flow(j,i)xⱼ(1−xᵢ)−Flow(i,j)xᵢ(1−xⱼ))+Σₘ₌ᵢ₊₁^{i+k}(Flow(m,i)xₘ(1−xᵢ)−Flow(i,m)xᵢ(1−xₘ))]+Attachment(i)(1−xᵢ)−Detachment(i)xᵢ

Elongation (i<=k): c.

In this case we have less than k sites before the i'th site and k sites after the i'th site.

Therefore, we sum all contribution of all k sites after the i'th site all k′ sites before the i'th site (k′<k, the maximum number of possible sites before the i'th site) to calculate the density of site i.

\dot{x}ₗ=[Σⱼ₌₁ⁱ⁼¹(Flow(j,i)xⱼ(1−xᵢ)−Flow(i,j)xᵢ(1−xⱼ))+Σₘ₌ᵢ₊₁^{i+k}(Flow(m,i)xₘ(1−xᵢ)−Flow(i,m)xᵢ(1−xₘ))]+Attachment(i)(1−xᵢ)−Detachment(i)xᵢ

Elongation (i>=n−k): d.

In this case we have k sites before the i'th site and less than k sites after the i'th site.

Therefore, we sum all contribution of all k sites before the i'th site all k′ sites after the i'th site (k′<k, the maximum number of possible sites after the i'th site) to calculate the density of site i.

\dot{x}ₜ=[Σⱼ₌ᵢ₋ₖⁱ⁻¹(Flow(j,i)xⱼ(1−xᵢ)−Flow(i,j)xᵢ(1−xⱼ))+Σₘ₌ᵢ₊₁ⁿ(Flow(m,i)xₘ(1−xᵢ)−Flow(i,m)xᵢ(1−xₘ))]+Attachment(i)(1−xᵢ)−Detachment(i)xᵢ

Termination: \dot{x}ₙ=Flow(n+1,n)(1−xₙ)+Attachment(n)(1−xₙ)−Flow(n,n+1)xₙ−Detachment(n)xₙ+Σⱼ₌ₙ₋ₖⁿ⁻¹Flow(j,n)xⱼ(1−xₙ)−Flow(n,j)xₙ(1−xⱼ) e.

The model of ribosomal movement during elongation. To initiate the movement of the ribosome we calculate the initiation rate considering the density from the small sub-unit model in the SD location in the 5′ UTR.

The movement of the ribosome depends on the rRNA-mRNA interaction of the relevant site and the effect of other features such as adaptation to the tRNA pool (denoted as typical decoding rate, TDR) on the elongation at the site codon.

initiation rate=mean(density(34:43)) 1.

$$Time ( i ) = \frac{1}{lambda ( i )} = ( \frac{{max}_{TDR}}{TDR ( i )} ) + exp ( \frac{mean ( interaction value ( i - 12 : i - 8 ) )}{max interaction value} )$$

Flow Model Results.

Parameters and model validation. To demonstrate our model, we created an artificial gene with 100 codons that all of its sites are weak sites (rRNA-mRNA interaction=0). From this basic variant we generated 5 additional variants via introducing in nucleotide 33 a gradient of different rRNA-mRNA interaction strength.

We simulated our complete model (the pre-initiation stage with k=20 and the elongation model) for all the variants. As can be seen the signal is convex: Initially stronger interactions improve the translation rate but when the interaction strength is stronger than a certain threshold (−2.7<=intermediate<=−1.8) there is a decrease in the translation rate.

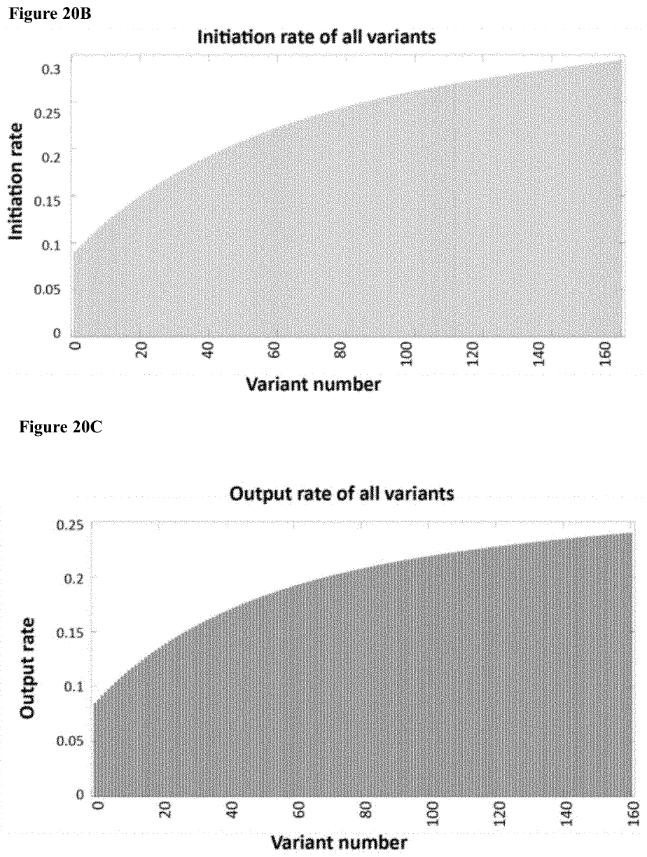

As can be seen (FIG. 20A), this is due to the fact that increasing the interaction strength the elongation rate decreases but the initiation rate increases.

TABLE 2

| | | Interaction = | Interaction = | Interaction = | Interaction = | Interaction = |
|---|---|---|---|---|---|---|
| K = 20 | Original | −1.8 | −2.7 | −3.7 | −5.3 | −8 |
| Init rate | 0.0992 | 0.1028 | 0.1028 | 0.1028 | 0.1028 | 0.1028 |
| Translation | 0.0930 | 0.0963 | 0.0963 | 0.0962 | 0.0962 | 0.0962 |
| rate | | | | | | |
| Elongation | 1.6 | 1.5590 | 1.5391 | 1.5176 | 1.4840 | 1.4302 |
| rate | | | | | | |

Adding intermediate interaction along the transcript improve the translation process. To show that adding many intermediate interactions along the transcript (as we see in endogenous genes) improve the translation rate we performed the following simulation: we started with a variant with one intermediate interaction close to the beginning of the coding sequence (3 nt after the start codon);_we gradually added intermediate downstream of start codon to improve the translation rate. Specifically, to make sure that even for long genes the intermediate effect exist we simulated a longer sequence with 500 nucleotides, and each added intermediate sequence was downstream of the previous one in a position that improve the translation.

The simulation result appear in FIGS. 20B and 20C and describe the increase in the initiation rate and translation rate for a set: each variant (index in the x-axis) is related to adding an additional intermediate interaction to the previous variant—larger index of the variant, is related to more intermediate interactions in the coding region. As we can see in FIGS. 20B and 20C, when adding intermediate interaction even at the end of the coding region we improve the initiation rate and due to that the translation rate. We can deduce that adding intermediate interaction along the transcript can indeed enhances the small sub-unit diffusion and the translation rate is increased.

Selection Against Strong Interaction at the End of the Coding Region—Read-Through Experiment.

Plasmids construction. We used plasmid pRX80 and modified it by deleting the lac I repressor gene and the CAT selectable marker. The resulting plasmid contained the RFP and GFP genes in tandem, both are expressed from a promoter with two consecutive lac operator domains. The plasmid contains also the pBR322 origin of replication and the Kanamycin resistance gene as a selectable marker. Because the 2 Operator sequences caused instability at the promoter region, we replaced the promoter region with a lacUV promoter with only one operator sequence. The resulting plasmid, pRCK28 was now used for the generation of variants which differ in the 40 last nucleotides of the RFP ORF. The variants include synonymous changes composed of both ribosome binding site at 3 energy ranges and which also alter the local folding energy (LFE) of the 40 last nucleotides of the RFP ORF end. The variable sequences where synthesized as G-blocks and Gibson assembly was used to replace the relevant region of the pRCK28 plasmid, generating 9 variants as described in FIG. 19B. The resulting variable plasmids were transformed into competent E. coli DH5α cells. Colonies were selected on LB Kanamycin plates. A few candidates were PCRed and sequenced to verify the synonymous changes in each variant.

Fluorescent Tests. Single colonies of each variant as well as of the original pRCK28 clone and of a negative control (an E. coli clone harboring a Kanamycin resistant plasmid at the same size of pRC28 but without any fluorescent gene) were grown overnight in LB-Kanamycin. Cells were then diluted and 10,000 cells were inoculated into 110 ul defined medium (1×M9 salts, 1 mM thiamine hydrochloride, 2% glucose, 0.2% casamino acids, 2 mM MgSO4, 0.1 mM CaCl2) in 96 well plates. For each variant 2 biological repeats and 4 technical repeats of each were used. A fluorimeter (Spark-Tecan) was used to run growth and fluorescence kinetics. For growth, OD at 600 nm data were collected. For red fluorescence, excitation at 555 nm and emission at 584 nm were used. For green fluorescence, excitation at 485 nm and emission at 535 nm were used. Data was analyzed and normalized by subtracting the auto fluorescence values of the negative control, and by calculating the fluorescence to growth intensity ratios.

Western blot analyses. Cells were grown overnight, 1 ml cultures were concentrated by centrifugation and lysed using the BioGold lysis buffer supplemented with lysozyme. Total protein lysates were resolved on Tris glycin 4-15% acrylamide mini protein TGX stain free gels (BioRad). Proteins were transferred to nitrocellulose membranes using the trans-blot Turbo apparatus and transfer pack. Membranes were incubated in blocking buffer (TBS+1% casein) for 1 hr at room temperature. Anti GFP and/or anti RFP antibodies (Biolegend) were used at 1:5K, for 1 hr in blocking buffer, at room temperature to probe the GFP and RFP expression. Goat anti-mouse 2nd antibody was then applied at 1:10K dilution. ECL was used to generate a binding signal.

### Results:

To understand the interactions between the 16S rRNA and mRNAs across the bacterial kingdom, a high-resolution computational model to predict the strength of rRNA-mRNA interactions was developed, where low hybridization free energy indicates a stronger interaction (See Methods). This model was used to analyze the entire transcriptome of 823 bacterial species, investigating all possible positions across all transcripts (i.e. 2,896,245 transcripts). To detect patterns of evolutionary selection, the distribution of rRNA-mRNA interaction strength was compared in each position along the transcriptome of each genome to the one expected by a null model. The null model preserves the codon frequencies, amino acid content, and GC content in each transcript (see Methods).

For each position along the transcriptome three statistical tests are performed to answer the following questions:
1) Does the nucleotide (nt) sequences in that position tend to produce stronger rRNA-mRNA interactions than expected by the null model?
2) Does the nt sequences in that position tend to produce weaker rRNA-mRNA interactions than expected by the null model?
3) Does the nt sequences in that position tend to produce intermediate (moderate strength: neither very strong nor very weak) rRNA-mRNA interactions in comparison to what is expected by a null model? (see FIG. 1A and Methods).

Herein there is reported the observed tendencies of sub-sequences within different transcript regions to produce strong, intermediate, and weak interactions with the 16S rRNA.

### Example 1: Selection for Strong rRNA-mRNA Interactions at the 5′UTR End and at the Beginning of the Coding Region to Regulate Translation Initiation and Early Translation Elongation

First, we analyzed the 5′UTRs of 551 bacteria with aSD (anti Shine Delgarno) sequence in the rRNA. It was suggested that translation initiation in prokaryotes is initiated by hybridization of the 16S rRNA to the mRNA. The 16S rRNA binds to the 5′UTR near and upstream of the START codon4 as depicted in FIG. 1C. Indeed, as can be seen in FIG. 1B (black box) in almost all of the analyzed bacteria, there is a significant signal of selection for strong rRNA-mRNA interactions at positions −8 through −17 relative to the START codon, in agreement with the Shine-Dalgarno model.

A second signal of selection for strong rRNA-mRNA interactions appears in the last nucleotide of the 5′UTR and the first five nucleotides of the coding sequence (FIG. 1B, blue box). Since the elongating ribosome is positioned around 11 nucleotides downstream of the position its rRNA interacts with the mRNA, it is likely that these rRNA-mRNA interactions are related to slowing down the early elongation phase of the ribosome.

It has been suggested that at the beginning of the coding region there are various features that slow down the early stages of translation elongation to improve organism fitness, e.g. via optimizing ribosomal allocation and chaperon recruitment (FIG. 1D). It is likely that this second novel signal is a mechanism of such regulation. Both of the reported signals above occur in 89% of the analyzed bacteria.

A comparison of highly and lowly expressed genes in E. coli (FIG. 1E) reveals that both signals are stronger in the highly expressed genes, which are under stronger selection to optimize translation. The difference between the Z-scores of highly and lowly expressed genes in the two reported signal regions was highly significant (nucleotides −8 through −17 in the 5′UTR: Wilcoxon rank-sum test p=7.9·10-5; last nucleotide of the 5′UTR and the first 5 nucleotides of the coding sequence: Wilcoxon rank-sum test p=9.3·10-4).

### Example 2: Selection Against Strong rRNA-mRNA Interactions in the Coding Regions that Prevents the Slowing Down of Translation Elongation

Ribo-seq analyses in E. coli have indicated that strong interactions between the 16S rRNA and the mRNA can lead to pauses during translation elongation, hindering translation (FIG. 2D). Avoiding such strong rRNA-mRNA interactions in the coding region should thus allow the ribosome to flow efficiently during translation elongation. The deleterious effects of such strong rRNA-mRNA interaction sequences may also be due to their role in encouraging internal translation initiation which would create truncated and frame-shifted protein products. The observation that the occurrence of AUG start codons is significantly depleted downstream of existing strong rRNA-mRNA interaction sequences in E. coli supports this claim.

Our analysis reveals evidence of significant selection against strong rRNA-mRNA interactions in the coding region (FIG. 2A). In 55% of the bacteria analyzed, at least 50% of the positions in the first 400 nucleotides of the coding region exhibit a signal of significant selection against strong rRNA-mRNA interactions. Importantly, this selection was also observed away from positions that are upstream of a nearby AUG, suggesting that such selection is also related to elongation, and not just to avoiding internal translation initiation. It has been suggested that the deleterious effects of strong rRNA-mRNA interaction sequences may be due to their role in encouraging internal translation initiation which would create truncated and frame-shifted protein products. Similarly, it has been observed that the occurrence of ATG start codons is significantly depleted downstream of existing strong rRNA-mRNA interaction sequences in E. coli. This result overlaps with our signal of selection against strong interaction in the coding region. But in our case, we also emphasize a different mechanism: preventing extreme slowing down of the ribosomes during elongation to enable a smooth (and efficient) as possible translation elongation process. In FIG. 17 we show that there is significant selection against strong rRNA-mRNA interaction even if there is no ATG downstream of it, suggesting that this signal may be also related to translation elongation.

We found evidence for selection against strong rRNA-mRNA interactions in the coding region throughout the bacteria phyla analyzed, except for in cyanobacteria and gram-positive bacteria which seem to exhibit selection for strong rRNA-mRNA interactions (FIG. 2A). It has been hypothesized that interactions between rRNA and mRNA are weaker in cyanobacteria as 16S ribosomal RNA is folded in such a way that subsequences that usually interact with the mRNA are situated within the RNA structure. Thus, in these organisms, it is expected that rRNA-mRNA interactions are less probable, resulting in lower selection pressure to eliminate sub-sequences that can interact with the rRNA in the coding region. A similar trend can be seen in the 3′UTR of genes (FIG. 2C). We postulate that similar to cyanobacteria, gram positive bacteria also have rRNA structures that result in less efficient rRNA-mRNA interactions.

Again, a comparison between highly and lowly expressed genes in E. coli reveals that selection against nucleotide sequences leading to strong interactions in the coding region is stronger for highly expressed genes which are under stronger selective pressure for more accurate and efficient translation (Wilcoxon rank-sum test p=1.5·10⁻³⁰; FIG. 2B).

In addition, as can be seen in FIG. 2E: At the beginning of the coding region (5-25 nucleotides), there is significant increased selection against strong and intermediate rRNA-mRNA interactions (typical p-value 0.0097). The presence of sub-sequences that interact in a strong/intermediate manner near the beginning of the coding region is probably more deleterious as it might promote with higher probability initiation from erroneous positions (see illustration in FIG. 2F); indeed, similar signals related to eukaryotic and prokaryotic initiation were reported.

### Example 3: Selection for Strong rRNA-mRNA Interactions at the End of the Coding Sequence to Improve the Fidelity of Translation Termination

In 82% of the analyzed bacterial species, in 50% of the positions at the last 20 nucleotides of the coding region, there is selection for strong rRNA-mRNA interactions (FIG. 3A). This constitutes a mechanism for slowing ribosome movement when approaching the stop codon and serves to ensure efficient and accurate termination and prevent translation read-through (FIG. 3F). It could be that this selection may have the function of assisting initiation of overlapping or nearby downstream genes in operons; however, we observed this phenomenon universally across all genes and bacteria, including the last genes in an operon which are not closely followed by other genes. (FIG. 3F).

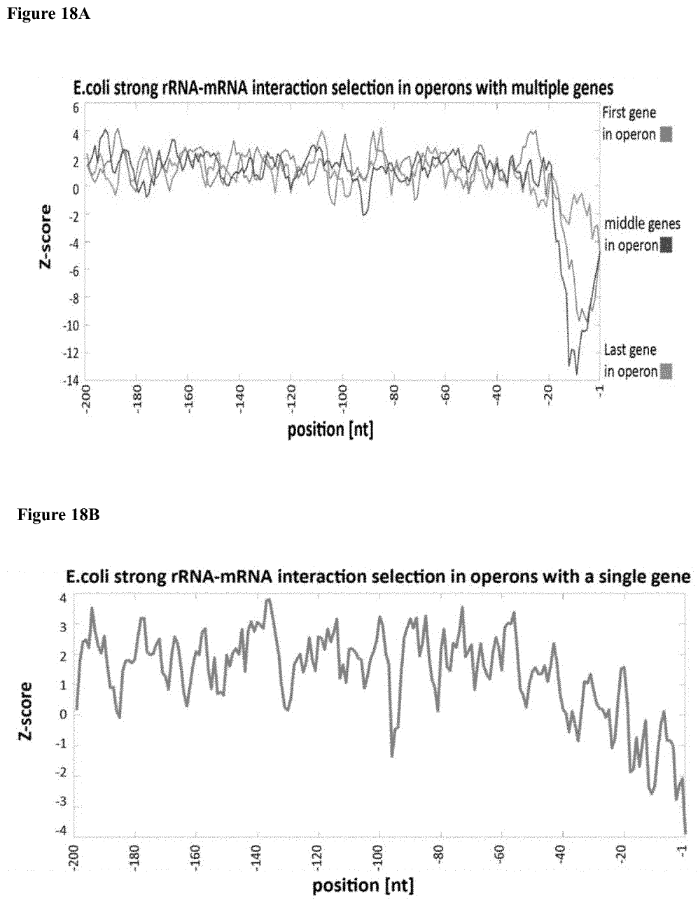

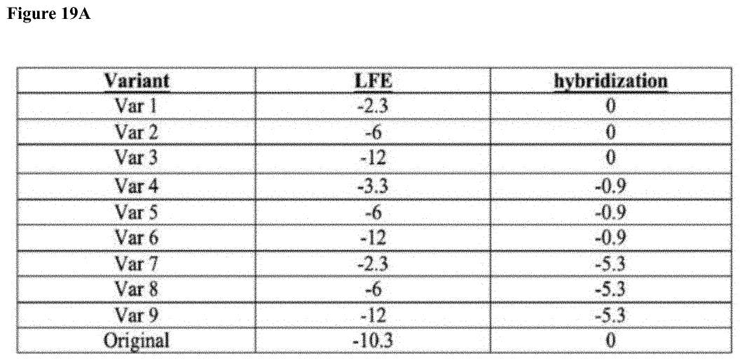

Many genes in bacteria are transcribed as operons. Specifically, in E. coli, 55% of the genes are grouped in operons. In operons, the downstream gene has a start codon near the stop codon of the upstream gene which can affect the selection for strong interaction at the end of the coding region. Therefore, we further validate this signal, by looking on operons and especially looking on genes at the begging/middle/ending of an operon. As can be seen in FIG. 18A, there is a strong selection for strong interactions at the end of the coding region in the first middle and last genes in operons. This result supports the hypothesis that this signal is related (at least partially) to termination. In FIG. 18B we can also see a selection for strong interactions at the end of the coding region in an operon with a single gene.

It has previously been found that when the rRNA binds to the mRNA the ribosome is generally decoding a codon located approximately 11 nt downstream of the binding site. To validate this, we inferred the positions with selection for the strongest interactions and identified those with minimum rRNA-mRNA interaction Z-scores within the last 20 nt of the coding region, in most of the analyzed bacteria (See Methods). We discovered that the strongest and most significant positions across all bacteria are indeed −9 through −12 relative to the STOP codon (FIGS. 3B and 3C). This supports our hypothesis that the interactions indeed function to halt the ribosome on the STOP codon and not to initiate the next open reading frame in the operon.

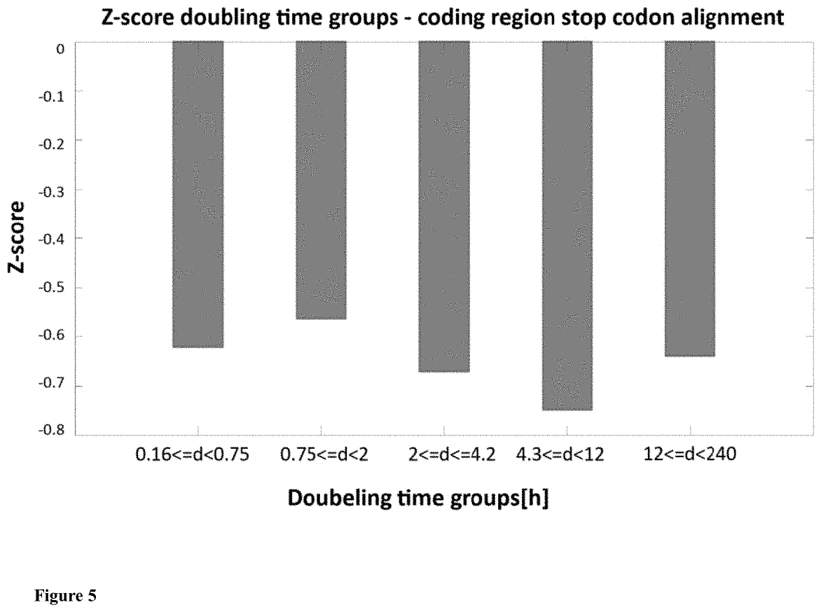

We examined the relationship between the strength of selection for strong interaction in the last 20 nt of coding regions with different levels of gene expression and found it to be convex: such selection is stronger for genes with intermediate expression and weaker for both lowly- and highly-expressed genes (FIG. 3D). We consider that the weaker selection in lowly-expressed genes may be due to lower selection pressure on the gene in general. Conversely, the weaker signal in highly-expressed genes may be due to stronger selection on translation elongation and termination rates: the ribosome density in these genes is higher, and if a ribosome is stalled in order to promote accurate termination it may cause ribosome queuing at the 3′-end, resulting in inefficient ribosomal allocation. Highly expressed genes may have other mechanisms for ensuring termination fidelity. The relation between the signals of selection for strong rRNA-mRNA interactions at the end of the coding region and doubling time in bacteria with known growth rates was also investigated. As can be seen in FIG. 5, the signal is stronger in bacteria with intermediate doubling time. This result is analogous to the relationship between signal strength and gene expression.

To test if strong rRNA-mRNA interactions just prior to the stop codon improve termination fidelity, we analyzed Ribo-seq data of E. coli (FIG. 3E and Methods). We expected that if such an interaction improves the fidelity of termination, mRNAs with a strong interaction will exhibit less read-through events and thus we will observe less Ribo-seq read counts (RC) downstream of the STOP codon. Indeed, we found that the average read count for the 20 nucleotides after the stop codon was lower following genes with strong rRNA-mRNA interactions in the last 20 nucleotides of the coding region, compared to genes with weaker interactions in this region (mean RC=0.334 and 0.514, respectively; Wilcoxon rank-sum test p=0.001).

To further experimentally test our hypothesis of strong rRNA-mRNA interactions just prior to the stop codon preventing stop-codon read-through, we used a construct mRNAs with a gene coding for red fluorescent protein (RFP) linked to a gene coding for green fluorescent protein (GFP; FIG. 3G). We positioned the GFP gene downstream such that its expression acts as an indicator of read-through expression, and variants with higher GFP fluorescence are indicative of higher rates of stop-codon read-through (See Methods). We designed nine variants with different rRNA-mRNA interaction strengths and local mRNA folding at the last 40 nt27 of the RFP, and measured their florescence. As hypothesized, we found that variants with stronger rRNA-mRNA interactions at the end of the RFP coding region tend to produce lower levels of GFP (FIG. 3H). We found that there is high correlation between the relative read-trough signal (the ration between the GFP florescence and the RFP florescence) and the predicted rRNA-mRNA interactions strength prior to the stop codon even when controlling for the local mRNA folding near the stop codon (partial Spearman correlation: r=0.7996 P=0.0097).

### Example 4: Selection for Intermediate rRNA-mRNA Interactions in the Coding Region and UTRs to Improve the Pre-Initiation Diffusion of the Small Subunit to the Initiation Site

The previous sections presented evidence for selection against strong interactions between the rRNA and mRNA throughout most of the coding region, but this doesn't mean that all interactions throughout this region are deleterious: other forces may act in differing directions. Prior to binding with mRNA, free ribosomal units travel by diffusion. Some interaction with the mRNA may assist to ‘guide’ the diffusing small subunit of the ribosome to remain near the transcript and ‘help’ them find the start codon, increasing their diffusion efficiency and consequently overall translation initiation efficiency (FIG. 4F, section 1).

Initiation is often the rate limiting stage of translation and the most limiting aspects probably appear to be the 3-dimensional diffusion of the small sub-unit to the SD region. One-dimensional diffusion (i.e. along the mRNA) may be faster: if mRNAs can ‘catch’ small ribosomal sub-units and then direct them to their start codons, they may be favored by evolution. The large amount of redundancy in the genetic code allows for mutations that may improve interactions between the rRNA and mRNA even in the coding region, without negatively affecting protein products; however as we have seen, strong interactions in the coding region are problematic. Based on these considerations; we hypothesized that evolution shapes coding regions to include intermediate rRNA-mRNA interactions, which are not strong enough to halt elongation, but can optimize pre-initiation diffusion.

To test this hypothesis, we created an unsupervised optimization model to identify sequences with intermediate rRNA-mRNA interactions by adaptively calculating rRNA-mRNA interaction-strength thresholds for each bacterium. The algorithm selects rRNA-mRNA interaction strength thresholds such that they delineate the maximum number of significant positions with rRNA-mRNA interactions between these thresholds (see Methods).

To verify that the thresholds are reasonable, we looked at the highest (per gene) rRNA-mRNA interaction strength distribution in the 5′UTR in two regions: 1) The canonical rRNA-mRNA interaction region during initiation (i.e. nucleotides −8 through −17 upstream to the start codon). 2) The region in the 5′UTR which is upstream to 1). We then defined each gene by two values: a. Minimum interaction strength (i.e. strongest interaction) from region 1) distribution. b. Minimum interaction strength from region 2) distribution. For each bacterium, we created distribution plots based on values a. and b. over its genes. FIG. 4A includes these two distributions for E. coli; as can be seen, the rRNA-mRNA intermediate interaction strength thresholds for this bacterium are in the overlapping region of the two distributions. Furthermore, we calculated the area between the optimized intermediate thresholds under the distribution of all values of rRNA-mRNA interaction strength in the aforementioned regions (1) and (2) (FIG. 4D). As expected, the area under distribution 1) is greater than the area under distribution 2) in most of the bacteria (the ratio is larger than 1 in 91 percent % of the bacteria). This provides confirmation that the range of interaction strengths identified corresponds to intermediate interactions and not to a lack of interaction.

Our analyses revealed that in 52% of the analyzed bacteria at least 50% of the positions are under significant selection for intermediate rRNA-mRNA interactions: according to the null model this would be expected to be the case for only 0.18% (FIG. 4B). A similar trend can be seen in the 3′UTR (FIG. 4C). The level of selection for intermediate interactions in the coding region varies among the bacterial Phylum and thus may be affected by various phylum-specific characteristics as growth rate, competition, and many aspects of translation regulation.

When looking on the intermediate selection signal, we can see that the signal can be observed in 52% of the analyzed bacteria, The groups of bacteria that exhibits that signal are: 47% of the Betaprotobacteria, 49% of the Cyano bacteria, 94% of the Delta bacteria, 43% of the Gamma bacteria, 83% of the Gram positive bacteria, 28% of the Purple bacteria, 100% of the Spirochete bacteria, and 26% of the Alpha bacteria and E. coli.

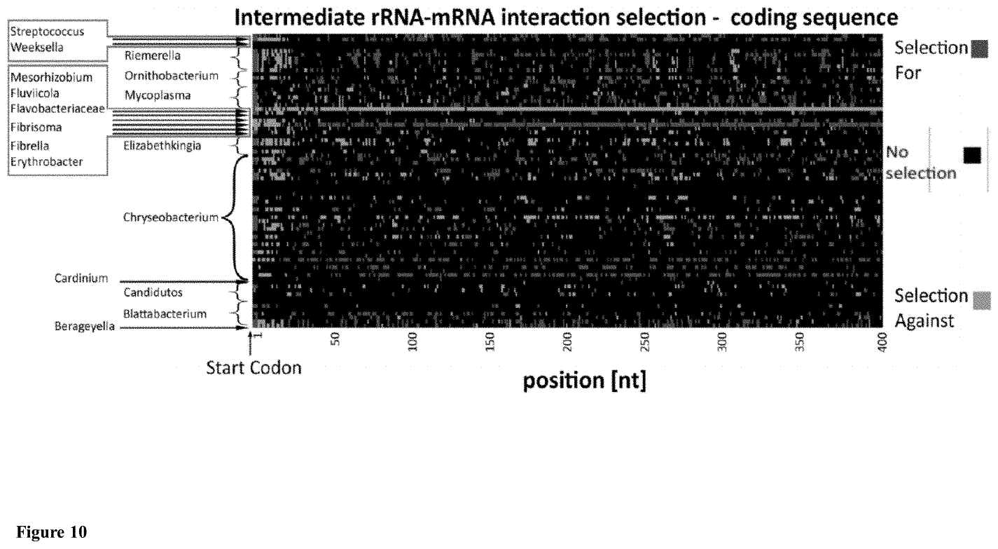

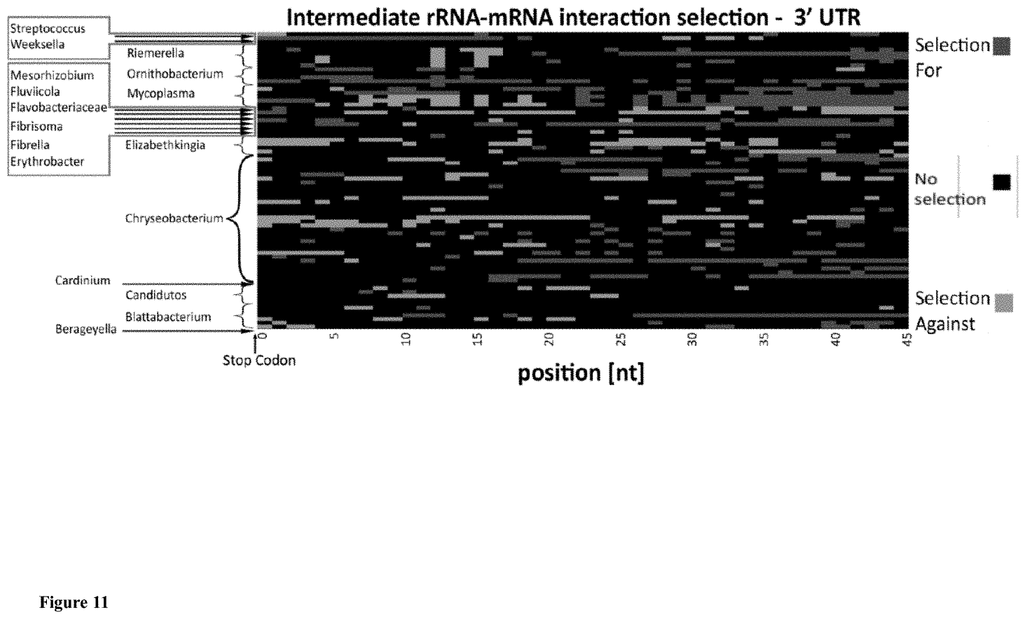

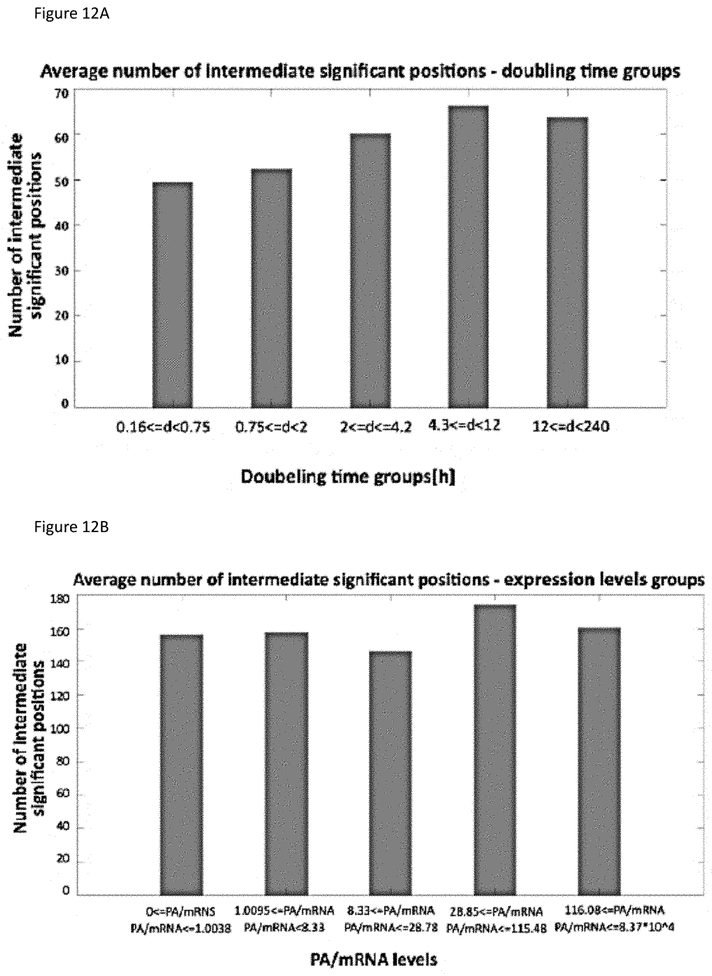

Selection for intermediate interactions in the coding region and 3′UTR can be seen in FIGS. 10 and 11 for bacteria with non-canonical aSD. Indeed, there is a trend of selection for such interactions in the coding region and 3′UTR, however, the signal is much weaker and not as consistent as in bacteria with canonical aSD.

Our null model preserves the protein itself, the codon bias and the GC content. Therefore, the observed selection cannot be favoring specific codons or amino acids. In addition, our rRNA-mRNA interaction profiles consider all three reading frames; hence, the amino acids are not the key factor that influences this signal. Furthermore, the fact that we see a similar pattern of selection in the UTRs (FIG. 4C) suggests that this pattern cannot be attributed only to selection for certain codon pairs.

We hypothesize that selection for intermediate rRNA-mRNA interactions in the coding region of a gene should improve its translation initiation efficiency and thus its protein levels. To demonstrate this, we calculated the partial Spearman correlations between the number of intermediate interaction sequences in the GFP variant (see previous Example) and the heterologous protein abundance (PA), based on 146 synonymous GFP variants that were expressed from the same promoter. The control variables were the codon adaptation index (CAI); a measure of codon usage bias, and mRNA folding energy (FE) near the start codon, known to affect translation initiation efficiency (the weaker the folding in the vicinity of the start codon the higher the fidelity and efficiency of translation initiation).

We defined an area of intermediate interactions according to the thresholds determined by our model in E. coli and calculated the correlation explained above. As expected, the correlation was positive and significant (r=0.35; P=0.2·10-4) indicating that variants with more sub-sequences in the coding region that bind to the rRNA with an intermediate interaction strength tend to have higher PA.

We found that this correlation is specifically very high (r=0.61; p=0.003) when the FE near the start codon is the strongest (FIG. 4E). The intermediate sequences are expected to have a stronger effect on initiation when this process is less efficient (i.e. when it is more rate limiting). Thus, according to our model we expect to see stronger correlation between protein levels and the number of intermediate sequences when the mRNA folding in the region surrounding the START codon is strong (FIG. 4F, section 2).

When calculating the partial Spearman correlation between the number of sub-sequences that interact in a weak manner with the rRNA and the PA of the GFP variants, the correlation is negative and significant (r=−0.32; p=8.5·10-5). This further validates our conjecture that translation efficiency in this case is indeed related to interactions that are neither very strong, nor very weak or absent. It also suggests that this effect on translation efficiency is related to the pre-initiation step and not the elongation step, otherwise we would expect positive correlation with weak interaction.

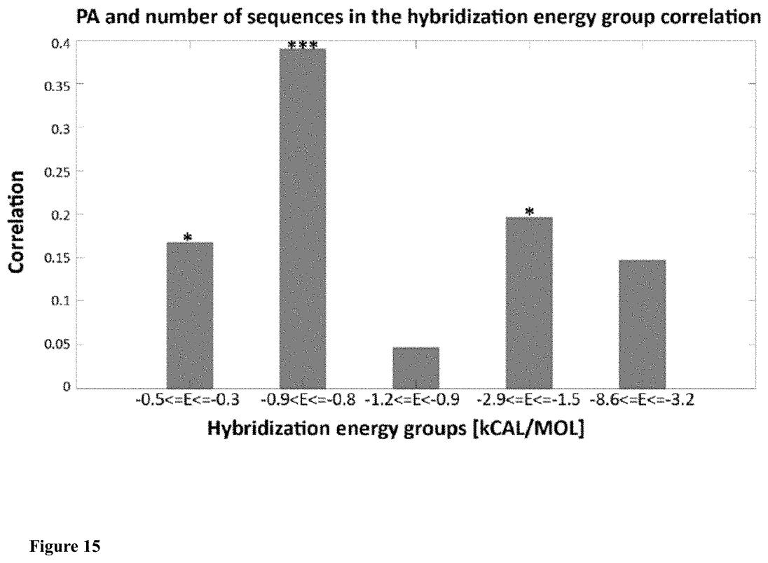

To validate the GFP correlation of intermediate interactions in an ‘unsupervised’ manner, we calculated the hybridization energy of all Ent sequences in the GFP variant and divided the sequences hybridization energy into five groups. Afterwards, we calculated the Spearman correlation between the number of sequences in a specific group of hybridization energy value and PA of the GFP variants. As can be seen in FIG. 15, the intermediate hybridization values (not the lowest or the highest ones) have the highest positive and significant correlation with protein levels.

We also analyzed E. coli genes by their mRNA half-life to assess how selection for intermediate interactions varies among them. We found that genes with shorter half-life tend to have more intermediate interaction. It is possible that these genes undergo stronger selection to include intermediate interactions since their corresponding mRNAs ‘have less time’ to initiate translation. Thus, the reported results discussed here suggest that the diffusion of the small ribosomal sub-unit is relatively fast.

To enhance our knowledge of the effect of intermediate interactions, we divided E. coli genes according to their mRNA half-life. For the top and bottom 20% we calculated the percentage of genes that have intermediate interaction in each position in the coding region. From this analysis we discovered that genes with shorter mRNA half-life tend to have more intermediate interactions (Wilcoxon test P=2.060·10⁻⁶). This result may be related to the fact that those mRNAs have ‘less time’ as genes to ‘catch’ ribosomes before they are degraded. Moreover, mRNA molecules of various genes tend to localized in certain regions in the cell; this may suggest that ‘catching’ ribosomes by one of the mRNA may improve their diffusion time to other close mRNAs once this specific mRNA has undergone degradation.

It is known that mRNAs tend to localize in certain regions in the cell, meaning that if we can keep the ribosome close to a certain mRNA we also keep it close to other mRNA's. If a certain mRNA ‘captures’ a ribosome then undergoes degradation this ribosome will likely remain close to other nearby mRNAs. It is also possible that due to compartmentalization and aggregation of many mRNA molecules the interaction with the small sub-unit of one mRNA can be ‘helpful’ for a nearby mRNA.

We further investigated the relation between the signals of selection for intermediate rRNA-mRNA interactions and doubling time. We divided the bacteria according to their doubling time and calculated the average number of intermediate significant positions in the coding region (FIG. 12A). The signal also seems to be convex (and analogous to the relation of the signal strength and gene expression FIG. 12B.): Organisms with very high growth rates have lower signals since it might decrease elongation rates; organisms with low growth rates have lower signals due to lower selection pressure. This result again demonstrates the complex convex relation between the selection pressure on intermediate rRNA-mRNA interactions inside the coding regions and growth rate and gene expression. Indeed, similar trends can be seen in E. coli, when dividing the genes according to their translation efficiency (PA/mRNA levels, FIG. 12B).

Finally, we created a computational biophysical model that describes the movement of the small ribosomal sub-unit along the transcript. In this model the movement is influenced by the intermediate interactions (FIGS. 4G and 4H). The model indicates that adding intermediate interaction along the transcript improves the initiation rate and termination rate even if the intermediate sequence is near the 3′ end of the gene. It also demonstrates the advantage of intermediate interactions over weak or strong ones in most of the transcript as intermediate interactions in the transcript optimize the translation rate. We conclude that intermediate rRNA-mRNA interactions along the transcript enhance small ribosomal sub-unit diffusion to the start codon with resultant improvements in the translation rate (see Methods).

### Example 5: Selection for Strong/Weak/Intermediate Interactions in Different Parts of the Transcripts in Bacteria with No Canonical aSD

To verify and further investigate the reported signals, we analyzed bacteria that do not have the canonical aSD in their 16S rRNA. As expected, while analyzing such bacteria, most of our reported signals could not be found. The results of this sub-section reinforce our model, and conjecture of the importance of rRNA-mRNA interactions in all stages and sub-stages of translation.

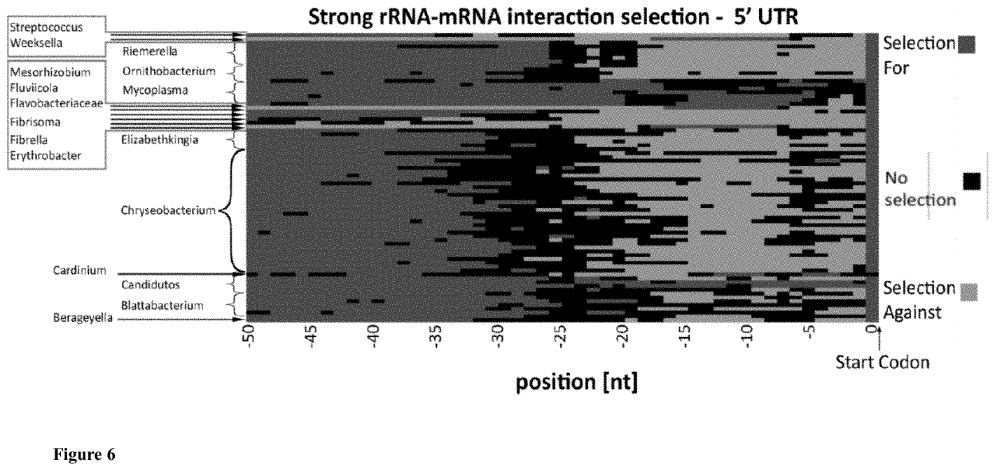

We looked at selection for strong interactions at the 5′UTR. Due to the fact that the bacteria do not have the canonical aSD sequence in their 16S rRNA, there was no clear evidence of selection for strong rRNA-mRNA interactions in positions −8 through −17 in the 5′UTR (FIG. 6). On the other hand, it can be seen in FIG. 6, selection for strong rRNA-mRNA interaction at the last nucleotide of the 5′UTR, which can slow down the movement of the ribosome during the early stages of translation elongation—a known signal in many organisms. When comparing the selection strength in the last nucleotide of the 5′UTR in the non-canonical bacteria and the 551 bacteria (the canonical), the selection is weaker in the non-canonical bacteria (regular bacteria: mean Z-score=−10.05, non-canonical bacteria mean Z-score=−7.69).

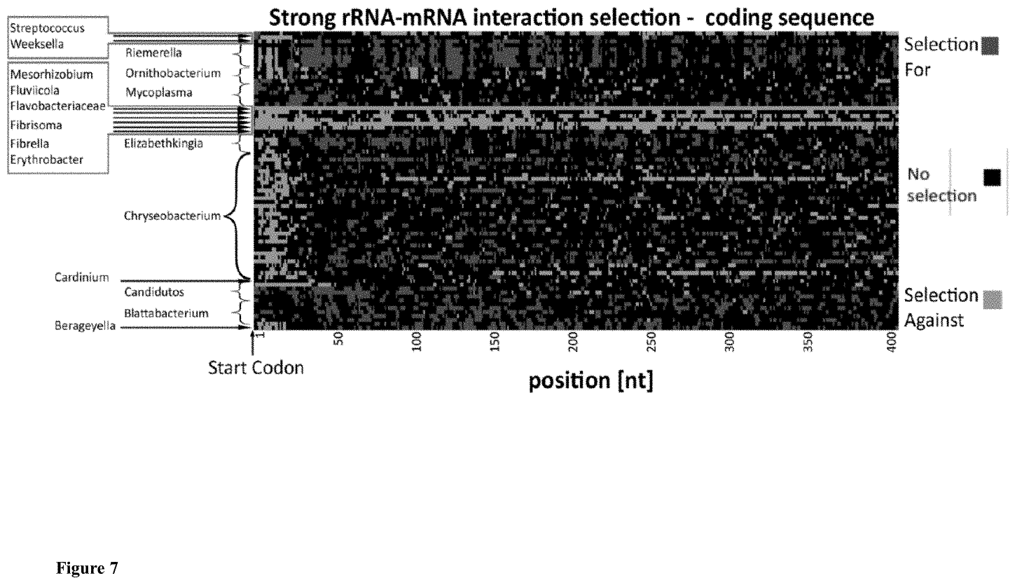

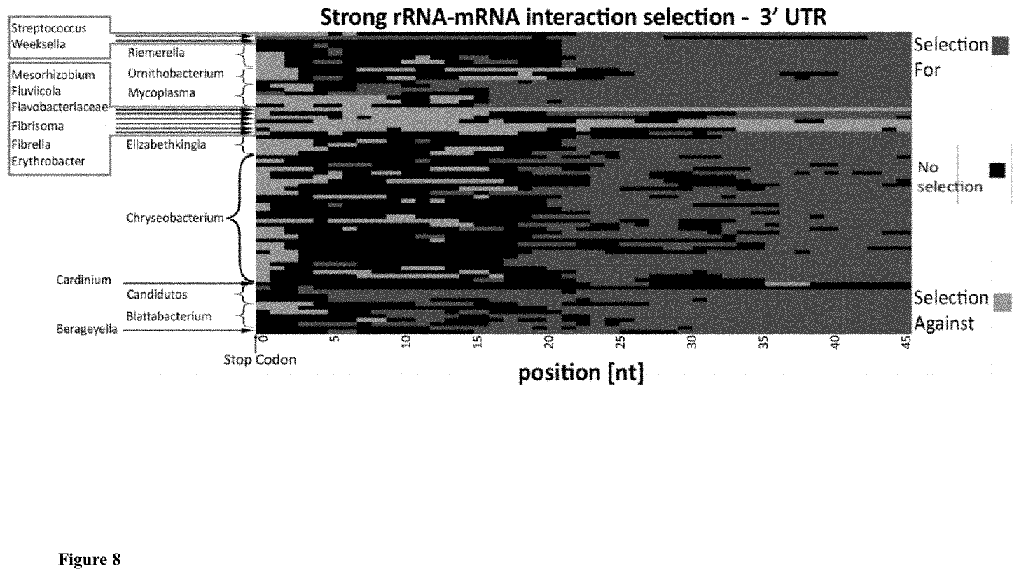

As can be seen in FIGS. 7 and 8, there is mostly selection for strong rRNA-mRNA interactions. In addition, when the signal is in the right direction, it is much weaker than in (‘regular’) organisms with the canonical aSD: The mean number of significant positions in which there is selection against strong interactions in ‘regular’ bacteria is 96.47 compared to 37.67 in the non-canonical bacteria).

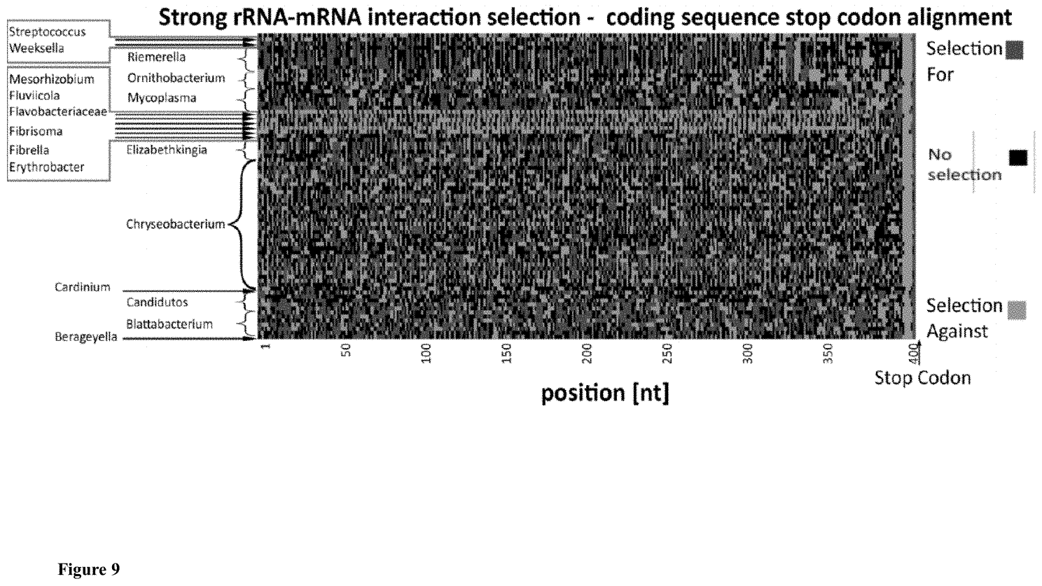

In bacteria with canonical aSD, at the end of the coding region, we detected a signal of selection for strong rRNA-mRNA interactions that enables stop codon recognition and prevents read-through. When we look at the bacteria with no canonical aSD (FIG. 9), we detected an opposite signal (i.e. selection for weak interaction) in all the positions, while a signal related to strong interaction (i.e. in the right direction) appears only in the last two nucleotides of the coding region (FIGS. 19A-C). The short signal at the last two nucleotides is probably not related to optimizing termination since we expect such a signal to appear approximately 11 nucleotides upstream of the stop codon (as reported in the main text), which is not the case here.

### Example 6: SD Sequence Optimization Model

The common assumption is that the SD and aSD sequences are usually the canonical ones. However, we believe that there may be organisms with different rRNA-mRNA interaction motifs. Thus, we developed an optimization model that finds the optimized SD and aSD sequences for a given bacterium in an unsupervised manner.

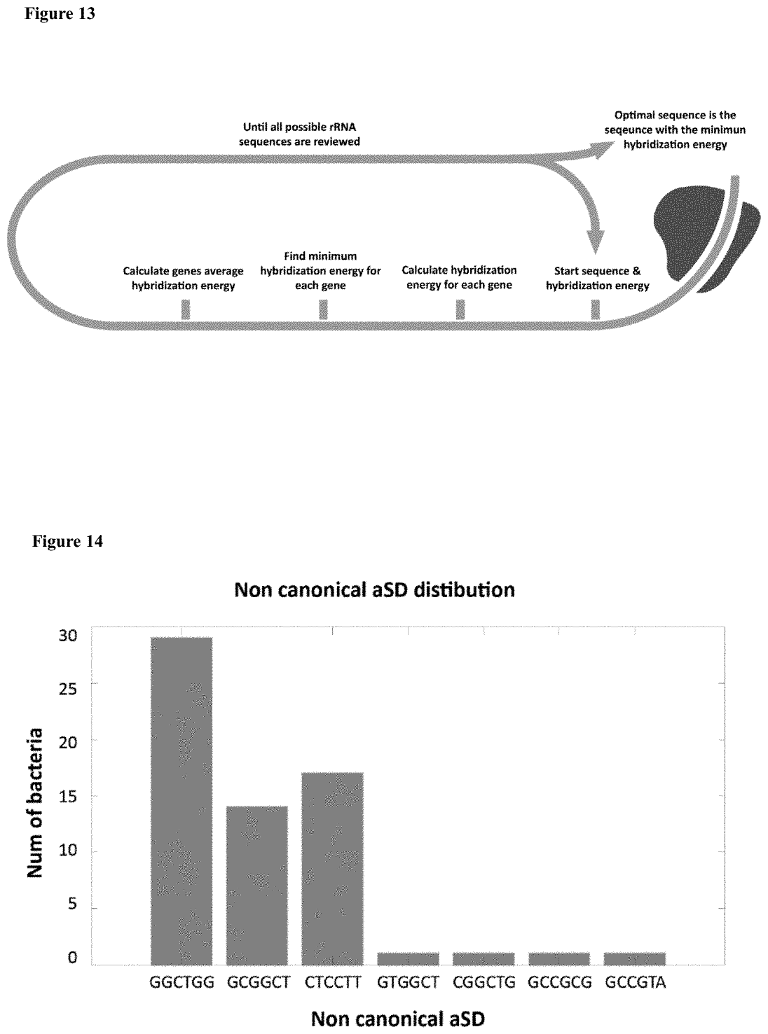

To find the optimal SD we devised the following algorithm (FIG. 13): For a certain organism, we considered all the Ent long sub-sequences at the last 20 nt of the 3′end of the 16S rRNA as a potential alternative “aSD”.

For each such potential alternative “aSD”, and for each gene in the organism, we considered all the sub-sequences in position −8 through −17 in the 5′UTR, to find the sub-sequence with the strongest rRNA-mRNA interaction, with the potential to be an alternative “aSD”. These values were averaged across the genes, and the potential alternative “aSD” that yields the lowest average (related to strongest predicted averaged rRNA-mRNA interaction strength) is predicted to be an alternative “aSD” sequence.

We executed the optimization model on 551 bacteria. As can be seen in FIG. 14, in only 64 out of the 551 bacteria, the optimal aSD wasn't the canonical aSD. Furthermore, there are three ‘alternative aSD sequences’ that are inferred to be optimal in most of those 64 bacteria (see the first three bars in FIG. 14). The reported results remain the same when we used the new aSD-SD model on these bacteria instead of the canonical aSD-SD interaction assumption.

### Example 7: Intermediate Sequences Validation in the GFP Variants

To validate the GFP correlation of intermediate interactions in an ‘unsupervised’ manner, we calculated the hybridization energy of all 6 nt sequences in the GFP variant and divided the sequences hybridization energy into five groups. Afterwards, we calculated the Spearman correlation between the number of sequences in a specific group of hybridization energy value and PA of the GFP variants. As can be seen in FIG. 15, the intermediate hybridization values (not the lowest or the highest ones) have the highest positive and significant correlation with protein levels.

1. A nucleic acid molecule comprising a coding sequence, wherein said nucleic acid molecule comprises at least one mutation within a region of said molecule, wherein said mutation modulates interaction strength of said nucleic acid molecule to a 16S ribosomal RNA (rRNA); and wherein said region is selected from the group consisting of:
a. positions −8 through −17 upstream of a translational start site (TSS) of said coding sequence and said mutation increases interaction strength;
b. positions −1 upstream of a TSS through position 5 downstream of said TSS of said coding sequence and said mutation increases interaction strength;
c. positions 6 through 25 downstream of a TSS of said coding sequence and said mutation decreases interaction strength;
d. positions 26 downstream of a TSS of said coding sequence through position −13 upstream of a translational termination site (TTS) of said coding sequence and said mutation modulates interaction strength to an intermediate interaction strength;
e. positions −8 through −17 upstream of a TTS of said coding sequence and said mutation increases interaction strength; and
f. a position downstream of a TTS of said coding sequence and said mutation increases interaction strength.

2. The nucleic acid molecule of claim 1, wherein
a. said mutation modulates interaction strength of a six-nucleotide sequence containing said mutation to said 16S rRNA;
b. said interaction strength to a 16S rRNA is to an anti-Shine Dalgarno (aSD) sequence of said 16S rRNA; or
c. said interaction strength to a 16S rRNA is to an anti-Shine Dalgarno (aSD) sequence of said 16S rRNA and is determined from Table 3.

3. (canceled)

4. (canceled)

5. The nucleic acid molecule of claim 1, wherein said increasing increases interaction strength to a strong interaction strength, decreasing decreases interaction strength to a weak interaction strength and wherein strong, weak and intermediate interaction strengths are determined from Table 1.

6. The nucleic acid molecule of claim 1, wherein
a. said region from position 26 downstream of the TSS through position −13 upstream of the TTS comprises the first 400 base pairs of said region;
b. said molecule comprises at least a second mutation, wherein said second mutation is in a different region than said at least one mutation;
c. said at least one mutation is within said coding sequence and mutates a codon of said coding sequence to a synonymous codon;
d. wherein said mutation improves the translation potential of said coding sequence;
e. wherein said mutation does at least one of: increasing translation initiation efficiency, increasing translation initiation rate, increasing diffusion of the small subunit to the initiation site, increasing elongation rate, optimization of ribosomal allocation, increasing chaperon recruitment, increasing termination accuracy, decreasing translational read-through and increasing protein yield;
f. said nucleic acid molecule is a messenger RNA (mRNA); or
g. a combination thereof.

7. (canceled)

8. (canceled)

9. (canceled)

10. (canceled)

11. (canceled)

12. A cell comprising a nucleic acid molecule of claim 1.

13. The cell of claim 12, wherein
a. said cell is a bacterial cell;
b. said cell is a cell of a bacterium recited in Table 1;
c. said cell is a cell of a bacterium selected from Escherichia Coli, Alphprotebacteria, Spriochaete, Purple bacteris, Gammaproteoaceteria, deltaproteobacteria and Betaproteobacteria; or
d. wherein said cell is a bacterial cell and said bacterium is not a Cyanobacteria or Gram-positive bacteria.

14. (canceled)

15. (canceled)

16. (canceled)

17. (canceled)

18. (canceled)

19. A method for improving the translation potential of a coding sequence, the method comprising introducing at least one mutation into a nucleic acid molecule comprising said coding sequence, wherein said mutation modulates interaction strength of said nucleic acid molecule to a 16S rRNA, thereby improving the translation potential of a coding sequence.

20. The method of claim 19, wherein said improving comprises at least one of: increasing translation initiation efficiency, increasing translation initiation rate, increasing diffusion of the small subunit to the initiation site, increasing elongation rate, optimization of ribosomal allocation, increasing chaperon recruitment, increasing termination accuracy, decreasing translational read-through and increasing protein yield.

21. The method of claim 19, wherein said mutation is located at a region selected from the group consisting of:
a. positions −8 through −17 upstream of a translational start site (TSS) of said coding sequence and said mutation increases interaction strength;
b. positions −1 upstream of a TSS through position 5 downstream of said TSS of said coding sequence and said mutation increases interaction strength;
c. positions 6 through 25 downstream of a TSS of said coding sequence and said mutation decreases interaction strength;
d. positions 26 downstream of a TSS of said coding sequence through position −13 upstream of a translational termination site (TTS) of said coding sequence and said mutation modulates interaction strength to an intermediate interaction strength;
e. positions −8 through −17 upstream of a TTS of said coding sequence and said mutation increases interaction strength; and
f. a position downstream of a TTS of said coding sequence and said mutation increases interaction strength.

22. (canceled)

23. (canceled)

24. The method of claim 19, further comprising introducing at least a second mutation in a different region from said at least one mutation.

25. The method of claim 19, wherein introducing a mutation comprises:
a. profiling interaction strengths of each 6-nucleotide long subregion of said nucleic acid molecule to said 16S rRNA;
b. profiling an interaction strength of each 6-nucleotide long subregion comprising a potential mutation of said nucleic acid molecule; and
c. introducing to said nucleic acid molecule said mutation wherein the cumulative change in interaction strength of all of said 6-nucleotide long subregions comprising said mutation modulates an interaction strength to said 16S ribosomal RNA.

26. The method of claim 19, wherein said mutation modulates interaction strength of a six-nucleotide sequence containing said mutation to said 16S rRNA.

27. The method of claim 26, wherein said interaction strength to a 16S rRNA is to an anti-Shine Dalgarno (aSD) sequence of said 16S rRNA.

28. The method of claim 27, wherein said interaction strength of a sequence of said nucleic acid molecule to said aSD sequence is determined from Table 3.

29. The method of claim 19, wherein said increasing increases interaction strength to a strong interaction strength, decreasing decreases interaction strength to a weak interaction strength and wherein strong, weak and intermediate interaction strengths are determined from Table 1.

30. A method of modifying a cell, the method comprising expressing a nucleic acid molecule of claim 1, within said cell, thereby modifying a cell.

31. The cell of claim 30, wherein
a. said cell is a bacterial cell;
b. said cell is a cell of a bacterium recited in Table 1;
c. said cell is a cell of a bacterium selected from Escherichia Coli, Alphprotebacteria, Spriochaete, Purple bacteris, Gammaproteoaceteria, deltaproteobacteria and Betaproteobacteria; or
d. wherein said cell is a bacterial cell and said bacterium is not a Cyanobacteria or Gram-positive bacteria.

32. (canceled)

33. (canceled)

34. (canceled)

35. (canceled)

36. (canceled)

37. A computer program product for modulating translation potential of a coding sequence in a nucleic acid molecule, comprising a non-transitory computer-readable storage medium having program code embodied thereon, the program code executable by at least one hardware processor to:
a. receive a sequence of said nucleic acid molecule;
b. calculate interaction strength of a 6-nucleotide long subregion of said nucleic acid molecule to an aSD of a 16S rRNA of a target bacterium;
c. calculate the cumulative alteration to interaction strength between said subregion and said aSD caused by a mutation within said subregion; and
d. provide an output modified sequence of said nucleic acid molecule comprising at least a mutation that increases or decreases translation potential.

38. The computer program product of claim 37, wherein said calculating comprises calculating interaction strength of a plurality of 6-nucleotide long subregions with a region of said nucleic acid molecule, wherein said region is selected from:
a. positions −8 through −17 upstream of a translational start site (TSS);
b. positions −1 upstream of a TSS through position 5 downstream of said TSS;
c. positions 6 through 25 downstream of a TSS;
d. positions 25 downstream of a TSS through position −13 upstream of a translational termination site (TTS);
e. positions −8 through −17 upstream of a TTS; and
f. a position downstream of a TTS.

39. The computer program product of claim 38, wherein
a. said computer program product comprises calculating the interaction strength of each 6-nucleotide long subregion within said region;
b. said output modified sequence of said nucleic acid molecule comprises at least the top 5 mutations within said nucleic acid molecule that increase or decrease translation potential; or
c. said output modified sequence of said nucleic acid molecule comprises at least the top 5 mutations within said region that increase or decrease translation potential.

40. (canceled)

41. (canceled)
